(19) United States
(12) Patent Application Publication
(10) Pub. No.: US 2011/0171641 A1
(43) Pub. Date: Jul. 14, 2011

(54) Gene Expression Markers of Recurrence Risk in Cancer Patients After Chemotherapy

(76) Inventors: JOFFRE BAKER, MONTARA, CA (US); ROBERT GRAY, BOSTON, MA (US); STEVEN SHAK, HILLSBOROUGH, CA (US); JOSEPH SPARANO, PLEASANTVILLE, NY (US); CARL YOSHIZAWA, REDWOOD CITY, CA (US)

(21) Appl. No.: 12/900,388

(22) Filed: Oct. 7, 2010

Related U.S. Application Data
(62) Division of application No. 12/192,825, filed on Aug. 15, 2008, now abandoned.
(60) Provisional application No. 60/956,380, filed on Aug. 16, 2007.
(60) Provisional application No. 60/970,188, filed on Sep. 5, 2007.
(60) Provisional application No. 60/970,490, filed on Sep. 6, 2007.

(51) Int. Cl. C12Q 1/68

(52) U.S. Cl. 435/6.11; 435/6.13; 435/6.12

(57) ABSTRACT

The present invention relates to genes, the expression levels of which are correlated with likelihood of breast cancer recurrence in patients after tumor resection and chemotherapy.

### CROSS REFERENCE TO RELATED APPLICATIONS

This application is a non-provisional application filed under 37 CFR 1.53(b)(1), claiming priority under 35 USC 119(e) to provisional application No. 60/970,490, filed Sep. 6, 2007; provisional application No. 60/970,188, filed Sep. 5, 2007, and provisional application No. 60/956,380, filed Aug. 16, 2007, the contents of which are incorporated herein by reference.

### FIELD OF THE INVENTION

The present invention relates to genes, the expression levels of which are correlated with likelihood of breast cancer recurrence in patients after tumor resection and chemotherapy.

### BACKGROUND OF THE INVENTION

The prognosis for breast cancer patients varies with various clinical parameters including tumor expression of estrogen receptor and presence of tumor cells in draining lymph nodes. Although the prognosis for estrogen receptor positive (ER⁺), lymph node negative (N⁻) patients is generally good, many of these patients elect to have chemotherapy. Of the patients who do receive chemotherapy, about 50% receive anthracycline+cyclophosphamide (AC) while about 30% receive a more aggressive combination of AC+ taxane (ACT). Although chemotherapy is more effective in patients who are at higher risk of recurrence without it, there is a subset of patients who experience recurrence even after chemotherapy with AC or ACT.

The prognosis for ER⁺N⁺ patients is less favorable than for ER⁺N⁻ patients. Therefore, these patients more often elect chemotherapy, with about 10% receiving AC and about 80% receiving ACT. Chemotherapy is also less effective in this ER⁺N⁺ group, in that N+ patients have higher recurrence rates than N− after chemotherapy.

In both ER⁺N⁺ and ER⁺N⁻ breast cancer patients, the ability to predict the likelihood of recurrence after standard anthracycline-based chemotherapy (residual risk) would be extremely useful. Patients shown to have high residual risk could elect an alternative therapeutic regimen. Treatment choices could include a more intensive (than standard) course of anthracycline-based chemotherapy, a different drug or drug combination, a different treatment modality, such as radiation, or no treatment at all.

Improved ability to predict residual risk would also extremely useful in carrying out clinical trials. For example, a drug developer might want to test the efficacy of a drug candidate added in combination with AC chemotherapy. In the absence of a recurrence risk prediction, a large number of patients would be required for such a trial because many of the patients enrolled would have a high likelihood of a positive outcome without the added drug. By applying a test for recurrence risk, the population enrolled in a trial can be enriched for patients having a low likelihood of a positive outcome without the added drug. This reduces the enrollment required to demonstrate the efficacy of the drug and thus reduces the time and cost of executing the trial.

### SUMMARY OF THE INVENTION

In one aspect, the invention concerns a method of predicting the clinical outcome for a patient receiving adjuvant anthracycline-based chemotherapy and having hormone receptor positive (HR+) breast cancer, the method comprising:

assaying an expression level of at least one RNA transcript listed in Tables 4A-B, or its expression product, in a biological sample comprising cancer cells obtained from the patient; and

determining a normalized expression level of the at least one RNA transcript, or its expression product,

wherein the normalized expression level of the at least one RNA transcript listed in Table 4A, or its expression product, correlates with a decreased likelihood of a positive clinical outcome; and

wherein the normalized expression level of the at least one RNA transcript listed in Table 4B, or its expression product, correlates with an increased likelihood of a positive clinical outcome.

In another aspect, the invention concerns a method of predicting the clinical outcome for a patient receiving adjuvant anthracycline-based chemotherapy and having hormone receptor negative (HR−) breast cancer, the method comprising:

assaying an expression level of at least one RNA transcript listed in Tables 5A-B, or its expression product, in a biological sample comprising cancer cells obtained from the patient; and

determining a normalized expression level of the at least one RNA transcript, or its expression product,

wherein the normalized expression level of the at least one RNA transcript listed in Table 5A, or its expression product, correlates with a decreased likelihood of a positive clinical outcome; and

wherein the normalized expression level of the at least one RNA transcript listed in Table 5B, or its expression product, correlates with an increased likelihood of a positive clinical outcome.

In yet another aspect, the invention concerns method of predicting the clinical outcome for a patient receiving adjuvant anthracycline-based chemotherapy and having hormone receptor positive (HR+), human epidermal growth factor receptor 2 negative (HER2−) breast cancer, the method comprising:

assaying an expression level of the at least one RNA transcript listed in Tables 6A-B, or its expression product, in a biological sample comprising cancer cells obtained from the patient; and

determining a normalized expression level of the at least one RNA transcript, or its expression product,

wherein the normalized expression level of the at least one RNA transcript listed in Table 6A, or its expression product, correlates with a decreased likelihood of a positive clinical outcome; and

wherein the normalized expression level of the at least one RNA transcript listed in Table 6B, or its expression product, correlates with an increased likelihood of a positive clinical outcome.

In a further aspect, the invention concerns a method of predicting the clinical outcome for a patient receiving adjuvant anthracycline-based chemotherapy and having hormone receptor negative (HR), human epidermal growth factor receptor 2 negative (HER2−) breast cancer, the method comprising:

assaying an expression level of at least one RNA transcript listed in Tables 7A-B, or its expression product, in a biological sample comprising cancer cells obtained from the patient; and

determining a normalized expression level of the at least one RNA transcript, or its expression product,

wherein the normalized expression level of the at least one RNA transcript listed in Table 7A, or its expression product, correlates with a decreased likelihood of a positive clinical outcome; and

wherein the normalized expression level of the at least one RNA transcript listed in Table 7B, or its expression product, correlates with an increased likelihood of a positive clinical outcome.

In a still further aspect, the invention concerns a method of predicting the clinical outcome for a patient receiving adjuvant anthracycline-based chemotherapy and having hormone receptor positive (HR+), human epidermal growth factor receptor 2 positive (HER2+) breast cancer, the method comprising:

assaying an expression level of at least one RNA transcript listed in Tables 8A-B, or its expression product, in a biological sample comprising cancer cells obtained from the patient; and

determining a normalized expression level of the at least one RNA transcript, or its expression product,

wherein the normalized expression level of the at least one RNA transcript listed in Table 8A, or its expression product, correlates with a decreased likelihood of a positive clinical outcome; and

wherein the normalized expression level of the at least one RNA transcript listed in Table 8B, or its expression product, correlates with an increased likelihood of a positive clinical outcome.

The invention further concerns a method of predicting the clinical outcome for a patient receiving adjuvant anthracycline-based chemotherapy and having hormone receptor negative (HR−), human epidermal growth factor receptor 2 positive (HER2+) breast cancer, the method comprising:

assaying an expression level of at least one RNA transcript listed in Tables 9A-B, or its expression product, in a biological sample comprising cancer cells obtained from the patient; and

determining a normalized expression level of the at least one RNA transcript, or its expression product,

wherein the normalized expression level of the at least one RNA transcript listed in Table 9A, or its expression product, correlates with a decreased likelihood of a positive clinical outcome; and

wherein the normalized expression level of the at least one RNA transcript listed in Table 9B, or its expression product, correlates with an increased likelihood of a positive clinical outcome.

In yet another aspect, the invention concerns a method of predicting the likelihood that a patient having hormone receptor positive (HR+) breast cancer will exhibit a clinical benefit in response to adjuvant treatment with an anthracycline-based chemotherapy, the method comprising:

assaying a biological sample obtained from a cancer tumor of the patient for an expression level of at least one RNA transcript listed in Tables 4A-B, 6A-B, and/or 8A-B, or its expression product,

determining a normalized expression level of the at least one RNA transcript in Tables 4A-B, 6A-B, and/or 8A-B, or its expression product,

wherein the normalized expression level of the at least one RNA transcript listed in Table 4A, 6A, and/or 8A, or its expression product, positively correlates with a clinical benefit in response to treatment with an anthracycline-based chemotherapy; and

wherein the normalized expression level of the at least one RNA transcript listed in Table 4B, 6B, and/or 8B, or its expression product, negatively correlates with a clinical benefit in response to treatment with an anthracycline-based chemotherapy.

In a different aspect, the invention concerns a method of predicting the likelihood that a patient having hormone receptor negative (HR−) breast cancer will exhibit a clinical benefit in response to adjuvant treatment with an anthracycline-based chemotherapy, the method comprising:

assaying a biological sample obtained from a cancer tumor of the patient for an expression level of at least one RNA transcript listed in Tables 5A-B, 7A-B, and/or 9A-B, or its expression product,

determining a normalized expression level of the at least one RNA transcript in Tables 5A-B, 7A-B, and/or 9A-B, or its expression product,

wherein the normalized expression level of the at least one RNA transcript listed in Table 5A, 7A, and/or 9A, or its expression product, positively correlates with a clinical benefit in response to treatment with an anthracycline-based chemotherapy; and

wherein the normalized expression level of the at least one RNA transcript listed in Table 5B, 7B, and/or 9B, or its expression product, negatively correlates with a clinical benefit in response to treatment with an anthracycline-based chemotherapy.

The clinical outcome of the method of the invention may be expressed, for example, in terms of Recurrence-Free Interval (RFI), Overall Survival (OS), Disease-Free Survival (DFS), or Distant Recurrence-Free Interval (DRFI).

In one aspect, the cancer is human epidermal growth factor receptor 2 (HER2) positive breast cancer.

In one aspect, the cancer is HER2 negative breast cancer.

For all aspects of the method of the invention, determining the expression level of at least one genes may be obtained, for example, by a method of gene expression profiling. The method of gene expression profiling may be, for example, a PCR-based method or digital gene expression.

For all aspects of the invention, the patient preferably is a human.

For all aspects of the invention, the method may further comprise creating a report based on the normalized expression level. The report may further contain a prediction regarding clinical outcome and/or recurrence. The report may further contain a treatment recommendation.

For all aspects of the invention, the determination of expression levels may occur more than one time. For all aspects of the invention, the determination of expression levels may occur before the patient is subjected to any therapy.

The prediction of clinical outcome may comprise an estimate of the likelihood of a particular clinical outcome for a subject or may comprise the classification of a subject into a risk group based on the estimate.

In another aspect, the invention concerns a kit comprising a set of gene specific probes and/or primers for quantifying the expression of one or more of the genes listed in any one of Tables 1, 2, 3, 4A-B, 5A-B, 6A-B, 7A-B, 8A-B, and 9A-B by quantitative RT-PCR.

In one embodiment, the kit further comprises one or more reagents for expression of RNA from tumor samples.

In another embodiment, the kit comprises one or more containers.

In yet another embodiment, the kit comprises one or more algorithms that yield prognostic or predictive information.

In a further embodiment, one or more of the containers present in the kit comprise pre-fabricated microarrays, a buffers, nucleotide triphosphates, reverse transcriptase, DNA polymerase, RNA polymerase, probes, or primers.

In a still further embodiment, the kit comprises a label and/or a package insert with instructions for use of its components.

In a further embodiment, the instructions comprise directions for use in the prediction or prognosis of breast cancer.

The invention further comprises a method of preparing a personalized genomics profile for a patient comprising the steps of: (a) determining the normalized expression levels of the RNA transcripts or the expression products of one or more genes listed in Tables 1, 2, 3, 4A-B, 5A-B, 6A-B, 7A-B, 8A-B, and 9A-B, in a cancer cell obtained from the patient; and (b) creating a report summarizing the data obtained by said gene expression analysis.

The method may further comprise the step of communicating the report to the patient or a physician of the patient.

The invention further concerns a report comprises the results of the gene expression analysis performed as described in any of the aspects and embodiments described above.

### BRIEF DESCRIPTION OF THE DRAWINGS

FIG. 1: E2197 Main Study Results—Disease-Free Survival

FIG. 2: E2197 Main Study Results—Overall Survival

### DETAILED DESCRIPTION OF THE INVENTION

### A. Definitions

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), and March, Advanced Organic Chemistry Reactions, Mechanisms and Structure 4th ed., John Wiley & Sons (New York, N.Y. 1992), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

A “biological sample” encompasses a variety of sample types obtained from an individual. The definition encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents; washed; or enrichment for certain cell populations, such as cancer cells. The definition also includes sample that have been enriched for particular types of molecules, e.g., nucleic acids, polypeptides, etc. The term “biological sample” encompasses a clinical sample, and also includes tissue obtained by surgical resection, tissue obtained by biopsy, cells in culture, cell supernatants, cell lysates, tissue samples, organs, bone marrow, blood, plasma, serum, and the like. A “biological sample” includes a sample obtained from a patient's cancer cell, e.g., a sample comprising polynucleotides and/or polypeptides that is obtained from a patient's cancer cell (e.g., a cell lysate or other cell extract comprising polynucleotides and/or polypeptides); and a sample comprising cancer cells from a patient. A biological sample comprising a cancer cell from a patient can also include non-cancerous cells.

The terms “cancer,” “neoplasm,” and “tumor” are used interchangeably herein to refer to the physiological condition in mammal cells that is typically characterized by an aberrant growth phenotype and a significant loss of control of cell proliferation. In general, cells of interest for detection, analysis, classification, or treatment in the present application include precancerous (e.g., benign), malignant, pre-metastatic, metastatic, and non-metastatic cells.

The term “hormone receptor positive (HR+) tumors” means tumors expressing either estrogen receptor (ER) or progesterone receptor (PR) as determined by standard methods (e.g., immunohistochemical staining of nuclei in the patients biological samples). The term “hormone receptor negative (HR−) tumors” means tumors expressing neither estrogen receptor (ER) nor progesterone receptor (PR) as determined by standard methods, including immunohistochemical staining. Such methods of immunohistochemical staining are routine and known to one of skill in the art.

The “pathology” of cancer includes all phenomena that compromise the well-being of the patient. This includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, invasion of surrounding or distant tissues or organs, such as lymph nodes, etc.

The term “prognosis” is used herein to refer to the prediction of the likelihood of cancer-attributable death or progression, including recurrence, metastatic spread, and drug resistance, of a neoplastic disease, such as breast cancer.

Prognostic factors are those variables related to the natural history of breast cancer, which influence the recurrence rates and outcome of patients once they have developed breast cancer. Clinical parameters that have been associated with a worse prognosis include, for example, lymph node involvement, and high grade tumors. Prognostic factors are frequently used to categorize patients into subgroups with different baseline recurrence risks.

The term “prediction” is used herein to refer to the likelihood that a patient will have a particular clinical outcome, whether positive or negative, following surgical removal of the primary tumor and treatment with anthracycline-based chemotherapy. The predictive methods of the present invention can be used clinically to make treatment decisions by choosing the most appropriate treatment modalities for any particular patient. The predictive methods of the present invention are valuable tools in predicting if a patient is likely to respond favorably to a treatment regimen, such as chemotherapy or surgical intervention.

“Positive patient response” or “positive clinical outcome” can be assessed using any endpoint indicating a benefit to the patient, including, without limitation, (1) inhibition, to some extent, of tumor growth, including slowing down and complete growth arrest; (2) reduction in the number of tumor cells; (3) reduction in tumor size; (4) inhibition (i.e., reduction, slowing down or complete stopping) of tumor cell infiltration into adjacent peripheral organs and/or tissues; (5) inhibition (i.e. reduction, slowing down or complete stopping) of metastasis; (6) enhancement of anti-tumor immune response, which may, but does not have to, result in the regression or rejection of the tumor; (7) relief, to some extent, of at least one symptoms associated with the tumor; (8) increase in the length of survival following treatment; and/or (9) decreased mortality at a given point of time following treatment. The term “positive clinical outcome” means an improvement in any measure of patient status, including those measures ordinarily used in the art, such as an increase in the duration of Recurrence-Free interval (RFI), an increase in the time of Overall Survival (OS), an increase in the time of Disease-Free Survival (DFS), an increase in the duration of Distant Recurrence-Free Interval (DRFI), and the like. An increase in the likelihood of positive clinical outcome corresponds to a decrease in the likelihood of cancer recurrence.

The term “residual risk” except when specified otherwise is used herein to refer to the probability or risk of cancer recurrence in breast cancer patients after surgical resection of their tumor and treatment with anthracycline-based chemotherapies.

The term “anthracycline-based chemotherapies” is used herein to refer to chemotherapies that comprise an anthracycline compound, for example doxorubicin, daunorubicin, epirubicin or idarubicin. Such anthracycline based chemotherapies may be combined with other chemotherapeutic compounds to form combination chemotherapies such as, without limitation, anthracycline+cyclophosphamide (AC), anthracycline+taxane (AT), or anthracycline+cyclophosphamide+taxane (ACT).

The term “long-term” survival is used herein to refer to survival for at least 3 years, more preferably for at least 5 years.

The term “Recurrence-Free Interval (RFI)” is used herein to refer to time in years to first breast cancer recurrence censoring for second primary cancer or death without evidence of recurrence.

The term “Overall Survival (OS)” is used herein to refer to time in years from surgery to death from any cause.

The term “Disease-Free Survival (DFS)” is used herein to refer to time in years to breast cancer recurrence or death from any cause.

The term “Distant Recurrence-Free Interval (DRFI)” is used herein to refer to the time (in years) from surgery to the first anatomically distant cancer recurrence, censoring for second primary cancer or death without evidence of recurrence.

The calculation of the measures listed above in practice may vary from study to study depending on the definition of events to be either censored or not considered.

The term “subject” or “patient” refers to a mammal being treated. In an embodiment the mammal is a human.

The term “microarray” refers to an ordered arrangement of hybridizable array elements, preferably polynucleotide probes, on a substrate.

The terms “gene product” and “expression product” are used interchangeably herein in reference to a gene, to refer to the RNA transcription products (transcripts) of the gene, including mRNA and the polypeptide translation products of such RNA transcripts, whether such product is modified post-translationally or not. The terms “gene product” and “expression product” are used interchangeably herein, in reference to an RNA, particularly an mRNA, to refer to the polypeptide translation products of such RNA, whether such product is modified post-translationally or not. A gene product can be, for example, an unspliced RNA, an mRNA, a splice variant mRNA, a polypeptide, a post-translationally modified polypeptide, a splice variant polypeptide, etc.

As used herein, the term “normalized expression level” refers to an expression level of a response indicator gene relative to the level of an expression product of a reference gene(s).

The term “polynucleotide,” when used in singular or plural, generally refers to any polyribonucleotide or polydeoxyribonucleotide, which may be unmodified RNA or DNA or modified RNA or DNA. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of at least one of the molecules, but more typically involve only a region of some of the molecules. One of the molecules of a triple-helical region often is an oligonucleotide. The term “polynucleotide” specifically includes cDNAs. The term includes DNAs (including cDNAs) and RNAs that contain at least one modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritiated bases, are included within the term “polynucleotides” as defined herein. In general, the term “polynucleotide” embraces all chemically, enzymatically and/or metabolically modified forms of unmodified polynucleotides, as well as the chemical forms of DNA and RNA characteristic of viruses and cells, including simple and complex cells.

The term “oligonucleotide” refers to a relatively short polynucleotide, including, without limitation, single-stranded deoxyribonucleotides, single- or double-stranded ribonucleotides, RNA:DNA hybrids and double-stranded DNAs. Oligonucleotides, such as single-stranded DNA probe oligonucleotides, are often synthesized by chemical methods, for example using automated oligonucleotide synthesizers that are commercially available. However, oligonucleotides can be made by a variety of other methods, including in vitro recombinant DNA-mediated techniques and by expression of DNAs in cells and organisms.

The terms “differentially expressed gene,” “differential gene expression” and their synonyms, which are used interchangeably, refer to a gene whose expression is activated to a higher or lower level in a subject suffering from a disease, specifically cancer, such as breast cancer, relative to its expression in a normal or control subject. The terms also include genes whose expression is activated to a higher or lower level at different stages of the same disease. It is also understood that a differentially expressed gene may be either activated or inhibited at the nucleic acid level or protein level, or may be subject to alternative splicing to result in a different polypeptide product. Such differences may be evidenced by a change in mRNA levels, surface expression, secretion or other partitioning of a polypeptide, for example. Differential gene expression may include a comparison of expression between two or more genes or their gene products, or a comparison of the ratios of the expression between two or more genes or their gene products, or even a comparison of two differently processed products of the same gene, which differ between normal subjects and subjects suffering from a disease, specifically cancer, or between various stages of the same disease. Differential expression includes both quantitative, as well as qualitative, differences in the temporal or cellular expression pattern in a gene or its expression products among, for example, normal and diseased cells, or among cells which have undergone different disease events or disease stages. For the purpose of this invention, “differential gene expression” is considered to be present when there is at least an about two-fold, preferably at least about four-fold, more preferably at least about six-fold, most preferably at least about ten-fold difference between the expression of a given gene in normal and diseased subjects, or in various stages of disease development in a diseased subject.

The term “over-expression” with regard to an RNA transcript is used to refer to the level of the transcript determined by normalization to the level of reference mRNAs, which might be all measured transcripts in the specimen or a particular reference set of mRNAs such as housekeeping genes. The assay typically measures and incorporates the expression of certain normalizing genes, including well known housekeeping genes, such as GAPDH and Cyp1. Alternatively, normalization can be based on the mean or median signal (Ct) of all of the assayed genes or a large subset thereof (global normalization approach). On a gene-by-gene basis, measured normalized amount of a patient tumor mRNA is compared to the amount found in a cancer tissue reference set. The number (N) of cancer tissues in this reference set should be sufficiently high to ensure that different reference sets (as a whole) behave essentially the same way. If this condition is met, the identity of the individual cancer tissues present in a particular set will have no significant impact on the relative amounts of the genes assayed. Usually, the cancer tissue reference set consists of at least about 30, preferably at least about 40 different FPE cancer tissue specimens.

As used herein, “gene expression profiling” refers to research methods that measure mRNA made from many different genes in various cell types. For example, this method may be used to monitor the expression of thousands of genes simultaneously using microarray technology. Gene expression profiling may be used as a diagnostic test to help identify subgroups of tumor types, to help predict which patients may respond to treatment, and which patients may be at increased risk for cancer relapse.

The phrase “gene amplification” refers to a process by which multiple copies of a gene or gene fragment are formed in a particular cell or cell line. The duplicated region (a stretch of amplified DNA) is often referred to as “amplicon.” Usually, the amount of the messenger RNA (mRNA) produced, i.e., the level of gene expression, also increases in the proportion of the number of copies made of the particular gene expressed.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, typically: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide, followed by a high-stringency wash consisting of 0.1×SSC containing EDTA at 55° C.

“Moderately stringent conditions” may be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

In the context of the present invention, reference to “at least one,” “at least two,” “at least five,” etc. of the genes listed in any particular gene set means any one or any and all combinations of the genes listed.

The term “node negative” cancer, such as “node negative” breast cancer, is used herein to refer to cancer that has not spread to the lymph nodes.

The terms “splicing” and “RNA splicing” are used interchangeably and refer to RNA processing that removes introns and joins exons to produce mature mRNA with continuous coding sequence that moves into the cytoplasm of an eukaryotic cell.

In theory, the term “exon” refers to any segment of an interrupted gene that is represented in the mature RNA product (B. Lewin. Genes IV Cell Press, Cambridge Mass. 1990). In theory the term “intron” refers to any segment of DNA that is transcribed but removed from within the transcript by splicing together the exons on either side of it. Operationally, exon sequences occur in the mRNA sequence of a gene as defined by Ref. Seq ID numbers on the Entrez Gene database maintained by the National Center for Biotechnology Information. Operationally, intron sequences are the intervening sequences within the genomic DNA of a gene, bracketed by exon sequences and having GT and AG splice consensus sequences at their 5′ and 3′ boundaries.

The term “expression cluster” is used herein to refer to a group of genes which demonstrate similar expression patterns when studied within samples from a defined set of patients. As used herein, the genes within an expression cluster show similar expression patterns when studied within samples from patients with invasive breast cancer.

The terms “correlate” and “correlation” refer to the simultaneous change in value of two numerically valued variables. For example, correlation may indicate the strength and direction of a linear relationship between two variables indicating that they are not independent. The correlation between the two such variables could be positive or negative.

### B.1 General Description of the Invention

The practice of the present invention will employ, unless otherwise indicated, conventional techniques of molecular biology (including recombinant techniques), microbiology, cell biology, and biochemistry, which are within the skill of the art. Such techniques are explained fully in the literature, such as, “Molecular Cloning: A Laboratory Manual”, 2nd edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology”, 4th edition (D. M. Weir & C. C. Blackwell, eds., Blackwell Science Inc., 1987); “Gene Transfer Vectors for Mammalian Cells” (J. M. Miller & M. P. Calos, eds., 1987); “Current Protocols in Molecular Biology” (F. M. Ausubel et al., eds., 1987); and “PCR: The Polymerase Chain Reaction”, (Mullis et al., eds., 1994).

Disruptions in the normal functioning of various physiological processes, including proliferation, apoptosis, angiogenesis and invasion, have been implicated in the pathology in cancer. The relative contribution of dysfunctions in particular physiological processes to the pathology of particular cancer types is not well characterized. Any physiological process integrates the contributions of numerous gene products expressed by the various cells involved in the process. For example, tumor cell invasion of adjacent normal tissue and intravasation of the tumor cell into the circulatory system are effected by an array of proteins that mediate various cellular characteristics, including cohesion among tumor cells, adhesion of tumor cells to normal cells and connective tissue, ability of the tumor cell first to alter its morphology and then to migrate through surrounding tissues, and ability of the tumor cell to degrade surrounding connective tissue structures.

Multi-analyte gene expression tests can measure the expression level of at least one genes involved in each of several relevant physiologic processes or component cellular characteristics. In some instances the predictive power of the test, and therefore its utility, can be improved by using the expression values obtained for individual genes to calculate a score which is more highly associated with outcome than is the expression value of the individual genes. For example, the calculation of a quantitative score (recurrence score) that predicts the likelihood of recurrence in estrogen receptor-positive, node-negative breast cancer is describe in U.S. Publication No. 20050048542, published Mar. 3, 2005, the entire disclosure of which is expressly incorporated by reference herein. The equation used to calculate such a recurrence score may group genes in order to maximize the predictive value of the recurrence score. The grouping of genes may be performed at least in part based on knowledge of their contribution to physiologic functions or component cellular characteristics such as discussed above. The formation of groups, in addition, can facilitate the mathematical weighting of the contribution of various expression values to the recurrence score. The weighting of a gene group representing a physiological process or component cellular characteristic can reflect the contribution of that process or characteristic to the pathology of the cancer and clinical outcome. Accordingly, in an important aspect, the present invention also provides specific groups of the prognostic genes identified herein, that together are more reliable and powerful predictors of outcome than the individual genes or random combinations of the genes identified.

Measurement of prognostic RNA transcript expression levels may be performed by using a software program executed by a suitable processor. Suitable software and processors are well known in the art and are commercially available. The program may be embodied in software stored on a tangible medium such as CD-ROM, a floppy disk, a hard drive, a DVD, or a memory associated with the processor, but persons of ordinary skill in the art will readily appreciate that the entire program or parts thereof could alternatively be executed by a device other than a processor, and/or embodied in firmware and/or dedicated hardware in a well known manner.

Following the measurement of the expression levels of the genes identified herein, or their expression products, and the determination that a subject is likely or not likely to respond to treatment with an anthracycline-based chemotherapy (e.g., anthracycline+cyclophosphamide (AC) or AC+taxane (ACT)), the assay results, findings, diagnoses, predictions and/or treatment recommendations are typically recorded and communicated to technicians, physicians and/or patients, for example. In certain embodiments, computers will be used to communicate such information to interested parties, such as, patients and/or the attending physicians. In some embodiments, the assays will be performed or the assay results analyzed in a country or jurisdiction which differs from the country or jurisdiction to which the results or diagnoses are communicated.

In a preferred embodiment, a diagnosis, prediction and/or treatment recommendation based on the expression level in a test subject of at least one of the biomarkers herein is communicated to the subject as soon as possible after the assay is completed and the diagnosis and/or prediction is generated. The results and/or related information may be communicated to the subject by the subject's treating physician. Alternatively, the results may be communicated directly to a test subject by any means of communication, including writing, electronic forms of communication, such as email, or telephone. Communication may be facilitated by use of a computer, such as in case of email communications. In certain embodiments, the communication containing results of a diagnostic test and/or conclusions drawn from and/or treatment recommendations based on the test, may be generated and delivered automatically to the subject using a combination of computer hardware and software which will be familiar to artisans skilled in telecommunications. One example of a healthcare-oriented communications system is described in U.S. Pat. No. 6,283,761; however, the present invention is not limited to methods which utilize this particular communications system. In certain embodiments of the methods of the invention, all or some of the method steps, including the assaying of samples, diagnosing of diseases, and communicating of assay results or diagnoses, may be carried out in diverse (e.g., foreign) jurisdictions.

The utility of a marker in predicting recurrence risk may not be unique to that marker. An alternative gene having expression values that are closely correlated with those of a known gene marker may be substituted for or used in addition to the known marker and have little impact on the overall predictive utility of the test. The correlated expression pattern of the two genes may result from involvement of both genes in a particular process and/or being under common regulatory control in breast tumor cells. The present invention specifically includes and contemplates the use of at least one such substitute genes in the methods of the present invention.

The markers of recurrence risk in breast cancer patients provided by the present invention have utility in the choice of treatment for patients diagnosed with breast cancer. While the rate of recurrence in early stage breast cancer is relatively low compared to recurrence rates in some other types of cancer, there is a subpopulation of these patients who have a relatively high recurrence rate (poor prognosis) if not treated with chemotherapy in addition to surgical resection of their tumors. Among these patients with poor prognosis are a smaller number of individuals who are unlikely to respond to chemotherapy, for example AC or ACT. The methods of this invention are useful for the identification of individuals with poor initial prognosis and low likelihood of response to standard chemotherapy which, taken together, result in high recurrence risk. In the absence of a recurrence risk prediction, these patients would likely receive and often fail to benefit from standard chemotherapy treatment. With an accurate test for prediction of recurrence risk, these patients may elect alternative treatment to standard chemotherapy and in doing so avoid the toxicity of standard chemotherapy and unnecessary delay in availing themselves of what may be a more effective treatment.

The markers and associated information provided by the present invention for predicting recurrence risk in breast cancer patients also have utility in screening patients for inclusion in clinical trials that test the efficacy of drug compounds. Experimental chemotherapy drugs are often tested in clinical trials by testing the experimental drug in combination with standard chemotherapeutic drugs and comparing the results achieved in this treatment group with the results achieved using standard chemotherapy alone. The presence in the trial of a significant subpopulation of patients who respond to the experimental treatment because it includes standard chemotherapy drugs already proven to be effective complicates the identification of patients who are responsive to the experimental drug and increases the number of patients that must be enrolled in the clinical trial to optimize the likelihood of demonstrating the efficacy of the experimental drug. A more efficient clinical trial could be designed if patients having a high degree of recurrence risk could be identified. The markers of this invention are useful for developing such a recurrence risk test, such that high recurrence risk could be used as an inclusion criteria for clinical trial enrollment.

In a particular embodiment, prognostic markers and associated information are used to design or produce a reagent that modulates the level or activity of the gene's transcript or its expression product. Said reagents may include but are not limited to an antisense RNA, a small inhibitory RNA, micro RNA, a ribozyme, a monoclonal or polyclonal antibody.

In various embodiments of the inventions, various technological approaches are available for determination of expression levels of the disclosed genes, including, without limitation, RT-PCR, microarrays, serial analysis of gene expression (SAGE) and Gene Expression Analysis by Massively Parallel Signature Sequencing (MPSS), which will be discussed in detail below. In particular embodiments, the expression level of each gene may be determined in relation to various features of the expression products of the gene including exons, introns, protein epitopes and protein activity. In other embodiments, the expression level of a gene may be inferred from analysis of the structure of the gene, for example from the analysis of the methylation pattern of the gene's promoter(s).

### B.2 Gene Expression Profiling

Methods of gene expression profiling include methods based on hybridization analysis of polynucleotides, methods based on sequencing of polynucleotides, and proteomics-based methods. The most commonly used methods known in the art for the quantification of mRNA expression in a sample include northern blotting and in situ hybridization (Parker & Barnes, Methods in Molecular Biology 106:247-283 (1999)); RNAse protection assays (Hod, Biotechniques 13:852-854 (1992)); and PCR-based methods, such as reverse transcription polymerase chain reaction (RT-PCR) (Weis et al., Trends in Genetics 8:263-264 (1992)). Alternatively, antibodies may be employed that can recognize sequence-specific duplexes, including DNA duplexes, RNA duplexes, and DNA RNA hybrid duplexes or DNA protein duplexes. Representative methods for sequencing-based gene expression analysis include Serial Analysis of Gene Expression (SAGE), and gene expression analysis by massively parallel signature sequencing (MPSS).

a. Reverse Transcriptase PCR

Of the techniques listed above, the most sensitive and most flexible quantitative method is quantitative real time polymerase chain reaction (qRT-PCR), which can be used to determine mRNA levels in various samples. The results can be used to compare gene expression patterns between sample sets, for example in normal and tumor tissues or in patients with or without drug treatment.

The first step is the isolation of mRNA from a target sample. The starting material is typically total RNA isolated from human tumors or tumor cell lines, and corresponding normal tissues or cell lines, respectively. Thus RNA can be isolated from a variety of primary tumors, including breast, lung, colon, prostate, brain, liver, kidney, pancreas, spleen, thymus, testis, ovary, uterus, etc., tumor, or tumor cell lines, with pooled DNA from healthy donors. If the source of mRNA is a primary tumor, mRNA can be extracted, for example, from frozen or archived paraffin-embedded and fixed (e.g. formalin-fixed) tissue samples.

General methods for mRNA extraction are well known in the art and are disclosed in standard textbooks of molecular biology, including Ausubel et al., Current Protocols of Molecular Biology, John Wiley and Sons (1997). Methods for RNA extraction from paraffin embedded tissues are disclosed, for example, in Rupp and Locker, Lab Invest. 56:A67 (1987), and De Andres et al., BioTechniques 18:42044 (1995). In particular, RNA isolation can be performed using a purification kit, buffer set and protease from commercial manufacturers, such as Qiagen, according to the manufacturer's instructions. For example, total RNA from cells in culture can be isolated using Qiagen RNeasy mini-columns. Other commercially available RNA isolation kits include MasterPure™ Complete DNA and RNA Purification Kit (EPICENTRE®, Madison, Wis.), and Paraffin Block RNA Isolation Kit (Ambion, Inc.). Total RNA from tissue samples can be isolated using RNA Stat-60 (Tel-Test). RNA prepared from tumor can be isolated, for example, by cesium chloride density gradient centrifugation.

As RNA cannot serve as a template for PCR, the first step in gene expression profiling by RT-PCR is the reverse transcription of the RNA template into cDNA, followed by its exponential amplification in a PCR reaction. The two most commonly used reverse transcriptases are avilo myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, Calif., USA), following the manufacturer's instructions. The derived cDNA can then be used as a template in the subsequent PCR reaction.

Although the PCR step can use a variety of thermostable DNA-dependent DNA polymerases, it typically employs the Taq DNA polymerase, which has a 5′-3′ nuclease activity but lacks a 3′-5′ proofreading endonuclease activity. Thus, TaqMan® PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non extendible by Taq DNA polymerase enzyme, and is labeled with a reporter fluorescent dye and a quencher fluorescent dye. Any laser induced emission from the reporter dye is quenched by the quenching dye when the two dyes are located close together as they are on the probe. During the amplification reaction, the Taq DNA polymerase enzyme cleaves the probe in a template dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TaqMan® RT-PCR can be performed using commercially available equipment, such as, for example, ABI PRISM 7700TM Sequence Detection System™ (Perkin-Elmer-Applied Biosystems, Foster City, Calif., USA), or Lightcycler (Roche Molecular Biochemicals, Mannheim, Germany). In a preferred embodiment, the 5′ nuclease procedure is run on a real-time quantitative PCR device such as the ABI PRISM 7700TM Sequence Detection System™. The system consists of a thermocycler, laser, charge coupled device (CCD), camera and computer. The system amplifies samples in a 96 well format on a thermocycler. During amplification, laser induced fluorescent signal is collected in real time through fiber optics cables for all 96 wells, and detected at the CCD. The system includes software for running the instrument and for analyzing the data.

5′-Nuclease assay data are initially expressed as Ct, or the threshold cycle. As discussed above, fluorescence values are recorded during every cycle and represent the amount of product amplified to that point in the amplification reaction. The point when the fluorescent signal is first recorded as statistically significant is the threshold cycle (Ct).

To minimize errors and the effect of sample-to-sample variation, RT-PCR is usually performed using an internal standard. The ideal internal standard is expressed at a constant level among different tissues, and is unaffected by the experimental treatment. RNAs most frequently used to normalize patterns of gene expression are mRNAs for the housekeeping genes glyceraldehyde-3-phosphate-dehydrogenase (GAPDH) and β-actin.

A more recent variation of the RT-PCR technique is the real time quantitative PCR, which measures PCR product accumulation through a dual-labeled fluorigenic probe (i.e., TaqMan® probe). Real time PCR is compatible both with quantitative competitive PCR, where internal competitor for each target sequence is used for normalization, and with quantitative comparative PCR using a normalization gene contained within the sample, or a housekeeping gene for RT-PCR. For further details see, e.g. Held et al., Genome Research 6:986-994 (1996).

The steps of a representative protocol for profiling gene expression using fixed, paraffin-embedded tissues as the RNA source, including mRNA isolation, purification, primer extension and amplification are given in various published journal articles (for example: T. E. Godfrey et al. J. Molec. Diagnostics 2: 84-91 (2000); K. Specht et al., Am. J. Pathol. 158: 419-29 (2001)). Briefly, a representative process starts with cutting about 10 μm thick sections of paraffin-embedded tumor tissue samples. The RNA is then extracted, and protein and DNA are removed. After analysis of the RNA concentration, RNA repair and/or amplification steps may be included, if necessary, and RNA is reverse transcribed using gene specific promoters followed by RT-PCR.

b. MassARRAY System

In the MassARRAY-based gene expression profiling method, developed by Sequenom, Inc. (San Diego, Calif.) following the isolation of RNA and reverse transcription, the obtained cDNA is spiked with a synthetic DNA molecule (competitor), which matches the targeted cDNA region in all positions, except a single base, and serves as an internal standard. The cDNA/competitor mixture is PCR amplified and is subjected to a post-PCR shrimp alkaline phosphatase (SAP) enzyme treatment, which results in the dephosphorylation of the remaining nucleotides. After inactivation of the alkaline phosphatase, the PCR products from the competitor and cDNA are subjected to primer extension, which generates distinct mass signals for the competitor- and cDNA-derived PCR products. After purification, these products are dispensed on a chip array, which is pre-loaded with components needed for analysis with matrix-assisted laser desorption ionization time-of-flight mass spectrometry (MALDI-TOF MS) analysis. The cDNA present in the reaction is then quantified by analyzing the ratios of the peak areas in the mass spectrum generated. For further details see, e.g. Ding and Cantor, Proc. Natl. Acad. Sci. USA 100:3059-3064 (2003).

c. Other PCR-Based Methods

Further PCR-based techniques include, for example, differential display (Liang and Pardee, Science 257:967-971 (1992)); amplified fragment length polymorphism (iAFLP) (Kawamoto et al., Genome Res. 12:1305-1312 (1999)); BeadArray™ technology (Illumina, San Diego, Calif.; Oliphant et al., Discovery of Markers for Disease (Supplement to Biotechniques), June 2002; Ferguson et al., Analytical Chemistry 72:5618 (2000)); BeadsArray for Detection of Gene Expression (BADGE), using the commercially available Luminex100 LabMAP system and multiple color-coded microspheres (Luminex Corp., Austin, Tex.) in a rapid assay for gene expression (Yang et al., Genome Res. 11:1888-1898 (2001)); and high coverage expression profiling (HiCEP) analysis (Fukumura et al., Nucl. Acids. Res. 31(16) e94 (2003)).

d. Microarrays

Differential gene expression can also be identified, or confirmed using the microarray technique. Thus, the expression profile of breast cancer-associated genes can be measured in either fresh or paraffin-embedded tumor tissue, using microarray technology. In this method, polynucleotide sequences of interest (including cDNAs and oligonucleotides) are plated, or arrayed, on a microchip substrate. The arrayed sequences are then hybridized with specific DNA probes from cells or tissues of interest. Just as in the RT-PCR method, the source of mRNA typically is total RNA isolated from human tumors or tumor cell lines, and corresponding normal tissues or cell lines. Thus RNA can be isolated from a variety of primary tumors or tumor cell lines. If the source of mRNA is a primary tumor, mRNA can be extracted, for example, from frozen or archived paraffin-embedded and fixed (e.g. formalin-fixed) tissue samples, which are routinely prepared and preserved in everyday clinical practice.

In a specific embodiment of the microarray technique, PCR amplified inserts of cDNA clones are applied to a substrate in a dense array. Preferably at least 10,000 nucleotide sequences are applied to the substrate. The microarrayed genes, immobilized on the microchip at 10,000 elements each, are suitable for hybridization under stringent conditions. Fluorescently labeled cDNA probes may be generated through incorporation of fluorescent nucleotides by reverse transcription of RNA extracted from tissues of interest. Labeled cDNA probes applied to the chip hybridize with specificity to each spot of DNA on the array. After stringent washing to remove non-specifically bound probes, the chip is scanned by confocal laser microscopy or by another detection method, such as a CCD camera. Quantitation of hybridization of each arrayed element allows for assessment of corresponding mRNA abundance. With dual color fluorescence, separately labeled cDNA probes generated from two sources of RNA are hybridized pair wise to the array. The relative abundance of the transcripts from the two sources corresponding to each specified gene is thus determined simultaneously. The miniaturized scale of the hybridization affords a convenient and rapid evaluation of the expression pattern for large numbers of genes. Such methods have been shown to have the sensitivity required to detect rare transcripts, which are expressed at a few copies per cell, and to reproducibly detect at least approximately two-fold differences in the expression levels (Schena et al., Proc. Natl. Acad. Sci. USA 93(2):106-149 (1996)). Microarray analysis can be performed by commercially available equipment, following manufacturer's protocols, such as by using the Affymetrix GenChip technology, or Incyte's microarray technology.

The development of microarray methods for large-scale analysis of gene expression makes it possible to search systematically for molecular markers of outcome predictions for a variety of chemotherapy treatments for a variety of tumor types.

e. Serial Analysis of Gene Expression (SAGE)

Serial analysis of gene expression (SAGE) is a method that allows the simultaneous and quantitative analysis of a large number of gene transcripts, without the need of providing an individual hybridization probe for each transcript. First, a short sequence tag (about 10-14 bp) is generated that contains sufficient information to uniquely identify a transcript, provided that the tag is obtained from a unique position within each transcript. Then, many transcripts are linked together to form long serial molecules, that can be sequenced, revealing the identity of the multiple tags simultaneously. The expression pattern of any population of transcripts can be quantitatively evaluated by determining the abundance of individual tags, and identifying the gene corresponding to each tag. For more details see, e.g. Velculescu et al., Science 270:484-487 (1995); and Velculescu et al., Cell 88:243-51 (1997).

f. Gene Expression Analysis by Massively Parallel Signature Sequencing (MPSS)

This method, described by Brenner et al., Nature Biotechnology 18:630-634 (2000), is a sequencing approach that combines non-gel-based signature sequencing with in vitro cloning of millions of templates on separate 5 μm diameter microbeads. First, a microbead library of DNA templates is constructed by in vitro cloning. This is followed by the assembly of a planar array of the template-containing microbeads in a flow cell at a high density (typically greater than 3×10⁶ microbeads/cm²). The free ends of the cloned templates on each microbead are analyzed simultaneously, using a fluorescence-based signature sequencing method that does not require DNA fragment separation. This method has been shown to simultaneously and accurately provide, in a single operation, hundreds of thousands of gene signature sequences from a yeast cDNA library.

g. Immunohistochemistry

Immunohistochemistry methods are also suitable for detecting the expression levels of the prognostic markers of the present invention. Thus, antibodies or antisera, preferably polyclonal antisera, and most preferably monoclonal antibodies specific for each marker are used to detect expression. The antibodies can be detected by direct labeling of the antibodies themselves, for example, with radioactive labels, fluorescent labels, hapten labels such as, biotin, or an enzyme such as horse radish peroxidase or alkaline phosphatase. Alternatively, unlabeled primary antibody is used in conjunction with a labeled secondary antibody, comprising antisera, polyclonal antisera or a monoclonal antibody specific for the primary antibody. Immunohistochemistry protocols and kits are well known in the art and are commercially available.

h. Proteomics

The term “proteome” is defined as the totality of the proteins present in a sample (e.g. tissue, organism, or cell culture) at a certain point of time. Proteomics includes, among other things, study of the global changes of protein expression in a sample (also referred to as “expression proteomics”). Proteomics typically includes the following steps: (1) separation of individual proteins in a sample by 2-D gel electrophoresis (2-D PAGE); (2) identification of the individual proteins recovered from the gel, e.g. by mass spectrometry or N-terminal sequencing, and (3) analysis of the data using bioinformatics. Proteomics methods are valuable supplements to other methods of gene expression profiling, and can be used, alone or in combination with other methods, to detect the products of the prognostic markers of the present invention.

i. Chromatin Structure Analysis

A number of methods for quantization of RNA transcripts (gene expression analysis) or their protein translation products are discussed herein. The expression level of genes may also be inferred from information regarding chromatin structure, such as for example the methylation status of gene promoters and other regulatory elements and the acetylation status of histones.

In particular, the methylation status of a promoter influences the level of expression of the gene regulated by that promoter. Aberrant methylation of particular gene promoters has been implicated in expression regulation, such as for example silencing of tumor suppressor genes. Thus, examination of the methylation status of a gene's promoter can be utilized as a surrogate for direct quantization of RNA levels.

Several approaches for measuring the methylation status of particular DNA elements have been devised, including methylation-specific PCR (Herman J. G. et al. (1996) Methylation-specific PCR: a novel PCR assay for methylation status of CpG islands. Proc. Natl. Acad. Sci. USA. 93, 9821-9826.) and bisulfate DNA sequencing (Frommer M. et al. (1992) A genomic sequencing protocol that yields a positive display of 5-methylcytosine residues in individual DNA strands. Proc. Natl. Acad. Sci. USA. 89, 1827-1831.). More recently, microarray-based technologies have been used to characterize promoter methylation status (Chen C. M. (2003) Methylation target array for rapid analysis of CpG island hypermethylation in multiple tissue genomes. Am. J. Pathol. 163, 37-45.).

j. General Description of the mRNA Isolation, Purification and Amplification

The steps of a representative protocol for profiling gene expression using fixed, paraffin-embedded tissues as the RNA source, including mRNA isolation, purification, primer extension and amplification are provided in various published journal articles (for example: T. E. Godfrey et al., J. Molec. Diagnostics 2: 84-91 (2000); K. Specht et al., Am. J. Pathol. 158: 419-29 (2001)). Briefly, a representative process starts with cutting about 10 μm thick sections of paraffin-embedded tumor tissue samples. The RNA is then extracted, and protein and DNA are removed. After analysis of the RNA concentration, RNA repair and/or amplification steps may be included, if necessary, and the RNA is reverse transcribed using gene specific promoters followed by RT-PCR. Finally, the data are analyzed to identify the best treatment option(s) available to the patient on the basis of the characteristic gene expression pattern identified in the tumor sample examined, dependent on the predicted likelihood of cancer recurrence.

k. Breast Cancer Gene Set, Assayed Gene Subsequences, and Clinical Application of Gene Expression Data

An important aspect of the present invention is to use the measured expression of certain genes by breast cancer tissue to provide prognostic information. For this purpose it is necessary to correct for (normalize away) both differences in the amount of RNA assayed and variability in the quality of the RNA used. Therefore, the assay typically measures and incorporates the expression of certain normalizing genes, including well known housekeeping genes, such as GAPDH and Cyp1. Alternatively, normalization can be based on the mean or median signal (Ct) of all of the assayed genes or a large subset thereof (global normalization approach). On a gene-by-gene basis, measured normalized amount of a patient tumor mRNA is compared to the amount found in a breast cancer tissue reference set. The number (N) of breast cancer tissues in this reference set should be sufficiently high to ensure that different reference sets (as a whole) behave essentially the same way. If this condition is met, the identity of the individual breast cancer tissues present in a particular set will have no significant impact on the relative amounts of the genes assayed. Usually, the breast cancer tissue reference set consists of at least about 30, preferably at least about 40 different FPE breast cancer tissue specimens. Unless noted otherwise, normalized expression levels for each mRNA/tested tumor/patient will be expressed as a percentage of the expression level measured in the reference set. More specifically, the reference set of a sufficiently high number (e.g. 40) of tumors yields a distribution of normalized levels of each mRNA species. The level measured in a particular tumor sample to be analyzed falls at some percentile within this range, which can be determined by methods well known in the art. Below, unless noted otherwise, reference to expression levels of a gene assume normalized expression relative to the reference set although this is not always explicitly stated.

l. Design of Intron-Based PCR Primers and Probes

According to one aspect of the present invention, PCR primers and probes are designed based upon intron sequences present in the gene to be amplified. Accordingly, the first step in the primer/probe design is the delineation of intron sequences within the genes. This can be done by publicly available software, such as the DNA BLAT software developed by Kent, W. J., Genome Res. 12(4):656-64 (2002), or by the BLAST software including its variations. Subsequent steps follow well established methods of PCR primer and probe design.

In order to avoid non-specific signals, it is important to mask repetitive sequences within the introns when designing the primers and probes. This can be easily accomplished by using the Repeat Masker program available on-line through the Baylor College of Medicine, which screens DNA sequences against a library of repetitive elements and returns a query sequence in which the repetitive elements are masked. The masked intron sequences can then be used to design primer and probe sequences using any commercially or otherwise publicly available primer/probe design packages, such as Primer Express (Applied Biosystems); MGB assay-by-design (Applied Biosystems); Primer3 (Steve Rozen and Helen J. Skaletsky (2000) Primer3 on the WWW for general users and for biologist programmers. In: Krawetz S, Misener S (eds) Bioinformatics Methods and Protocols: Methods in Molecular Biology. Humana Press, Totowa, N.J., pp 365-386).

The most important factors considered in PCR primer design include primer length, melting temperature (Tm), and G/C content, specificity, complementary primer sequences, and 3′-end sequence. In general, optimal PCR primers are generally 17-30 bases in length, and contain about 20-80%, such as, for example, about 50-60% G+C bases. Tm's between 50 and 80° C., e.g. about 50 to 70° C. are typically preferred.

For further guidelines for PCR primer and probe design see, e.g. Dieffenbach, C. W. et al., “General Concepts for PCR Primer Design” in: PCR Primer, A Laboratory Manual, Cold Spring Harbor Laboratory Press, New York, 1995, pp. 133-155; Innis and Gelfand, “Optimization of PCRs” in: PCR Protocols, A Guide to Methods and Applications, CRC Press, London, 1994, pp. 5-11; and Plasterer, T. N. Primerselect: Primer and probe design. Methods Mol. Biol. 70:520-527 (1997), the entire disclosures of which are hereby expressly incorporated by reference.

m. Kits of the Invention

The materials for use in the methods of the present invention are suited for preparation of kits produced in accordance with well known procedures. The invention thus provides kits comprising agents, which may include gene-specific or gene-selective probes and/or primers, for quantitating the expression of the disclosed genes for predicting prognostic outcome or response to treatment. Such kits may optionally contain reagents for the extraction of RNA from tumor samples, in particular fixed paraffin-embedded tissue samples and/or reagents for RNA amplification. In addition, the kits may optionally comprise the reagent(s) with an identifying description or label or instructions relating to their use in the methods of the present invention. The kits may comprise containers (including microtiter plates suitable for use in an automated implementation of the method), each with at least one of the various reagents (typically in concentrated form) utilized in the methods, including, for example, pre-fabricated microarrays, buffers, the appropriate nucleotide triphosphates (e.g., dATP, dCTP, dGTP and dTTP; or rATP, rCTP, rGTP and UTP), reverse transcriptase, DNA polymerase, RNA polymerase, and at least one probes and primers of the present invention (e.g., appropriate length poly(T) or random primers linked to a promoter reactive with the RNA polymerase). Mathematical algorithms used to estimate or quantify prognostic or predictive information are also properly potential components of kits.

The methods provided by the present invention may also be automated in whole or in part.

n. Reports of the Invention

The methods of the present invention are suited for the preparation of reports summarizing the predictions resulting from the methods of the present invention. The invention thus provides for methods of creating reports and the reports resulting therefrom. The report may include a summary of the expression levels of the RNA transcripts or the expression products for certain genes in the cells obtained from the patients tumor tissue. The report may include a prediction that said subject has an increased likelihood of response to treatment with a particular chemotherapy or the report may include a prediction that the subject has a decreased likelihood of response to the chemotherapy. The report may include a recommendation for treatment modality such as surgery alone or surgery in combination with chemotherapy. The report may be presented in electronic format or on paper.

All aspects of the present invention may also be practiced such that a limited number of additional genes that are co-expressed with the disclosed genes, for example as evidenced by high Pearson correlation coefficients, are included in a prognostic or predictive test in addition to and/or in place of disclosed genes.

Having described the invention, the same will be more readily understood through reference to the following Example, which is provided by way of illustration, and is not intended to limit the invention in any way.

### Example 1

### Identifying Genomic Predictors of Recurrence after Adjuvant Chemotherapy

Clinical specimens were obtained from patients with operable breast cancer enrolled in clinical trial E2197 conducted by the East Coast Oncology Cooperative Group (ECOG). Goldstein and colleagues for ECOG and the North American Breast Cancer Intergroup reported the results of E2197 at ASCO 2005. (Goldstein, L. J., O'Neill, A., Sparano, J. A., Perez, E. A., Schulman, Martino, S., Davidson, N. E.: E2197: Phase III AT (doxorubucin/docetaxel) vs. AC (doxorubucin/cyclophosphamide) in the Adjuvant Treatment of Node Positive and High Risk Node Negative Breast Cancer [abstract]. Proceedings of ASCO 2005)

The expression level of each of 371 genes, including five reference genes, was determined in tumor samples obtained from breast cancer patients prior to surgical resection of the tumor and treatment of the patients with either AC or AT chemotherapy. Outcome data was available for these patients so that associations between gene expression values and outcome could be established. To form the sample for this project, the E2197 cohort was divided into 8 strata defined by hormone receptor (HR) status (estrogen receptor (ER) or progesterone receptor (PR) positive vs. both negative), axillary nodal status (positive vs. negative), and treatment arm (AT vs. AC). Within each stratum, a sub-sample was created including all recurrences with suitable tissue available and a random sample of the non-recurrences containing approximately 3.5 times as many subjects as the recurrence group.

The primary objective of the study presented in this example was to identify individual genes whose RNA expression is associated with an increased risk of recurrence of breast cancer (including all cases and controls in both AC and AT arms).

Nucleic acid from cancer cells from the patients was analyzed to measure the expression level of a test gene(s) and a reference gene(s). The expression level of the test gene(s) was then normalized to the expression level of the reference gene(s), thereby generating a normalized expression level (a “normalized expression value”) of the test gene. Normalization was carried out to correct for variation in the absolute level of gene product in a cancer cell. The cycle threshold measurement (Ct) was on a log base 2 scale, thus every unit of Ct represents a two-fold difference in gene expression.

Finally, statistical correlations were made between normalized expression values of each gene and at least one measures of clinical outcome following resection and anthracycline-based chemotherapy treatment that reflect a likelihood of (a) increased risk of recurrence of breast cancer; and (b) beneficial effect of anthracycline-based chemotherapy.

Comparative Use of AC Vs. AT does not Significantly Affect Outcome

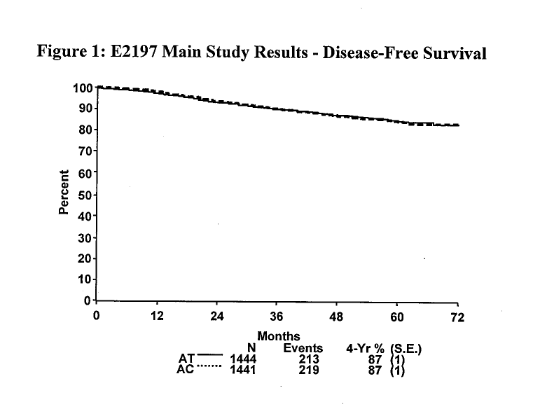

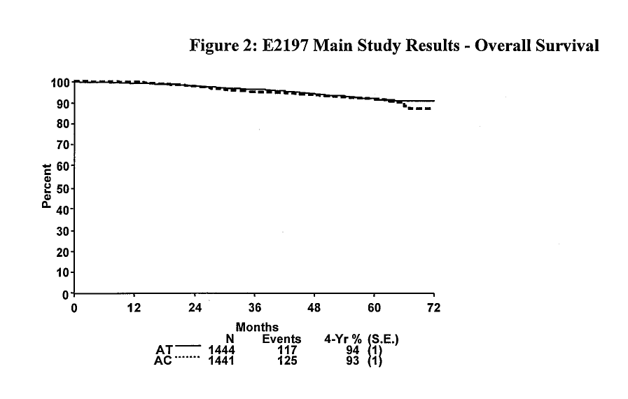

The results of the original E2197 study outlined that there is no significant difference in outcome between AC versus AT arms with regard to disease free and overall survival. See Table 1 below and FIGS. 1-2. Therefore, data from these treatment aims was combined for statistical analysis to identify prognostic genes.

TABLE 1
Results of E2197

| | AC q 3 wks × 4 | AT q 3 wks × 4 |
|---|---|---|
| | (n = 1441) | (n = 1444) |
| 4 year DFS | 87% | 87% |
| 4 year OS | 94% | 93% |

Abbreviations:
AC - doxorubicin 60 mg/m², cyclophosphamide 600 mg/m²;
AT - doxorubicin 60 mg/m², docetaxel 60 mg/m²;
DFS - disease free survival;
OSO—overall survival

Genes Associated with Clinical Outcome

Methods to predict the likelihood of recurrence in patients with invasive breast cancer treated with non-anthracycline-based treatment (e.g., tamoxifen) can be found, for example, in U.S. Pat. No. 7,056,674 and U.S. Application Publication No. 20060286565, published Dec. 21, 2006, the entire disclosures of which are expressly incorporated by reference herein.

Inclusion and Exclusion Criteria

Samples were obtained from a subset of patients enrolled in clinical trial E2197 conducted by the East Coast Oncology Cooperative (ECOG). Goldstein and colleagues for the Eastern Cooperative Oncology Group (ECOG) and the North American Breast Cancer Intergroup reported the results of E2197 at ASCO 2005 (Goldstein, L. J., O'Neill, A., Sparano, J. A., Perez, E. A., Schulman, L. N., Martino, S., Davidson, N. E.: E2197: Phase III AT (doxorubucin/docetaxel) vs. AC (doxorubucin/cyclophosphamide) in the Adjuvant Treatment of Node Positive and High Risk Node Negative Breast Cancer [abstract]. Proceedings of ASCO 2005. Abstract 512.). Genomic data was collected from 776 patients from the E2197 trial. Inclusion and exclusion criteria for the studies presented herein were as follows:

### Inclusion Criteria

Tumor samples from patients enrolled on E2197 and who meet the other eligibility criteria specific below.
Adequate tumor material available in ECOG Pathology Coordinating Center.
Patient previously consented to future cancer-related research.
Meet criteria for case and control selection outlined in statistical section.

### Exclusion Criteria

A patient that was not enrolled in E2197.
No patient sample available in the ECOG Pathology Archive
Insufficient RNA (<642 ng) for the RT-PCR analysis.
Average non-normalized C_{T} for the 5 reference genes>35.

### Probes and Primers

For each sample included in the study, the expression level for each gene listed in Table 1 was assayed by qRT-PCR as previously described in Paik et al. N. Engl. J. Med. 351: 2817-2826 (2004). Probe and primer sequences utilized in qRT-PCR assays are also provided in Table 1. Sequences for the amplicons that result from the use of the primers given in Table 2 are listed in Table 3.

Identification of Genes that are Indicators of Clinical Outcome

Statistical analyses were carried out using tumor samples from patients enrolled in the E2197 study who met the inclusion criteria. The patient samples were classified based on estrogen receptor (ER) expression (positive, negative), progesterone receptor (PR) expression (positive, negative), and human epidermal growth factor receptor 2 (HER2) expression (negative [0, 1+], weakly positive [2+], or positive [3+]) (Herceptest™, Dako USA, Carpinteria). The cut points for ER, PR, and HER2 positivity were 6.5, 5.5 and 11.5, respectively. For example, samples having a normalized ER expression of >6.5Ct were classified as ER+. These quantitative RT-PCR (e.g., qRT-PCR as described in U.S. Application Publ. No. 20050095634) cut points were established in reference to three independent prior determinations of ER, PR and HER2 expression as determined by immunohistochemistry. Tumors testing positive for either ER or PR were classified as hormone receptor positive (HR+). Because there was no significant difference between the two chemotherapy treatments (AC, AT) in the E2197 study, data from these two treatment arms were combined for this statistical analysis.

Recurrence Free Interval is defined as the time from study entry to the first evidence of breast cancer recurrence, defined as invasive breast cancer in local, regional or distant sites, including the ipsilateral breast, but excluding new primary breast cancers in the opposite breast. Follow-up for recurrence was censored at the time of death without recurrence, new primary cancer in the opposite breast, or at the time of the patient was last evaluated for recurrence.

Raw expression data expressed as C_{T} values were normalized using GAPDH, GUS, TFRC, Beta-actin, and RPLP0 as reference genes. Further analysis to identify statistically meaningful associations between expression levels of particular genes or gene sets and particular clinical outcomes was carried out using the normalized expression values.

### Example Analysis 1

A statistical analysis was performed using Univariate Cox Regression models (SAS version 9.1.3). When examining the relationship between Recurrence-Free Interval and the expression level of individual genes, the expression levels were treated as continuous variables. Follow-up for recurrence was censored at the time of death without recurrence, new primary cancer in the opposite breast, or at the time of the patient was last evaluated for recurrence. All hypothesis tests were reported using two-sided p-values, and p-values of <0.05 was considered statistically significant.

To form the sample for this project, the E2197 cohort was divided into 8 strata defined hormone status (ER or PR positive vs. both negative) using local IHC, axillary nodal status (positive vs. negative) and treatment arm (AT vs. AC). Within each stratum, a sub-sample was created including all recurrences with suitable tissue available and a random sample of the non-recurrences containing approximately 3.5 times as many subjects as the recurrence groups.

Sampling weights for each of the 16 groups in the case-control sample are defined by the number of patients in the E2197 study in that group divided by the number in the sample. In the weighted analyses, contributions to estimators and other quantities, such as partial likelihoods, are multiplied by these weights. If the patients included in the case-control sample are a random subset of the corresponding group from E2197, then the weighted estimators give consistent estimates of the corresponding quantities from the full E2197 sample. The weighted partial likelihood computed in this fashion is used for estimating hazard ratios and testing effects. This essentially gives the weighted pseudo-likelihood estimator of Chen and Lo. (K. Chen, S. H. Lo, Biometrika, 86:755-764 (1999)) The primary test for the effect of gene expression on recurrence risk was pre-specified as the weighted partial likelihood Wald test. The variance of the partial likelihood estimators is estimated using the general approach of Lin (D. Y. Lin, Biometrika, 87:37-47 (2000)), which leads to a generalization of the variance estimator from Borgan et. al. to allow subsampling of cases. (Borgan et al., Lifetime Data Analysis, 6:39-58 (2000)).

### Example Analysis 2

Statistical analyses were performed by Univariate Cox proportional hazards regression models, using stratum-specific sampling weights to calculate weighted partial likelihoods, to estimate hazard ratios, and an adjusted variance estimate was used to calculate confidence intervals and perform hypothesis tests. When examining the relationship between Recurrence-Free Interval and the expression level of individual genes, the expression levels were treated as continuous variables. All hypothesis tests were reported using the approach of Korn et al. that is used to address the multiple testing issue within each population providing strong control of the number of false discoveries. (E. L. Korn, et al., Journal of Statistical Planning and Inference, Vol. 124(2):379-398 (September 2004)) The adjusted p-values give the level of confidence that the false discovery proportion (FDP) is less than or equal to 10% in the sense that the p-value is the proportion of experiments where the true FDP is expected to exceed the stated rate. If genes with adjusted p-values<α are selected as significant, then the chance (in an average sense over replicate experiments) that the number of false discoveries is greater than the specified number is <α. In this algorithm, 500 permutations are used. For each permutation, the subject label of the gene expression levels is randomly permuted relative to the other data.

Sampling weights for each of the 16 groups in the case-control sample are defined by the number of patients in E2197 study in that group divided by the number in the sample. In the weighted analyses, contributions to estimators and other quantities, such as partial likelihoods are multiplied by these weights. (R. Gray, Lifetime Data Analysis, 9:123-138 (2003)). If the patients included in the case-control sample are a random subset of the corresponding group from E2197, then the weighted estimators give consistent estimates of the corresponding quantities from the full E2197 sample. The weighted partial likelihood computed in this fashion is used for estimating hazard ratios and testing effects. This essentially gives the weighted pseudo-likelihood estimator of Chen and Lo. (K. Chen, S. H. Lo, Biometrika, 86:755-764 (1999))

Weighted Kaplan-Meier estimators are used to estimate unadjusted survival plots and unadjusted event-free rates. The Cox proportional hazards regression model may be used to estimate covariate-adjusted survival plots and event-free rates. The empirical cumulative hazard estimate of survival, rather than the Kaplan-Meier product limit estimate, may be employed for these analyses with the Cox model.

Weighted averages, with proportions estimated using weighted averages of indicator variables, may also be used for estimating the distribution of factors and for comparing the distributions between the overall E2197 study population and the genomic sample. Tests comparing factor distributions are based on asymptotic normality of the difference in weighted averages.

### Example Analysis 3

Recurrence risk was examined in the combined HR+population (without and with adjustment for Recurrence Score [RS]), in the HR+, HER2− population, in the combined HR− population, and in the HR−, HER2− population. (Recurrence Score is described in detail in copending U.S. application Ser. No. 10/883,303 and in S. Paik, et al., N. Engl. J. Med., 351: 2817-2826 (2004).) Since the finite population sub-sampling in the genomic data set produces some dependence among observations within a stratum, the following procedure was used to generate K independent sets for cross-validation. First, the subjects within each stratum in the 776-patient genomic data set are randomly divided into K subsets (with as close to equal numbers in each group as possible), without regard to outcome (recurrence) status. Then subjects within each stratum in the 2952-patient E2197 cohort who are not in the genomic sample are randomly divided into K subsets. For each of the K subsets, sampling weights (the inverse of the sampling fraction in each of the stratum-recurrence status combinations) are recomputed using just the data in that subset. These weights are used for the sampling weights in the validation analyses. For each of the K subsets, a set of sampling weights is recomputed using the complementary (K−1)/K portion of the data. These are used as the sampling weights in the training set analyses (with different weights when each of the K subsets is omitted).

The supervised principal components procedure (SPC) is described in detail in Bair et al (Bair E, et al., J. Amer. Stat. Assoc., 101:119-137 (2006)). In this procedure, variables (genes and other factors, if considered) are ranked in terms of their significance for the outcome of interest when considered individually. The ranking here is done using Cox model Wald statistics using the adjusted variance computed using the general theory in Lin. (D. Y. Lin, Biometrika, 87:37-47 (2000)) Univariate analysis of Hazard Ratios for each single gene are calculated (no exclusions) to assess which genes are associated with higher or lower risk of recurrence. The singular value decomposition (SVD) is then applied to the design matrix formed using the m most significant of the variables. In the design matrix, each variable is first centered to have mean 0. The leading left singular vector from this decomposition (also called the leading principal component) is then used as the continuous predictor of the outcome of interest. This continuous predictor can then be analyzed as a continuous variable or grouped to form prognostic or predictive classes. The contributions (factor loadings) of the individual variables to the predictor can also be examined, and those variables with loadings smaller in magnitude than a specified threshold could be eliminated to obtain a more parsimonious predictor.

The supervised principal components procedure has several possible tuning parameters. Most important is the number in of most individually significant variables to include. The threshold for elimination of variables with low contributions is another potential tuning parameter.

A nested cross-validation approach is used. At the top level, the subjects are randomly divided into K disjoint subsets (K=5 is used in the analyses). First, the first subset is omitted. The supervised principal components procedure described below is then applied to develop a predictor or classifier using the remaining (K−1)/K portion of the data. This predictor or classifier is then applied to the omitted 1/K portion of the data to evaluate how well it predicts or classifies in an independent set (that is, the omitted 1/K portion is used as a validation sample). This process is repeated with each of the K subsets omitted in turn. The predictor/classifier developed is different for each omitted subset, but the results from the validation analyses can be aggregated to give an overall estimate of the accuracy of the procedure when applied to the full data set.

A nested cross-validation procedure is used to attempt to optimize the tuning parameters. In this procedure, K-fold cross-validation is applied to the training sample at each step of the top level cross-validation procedure. The K subsets of the training sample are generated as indicated above, except that the top-level coefficient of variation (CV) training subset (both the subjects in the genomic sample and those from E2197 not in the genomic sample) take the role of the full E2197 cohort. Within this second level of cross validation, the SPC procedure is applied to each training sample for a sequence of tuning parameter values, and the parameters are chosen to optimize some measure of performance (such as the value of the pseudo-likelihood or a Wald statistic) averaged over the validation samples. For the pseudo-likelihood, values are scaled by subtracting the log of the null model likelihood from the log pseudo likelihood for each model. The SPC procedure with these optimized tuning parameters is then applied to the full top-level CV training sample to generate the continuous predictor to evaluate on the omitted top level validation sample. Within this procedure, different optimized tuning parameters are therefore used for each step in the top-level CV procedure. Generally below, only the number of genes m is optimized in this fashion.

The primary analyses focus on the endpoint of recurrence, with follow-up censored at the time last known free of recurrence for patients without recurrence reported (including at death without recurrence). For analyses developing a prognostic classifier on the combined treatment arms, two analyses are performed on the validation sample. First, the continuous predictor is fit on the validation sample using the proportional hazards model (maximizing the weighted pseudo partial likelihood). This gives an estimated coefficient, standard error and p-value for each validation set. The average coefficient and approximate standard error over the validation sets are also computed. Second, three prognostic groups are defined using tertiles of the continuous predictor (defined on the training set), and each subject in the validation set is assigned to a prognostic group on the basis of this classifier. The weighted Kaplan-Meier estimates of the event-free probabilities are then computed within each prognostic group (within each validation set). These estimates from each tertile are then averaged over the validation sets to obtain an overall average estimate of performance. All analyses were run on 764 patients.

handling outlying gene expression values

To avoid problems with excessive influence from outlying gene expression values, substitution methods may be used for each gene. For example, two different methods were used in the above-described analyses. Specifically, for Analysis 1, the minimum value of gene expression was replaced by the 2^{nd} smallest value if the inter-quartile range (IQR) was higher than 0.3 and the difference between the two smallest values was more than 2× the IQR. Since some genes have little variation, if the IQR were less than 0.3, the minimum was replaced by the 2^{nd} smallest value if the difference between the two smallest values was more than 2×0.3. Similarly, if the largest value was more than 2×max {0.3, IQR} above the 2^{nd} largest, then the largest value was set to the same as the 2^{nd} largest. The same criteria were used to assess whether the second most extreme value had to be replaced.

For Analyses 2 and 3, if the minimum value for a gene was more than 2×max {0.3, IQR} for the gene below the 2^{nd} smallest value, then the minimum was replaced by a missing value. Similarly, if the largest value was more than 2×max {0.3, IQR} above the 2^{nd} largest, then the largest value was set to missing. Missing values then were replaced by the mean of the non-missing values for that gene.

### Example: Summary of Results

The results of these exemplar analyses are listed in Tables 3A-8B, below. The endpoint measured was Recurrence Free Interval. As used in these tables, “HR” means hazard ratio per standard deviation of gene expression. The hazard ratio is used to assess each gene's influence on the recurrence rate. If HR>1, then elevated expression of a particular gene transcript or its expression product is associated with a higher recurrence rate and a negative clinical outcome. Similarly, if HR<1, then elevated expression of a particular gene transcript or its expression product is associated with a lower recurrence rate and a beneficial clinical outcome.

TABLE 4A
(Hormone Receptor Positive (HR+), Any HER2) Genes with higher risk of
recurrence with higher expression

| | | | | | Analysis 3 |
|---|---|---|---|---|---|
| | | | Analysis 2 | | (SPC predictor |
| | Analysis 1 | | (Adjusted) | | of recurrence, |
| | (Unadjusted) | | | Korn adj. | adj. for RS) |
| gene | HR | p value | HR | p value | HR |
| NUSAP1 | 1.587 | 3.442E−07 | 1.5872 | 0.000 | 1.5872 |
| DEPDC1 | 1.672 | 2.063E−06 | 1.6720 | 0.000 | — |
| TOP2A | 1.476 | 9.244E−06 | 1.4722 | 0.000 | — |
| AURKB | 1.498 | 0.0000384 | 1.4978 | 0.002 | — |
| BIRC5 | 1.422 | 0.0000422 | 1.3951 | 0.002 | — |
| GAPDH | 2.350 | 0.0000516 | 2.3467 | 0.002 | 2.3467 |
| PTTG1 | 1.568 | 0.0000788 | 1.5683 | 0.002 | — |
| CDC2 | 1.437 | 0.0001058 | 1.4376 | 0.002 | — |
| KIFC1 | 1.501 | 0.0001395 | 1.5008 | 0.004 | — |
| MKI67 | 1.527 | 0.0001604 | 1.4963 | 0.004 | — |
| BUB1B | 7.128 | 0.0002369 | 7.1278 | 0.002 | — |
| PLK1 | 1.414 | 0.0003211 | 1.4134 | 0.004 | — |
| BUB1 | 1.464 | 0.0003938 | 1.4637 | 0.004 | — |
| MAD2L1 | 1.513 | 0.0004665 | 1.5129 | 0.004 | — |
| TACC3 | 1.609 | 0.0005893 | 1.6080 | 0.006 | — |
| CENPF | 1.411 | 0.0006091 | 1.4106 | 0.006 | — |
| NEK2 | 1.437 | 0.0008261 | 1.4376 | 0.010 | — |
| CDC20 | 1.352 | 0.0011946 | 1.3512 | 0.016 | — |
| TYMS | 1.493 | 0.0013813 | 1.4933 | 0.020 | — |
| TTK | 1.418 | 0.0017844 | 1.4176 | 0.024 | — |
| CENPA | 1.411 | 0.0017901 | 1.4106 | 0.030 | — |
| FOXM1 | 1.418 | 0.0019025 | 1.4176 | 0.042 | — |
| TPX2 | 1.348 | 0.0020794 | — | ** | — |
| CDCA8 | 1.420 | 0.0023514 | 1.4205 | 0.034 | — |
| MYBL2 | 1.299 | 0.0030240 | — | ** | — |
| CCNB1 | 1.595 | 0.0051888 | — | ** | — |
| KIF11 | 1.371 | 0.0052941 | — | ** | — |
| ZWILCH | 1.634 | 0.0057525 | — | ** | — |
| GPR56 | 1.532 | 0.0060626 | — | ** | — |
| ZWINT | 1.358 | 0.0081847 | — | ** | — |
| KIF2C | 1.336 | 0.0101481 | — | ** | — |
| ESPL1 | 1.287 | 0.0111571 | — | ** | — |
| GRB7 | 1.259 | 0.0120361 | — | ** | — |
| HSP90AA1 | 1.627 | 0.0129320 | — | ** | — |
| CHGA | 1.159 | 0.0153291 | — | ** | 1.1584 |
| PGK1 | 1.646 | 0.0158863 | — | ** | — |
| MMP12 | 1.271 | 0.0164373 | — | ** | — |
| MAGEA2 | 1.369 | 0.0173455 | — | ** | — |
| SLC7A5 | 1.250 | 0.0183518 | — | ** | — |
| CCND1 | 1.233 | 0.0202102 | — | ** | 1.2324 |
| BRCA2 | 1.549 | 0.0276566 | — | ** | — |
| AURKA | 1.394 | 0.0402357 | — | ** | — |
| RAD54L | 1.302 | 0.0450509 | — | ** | — |
| ERBB2 | 1.199 | 0.0470906 | — | ** | — |

** Korn adj. p value >0.05

TABLE 4B
(Hormone Receptor Positive (HR+), Any HER2) Genes with lower risk of
recurrence with higher expression

| | | | | | Analysis 3 |
|---|---|---|---|---|---|
| | | | Analysis 2 | | (SPC predictor |
| | Analysis 1 | | (Adjusted) | | of recurrence, |
| | (Unadjusted) | | | Korn Adj | adj. for RS) |
| gene | HR | p value | HR | p value | HR |
| PFDN5 | 0.601 | 2.014E−07 | — | ** | — |
| STK11 | 0.399 | 4.404E−07 | 0.3985 | 0.000 | 0.3985 |
| SCUBE2 | 0.805 | 9.808E−06 | 0.8138 | 0.002 | — |
| ZW10 | 0.430 | 0.0000102 | 0.4304 | 0.002 | — |
| RASSF1 | 0.464 | 0.0000536 | 0.4639 | 0.002 | 0.4639 |
| ID1 | 0.583 | 0.0000757 | 0.5827 | 0.004 | — |
| ABCA9 | 0.702 | 0.0001145 | 0.7026 | 0.002 | — |
| GSTM1 | 0.713 | 0.0001248 | 0.7218 | 0.004 | — |
| PGR | 0.815 | 0.0001459 | 0.8138 | 0.004 | — |
| PRDM2 | 0.620 | 0.0001680 | 0.6206 | 0.004 | — |
| RELA | 0.496 | 0.0002484 | 0.4956 | 0.004 | 0.4956 |
| FHIT | 0.661 | 0.0002685 | 0.6610 | 0.004 | — |
| ERCC1 | 0.497 | 0.0002786 | 0.4971 | 0.004 | — |
| ESR1 | 0.814 | 0.0004879 | 0.8245 | 0.032 | — |
| AKT3 | 0.629 | 0.0007087 | 0.6288 | 0.006 | — |
| SLC1A3 | 0.614 | 0.0011054 | 0.6139 | 0.016 | 0.6139 |
| CSF1 | 0.559 | 0.0011623 | 0.5593 | 0.012 | — |
| AKT2 | 0.491 | 0.0013291 | 0.4916 | 0.016 | — |
| PECAM1 | 0.614 | 0.0014795 | 0.6145 | 0.022 | — |
| PIK3C2A | 0.533 | 0.0015982 | 0.5331 | 0.022 | — |
| MAPT | 0.822 | 0.0016329 | 0.8220 | 0.032 | — |
| MRE11A | 0.585 | 0.0018207 | 0.5851 | 0.030 | — |
| MYH11 | 0.788 | 0.0018833 | 0.7882 | 0.022 | — |
| NPC2 | 0.524 | 0.0019133 | 0.5241 | 0.024 | — |
| GADD45B | 0.614 | 0.0019389 | 0.6145 | 0.022 | — |
| PTPN21 | 0.706 | 0.0019855 | 0.7061 | 0.032 | — |
| COL1A1 | 0.741 | 0.0020877 | 0.7408 | 0.034 | — |
| ROCK1 | 0.550 | 0.0025041 | 0.5499 | 0.034 | — |
| ABAT | 0.793 | 0.0025380 | 0.7937 | 0.034 | — |
| COL1A2 | 0.769 | 0.0028633 | 0.7408 | 0.034 | — |
| PIM2 | 0.713 | 0.0029396 | 0.7132 | 0.032 | 0.7132 |
| CDKN1C | 0.697 | 0.0031276 | 0.6970 | 0.044 | — |
| SEMA3F | 0.720 | 0.0032523 | — | ** | — |
| PMS2 | 0.538 | 0.0035689 | 0.5379 | 0.050 | — |
| MGC52057 | 0.720 | 0.0037128 | 0.7204 | 0.024 | — |
| FAS | 0.674 | 0.0037460 | 0.6744 | 0.050 | — |
| ELP3 | 0.553 | 0.0040295 | — | ** | — |
| BAX | 0.506 | 0.0046591 | — | ** | — |
| PRKCH | 0.637 | 0.0050308 | — | ** | — |
| CD247 | 0.735 | 0.0052363 | — | ** | 0.7349 |
| NME6 | 0.615 | 0.0053468 | — | ** | — |
| GGPS1 | 0.621 | 0.0056877 | — | ** | — |
| ACTR2 | 0.459 | 0.0057060 | — | ** | 0.4593 |
| STAT3 | 0.715 | 0.0058238 | 0.7153 | 0.008 | — |
| BIRC3 | 0.756 | 0.0065975 | — | ** | 0.7558 |
| ABCB1 | 0.581 | 0.0066902 | — | ** | — |
| RPLP0 | 0.439 | 0.0067008 | — | ** | — |
| CLU | 0.771 | 0.0068700 | — | ** | — |
| FYN | 0.652 | 0.0068877 | — | ** | — |
| MAP4 | 0.512 | 0.0076104 | — | ** | — |
| IGFBP2 | 0.776 | 0.0081400 | — | ** | — |
| RELB | 0.695 | 0.0081769 | — | ** | — |
| WNT5A | 0.700 | 0.0084988 | — | ** | — |
| LIMK1 | 0.634 | 0.0088995 | — | ** | — |
| CYP1B1 | 0.727 | 0.0105903 | — | ** | — |
| LILRB1 | 0.721 | 0.0106359 | — | ** | — |
| PPP2CA | 0.559 | 0.0111439 | — | ** | — |
| ABCG2 | 0.660 | 0.0115255 | — | ** | — |
| EGFR | 0.754 | 0.0124036 | — | ** | — |
| BBC3 | 0.719 | 0.0139470 | — | ** | — |
| TNFRSF10B | 0.700 | 0.0144998 | — | ** | — |
| CYP2C8 | 0.483 | 0.0145393 | — | ** | — |
| CTNNB1 | 0.611 | 0.0166914 | — | ** | — |
| SGK3 | 0.757 | 0.0168533 | — | ** | — |
| BIRC4 | 0.625 | 0.0172627 | — | ** | — |
| MAPK3 | 0.710 | 0.0202294 | — | ** | — |
| ARAF | 0.657 | 0.0202552 | — | ** | — |
| IRS1 | 0.776 | 0.0208563 | — | ** | — |
| APOD | 0.852 | 0.0213176 | — | ** | — |
| CAV1 | 0.650 | 0.0213454 | — | ** | — |
| MMP2 | 0.827 | 0.0217710 | — | ** | — |
| KNS2 | 0.659 | 0.0230028 | — | ** | — |
| PIM1 | 0.756 | 0.0235704 | — | ** | — |
| VCAM1 | 0.742 | 0.0237609 | — | ** | — |
| FASLG | 0.489 | 0.0240244 | — | ** | 0.4892 |
| MAD1L1 | 0.667 | 0.0261089 | — | ** | — |
| RPL37A | 0.592 | 0.0265180 | — | ** | — |
| FLAD1 | 0.633 | 0.0266318 | — | ** | — |
| MAPK14 | 0.591 | 0.0272216 | — | ** | — |
| CDKN1B | 0.694 | 0.0272468 | — | ** | — |
| DICER1 | 0.748 | 0.0286966 | — | ** | — |
| PDGFRB | 0.759 | 0.0288255 | — | ** | — |
| NFKB1 | 0.643 | 0.0309325 | — | ** | — |
| VEGFB | 0.757 | 0.0328536 | — | ** | — |
| FUS | 0.651 | 0.0363513 | — | ** | — |
| SNAI2 | 0.771 | 0.0380711 | — | ** | — |
| TUBD1 | 0.749 | 0.0405564 | — | ** | — |
| CAPZA1 | 0.558 | 0.0407558 | — | ** | — |
| BCL2 | 0.782 | 0.0415340 | — | ** | — |
| GATA3 | 0.851 | 0.0421418 | — | ** | — |
| STK10 | 0.724 | 0.0436867 | — | ** | — |
| CNN1 | 0.816 | 0.0437974 | — | ** | — |
| SRI | 0.602 | 0.0438974 | — | ** | — |
| FOXA1 | 0.863 | 0.0440180 | — | ** | — |
| GBP2 | 0.741 | 0.0447335 | — | ** | — |
| RPN2 | 0.765 | 0.0447404 | — | ** | — |
| ANXA4 | 0.745 | 0.0489155 | — | ** | — |
| MCL1 | 0.680 | 0.0494269 | — | ** | — |
| GBP1 | — | — | — | ** | 0.8428 |
| STAT1 | — | — | — | ** | 0.8294 |
| LILRB1 | — | — | — | ** | 0.7211 |
| ZW10 | — | — | — | ** | 0.4304 |

** Korn adj. p value >0.05

TABLE 5A
(Hormone Receptor Negative (HR−), Any HER2) Genes with higher risk
of recurrence with higher expression

| | | | | | Analysis 3 (SPC |
|---|---|---|---|---|---|
| | | | | | predictor |
| | Analysis 1 | | Analysis 2 | | of recurrence, |
| | (Unadjusted) | | (Adjusted) | | adj. for RS) |
| gene | HR | p value | HR | Korn adj. p | HR |
| MYBL2 | 1.695 | 0.0019573 | — | ** | 1.7006 |
| GPR126 | 1.380 | 0.0068126 | — | ** | — |
| GPR56 | 1.358 | 0.0131494 | — | ** | — |
| GRB7 | 1.154 | 0.0190295 | — | ** | — |
| CKAP1 | 1.515 | 0.0216536 | — | ** | — |
| NEK2 | 1.331 | 0.0219334 | — | ** | — |
| L1CAM | 1.184 | 0.0231607 | — | ** | — |
| TUBA3 | 1.383 | 0.0294187 | — | ** | — |
| LAPTM4B | 1.300 | 0.0381478 | — | ** | — |
| TBCE | 1.468 | 0.0401742 | — | ** | — |

** Korn adj. p value >0.05

TABLE 5B
(Hormone Receptor Negative (HR−), Any HER2) Genes with lower risk
of recurrence with higher expression

| | | | | | Analysis 3 (SPC |
|---|---|---|---|---|---|
| | | | Analysis 2 | | predictor of |
| | Analysis 1 | | (Adjusted) | | recurrence, adj. |
| | (Unadjusted) | | | Korn adj. | for RS) |
| gene | HR | p value | HR | p value | HR |
| CD68 | 0.652 | 0.0000543 | — | ** | 0.6525 |
| ACTR2 | 0.695 | 0.0000610 | — | ** | — |
| ESR2 | 0.142 | 0.0003262 | 0.1418 | 0.000 | 0.1418 |
| BIRC3 | 0.710 | 0.0003312 | 0.7103 | 0.008 | 0.7103 |
| PIM2 | 0.721 | 0.0003382 | 0.7211 | 0.000 | 0.7211 |
| VCAM1 | 0.716 | 0.0004912 | 0.7161 | 0.014 | 0.7161 |
| RELB | 0.572 | 0.0005896 | 0.5718 | 0.014 | 0.5718 |
| IL7 | 0.606 | 0.0007567 | 0.6053 | 0.014 | 0.6053 |
| APOC1 | 0.726 | 0.0011094 | 0.7254 | 0.042 | 0.7254 |
| XIST | 0.730 | 0.0013534 | — | ** | 0.7298 |
| CST7 | 0.727 | 0.0020814 | — | ** | 0.7276 |
| GBP2 | 0.695 | 0.0022400 | — | ** | 0.6956 |
| PRKCH | 0.602 | 0.0022573 | — | ** | 0.6023 |
| LILRB1 | 0.706 | 0.0029297 | — | ** | 0.07061 |
| FASLG | 0.458 | 0.0041478 | 0.4584 | 0.022 | 0.4584 |
| CSF1 | 0.676 | 0.0042618 | — | ** | 0.6757 |
| CD247 | 0.734 | 0.0042817 | — | ** | 0.7334 |
| BIN1 | 0.711 | 0.0043244 | — | ** | 0.7103 |
| WNT5A | 0.483 | 0.0045915 | — | ** | — |
| PRKCA | 0.730 | 0.0051254 | — | ** | 0.7298 |
| STAT1 | 0.723 | 0.0061824 | — | ** | 0.7233 |
| PGR | 0.604 | 0.0068937 | — | ** | 0.6169 |
| IRAK2 | 0.634 | 0.0073992 | — | ** | 0.6338 |
| CYBA | 0.711 | 0.0077397 | — | ** | 0.7103 |
| SCUBE2 | 0.783 | 0.0087744 | — | ** | 0.7851 |
| ERCC1 | 0.505 | 0.0089315 | — | ** | — |
| CAPZA1 | 0.574 | 0.0091684 | — | ** | 0.5735 |
| IL2RA | 0.634 | 0.0098419 | — | ** | 0.6338 |
| GBP1 | 0.779 | 0.0104451 | — | ** | 0.7788 |
| PECAM1 | 0.694 | 0.0130612 | — | ** | 0.6942 |
| CCL2 | 0.729 | 0.0136238 | — | ** | 0.7291 |
| STAT3 | 0.530 | 0.0152545 | — | ** | 0.5305 |
| NFKB1 | 0.596 | 0.0161377 | — | ** | 0.5963 |
| CD14 | 0.692 | 0.0161533 | — | ** | 0.6921 |
| TNFSF10 | 0.782 | 0.0167007 | — | ** | 0.7819 |
| TFF1 | 0.811 | 0.0197258 | — | ** | — |
| GADD45A | 0.720 | 0.0228062 | — | ** | — |
| SLC1A3 | 0.769 | 0.0228194 | — | ** | — |
| BAD | 0.645 | 0.0230521 | — | ** | — |
| FYN | 0.745 | 0.0245100 | — | ** | 0.7453 |
| CTSL | 0.722 | 0.0247385 | — | ** | — |
| DIAPH1 | 0.623 | 0.0251948 | — | ** | — |
| ABAT | 0.737 | 0.0277218 | — | ** | — |
| ABCG2 | 0.544 | 0.0300971 | — | ** | — |
| PRKCG | 0.349 | 0.0314412 | — | ** | — |
| PLD3 | 0.654 | 0.0332019 | — | ** | — |
| KNTC1 | 0.742 | 0.0335689 | — | ** | — |
| GSR | 0.712 | 0.0345107 | — | ** | — |
| CSAG2 | 0.840 | 0.0350118 | — | ** | — |
| CHFR | 0.671 | 0.0380636 | — | ** | — |
| MSH3 | 0.700 | 0.0460279 | — | ** | — |
| TPT1 | 0.713 | 0.0483077 | — | ** | — |
| BAX | 0.601 | 0.0488665 | — | ** | — |
| CLU | 0.855 | 0.0492894 | — | ** | — |
| ABCA9 | 0.809 | 0.0494329 | — | ** | — |
| STK10 | 0.737 | 0.0498826 | — | ** | — |
| APOE | — | — | — | ** | 0.8353 |

** Korn adj. p value >0.05

TABLE 6A
(Hormone Receptor Positive (HR+), HER2 Negative (HER2−)) Genes
with higher risk of recurrence with higher expression

| | |
|---|---|
| | Analysts 2 |
| Analysis 1 | (Adjusted) |

| | |
|---|---|
| (Unadjusted) | Korn adj. |

| gene | HR | p value | HR | p value |
|---|---|---|---|---|
| NUSAP1 | 1.640 | 5.151E−07 | 1.6703 | 0.000 |
| DEPDC1 | 1.671 | 9.82E−06 | 1.6703 | 0.000 |
| TOP2A | 1.554 | 0.0000134 | 1.5543 | 0.000 |
| AURKB | 1.591 | 0.0000153 | 1.5904 | 0.000 |
| GAPDH | 2.726 | 0.0000175 | 2.5498 | 0.004 |
| KIFC1 | 1.586 | 0.0000699 | 1.5857 | 0.004 |
| BIRC5 | 1.420 | 0.0002009 | 1.3979 | 0.008 |
| PLK1 | 1.446 | 0.0003978 | 1.4463 | 0.008 |
| TYMS | 1.588 | 0.0004860 | 1.5872 | 0.008 |
| PTTG1 | 1.543 | 0.0006240 | 1.5434 | 0.008 |
| CENPF | 1.453 | 0.0007597 | 1.4521 | 0.010 |
| MKI67 | 1.522 | 0.0007619 | 1.4933 | 0.032 |
| CDC2 | 1.405 | 0.0008394 | 1.4049 | 0.016 |
| BUB1B | 6.708 | 0.0008669 | 6.6993 | 0.006 |
| FOXM1 | 1.477 | 0.0012756 | — | ** |
| ESPL1 | 1.418 | 0.0013859 | 1.4191 | 0.030 |
| TACC3 | 1.605 | 0.0014749 | 1.6048 | 0.032 |
| NEK2 | 1.448 | 0.0018204 | 1.4477 | 0.040 |
| MAD2L1 | 1.480 | 0.0023430 | 1.4799 | 0.042 |
| TTK | 1.442 | 0.0023457 | — | ** |
| BUB1 | 1.426 | 0.0024859 | 1.4262 | 0.044 |
| MYBL2 | 1.344 | 0.0033909 | — | ** |
| TPX2 | 1.361 | 0.0037780 | — | ** |
| CENPA | 1.407 | 0.0047243 | — | ** |
| CDC20 | 1.335 | 0.0055217 | — | ** |
| CDCA8 | 1.404 | 0.0060377 | — | ** |
| CCND1 | 1.323 | 0.0063660 | — | ** |
| ZWINT | 1.414 | 0.0084107 | — | ** |
| CCNB1 | 1.639 | 0.0085470 | — | ** |
| ZWILCH | 1.649 | 0.0106632 | — | ** |
| CENPE | 1.715 | 0.0106842 | — | ** |
| KIF11 | 1.371 | 0.0113708 | — | ** |
| BRCA1 | 1.430 | 0.0150194 | — | ** |
| CHGA | 1.157 | 0.0257914 | — | ** |
| HSPA5 | 1.982 | 0.0264599 | — | ** |
| MAGEA2 | 1.394 | 0.0268474 | — | ** |
| KIF2C | 1.304 | 0.0315403 | — | ** |
| RAD54L | 1.346 | 0.0368698 | — | ** |
| CA9 | 1.213 | 0.0420931 | — | ** |

** Korn adj. p value >0.05

TABLE 6B
(Hormone Receptor Positive (HR+), HER2 Negative (HER2−)) Genes
with lower risk of recurrence with higher expression

| | |
|---|---|
| | Analysis 2 |
| Analysis 1 | (Adjusted) |

| | |
|---|---|
| (Unadjusted) | Korn adj. |

| gene | HR | p value | HR | p value |
|---|---|---|---|---|
| STK11 | 0.407 | 9.027E−06 | 0.4070 | 0.002 |
| ACTR2 | 0.283 | 0.0000563 | 0.2825 | 0.004 |
| ZW10 | 0.446 | 0.0000654 | 0.4466 | 0.002 |
| RASSF1 | 0.451 | 0.0000826 | 0.4507 | 0.004 |
| ID1 | 0.586 | 0.0001968 | 0.5863 | 0.008 |
| MMP2 | 0.719 | 0.0002211 | 0.7189 | 0.010 |
| NPC2 | 0.435 | 0.0003018 | — | ** |
| GADD45B | 0.530 | 0.0003473 | 0.5305 | 0.006 |
| COL1A2 | 0.728 | 0.0004219 | 0.7283 | 0.016 |
| SLC1A3 | 0.568 | 0.0006126 | 0.5684 | 0.014 |
| SCUBE2 | 0.829 | 0.0006362 | — | ** |
| RELA | 0.486 | 0.0006912 | 0.4863 | 0.020 |
| PTPN21 | 0.658 | 0.0007339 | 0.6577 | 0.016 |
| GSTM1 | 0.713 | 0.0009258 | 0.7225 | 0.040 |
| COL1A1 | 0.710 | 0.0009907 | 0.7096 | 0.026 |
| PRDM2 | 0.625 | 0.0011046 | 0.6250 | 0.018 |
| AKT3 | 0.612 | 0.0011152 | 0.6114 | 0.026 |
| CSF1 | 0.507 | 0.0012382 | 0.5071 | 0.020 |
| FAS | 0.615 | 0.0012471 | 0.6145 | 0.020 |
| ABCA9 | 0.726 | 0.0012999 | 0.7261 | 0.028 |
| ROCK1 | 0.474 | 0.0018953 | 0.4738 | 0.044 |
| VCAM1 | 0.650 | 0.0022313 | — | ** |
| PIM2 | 0.686 | 0.0023819 | 0.6859 | 0.040 |
| CD247 | 0.685 | 0.0024093 | 0.6845 | 0.036 |
| PECAM1 | 0.605 | 0.0024170 | — | ** |
| PIK3C2A | 0.501 | 0.0026879 | — | ** |
| FYN | 0.597 | 0.0034648 | — | ** |
| CYP2C8 | 0.353 | 0.0034744 | — | ** |
| MAP4 | 0.456 | 0.0036702 | — | ** |
| PPP2CA | 0.478 | 0.0039174 | — | ** |
| CDKN1C | 0.677 | 0.0039353 | — | ** |
| PRKCH | 0.621 | 0.0043849 | — | ** |
| ERCC1 | 0.546 | 0.0048268 | — | ** |
| BAX | 0.471 | 0.0050208 | — | ** |
| PDGFRB | 0.692 | 0.0053268 | — | ** |
| STK10 | 0.545 | 0.0054089 | — | ** |
| CXCR4 | 0.670 | 0.0072433 | — | ** |
| FHIT | 0.714 | 0.0073859 | — | ** |
| ELP3 | 0.544 | 0.0076927 | — | ** |
| ITGB1 | 0.447 | 0.0086595 | — | ** |
| PGR | 0.853 | 0.0088435 | — | ** |
| BIRC3 | 0.742 | 0.0090302 | — | ** |
| RPN2 | 0.734 | 0.0091664 | — | ** |
| MYH11 | 0.799 | 0.0093865 | — | ** |
| NME6 | 0.623 | 0.0095259 | — | ** |
| GGPS1 | 0.620 | 0.0099960 | — | ** |
| CAPZA1 | 0.444 | 0.0105944 | — | ** |
| MRE11A | 0.622 | 0.0112118 | — | ** |
| BIRC4 | 0.581 | 0.0114118 | — | ** |
| ABAT | 0.814 | 0.0117097 | — | ** |
| TNFRSF10B | 0.669 | 0.0118729 | — | ** |
| ACTB | 0.490 | 0.0119353 | — | ** |
| SEMA3F | 0.733 | 0.0122670 | — | ** |
| WNT5A | 0.683 | 0.0124795 | — | ** |
| EGFR | 0.728 | 0.0128218 | — | ** |
| PIM1 | 0.713 | 0.0130874 | — | ** |
| RELB | 0.698 | 0.0151985 | — | ** |
| LILRB1 | 0.712 | 0.0153494 | — | ** |
| S100A10 | 0.638 | 0.0154250 | — | ** |
| MAD1L1 | 0.613 | 0.0162166 | — | ** |
| LIMK1 | 0.640 | 0.0166726 | — | ** |
| SNAI2 | 0.725 | 0.0179736 | — | ** |
| CYP1B1 | 0.728 | 0.0181752 | — | ** |
| CTNNB1 | 0.586 | 0.0187559 | — | ** |
| KNS2 | 0.617 | 0.0191798 | — | ** |
| STAT3 | 0.741 | 0.0208400 | — | ** |
| ESR1 | 0.851 | 0.0220104 | — | ** |
| CCL2 | 0.734 | 0.0229520 | — | ** |
| BBC3 | 0.713 | 0.0235615 | — | ** |
| AKT2 | 0.577 | 0.0243569 | — | ** |
| MAPK14 | 0.522 | 0.0245184 | — | ** |
| CALD1 | 0.666 | 0.0256356 | — | ** |
| FASLG | 0.408 | 0.0256649 | — | ** |
| ABCG2 | 0.668 | 0.0265022 | — | ** |
| CAV1 | 0.623 | 0.0276134 | — | ** |
| ABCB1 | 0.620 | 0.0276165 | — | ** |
| HIF1A | 0.620 | 0.0284583 | — | ** |
| MAPK3 | 0.698 | 0.0284801 | — | ** |
| GBP1 | 0.773 | 0.0300251 | — | ** |
| PMS2 | 0.601 | 0.0302953 | — | ** |
| RHOC | 0.668 | 0.0324165 | — | ** |
| PRKCD | 0.642 | 0.0340220 | — | ** |
| ANXA4 | 0.719 | 0.0353797 | — | ** |
| GBP2 | 0.700 | 0.0356809 | — | ** |
| CLU | 0.805 | 0.0376120 | — | ** |
| IL7 | 0.725 | 0.0390326 | — | ** |
| COL6A3 | 0.826 | 0.0436450 | — | ** |
| HSPA1L | 0.276 | 0.0478052 | — | ** |
| MGC52057 | 0.791 | 0.0478901 | — | ** |

** Korn adj. p value >0.05

TABLE 7A
(Hormone Receptor Negative (HR−), HER2 Negative (HER2−)) Genes
with higher risk of recurrence with higher expression

| | | | | | Analysis 3 (SPC |
|---|---|---|---|---|---|
| | | | Analysis 2 | | predictor of |
| | Analysis 1 | | (Adjusted) | | recurrence, adj. |
| | (Unadjusted) | | | Korn adj. | for RS) |
| gene | HR | p value | HR | p value | HR |
| GRB7 | 1.906 | 0.0000224 | 1.8908 | 0.000 | 1.8908 |
| GAGE1 | 1.648 | 0.0043470 | — | ** | 1.6487 |
| GPR126 | 1.442 | 0.0055425 | — | ** | — |
| CYP2C8 | 2.363 | 0.0083138 | — | ** | — |
| NEK2 | 1.460 | 0.0091325 | — | ** | — |
| KRT19 | 1.354 | 0.0156629 | — | ** | — |
| MYBL2 | 1.604 | 0.0194619 | — | ** | — |
| MYC | 1.379 | 0.0299718 | — | ** | — |
| CKAP1 | 1.560 | 0.0304028 | — | ** | — |
| TUBA3 | 1.423 | 0.0311994 | — | ** | — |
| L1CAM | 1.197 | 0.0331190 | — | ** | — |
| ERBB2 | 1.381 | 0.0362432 | — | ** | — |
| CCND1 | 1.259 | 0.0499983 | — | ** | — |

** Korn adj. p value >0.05

TABLE 7B
(Hormone Receptor Negative (HR−), HER2 Negative (HER2−)) Genes
with lower risk of recurrence with higher expression

| | | | Analysis 2 | | Analysis 3 |
|---|---|---|---|---|---|
| | Analysis 1 | | (Adjusted) | | (SPC predictor |
| | (Unadjusted) | | | Korn adj. | of recurrence, |
| gene | HR | p value | HR | p value | adj. for RS) |
| CD68 | 0.592 | 2.116E−07 | 0.5945 | 0.002 | 0.5945 |
| ACTR2 | 0.656 | 1.36E−06 | — | ** | — |
| XIST | 0.654 | 0.0000296 | 0.6544 | 0.022 | 0.6544 |
| APOC1 | 0.637 | 0.0000523 | 0.6364 | 0.000 | 0.6364 |
| BIRC3 | 0.662 | 0.0001202 | 0.6623 | 0.002 | 0.6623 |
| ESR2 | 0.084 | 0.0001243 | 0.0840 | 0.000 | 0.0840 |
| PIM2 | 0.671 | 0.0001318 | 0.6710 | 0.002 | 0.6710 |
| SLC1A3 | 0.620 | 0.0002156 | 0.6200 | 0.014 | 0.6200 |
| BIN1 | 0.626 | 0.0002500 | 0.6256 | 0.012 | 0.6256 |
| PRKCH | 0.502 | 0.0004783 | 0.5021 | 0.014 | 0.5021 |
| LILRB1 | 0.639 | 0.0006606 | 0.6395 | 0.012 | 0.6395 |
| CST7 | 0.671 | 0.0006776 | 0.6710 | 0.018 | 0.6710 |
| RELB | 0.526 | 0.0007769 | 0.5262 | 0.020 | 0.5262 |
| VCAM1 | 0.700 | 0.0009248 | 0.6998 | 0.026 | 0.6998 |
| CAPZA1 | 0.449 | 0.0011732 | — | ** | 0.4489 |
| GBP2 | 0.635 | 0.0011762 | 0.6351 | 0.034 | 0.6351 |
| PLD3 | 0.523 | 0.0013142 | — | ** | 0.5231 |
| IRAK2 | 0.512 | 0.0016339 | — | ** | 0.5117 |
| IL7 | 0.584 | 0.0018037 | 0.5839 | 0.032 | 0.5839 |
| CTSL | 0.660 | 0.0026678 | — | ** | — |
| CSF1 | 0.633 | 0.0027615 | — | ** | 0.6325 |
| CD247 | 0.688 | 0.0028163 | — | ** | 0.6880 |
| FASLG | 0.356 | 0.0030677 | 0.3563 | 0.014 | 0.3563 |
| GNS | 0.483 | 0.0035073 | — | ** | 0.4834 |
| CYBA | 0.664 | 0.0044996 | — | ** | 0.6643 |
| NFKB1 | 0.502 | 0.0046224 | — | ** | 0.5016 |
| DIAPH1 | 0.512 | 0.0047600 | — | ** | 0.5117 |
| IL2RA | 0.558 | 0.0052120 | — | ** | 0.5577 |
| STAT1 | 0.682 | 0.0053202 | — | ** | 0.6818 |
| PECAM1 | 0.628 | 0.0056997 | — | ** | 0.6281 |
| PLAU | 0.646 | 0.0059777 | — | ** | 0.6466 |
| ERCC1 | 0.432 | 0.0067565 | — | ** | 0.4321 |
| ABCC3 | 0.648 | 0.0074137 | — | ** | 0.6479 |
| WNT5A | 0.455 | 0.0074176 | — | ** | — |
| CCL2 | 0.682 | 0.0074775 | — | ** | 0.6818 |
| CD14 | 0.639 | 0.0087980 | — | ** | — |
| MMP9 | 0.763 | 0.0089472 | — | ** | — |
| BAD | 0.568 | 0.0099167 | — | ** | — |
| GBP1 | 0.749 | 0.0100726 | — | ** | — |
| GADD45A | 0.658 | 0.0108479 | — | ** | — |
| CDKN1A | 0.632 | 0.0110232 | — | ** | — |
| ECGF1 | 0.702 | 0.0111429 | — | ** | — |
| STK10 | 0.654 | 0.0116239 | — | ** | — |
| PRKCA | 0.731 | 0.0121695 | — | ** | — |
| MMP2 | 0.738 | 0.0129347 | — | ** | — |
| GSR | 0.625 | 0.0164580 | — | ** | — |
| PLAUR | 0.656 | 0.0194483 | — | ** | — |
| BAX | 0.482 | 0.0221901 | — | ** | — |
| PRKCG | 0.263 | 0.0223421 | — | ** | — |
| FYN | 0.725 | 0.0227879 | — | ** | 0.7254 |
| APOE | 0.799 | 0.0229649 | — | ** | 0.7993 |
| ACTB | 0.502 | 0.0241365 | — | ** | — |
| GLRX | 0.271 | 0.0256879 | — | ** | — |
| TYRO3 | 0.627 | 0.0270209 | — | ** | — |
| SCUBE2 | 0.780 | 0.0271519 | — | ** | — |
| STAT3 | 0.517 | 0.0281809 | — | ** | — |
| CLU | 0.827 | 0.0283483 | — | ** | — |
| PRDM2 | 0.721 | 0.0287352 | — | ** | — |
| KALPHA1 | 0.549 | 0.0345194 | — | ** | — |
| RELA | 0.591 | 0.0372553 | — | ** | — |
| KNS2 | 0.634 | 0.0391500 | — | ** | — |
| COL1A1 | 0.791 | 0.0405529 | — | ** | — |
| MET | 0.714 | 0.0415376 | — | ** | — |
| NPC2 | 0.632 | 0.0415918 | — | ** | — |
| SNAI2 | 0.734 | 0.0420155 | — | ** | — |
| ABCG2 | 0.529 | 0.0456976 | — | ** | — |
| GPX1 | 0.614 | 0.0459149 | — | ** | — |
| PGR | 0.632 | 0.0459791 | — | ** | — |
| IGFBP3 | 0.744 | 0.0465884 | — | ** | — |
| TNFSF10 | 0.793 | 0.0486299 | — | ** | — |

** Korn adj. p value >0.05

TABLE 8A
(Hormone Receptor Positive (HR+), HER2 Positive (HER2+)) Genes
with higher risk of recurrence with higher expression

| | |
|---|---|
| | Analysis 2 |
| Analysis 1 | (Adjusted) |

| | |
|---|---|
| (Unadjusted) | Korn adj. |

| gene | HR | p value | HR | p value |
|---|---|---|---|---|
| ERBB2 | 1.864 | 0.0014895 | 1.8814 | 0.00 |
| TUBB3 | 1.779 | 0.0017456 | — | ** |
| VEGFC | 2.909 | 0.0034593 | — | ** |
| GRB7 | 1.702 | 0.0042453 | 1.6955 | 0.044 |
| GPR56 | 2.997 | 0.0048843 | — | ** |
| PGK1 | 4.246 | 0.0051870 | — | ** |
| SLC7A5 | 1.935 | 0.0058417 | — | ** |
| CDH1 | 2.213 | 0.0132978 | — | ** |
| PLAUR | 2.141 | 0.0155687 | — | ** |
| THBS1 | 2.203 | 0.0189764 | — | ** |
| APRT | 3.447 | 0.0206994 | — | ** |
| VIM | 2.361 | 0.0238545 | — | ** |
| SL | 1.916 | 0.0248133 | — | ** |
| MMP12 | 1.614 | 0.0251326 | — | ** |
| HSP90AA1 | 2.783 | 0.0267250 | — | ** |
| PLAU | 1.885 | 0.0342672 | — | ** |
| ABCC3 | 1.635 | 0.0368664 | — | ** |
| C14ORF10 | 2.140 | 0.0399352 | — | ** |
| PTTG1 | 1.624 | 0.0493965 | — | ** |

** Korn adj. p value >0.05

TABLE 8B
(Hormone Receptor Positive (HR+), HER2 Positive (HER2+)) Genes
with lower risk of recurrence with higher expression

| | |
|---|---|
| | Analysis 2 |
| Analysis 1 | (Adjusted) |

| | |
|---|---|
| (Unadjusted) | Korn adj. |

| gene | HR | p value | HR | p value |
|---|---|---|---|---|
| PFDN5 | 0.636 | 9.018E−06 | — | ** |
| RPLP0 | 0.081 | 0.0001399 | 0.0711 | 0.034 |
| MAPT | 0.612 | 0.0005146 | 0.6126 | 0.00 |
| ESR1 | 0.721 | 0.0019943 | — | ** |
| APOD | 0.658 | 0.0032732 | — | ** |
| IGFBP2 | 0.632 | 0.0035894 | — | ** |
| SGK3 | 0.418 | 0.0048934 | — | ** |
| SCUBE2 | 0.692 | 0.0056279 | — | ** |
| PGR | 0.712 | 0.0059421 | 0.6663 | 0.036 |
| IRS1 | 0.528 | 0.0065563 | — | ** |
| KLK10 | 0.174 | 0.0094446 | — | ** |
| CHEK2 | 0.294 | 0.0141015 | — | ** |
| MGC52057 | 0.381 | 0.0142698 | — | ** |
| FHIT | 0.523 | 0.0157057 | — | ** |
| AKT2 | 0.260 | 0.0220461 | — | ** |
| FASN | 0.609 | 0.0284812 | — | ** |
| ERCC1 | 0.345 | 0.0347430 | — | ** |
| ABCA9 | 0.611 | 0.0360546 | — | ** |
| GATA3 | 0.728 | 0.0374971 | — | ** |
| STK11 | 0.379 | 0.0395275 | — | ** |
| TUBD1 | 0.552 | 0.0414193 | — | ** |

** Korn adj. p value >0.05

TABLE 9A
(Hormone Receptor Negative (HR−), HER2 Positive (HER2+)) Genes
with higher risk of recurrence with higher expression

| | |
|---|---|
| | Analysis 2 |
| Analysis 1 | (Adjusted) |

| | |
|---|---|
| (Unadjusted) | Korn adj. |

| gene | HR | p value | HR | p value |
|---|---|---|---|---|
| MYBL2 | 2.4606679 | 0.0088333 | — | ** |
| AURKB | 2.1954949 | 0.0070310 | — | ** |
| BRCA2 | 1.9594455 | 0.0255585 | — | ** |
| PTTG1 | 1.9428582 | 0.0110666 | — | ** |
| KIFC1 | 1.8397539 | 0.0323052 | — | ** |
| CDC20 | 1.7849698 | 0.0190490 | — | ** |
| ESPL1 | 1.7654602 | 0.0254649 | — | ** |
| DEPDC1 | 1.6955687 | 0.0089039 | — | ** |
| EGFR | 1.6497619 | 0.0366391 | — | ** |
| LAPTM4B | 1.5456666 | 0.0397772 | — | ** |
| MMP12 | 1.463091 | 0.0376501 | — | ** |

** Korn adj. p value >0.05

TABLE 9B
(Hormone Receptor Negative (HR−), HER2 Positive (HER2+)) Genes
with lower risk of recurrence with higher expression

| | |
|---|---|
| | Analysis 2 |
| Analysis 1 | (Adjusted) |

| | |
|---|---|
| (Unadjusted) | Korn adj. |

| gene | HR | p value | HR | p value |
|---|---|---|---|---|
| APOD | 0.7736676 | 0.0435824 | — | ** |
| MUC1 | 0.7606705 | 0.0346312 | — | ** |
| FOXA1 | 0.7438023 | 0.0130209 | — | ** |
| GRB7 | 0.7054072 | 0.0039900 | — | ** |
| SCUBE2 | 0.682751 | 0.0195569 | — | ** |
| ERBB2 | 0.6675413 | 0.0191915 | — | ** |
| TFF1 | 0.6380236 | 0.0039543 | 0.6383 | 0.00 |
| TPT1 | 0.6367527 | 0.0027398 | — | ** |
| SEMA3F | 0.6309245 | 0.0472318 | — | ** |
| GATA3 | 0.6225757 | 0.0295526 | — | ** |
| ERBB4 | 0.6097751 | 0.0215173 | — | ** |
| RAB27B | 0.6055422 | 0.0064456 | — | ** |
| RHOB | 0.6008914 | 0.0436872 | — | ** |
| TNFSF10 | 0.5863233 | 0.0011459 | — | ** |
| KRT19 | 0.5577157 | 0.0000444 | — | ** |
| PGR | 0.4937589 | 0.0303120 | — | ** |
| TNFRSF10A | 0.4406017 | 0.0206329 | — | ** |
| ABAT | 0.4372501 | 0.0008712 | — | ** |
| MSH3 | 0.4368676 | 0.0143446 | — | ** |
| ESR1 | 0.4104291 | 0.0022777 | 0.4173 | 0.000 |
| CHFR | 0.341955 | 0.0088551 | — | ** |
| PIK3C2A | 0.3276976 | 0.0366853 | — | ** |
| SLC25A3 | 0.246417 | 0.0168162 | — | ** |
| CYP2C8 | 0.1471685 | 0.0237797 | — | ** |
| HSPA1L | 0.047539 | 0.0341650 | — | ** |

** Korn adj. p value >0.05

TABLE 2

| | | | | |
|---|---|---|---|---|
| | | | | SEQ ID |
| Gene Name | Accession # | Oligo Name | Oligo Sequence | NO |

| | | | | |
|---|---|---|---|---|
| ABCA9 | NM_172386 | T2132/ABCA9.f1 | TTACCCGTGGGAACTGTCTC | 1 |
| ABCA9 | NM_172386 | T2133/ABCA9.r1 | GACCAGTAAATGGGTCAGAGGA | 2 |
| ABCA9 | NM_172386 | T2134/ABCA9.p1 | TCCTCTCACCAGGACAACAACCACA | 3 |
| ABCB1 | NM_000927 | S8730/ABCB1.f5 | AAACACCACTGGAGCATTGA | 4 |
| ABCB1 | NM_000927 | S8731/ABCB1.r5 | CAAGCCTGGAACCTATAGCC | 5 |
| ABCB1 | NM_000927 | S8732/ABCB1.p5 | CTCGCCAATGATGCTGCTCAAGTT | 6 |
| ABCB5 | NM_178559 | T2072/ABCB5.f1 | AGACAGTCGCCTTGGTCG | 7 |
| ABCB5 | NM_178559 | T2073/ABCB5.r1 | AACCTCTGCAGAAGCTGGAC | 8 |
| ABCB5 | NM_178559 | T2074/ABCB5.p1 | CCGTACTCTTCCCACTGCCATTGA | 9 |
| ABCC10 | NM_033450 | S9064/ABCC10.f1 | ACCAGTGCCACAATGCAG | 10 |
| ABCC10 | NM_033450 | S9065/ABCC10.r1 | ATAGCGCTGACCACTGCC | 11 |
| ABCC10 | NM_033450 | S9066/ABCC10.p1 | CCATGAGCTGTAGCCGAATGTCCA | 12 |
| ABCC11 | NM_032583 | T2066/ABCC11.f1 | AAGCCACAGCCTCCATTG | 13 |
| ABCC11 | NM_032583 | T2067/ABCC11.r1 | GGAAGGCTTCACGGATTGT | 14 |
| ABCC11 | NM_032583 | T2068/ABCC11.p1 | TGGAGACAGACACCCTGATCCAGC | 15 |
| ABCC5 | NM_005688 | S5605/ABCC5.f1 | TGCAGACTGTACCATGCTGA | 16 |
| ABCC5 | NM_005688 | S5606/ABCC5.r1 | GGCCAGCACCATAATCCTAT | 17 |
| ABCC5 | NM_005688 | S5607/ABCC5.p1 | CTGCACACGGTTCTAGGCTCCG | 18 |
| ABCD1 | NM_000033 | T1991/ABCD1.f1 | TCTGTGGCCCACCTCTACTC | 19 |
| ABCD1 | NM_000033 | T1992/ABCD1.r1 | GGGTGTAGGAAGTCACAGCC | 20 |
| ABCD1 | NM_000033 | T1993/ABCD1.p1 | AACCTGACCAAGCCACTCCTGGAC | 21 |
| ACTG2 | NM_001615 | S4543/ACTG2.f3 | ATGTACGTCGCCATTCAAGCT | 22 |
| ACTG2 | NM_001615 | S4544/ACTG2.r3 | ACGCCATCACCTGAATCCA | 23 |
| ACTG2 | NM_001615 | S4545/ACTG2.p3 | CTGGCCGCACGACAGGCATC | 24 |
| ACTR2 | NM_005722 | T2380/ACTR2.f1 | ATCCGCATTGAAGACCCA | 25 |
| ACTR2 | NM_005722 | T2381/ACTR2.r1 | ATCCGCTAGAACTGCACCAC | 26 |
| ACTR2 | NM_005722 | T2382/ACTR2.p1 | CCCGCAGAAAGCACATGGTATTCC | 27 |
| ACTR3 | NM_005721 | T2383/ACTR3.f1 | CAACTGCTGAGAGACCGAGA | 28 |
| ACTR3 | NM_005721 | T2384/ACTR3.r1 | CGCTCCTTTACTGCCTTAGC | 29 |
| ACTR3 | NM_005721 | T2385/ACTR3.p1 | AGGAATCCCTCCAGAACAATCCTTGG | 30 |
| AK055699 | NM_194317 | S2097/AK0556.f1 | CTGCATGTGATTGAATAAGAAACAAGA | 31 |
| AK055699 | NM_194317 | S2098/AK0556.r1 | TGTGGACCTGATCCCTGTACAC | 32 |
| AK055699 | NM_194317 | S5057/AK0556.p1 | TGACCACACCAAAGCCTCCCTGG | 33 |
| AKT1 | NM_005163 | S0010/AKT1.f3 | CGCTTCTATGGCGCTGAGAT | 34 |
| AKT1 | NM_005163 | S0012/AKT1.r3 | TCCCGGTACACCACGTTCTT | 35 |
| AKT1 | NM_005163 | S4776/AKT1.p3 | CAGCCCTGGACTACCTGCACTCGG | 36 |
| AKT2 | NM_001626 | S0828/AKT2.f3 | TCCTGCCACCCTTCAAACC | 37 |
| AKT2 | NM_001626 | S0829/AKT2.r3 | GGCGGTAAATTCATCATCGAA | 38 |
| AKT2 | NM_001626 | S4727/AKT2.p3 | CAGGTCACGTCCGAGGTCGACACA | 39 |
| AKT3 | NM_005465 | S0013/AKT3.f2 | TTGTCTCTGCCTTGGACTATCTACA | 40 |
| AKT3 | NM_005465 | S0015/AKT3.r2 | CCAGCATTAGATTCTCCAACTTGA | 41 |
| AKT3 | NM_005465 | S4884/AKT3.p2 | TCACGGTACACAATCTTTCCGGA | 42 |
| ANXA4 | NM_001153 | T1017/ANXA4.f1 | TGGGAGGGATGAAGGAAAT | 43 |
| ANXA4 | NM_001153 | T1018/ANXA4.r1 | CTCATACAGGTCCTGGGCA | 44 |
| ANXA4 | NM_001153 | T1019/ANXA4.p1 | TGTCTCACGAGAGCATCGTCCAGA | 45 |
| APC | NM_000038 | S0022/APC.f4 | GGACAGCAGGAATGTGTTTC | 46 |
| APC | NM_000038 | S0024/APC.r4 | ACCCACTCGATTTGTTTCTG | 47 |
| APC | NM_000038 | S4888/APC.p4 | CATTGGCTCCCCGTGACCTGTA | 48 |
| APEX-1 | NM_001641 | S9947/APEX-1.f1 | GATGAAGCCTTTCGCAAGTT | 49 |
| APEX-1 | NM_001641 | S9948/APEX-1.r1 | AGGTCTCCACACAGCACAAG | 50 |
| APEX-1 | NM_001641 | S9949/APEX-1.p1 | CTTTCGGGAAGCCAGGCCCTT | 51 |
| APOC1 | NM_001645 | S9667/APOC1.f2 | GGAAACACACTGGAGGACAAG | 52 |
| APOC1 | NM_001645 | S9668/APOC1.r2 | CGCATCTTGGCAGAAAGTT | 53 |
| APOC1 | NM_001645 | S9669/APOC1.p2 | TCATCAGCCGCATCAAACAGAGTG | 54 |
| APOD | NM_001647 | T0536/APOD.f1 | GTTTATGCCATCGGCACC | 55 |
| APOD | NM_001647 | T0537/APOD.r1 | GGAATACACGAGGGCATAGTTC | 56 |
| APOD | NM_001647 | T0538/APOD.p1 | ACTGGATCCTGGCCACCGACTATG | 57 |
| APOE | NM_000041 | T1994/APOE.f1 | GCCTCAAGAGCTGGTTCG | 58 |
| APOE | NM_000041 | T1995/APOE.r1 | CCTGCACCTTCTCCACCA | 59 |
| APOE | NM_000041 | T1996/APOE.p1 | ACTGGCGCTGCATGTCTTCCAC | 60 |
| APRT | NM_000485 | T1023/APRT.f1 | GAGGTCCTGGAGTGCGTG | 61 |
| APRT | NM_000485 | T1024/APRT.r1 | AGGTGCCAGCTTCTCCCT | 62 |
| APRT | NM_000485 | T1025/APRT.p1 | CCTTAAGCGAGGTCAGCTCCACCA | 63 |
| ARHA | NM_001664 | S8372/ARHA.f1 | GGTCCTCCGTCGGTTCTC | 64 |
| ARHA | NM_001664 | S8373/ARHA.r1 | GTCGCAAACTCGGAGACG | 65 |
| ARHA | NM_001664 | S8374/ARHA.p1 | CCACGGTCTGGTCTTCAGCTACCC | 66 |
| AURKB | NM_004217 | S7250/AURKB.f1 | AGCTGCAGAAGAGCTGCACAT | 67 |
| AURKB | NM_004217 | S7251/AURKB.r1 | GCATCTGCCAACTCCTCCAT | 68 |
| AURKB | NM_004217 | S7252/AURKB.p1 | TGACGAGCAGCGAACAGCCACG | 69 |
| B-actin | NM_001101 | S0034/B-acti.f2 | CAGCAGATGTGGATCAGCAAG | 70 |
| B-actin | NM_001101 | S0036/B-acti.r2 | GCATTTGCGGTGGACGAT | 71 |
| B-actin | NM_001101 | S4730/B-acti.p2 | AGGAGTATGACGAGTCCGGCCCC | 72 |
| B-Catenin | NM_001904 | S2150/B-Cate.f3 | GGCTCTTGTGCGTACTGTCCTT | 73 |
| B-Catenin | NM_001904 | S2151/B-Cate.r3 | TCAGATGACGAAGAGCACAGATG | 74 |
| B-Catenin | NM_001904 | S5046/B-Cate.p3 | AGGCTCAGTGATGTCTTCCCTGTCACCAG | 75 |
| BAD | NM_032989 | S2011/BAD.f1 | GGGTCAGGTGCCTCGAGAT | 76 |
| BAD | NM_032989 | S2012/BAD.r1 | CTGCTCACTCGGCTCAAACTC | 77 |
| BAD | NM_032989 | S5058/BAD.p1 | TGGGCCCAGAGCATGTTCCAGATC | 78 |
| BAG1 | NM_004323 | S1386/BAG1.f2 | CGTTGTCAGCACTTGGAATACAA | 79 |
| BAG1 | NM_004323 | S1387/BAG1.r2 | GTTCAACCTCTTCCTGTGGACTGT | 80 |
| BAG1 | NM_004323 | S4731/BAG1.p2 | CCCAATTAACATGACCCGGCAACCAT | 81 |
| Bak | NM_001188 | S0037/Bak.f2 | CCATTCCCACCATTCTACCT | 82 |
| Bak | NM_001188 | S0039/Bak.r2 | GGGAACATAGACCCACCAAT | 83 |
| Bak | NM_001188 | S4724/Bak.p2 | ACACCCCAGACGTCCTGGCCT | 84 |
| Bax | NM_004324 | S0040/Bax.f1 | CCGCCGTGGACACAGACT | 85 |
| Bax | NM_004324 | S0042/Bax.r1 | TTGCCGTCAGAAAACATGTCA | 86 |
| Bax | NM_004324 | S4897/Bax.p1 | TGCCACTCGGAAAAAGACCTCTCGG | 87 |
| BBC3 | NM_014417 | S1584/BBC3.f2 | CCTGGAGGGTCCTGTACAAT | 88 |
| BBC3 | NM_014417 | S1585/BBC3.r2 | CTAATTGGGCTCCATCTCG | 89 |
| BBC3 | NM_014417 | S4890/BBC3.p2 | CATCATGGGACTCCTGCCCTTACC | 90 |
| Bcl2 | NM_000633 | S0043/Bcl2.f2 | CAGATGGACCTAGTACCCACTGAGA | 91 |
| Bcl2 | NM_000633 | S0045/Bcl2.r2 | CCTATGATTTAAGGGCATTTTTCC | 92 |
| Bcl2 | NM_000633 | S4732/Bcl2.p2 | TTCCACGCCGAAGGACAGCGAT | 93 |
| BCL2L11 | NM_138621 | S7139/BCL2L1.f1 | AATTACCAAGCAGCCGAAGA | 94 |
| BCL2L11 | NM_138621 | S7140/BCL2L1.r1 | CAGGCGGACAATGTAACGTA | 95 |
| BCL2L11 | NM_138621 | S7141/BCL2L1.p1 | CCACCCACGAATGGTTATCTTACGACTG | 96 |
| BCL2L13 | NM_015367 | S9025/BCL2L1.f1 | CAGCGACAACTCTGGACAAG | 97 |
| BCL2L13 | NM_015367 | S9026/BCL2L1.r1 | GCTCTCAGACTGCCAGGAA | 98 |
| BCL2L13 | NM_015367 | S9027/BCL2L1.p1 | CCCCAGAGTCTCCAACTGTGACCA | 99 |
| Bclx | NM_001191 | S0046/Bclx.f2 | CTTTTGTGGAACTCTATGGGAACA | 100 |
| Bclx | NM_001191 | S0048/Bclx.r2 | CAGCGGTTGAAGCGTTCCT | 101 |
| Bclx | NM_001191 | S4898/Bclx.p2 | TTCGGCTCTCGGCTGCTGCA | 102 |
| BCRP | NM_004827 | S0840/BCRP.f1 | TGTACTGGCGAAGAATATTTGGTAAA | 103 |
| BCRP | NM_004827 | S0841/BCRP.r1 | GCCACGTGATTCTTCCACAA | 104 |
| BCRP | NM_004827 | S4836/BCRP.p1 | CAGGGCATCGATCTCTCACCCTGG | 105 |
| BID | NM_001196 | S6273/BID.f3 | GGACTGTGAGGTCAACAACG | 106 |
| BID | NM_001196 | S6274/BID.r3 | GGAAGCCAAACACCAGTAGG | 107 |
| BID | NM_001196 | S6275/BID.p3 | TGTGATGCACTCATCCCTGAGGCT | 108 |
| BIN1 | NM_004305 | S2651/BIN1.f3 | CCTGCAAAAGGGAACAAGAG | 109 |
| BIN1 | NM_004305 | S2652/BIN1.r3 | CGTGGTTGACTCTGATCTCG | 110 |
| BIN1 | NM_004305 | S4954/BIN1.p3 | CTTCGCCTCCAGATGGCTCCC | 111 |
| BRCA1 | NM_007295 | S0049/BRCA1.f2 | TCAGGGGGCTAGAAATCTGT | 112 |
| BRCA1 | NM_007295 | S0051/BRCA1.r2 | CCATTCCAGTTGATCTGTGG | 113 |
| BRCA1 | NM_007295 | S4905/BRCA1.p2 | CTATGGGCCCTTCACCAACATGC | 114 |
| BRCA2 | NM_000059 | S0052/BRCA2.f2 | AGTTCGTGCTTTGCAAGATG | 115 |
| BRCA2 | NM_000059 | S0054/BRCA2.r2 | AAGGTAAGCTGGGTCTGCTG | 116 |
| BRCA2 | NM_000059 | S4985/BRCA2.p2 | CATTCTTCACTGCTTCATAAAGCTCTGCA | 117 |
| BUB1 | NM_004336 | S4294/BUB1.f1 | CCGAGGTTAATCCAGCACGTA | 118 |
| BUB1 | NM_004336 | S4295/BUB1.r1 | AAGACATGGCGCTCTCAGTTC | 119 |
| BUB1 | NM_004336 | S4296/BUB1.p1 | TGCTGGGAGCCTACACTTGGCCC | 120 |
| BUB1B | NM_001211 | S8060/BUB1B.f1 | TCAACAGAAGGCTGAACCACTAGA | 121 |
| BUB1B | NM_001211 | S8061/BUB1B.r1 | CAACAGAGTTTGCCGAGACACT | 122 |
| BUB1B | NM_001211 | S8062/BUB1B.p1 | TACAGTCCCAGCACCGACAATTCC | 123 |
| BUB3 | NM_004725 | S8475/BUB3.f1 | CTGAAGCAGATGGTTCATCATT | 124 |
| BUB3 | NM_004725 | S8476/BUB3.r1 | GCTGATTCCCAAGAGTCTAACC | 125 |
| BUB3 | NM_004725 | S8477/BUB3.p1 | CCTCGCTTTGTTTAACAGCCCAGG | 126 |
| c-Src | NM_005417 | S7320/c-Src.f1 | TGAGGAGTGGTATTTTGGCAAGA | 127 |
| c-Src | NM_005417 | S7321/c-Src.r1 | CTCTCGGGTTCTCTGCATTGA | 128 |
| c-Src | NM_005417 | S7322/c-Src.p1 | AACCGCTCTGACTCCCGTCTGGTG | 129 |
| C14orf10 | NM_017917 | T2054/C14orf.f1 | GTCAGCGTGGTAGCGGTATT | 130 |
| C14orf10 | NM_017917 | T2055/C14orf.r1 | GGAAGTCTTGGCTAAAGAGGC | 131 |
| C14orf10 | NM_017917 | T2056/C14orf.p1 | AACAATTACTGTCACTGCCGCGGA | 132 |
| C20 orf1 | NM_012112 | S3560/C20 or.f1 | TCAGCTGTGAGCTGCGGATA | 133 |
| C20 orf1 | NM_012112 | S3561/C20 or.r1 | ACGGTCCTAGGTTTGAGGTTAAGA | 134 |
| C20 orf1 | NM_012112 | S3562/C20 or.p1 | CAGGTCCCATTGCCGGGCG | 135 |
| CA9 | NM_001216 | S1398/CA9.f3 | ATCCTAGCCCTGGTTTTTGG | 136 |
| CA9 | NM_001216 | S1399/CA9.r3 | CTGCCTTCTCATCTGCACAA | 137 |
| CA9 | NM_001216 | S4938/CA9.p3 | TTTGCTGTCACCAGCGTCGC | 138 |
| CALD1 | NM_004342 | S4683/CALD1.f2 | CACTAAGGTTTGAGACAGTTCCAGAA | 139 |
| CALD1 | NM_004342 | S4684/CALD1.r2 | GCGAATTAGCCCTCTACAACTGA | 140 |
| CALD1 | NM_004342 | S4685/CALD1.p2 | AACCCAAGCTCAAGACGCAGGACGAG | 141 |
| CAPZA1 | NM_006135 | T2228/CAPZA1.f1 | TCGTTGGAGATCAGAGTGGA | 142 |
| CAPZA1 | NM_006135 | T2229/CAPZA1.r1 | TTAAGCACGCCAACCACC | 143 |
| CAPZA1 | NM_006135 | T2230/CAPZA1.p1 | TCACCATCACACCACCTACAGCCC | 144 |
| CAV1 | NM_001753 | S7151/CAV1.f1 | GTGGCTCAACATTGTGTTCC | 145 |
| CAV1 | NM_001753 | S7152/CAV1.r1 | CAATGGCCTCCATTTTACAG | 146 |
| CAV1 | NM_001753 | S7153/CAV1.p1 | ATTTCAGCTGATCAGTGGGCCTCC | 147 |
| CCNB1 | NM_031966 | S1720/CCNB1.f2 | TTCAGGTTGTTGCAGGAGAC | 148 |
| CCNB1 | NM_031966 | S1721/CCNB1.r2 | CATCTTCTTGGGCACACAAT | 149 |
| CCNB1 | NM_031966 | S4733/CCNB1.p2 | TGTCTCCATTATTGATCGGTTCATGCA | 150 |
| CCND1 | NM_053056 | S0058/CCND1.f3 | GCATGTTCGTGGCCTCTAAGA | 151 |
| CCND1 | NM_053056 | S0060/CCND1.r3 | CGGTGTAGATGCACAGCTTCTC | 152 |
| CCND1 | NM_053056 | S4986/CCND1.p3 | AAGGAGACCATCCCCCTGACGGC | 153 |
| CCNE2 | NM_057749 | S1458/CCNE2.f2 | ATGCTGTGGCTCCTTCCTAACT | 154 |
| CCNE2 | NM_057749 | S1459/CCNE2.r2 | ACCCAAATTGTGATATACAAAAAGGTT | 155 |
| CCNE2 | NM_057749 | S4945/CCNE2.p2 | TACCAAGCAACCTACATGTCAAGAAAGCCC | 156 |
| CCT3 | NM_001008800 | T1053/CCT3.f1 | ATCCAAGGCCATGACTGG | 157 |
| CCT3 | NM_001008800 | T1054/CCT3.r1 | GGAATGACCTCTAGGGCCTG | 158 |
| CCT3 | NM_001008800 | T1055/CCT3.p1 | ACAGCCCTGTATGGCCATTGTTCC | 159 |
| CD14 | NM_000591 | T1997/CD14.f1 | GTGTGCTAGCGTACTCCCG | 160 |
| CD14 | NM_000591 | T1998/CD14.r1 | GCATGGTGCCGGTTATCT | 161 |
| CD14 | NM_000591 | T1999/CD14.p1 | CAAGGAACTGACGCTCGAGGACCT | 162 |
| CD31 | NM_000442 | S1407/CD31.f3 | TGTATTTCAAGACCTCTGTGCACTT | 163 |
| CD31 | NM_000442 | S1408/CD31.r3 | TTAGCCTGAGGAATTGCTGTGTT | 164 |
| CD31 | NM_000442 | S4939/CD31.p3 | TTTATGAACCTGCCCTGCTCCCACA | 165 |
| CD3z | NM_000734 | S0064/CD3z.f1 | AGATGAAGTGGAAGGCGCTT | 166 |
| CD3z | NM_000734 | S0066/CD3z.r1 | TGCCTCTGTAATCGGCAACTG | 167 |
| CD3z | NM_000734 | S4988/CD3z.p1 | CACCGCGGCCATCCTGCA | 168 |
| CD63 | NM_001780 | T1988/CD63.f1 | AGTGGGACTGATTGCCGT | 169 |
| CD63 | NM_001780 | T1989/CD63.r1 | GGGTAGCCCCCTGGATTAT | 170 |
| CD63 | NM_001780 | T1990/CD63.p1 | TCTGACTCAGGACAAGCTGTGCCC | 171 |
| CD68 | NM_001251 | S0067/CD68.f2 | TGGTTCCCAGCCCTGTGT | 172 |
| CD68 | NM_001251 | S0069/CD68.r2 | CTCCTCCACCCTGGGTTGT | 173 |
| CD68 | NM_001251 | S4734/CD68.p2 | CTCCAAGCCCAGATTCAGATTCGAGTCA | 174 |
| CDC2 | NM_001786 | S7238/CDC2.f1 | GAGAGCGACGCGGTTGTT | 175 |
| CDC2 | NM_001786 | S7239/CDC2.r1 | GTATGGTAGATCCCGGCTTATTATTC | 176 |
| CDC2 | NM_001786 | S7240/CDC2.p1 | TAGCTGCCGCTGCGGCCG | 177 |
| CDC20 | NM_001255 | S4447/CDC20.f1 | TGGATTGGAGTTCTGGGAATG | 178 |
| CDC20 | NM_001255 | S4448/CDC20.r1 | GCTTGCACTCCACAGGTACACA | 179 |
| CDC20 | NM_001255 | S4449/CDC20.p1 | ACTGGCCGTGGCACTGGACAACA | 180 |
| CDC25B | NM_021873 | S1160/CDC25B.f1 | AAACGAGCAGTTTGCCATCAG | 181 |
| CDC25B | NM_021873 | S1161/CDC25B.r1 | GTTGGTGATGTTCCGAAGCA | 182 |
| CDC25B | NM_021873 | S4842/CDC25B.p1 | CCTCACCGGCATAGACTGGAAGCG | 183 |
| CDCA8 | NM_018101 | T2060/CDCA8.f1 | GAGGCACAGTATTGCCCAG | 184 |
| CDCA8 | NM_018101 | T2061/CDCA8.r1 | GAGACGGTTGGAGAGCTTCTT | 185 |
| CDCA8 | NM_018101 | T2062/CDCA8.p1 | ATGTTTCCCAAGGCCTCTGGATCC | 186 |
| CDH1 | NM_004360 | S0073/CDH1.f3 | TGAGTGTCCCCCGGTATCTTC | 187 |
| CDH1 | NM_004360 | S0075/CDH1.r3 | CAGCCGCTTTCAGATTTTCAT | 188 |
| CDH1 | NM_004360 | S4990/CDH1.p3 | TGCCAATCCCGATGAAATTGGAAATTT | 189 |
| CDK5 | NM_004935 | T2000/CDK5.f1 | AAGCCCTATCCGATGTACCC | 190 |
| CDK5 | NM_004935 | T2001/CDK5.r1 | CTGTGGCATTGAGTTTGGG | 191 |
| CDK5 | NM_004935 | T2002/CDK5.p1 | CACAACATCCCTGGTGAACGTCGT | 192 |
| CDKN1C | NM_000076 | T2003/CDKN1C.f1 | CGGCGATCAAGAAGCTGT | 193 |
| CDKN1C | NM_000076 | T2004/CDKN1C.r1 | CAGGCGCTGATCTCTTGC | 194 |
| CDKN1C | NM_000076 | T2005/CDKN1C.p1 | CGGGCCTCTGATCTCCGATTTCTT | 195 |
| CEGP1 | NM_020974 | S1494/CEGP1.f2 | TGACAATCAGCACACCTGCAT | 196 |
| CEGP1 | NM_020974 | S1495/CEGP1.r2 | TGTGACTACAGCCGTGATCCTTA | 197 |
| CEGP1 | NM_020974 | S4735/CEGP1.p2 | CAGGCCCTCTTCCGAGCGGT | 198 |
| CENPA | NM_001809 | S7082/CENPA.f1 | TAAATTCACTCGTGGTGTGGA | 199 |
| CENPA | NM_001809 | S7083/CENPA.r1 | GCCTCTTGTAGGGCCAATAG | 200 |
| CENPA | NM_001809 | S7084/CENPA.p1 | CTTCAATTGGCAAGCCCAGGC | 201 |
| CENPE | NM_001813 | S5496/CENPE.f3 | GGATGCTGGTGACCTCTTCT | 202 |
| CENPE | NM_001813 | S5497/CENPE.r3 | GCCAAGGCACCAAGTAACTC | 203 |
| CENPE | NM_001813 | S5498/CENPE.p3 | TCCCTCACGTTGCAACAGGAATTAA | 204 |
| CENPF | NM_016343 | S9200/CENPF.f1 | CTCCCGTCAACAGCGTTC | 205 |
| CENPF | NM_016343 | S9201/CENPF.r1 | GGGTGAGTCTGGCCTTCA | 206 |
| CENPF | NM_016343 | S9202/CENPF.p1 | ACACTGGACCAGGAGTGCATCCAG | 207 |
| CGA (CHGA | NM_001275 | S3221/CGA (C.f3 | CTGAAGGAGCTCCAAGACCT | 208 |
| official) | | | | |
| CGA (CHGA | NM_001275 | S3222/CGA (C.r3 | CAAAACCGCTGTGTTTCTTC | 209 |
| official) | | | | |
| CGA (CHGA | NM_001275 | S3254/CGA (C.p3 | TGCTGATGTGCCCTCTCCTTGG | 210 |
| official) | | | | |
| CHFR | NM_018223 | S7085/CHFR.f1 | AAGGAAGTGGTCCCTCTGTG | 211 |
| CHFR | NM_018223 | S7086/CHFR.r1 | GACGCAGTCTTTCTGTCTGG | 212 |
| CHFR | NM_018223 | S7087/CHFR.p1 | TGAAGTCTCCAGCTTTGCCTCAGC | 213 |
| Chk1 | NM_001274 | S1422/Chk1.f2 | GATAAATTGGTACAAGGGATCAGCTT | 214 |
| Chk1 | NM_001274 | S1423/Chk1.r2 | GGGTGCCAAGTAACTGACTATTCA | 215 |
| Chk1 | NM_001274 | S4941/Chk1.p2 | CCAGCCCACATGTCCTGATCATATGC | 216 |
| Chk2 | NM_007194 | S1434/Chk2.f3 | ATGTGGAACCCCCACCTACTT | 217 |
| Chk2 | NM_007194 | S1435/Chk2.r3 | CAGTCCACAGCACGGTTATACC | 218 |
| Chk2 | NM_007194 | S4942/Chk2.p3 | AGTCCCAACAGAAACAAGAACTTCAGGCG | 219 |
| cIAP2 | NM_001165 | S0076/cIAP2.f2 | GGATATTTCCGTGGCTCTTATTCA | 220 |
| cIAP2 | NM_001165 | S0078/cIAP2.r2 | CTTCTCATCAAGGCAGAAAAATCTT | 221 |
| cIAP2 | NM_001165 | S4991/cIAP2.p2 | TCTCCATCAAATCCTGTAAACTCCAGAGCA | 222 |
| CKAP1 | NM_001281 | T2293/CKAP1.f1 | TCATTGACCACAGTGGCG | 223 |
| CKAP1 | NM_001281 | T2294/CKAP1.r1 | TCGTGTACTTCTCCACCCG | 224 |
| CKAP1 | NM_001281 | T2295/CKAP1.p1 | CACGTCCTCATACTCACCAAGGCG | 225 |
| CLU | NM_001831 | S5666/CLU.f3 | CCCCAGGATACCTACCACTACCT | 226 |
| CLU | NM_001831 | S5667/CLU.r3 | TGCGGGACTTGGGAAAGA | 227 |
| CLU | NM_001831 | S5668/CLU.p3 | CCCTTCAGCCTGCCCCACCG | 228 |
| cMet | NM_000245 | S0082/cMet.f2 | GACATTTCCAGTCCTGCAGTCA | 229 |
| cMet | NM_000245 | S0084/cMet.r2 | CTCCGATCGCACACATTTGT | 230 |
| cMet | NM_000245 | S4993/cMet.p2 | TGCCTCTCTGCCCCACCCTTTGT | 231 |
| cMYC | NM_002467 | S0085/cMYC.f3 | TCCCTCCACTCGGAAGGACTA | 232 |
| cMYC | NM_002467 | S0087/cMYC.r3 | CGGTTGTTGCTGATCTGTCTCA | 233 |
| cMYC | NM_002467 | S4994/cMYC.p3 | TCTGACACTGTCCAACTTGACCCTCTT | 234 |
| CNN | NM_001299 | S4564/CNN.f1 | TCCACCCTCCTGGCTTTG | 235 |
| CNN | NM_001299 | S4565/CNN.r1 | TCACTCCCACGTTCACCTTGT | 236 |
| CNN | NM_001299 | S4566/CNN.p1 | TCCTTTCGTCTTCGCCATGCTGG | 237 |
| COL1A1 | NM_000088 | S4531/COL1A1.f1 | GTGGCCATCCAGCTGACC | 238 |
| COL1A1 | NM_000088 | S4532/COL1A1.r1 | CAGTGGTAGGTGATGTTCTGGGA | 239 |
| COL1A1 | NM_000088 | S4533/COL1A1.p1 | TCCTGCGCCTGATGTCCACCG | 240 |
| COL1A2 | NM_000089 | S4534/COL1A2.f1 | CAGCCAAGAACTGGTATAGGAGCT | 241 |
| COL1A2 | NM_000089 | S4535/COL1A2.r1 | AAACTGGCTGCCAGCATTG | 242 |
| COL1A2 | NM_000089 | S4536/COL1A2.p1 | TCTCCTAGCCAGACGTGTTTCTTGTCCTTG | 243 |
| COL6A3 | NM_004369 | T1062/COL6A3.f1 | GAGAGCAAGCGAGACATTCTG | 244 |
| COL6A3 | NM_004369 | T1063/COL6A3.r1 | AACAGGGAACTGGCCCAC | 245 |
| COL6A3 | NM_004369 | T1064/COL6A3.p1 | CCTCTTTGACGGCTCAGCCAATCT | 246 |
| Contig 51037 | NM_198477 | S2070/Contig.f1 | CGACAGTTGCGATGAAAGTTCTAA | 247 |
| Contig 51037 | NM_198477 | S2071/Contig.r1 | GGCTGCTAGAGACCATGGACAT | 248 |
| Contig 51037 | NM_198477 | S5059/Contig.p1 | CCTCCTCCTGTTGCTGCCACTAATGCT | 249 |
| COX2 | NM_000963 | S0088/COX2.f1 | TCTGCAGAGTTGGAAGCACTCTA | 250 |
| COX2 | NM_000963 | S0090/COX2.r1 | GCCGAGGCTTTTCTACCAGAA | 251 |
| COX2 | NM_000963 | S4995/COX2.p1 | CAGGATACAGCTCCACAGCATCGATGTC | 252 |
| COX7C | NM_001867 | T0219/COX7C.f1 | ACCTCTGTGGTCCGTAGGAG | 253 |
| COX7C | NM_001867 | T0220/COX7C.r1 | CGACCACTTGTTTTCCACTG | 254 |
| COX7C | NM_001867 | T0221/COX7C.p1 | TCTTCCCAGGGCCCTCCTCATAGT | 255 |
| CRABP1 | NM_004378 | S5441/CRABP1.f3 | AACTTCAAGGTCGGAGAAGG | 256 |
| CRABP1 | NM_004378 | S5442/CRABP1.r3 | TGGCTAAACTCCTGCACTTG | 257 |
| CRABP1 | NM_004378 | S5443/CRABP1.p3 | CCGTCCACGGTCTCCTCCTCA | 258 |
| CRIP2 | NM_001312 | S5676/CRIP2.f3 | GTGCTACGCCACCCTGTT | 259 |
| CRIP2 | NM_001312 | S5677/CRIP2.r3 | CAGGGGCTTCTCGTAGATGT | 260 |
| CRIP2 | NM_001312 | S5678/CRIP2.p3 | CCGATGTTCACGCCTTTGGGTC | 261 |
| CRYAB | NM_001885 | S8302/CRYAB.f1 | GATGTGATTGAGGTGCATGG | 262 |
| CRYAB | NM_001885 | S8303/CRYAB.r1 | GAACTCCCTGGAGATGAAACC | 263 |
| CRYAB | NM_001885 | S8304/CRYAB.p1 | TGTTCATCCTGGCGCTCTTCATGT | 264 |
| CSF1 | NM_000757 | S1482/CSF1.f1 | TGCAGCGGCTGATTGACA | 265 |
| CSF1 | NM_000757 | S1483/CSF1.r1 | CAACTGTTCCTGGTCTACAAACTCA | 266 |
| CSF1 | NM_000757 | S4948/CSF1.p1 | TCAGATGGAGACCTCGTGCCAAATTACA | 267 |
| CSNK1D | NM_001893 | S2332/CSNK1D.f3 | AGCTTTTCCGGAATCTGTTC | 268 |
| CSNK1D | NM_001893 | S2333/CSNK1D.r3 | ATTTGAGCATGTTCCAGTCG | 269 |
| CSNK1D | NM_001893 | S4850/CSNK1D.p3 | CATCGCCAGGGCTTCTCCTATGAC | 270 |
| CST7 | NM_003650 | T2108/CST7.f1 | TGGCAGAACTACCTGCAAGA | 271 |
| CST7 | NM_003650 | T2109/CST7.r1 | TGCTTCAAGGTGTGGTTGG | 272 |
| CST7 | NM_003650 | T2110/CST7.p1 | CACCTGCGTCTGGATGACTGTGAC | 273 |
| CTSD | NM_001909 | S1152/CTSD.f2 | GTACATGATCCCCTGTGAGAAGGT | 274 |
| CTSD | NM_001909 | S1153/CTSD.r2 | GGGACAGCTTGTAGCCTTTGC | 275 |
| CTSD | NM_001909 | S4841/CTSD.p2 | ACCCTGCCCGCGATCACACTGA | 276 |
| CTSL | NM_001912 | S1303/CTSL.f2 | GGGAGGCTTATCTCACTGAGTGA | 277 |
| CTSL | NM_001912 | S1304/CTSL.r2 | CCATTGCAGCCTTCATTGC | 278 |
| CTSL | NM_001912 | S4899/CTSL.p2 | TTGAGGCCCAGAGCAGTCTACCAGATTCT | 279 |
| CTSL2 | NM_001333 | S4354/CTSL2.f1 | TGTCTCACTGAGCGAGCAGAA | 280 |
| CTSL2 | NM_001333 | S4355/CTSL2.r1 | ACCATTGCAGCCCTGATTG | 281 |
| CTSL2 | NM_001333 | S4356/CTSL2.p1 | CTTGAGGACGCGAACAGTCCACCA | 282 |
| CXCR4 | NM_003467 | S5966/CXCR4.f3 | TGACCGCTTCTACCCCAATG | 283 |
| CXCR4 | NM_003467 | S5967/CXCR4.r3 | AGGATAAGGCCAACCATGATGT | 284 |
| CXCR4 | NM_003467 | S5968/CXCR4.p3 | CTGAAACTGGAACACAACCACCCACAAG | 285 |
| CYBA | NM_000101 | S5300/CYBA.f1 | GGTGCCTACTCCATTGTGG | 286 |
| CYBA | NM_000101 | S5301/CYBA.r1 | GTGGAGCCCTTCTTCCTCTT | 287 |
| CYBA | NM_000101 | S5302/CYBA.p1 | TACTCCAGCAGGCACACAAACACG | 288 |
| CYP1B1 | NM_000104 | S0094/CYP1B1.f3 | CCAGCTTTGTGCCTGTCACTAT | 289 |
| CYP1B1 | NM_000104 | S0096/CYP1B1.r3 | GGGAATGTGGTAGCCCAAGA | 290 |
| CYP1B1 | NM_000104 | S4996/CYP1B1.p3 | CTCATGCCACCACTGCCAACACCTC | 291 |
| CYP2C8 | NM_000770 | S1470/CYP2C8.f2 | CCGTGTTCAAGAGGAAGCTC | 292 |
| CYP2C8 | NM_000770 | S1471/CYP2C8.r2 | AGTGGGATCACAGGGTGAAG | 293 |
| CYP2C8 | NM_000770 | S4946/CYP2C8.p2 | TTTTCTCAACTCCTCCACAAGGCA | 294 |
| CYP3A4 | NM_017460 | S1620/CYP3A4.f2 | AGAACAAGGACAACATAGATCCTTACATAT | 295 |
| CYP3A4 | NM_017460 | S1621/CYP3A4.r2 | GCAAACCTCATGCCAATGC | 296 |
| CYP3A4 | NM_017460 | S4906/CYP3A4.p2 | CACACCCTTTGGAAGTGGACCCAGAA | 297 |
| DDR1 | NM_001954 | T2156/DDR1.f1 | CCGTGTGGCTCGCTTTCT | 298 |
| DDR1 | NM_001954 | T2157/DDR1.r1 | GGAGATTTCGCTGAAGAGTAACCA | 299 |
| DDR1 | NM_001954 | T2158/DDR1.p1 | TGCCGCTTCCTCTTTGCGGG | 300 |
| DIABLO | NM_019887 | S0808/DIABLO.f1 | CACAATGGCGGCTCTGAAG | 301 |
| DIABLO | NM_019887 | S0809/DIABLO.r1 | ACACAAACACTGTCTGTACCTGAAGA | 302 |
| DIABLO | NM_019887 | S4813/DIABLO.p1 | AAGTTACGCTGCGCGACAGCCAA | 303 |
| DIAPH1 | NM_005219 | S7608/DIAPH1.f1 | CAAGCAGTCAAGGAGAACCA | 304 |
| DIAPH1 | NM_005219 | S7609/DIAPH1.r1 | AGTTTTGCTCGCCTCATCTT | 305 |
| DIAPH1 | NM_005219 | S7610/DIAPH1.p1 | TTCTTCTGTCTCCCGCCGCTTC | 306 |
| DICER1 | NM_177438 | S5294/DICER1.f2 | TCCAATTCCAGCATCACTGT | 307 |
| DICER1 | NM_177438 | S5295/DICER1.r2 | GGCAGTGAAGGCGATAAAGT | 308 |
| DICER1 | NM_177438 | S5296/DICER1.p2 | AGAAAAGCTGTTTGTCTCCCCAGCA | 309 |
| DKFZp564D0462; | NM_198569 | S4405/DKFZp5.f2 | CAGTGCTTCCATGGACAAGT | 310 |
| DKFZp564D0462; | NM_198569 | S4406/DKFZp5.r2 | TGGACAGGGATGATTGATGT | 311 |
| DKFZp564D0462; | NM_198569 | S4407/DKFZp5.p2 | ATCTCCATCAGCATGGGCCAGTTT | 312 |
| DR4 | NM_003844 | S2532/DR4.f2 | TGCACAGAGGGTGTGGGTTAC | 313 |
| DR4 | NM_003844 | S2533/DR4.r2 | TCTTCATCTGATTTACAAGCTGTACATG | 314 |
| DR4 | NM_003844 | S4981/DR4.p2 | CAATGCTTCCAACAATTTGTTTGCTTGCC | 315 |
| DR5 | NM_003842 | S2551/DR5.f2 | CTCTGAGACAGTGCTTCGATGACT | 316 |
| DR5 | NM_003842 | S2552/DR5.r2 | CCATGAGGCCCAACTTCCT | 317 |
| DR5 | NM_003842 | S4979/DR5.p2 | CAGACTTGGTGCCCTTTGACTCC | 318 |
| DUSP1 | NM_004417 | S7476/DUSP1.f1 | AGACATCAGCTCCTGGTTCA | 319 |
| DUSP1 | NM_004417 | S7477/DUSP1.r1 | GACAAACACCCTTCCTCCAG | 320 |
| DUSP1 | NM_004417 | S7478/DUSP1.p1 | CGAGGCCATTGACTTCATAGACTCCA | 321 |
| EEF1D | NM_001960 | T2159/EEF1D.f1 | CAGAGGATGACGAGGATGATGA | 322 |
| EEF1D | NM_001960 | T2160/EEF1D.r1 | CTGTGCCGCCTCCTTGTC | 323 |
| EEF1D | NM_001960 | T2161/EEF1D.p1 | CTCCTCATTGTCACTGCCAAACAGGTCA | 324 |
| EGFR | NM_005228 | S0103/EGFR.f2 | TGTCGATGGACTTCCAGAAC | 325 |
| EGFR | NM_005228 | S0105/EGFR.r2 | ATTGGGACAGCTTGGATCA | 326 |
| EGFR | NM_005228 | S4999/EGFR.p2 | CACCTGGGCAGCTGCCAA | 327 |
| EIF4E | NM_001968 | S0106/EIF4E.f1 | GATCTAAGATGGCGACTGTCGAA | 328 |
| EIF4E | NM_001968 | S0108/EIF4E.r1 | TTAGATTCCGTTTTCTCCTCTTCTG | 329 |
| EIF4E | NM_001968 | S5000/EIF4E.p1 | ACCACCCCTACTCCTAATCCCCCGACT | 330 |
| EIF4EL3 | NM_004846 | S4495/EIF4EL.f1 | AAGCCGCGGTTGAATGTG | 331 |
| EIF4EL3 | NM_004846 | S4496/EIF4EL.r1 | TGACGCCAGCTTCAATGATG | 332 |
| EIF4EL3 | NM_004846 | S4497/EIF4EL.p1 | TGACCCTCTCCCTCTCTGGATGGCA | 333 |
| ELP3 | NM_018091 | T2234/ELP3.f1 | CTCGGATCCTAGCCCTCG | 334 |
| ELP3 | NM_018091 | T2235/ELP3.r1 | GGCATTGGAATATCCCTCTGTA | 335 |
| ELP3 | NM_018091 | T2236/ELP3.p1 | CCTCCATGGACTCGAGTGTACCGA | 336 |
| ER2 | NM_001437 | S0109/ER2.f2 | TGGTCCATCGCCAGTTATCA | 337 |
| ER2 | NM_001437 | S0111/ER2.r2 | TGTTCTAGCGATCTTGCTTCACA | 338 |
| ER2 | NM_001437 | S5001/ER2.p2 | ATCTGTATGCGGAACCTCAAAAGAGTCCCT | 339 |
| ErbB3 | NM_001982 | S0112/ErbB3.f1 | CGGTTATGTCATGCCAGATACAC | 340 |
| ErbB3 | NM_001982 | S0114/ErbB3.r1 | GAACTGAGACCCACTGAAGAAAGG | 341 |
| ErbB3 | NM_001982 | S5002/ErbB3.p1 | CCTCAAAGGTACTCCCTCCTCCCGG | 342 |
| ERBB4 | NM_005235 | S1231/ERBB4.f3 | TGGCTCTTAATCAGTTTCGTTACCT | 343 |
| ERBB4 | NM_005235 | S1232/ERBB4.r3 | CAAGGCATATCGATCCTCATAAAGT | 344 |
| ERBB4 | NM_005235 | S4891/ERBB4.p3 | TGTCCCACGAATAATGCGTAAATTCTCCAG | 345 |
| ERCC1 | NM_001983 | S2437/ERCC1.f2 | GTCCAGGTGGATGTGAAAGA | 346 |
| ERCC1 | NM_001983 | S2438/ERCC1.r2 | CGGCCAGGATACACATCTTA | 347 |
| ERCC1 | NM_001983 | S4920/ERCC1.p2 | CAGCAGGCCCTCAAGGAGCTG | 348 |
| ERK1 | NM_002746 | S1560/ERK1.f3 | ACGGATCACAGTGGAGGAAG | 349 |
| ERK1 | NM_002746 | S1561/ERK1.r3 | CTCATCCGTCGGGTCATAGT | 350 |
| ERK1 | NM_002746 | S4882/ERK1.p3 | CGCTGGCTCACCCCTACCTG | 351 |
| ESPL1 | NM_012291 | S5686/ESPL1.f3 | ACCCCCAGACCGGATCAG | 352 |
| ESPL1 | NM_012291 | S5687/ESPL1.r3 | TGTAGGGCAGACTTCCTCAAACA | 353 |
| ESPL1 | NM_012291 | S5688/ESPL1.p3 | CTGGCCCTCATGTCCCCTTCACG | 354 |
| EstR1 | NM_000125 | S0115/EstR1.f1 | CGTGGTGCCCCTCTATGAC | 355 |
| EstR1 | NM_000125 | S0117/EstR1.r1 | GGCTAGTGGGCGCATGTAG | 356 |
| EstR1 | NM_000125 | S4737/EstR1.p1 | CTGGAGATGCTGGACGCCC | 357 |
| fas | NM_000043 | S0118/fas.f1 | GGATTGCTCAACAACCATGCT | 358 |
| fas | NM_000043 | S0120/fas.r1 | GGCATTAACACTTTTGGACGATAA | 359 |
| fas | NM_000043 | S5003/fas.p1 | TCTGGACCCTCCTACCTCTGGTTCTTACGT | 360 |
| fasl | NM_000639 | S0121/fasl.f2 | GCACTTTGGGATTCTTTCCATTAT | 361 |
| fasl | NM_000639 | S0123/fasl.r2 | GCATGTAAGAAGACCCTCACTGAA | 362 |
| fasl | NM_000639 | S5004/fasl.p2 | ACAACATTCTCGGTGCCTGTAACAAAGAA | 363 |
| FASN | NM_004104 | S8287/FASN.f1 | GCCTCTTCCTGTTCGACG | 364 |
| FASN | NM_004104 | S8288/FASN.r1 | GCTTTGCCCGGTAGCTCT | 365 |
| FASN | NM_004104 | S8289/FASN.p1 | TCGCCCACCTACGTACTGGCCTAC | 366 |
| FBXO5 | NM_012177 | S2017/FBXO5.r1 | GGATTGTAGACTGTCACCGAAATTC | 367 |
| FBXO5 | NM_012177 | S2018/FBXO5.f1 | GGCTATTCCTCATTTTCTCTACAAAGTG | 368 |
| FBXO5 | NM_012177 | S5061/FBXO5.p1 | CCTCCAGGAGGCTACCTTCTTCATGTTCAC | 369 |
| FDFT1 | NM_004462 | T2006/FDFT1.f1 | AAGGAAAGGGTGCCTCATC | 370 |
| FDFT1 | NM_004462 | T2007/FDFT1.r1 | GAGCCACAAGCAGCACAGT | 371 |
| FDFT1 | NM_004462 | T2008/FDFT1.p1 | CATCACCCACAAGGACAGGTTGCT | 372 |
| FGFR1 | NM_023109 | S0818/FGFR1.f3 | CACGGGACATTCACCACATC | 373 |
| FGFR1 | NM_023109 | S0819/FGFR1.r3 | GGGTGCCATCCACTTCACA | 374 |
| FGFR1 | NM_023109 | S4816/FGFR1.p3 | ATAAAAAGACAACCAACGGCCGACTGC | 375 |
| FHIT | NM_002012 | S2443/FHIT.f1 | CCAGTGGAGCGCTTCCAT | 376 |
| FHIT | NM_002012 | S2444/FHIT.r1 | CTCTCTGGGTCGTCTGAAACAA | 377 |
| FHIT | NM_002012 | S4921/FHIT.p1 | TCGGCCACTTCATCAGGACGCAG | 378 |
| FIGF | NM_004469 | S8941/FIGF.f1 | GGTTCCAGCTTTCTGTAGCTGT | 379 |
| FIGF | NM_004469 | S8942/FIGF.r1 | GCCGCAGGTTCTAGTTGCT | 380 |
| FIGF | NM_004469 | S8943/FIGF.p1 | ATTGGTGGCCACACCACCTCCTTA | 381 |
| FLJ20354 | NM_017779 | S4309/FLJ203.f1 | GCGTATGATTTCCCGAATGAG | 382 |
| (DEPDC1 official) | | | | |
| FLJ20354 | NM_017779 | S4310/FLJ203.r1 | CAGTGACCTCGTACCCATTGC | 383 |
| (DEPDC1 official) | | | | |
| FLJ20354 | NM_017779 | S4311/FLJ203.p1 | ATGTTGATATGCCCAAACTTCATGA | 384 |
| (DEPDC1 official) | | | | |
| FOS | NM_005252 | S6726/FOS.f1 | CGAGCCCTTTGATGACTTCCT | 385 |
| FOS | NM_005252 | S6727/FOS.r1 | GGAGCGGGCTGTCTCAGA | 386 |
| FOS | NM_005252 | S6728/FOS.p1 | TCCCAGCATCATCCAGGCCCAG | 387 |
| FOXM1 | NM_021953 | S2006/FOXM1.f1 | CCACCCCGAGCAAATCTGT | 388 |
| FOXM1 | NM_021953 | S2007/FOXM1.r1 | AAATCCAGTCCCCCTACTTTGG | 389 |
| FOXM1 | NM_021953 | S4757/FOXM1.p1 | CCTGAATCCTGGAGGCTCACGCC | 390 |
| FUS | NM_004960 | S2936/FUS.f1 | GGATAATTCAGACAACAACACCATCT | 391 |
| FUS | NM_004960 | S2937/FUS.r1 | TGAAGTAATCAGCCACAGACTCAAT | 392 |
| FUS | NM_004960 | S4801/FUS.p1 | TCAATTGTAACATTCTCACCCAGGCCTTG | 393 |
| FYN | NM_002037 | S5695/FYN.f3 | GAAGCGCAGATCATGAAGAA | 394 |
| FYN | NM_002037 | S5696/FYN.r3 | CTCCTCAGACACCACTGCAT | 395 |
| FYN | NM_002037 | S5697/FYN.p3 | CTGAAGCACGACAAGCTGGTCCAG | 396 |
| G1P3 | NM_002038 | T1086/G1P3.f1 | CCTCCAACTCCTAGCCTCAA | 397 |
| G1P3 | NM_002038 | T1087/G1P3.r1 | GGCGCATGCTTGTAATCC | 398 |
| G1P3 | NM_002038 | T1088/G1P3.p1 | TGATCCTCCTGTCTCAACCTCCCA | 399 |
| GADD45 | NM_001924 | S5835/GADD45.f3 | GTGCTGGTGACGAATCCA | 400 |
| GADD45 | NM_001924 | S5836/GADD45.r3 | CCCGGCAAAAACAAATAAGT | 401 |
| GADD45 | NM_001924 | S5837/GADD45.p3 | TTCATCTCAATGGAAGGATCCTGCC | 402 |
| GADD45B | NM_015675 | S6929/GADD45.f1 | ACCCTCGACAAGACCACACT | 403 |
| GADD45B | NM_015675 | S6930/GADD45.r1 | TGGGAGTTCATGGGTACAGA | 404 |
| GADD45B | NM_015675 | S6931/GADD45.p1 | AACTTCAGCCCCAGCTCCCAAGTC | 405 |
| GAGE1 | NM_001468 | T2162/GAGE1.f1 | AAGGGCAATCACAGTGTTAAAAGAA | 406 |
| GAGE1 | NM_001468 | T2163/GAGE1.r1 | GGAGAACTTCAATGAAGAATTTTCCA | 407 |
| GAGE1 | NM_001468 | T2164/GAGE1.p1 | CATAGGAGCAGCCTGCAACATTTCAGCAT | 408 |
| GAPDH | NM_002046 | S0374/GAPDH.f1 | ATTCCACCCATGGCAAATTC | 409 |
| GAPDH | NM_002046 | S0375/GAPDH.r1 | GATGGGATTTCCATTGATGACA | 410 |
| GAPDH | NM_002046 | S4738/GAPDH.p1 | CCGTTCTCAGCCTTGACGGTGC | 411 |
| GATA3 | NM_002051 | S0127/GATA3.f3 | CAAAGGAGCTCACTGTGGTGTCT | 412 |
| GATA3 | NM_002051 | S0129/GATA3.r3 | GAGTCAGAATGGCTTATTCACAGATG | 413 |
| GATA3 | NM_002051 | S5005/GATA3.p3 | TGTTCCAACCACTGAATCTGGACC | 414 |
| GBP1 | NM_002053 | S5698/GBP1.f1 | TTGGGAAATATTTGGGCATT | 415 |
| GBP1 | NM_002053 | S5699/GBP1.r1 | AGAAGCTAGGGTGGTTGTCC | 416 |
| GBP1 | NM_002053 | S5700/GBP1.p1 | TTGGGACATTGTAGACTTGGCCAGAC | 417 |
| GBP2 | NM_004120 | S5707/GBP2.f2 | GCATGGGAACCATCAACCA | 418 |
| GBP2 | NM_004120 | S5708/GBP2.r2 | TGAGGAGTTTGCCTTGATTCG | 419 |
| GBP2 | NM_004120 | S5709/GBP2.p2 | CCATGGACCAACTTCACTATGTGACAGAGC | 420 |
| GCLC | NM_001498 | S0772/GCLC.f3 | CTGTTGCAGGAAGGCATTGA | 421 |
| GCLC | NM_001498 | S0773/GCLC.r3 | GTCAGTGGGTCTCTAATAAAGAGATGAG | 422 |
| GCLC | NM_001498 | S4803/GCLC.p3 | CATCTCCTGGCCCAGCATGTT | 423 |
| GDF15 | NM_004864 | S7806/GDF15.f1 | CGCTCCAGACCTATGATGACT | 424 |
| GDF15 | NM_004864 | S7807/GDF15.r1 | ACAGTGGAAGGACCAGGACT | 425 |
| GDF15 | NM_004864 | S7808/GDF15.p1 | TGTTAGCCAAAGACTGCCACTGCA | 426 |
| GGPS1 | NM_004837 | S1590/GGPS1.f1 | CTCCGACGTGGCTTTCCA | 427 |
| GGPS1 | NM_004837 | S1591/GGPS1.r1 | CGTAATTGGCAGAATTGATGACA | 428 |
| GGPS1 | NM_004837 | S4896/GGPS1.p1 | TGGCCCACAGCATCTATGGAATCCC | 429 |
| GLRX | NM_002064 | T2165/GLRX.f1 | GGAGCTCTGCAGTAACCACAGAA | 430 |
| GLRX | NM_002064 | T2166/GLRX.r1 | CAATGCCATCCAGCTCTTGA | 431 |
| GLRX | NM_002064 | T2167/GLRX.p1 | AGGCCCCATGCTGACGTCCCTC | 432 |
| GNS | NM_002076 | T2009/GNS.f1 | GGTGAAGGTTGTCTCTTCCG | 433 |
| GNS | NM_002076 | T2010/GNS.r1 | CAGCCCTTCCACTTGTCTG | 434 |
| GNS | NM_002076 | T2011/GNS.p1 | AAGAGCCCTGTCTTCAGAAGGCCC | 435 |
| GPR56 | NM_005682 | T2120/GPR56.f1 | TACCCTTCCATGTGCTGGAT | 436 |
| GPR56 | NM_005682 | T2121/GPR56.r1 | GCTGAAGAGGCCCAGGTT | 437 |
| GPR56 | NM_005682 | T2122/GPR56.p1 | CGGGACTCCCTGGTCAGCTACATC | 438 |
| GPX1 | NM_000581 | S8296/GPX1.f2 | GCTTATGACCGACCCCAA | 439 |
| GPX1 | NM_000581 | S8297/GPX1.r2 | AAAGTTCCAGGCAACATCGT | 440 |
| GPX1 | NM_000581 | S8298/GPX1.p2 | CTCATCACCTGGTCTCCGGTGTGT | 441 |
| GRB7 | NM_005310 | S0130/GRB7.f2 | CCATCTGCATCCATCTTGTT | 442 |
| GRB7 | NM_005310 | S0132/GRB7.r2 | GGCCACCAGGGTATTATCTG | 443 |
| GRB7 | NM_005310 | S4726/GRB7.p2 | CTCCCCACCCTTGAGAAGTGCCT | 444 |
| GSK3B | NM_002093 | T0408/GSK3B.f2 | GACAAGGACGGCAGCAAG | 445 |
| GSK3B | NM_002093 | T0409/GSK3B.r2 | TTGTGGCCTGTCTGGACC | 446 |
| GSK3B | NM_002093 | T0410/GSK3B.p2 | CCAGGAGTTGCCACCACTGTTGTC | 447 |
| GSR | NM_000637 | S8633/GSR.f1 | GTGATCCCAAGCCCACAATA | 448 |
| GSR | NM_000637 | S8634/GSR.r1 | TGTGGCGATCAGGATGTG | 449 |
| GSR | NM_000637 | S8635/GSR.p1 | TCAGTGGGAAAAAGTACACCGCCC | 450 |
| GSTM1 | NM_000561 | S2026/GSTM1.r1 | GGCCCAGCTTGAATTTTTCA | 451 |
| GSTM1 | NM_000561 | S2027/GSTM1.f1 | AAGCTATGAGGAAAAGAAGTACACGAT | 452 |
| GSTM1 | NM_000561 | S4739/GSTM1.p1 | TCAGCCACTGGCTTCTGTCATAATCAGGAG | 453 |
| GSTp | NM_000852 | S0136/GSTp.f3 | GAGACCCTGCTGTCCCAGAA | 454 |
| GSTp | NM_000852 | S0138/GSTp.r3 | GGTTGTAGTCAGCGAAGGAGATC | 455 |
| GSTp | NM_000852 | S5007/GSTp.p3 | TCCCACAATGAAGGTCTTGCCTCCCT | 456 |
| GUS | NM_000181 | S0139/GUS.f1 | CCCACTCAGTAGCCAAGTCA | 457 |
| GUS | NM_000181 | S0141/GUS.r1 | CACGCAGGTGGTATCAGTCT | 458 |
| GUS | NM_000181 | S4740/GUS.p1 | TCAAGTAAACGGGCTGTTTTCCAAACA | 459 |
| HDAC6 | NM_006044 | S9451/HDAC6.f1 | TCCTGTGCTCTGGAAGCC | 460 |
| HDAC6 | NM_006044 | S9452/HDAC6.r1 | CTCCACGGTCTCAGTTGATCT | 461 |
| HDAC6 | NM_006044 | S9453/HDAC6.p1 | CAAGAACCTCCCAGAAGGGCTCAA | 462 |
| HER2 | NM_004448 | S0142/HER2.f3 | CGGTGTGAGAAGTGCAGCAA | 463 |
| HER2 | NM_004448 | S0144/HER2.r3 | CCTCTCGCAAGTGCTCCAT | 464 |
| HER2 | NM_004448 | S4729/HER2.p3 | CCAGACCATAGCACACTCGGGCAC | 465 |
| HIF1A | NM_001530 | S1207/HIF1A.f3 | TGAACATAAAGTCTGCAACATGGA | 466 |
| HIF1A | NM_001530 | S1208/HIF1A.r3 | TGAGGTTGGTTACTGTTGGTATCATATA | 467 |
| HIF1A | NM_001530 | S4753/HIF1A.p3 | TTGCACTGCACAGGCCACATTCAC | 468 |
| HNF3A | NM_004496 | S0148/HNF3A.f1 | TCCAGGATGTTAGGAACTGTGAAG | 469 |
| HNF3A | NM_004496 | S0150/HNF3A.r1 | GCGTGTCTGCGTAGTAGCTGTT | 470 |
| HNF3A | NM_004496 | S5008/HNF3A.p1 | AGTCGCTGGTTTCATGCCCTTCCA | 471 |
| HRAS | NM_005343 | S8427/HRAS.f1 | GGACGAATACGACCCCACT | 472 |
| HRAS | NM_005343 | S8428/HRAS.r1 | GCACGTCTCCCCATCAAT | 473 |
| HRAS | NM_005343 | S8429/HRAS.p1 | ACCACCTGCTTCCGGTAGGAATCC | 474 |
| HSPA1A | NM_005345 | S6708/HSPA1A.f1 | CTGCTGCGACAGTCCACTA | 475 |
| HSPA1A | NM_005345 | S6709/HSPA1A.r1 | CAGGTTCGCTCTGGGAAG | 476 |
| HSPA1A | NM_005345 | S6710/HSPA1A.p1 | AGAGTGACTCCCGTTGTCCCAAGG | 477 |
| HSPA1B | NM_005346 | S6714/HSPA1B.f1 | GGTCCGCTTCGTCTTTCGA | 478 |
| HSPA1B | NM_005346 | S6715/HSPA1B.r1 | GCACAGGTTCGCTCTGGAA | 479 |
| HSPA1B | NM_005346 | S6716/HSPA1B.p1 | TGACTCCCGCGGTCCCAAGG | 480 |
| HSPA1L | NM_005527 | T2015/HSPA1L.f1 | GCAGGTGTGATTGCTGGAC | 481 |
| HSPA1L | NM_005527 | T2016/HSPA1L.r1 | ACCATAGGCAATGGCAGC | 482 |
| HSPA1L | NM_005527 | T2017/HSPA1L.p1 | AAGAATCATCAATGAGCCCACGGC | 483 |
| HSPA5 | NM_005347 | S7166/HSPA5.f1 | GGCTAGTAGAACTGGATCCCAACA | 484 |
| HSPA5 | NM_005347 | S7167/HSPA5.r1 | GGTCTGCCCAAATGCTTTTC | 485 |
| HSPA5 | NM_005347 | S7168/HSPA5.p1 | TAATTAGACCTAGGCCTCAGCTGCACTGCC | 486 |
| HSPA9B | NM_004134 | T2018/HSPA9B.f1 | GGCCACTAAAGATGCTGGC | 487 |
| HSPA9B | NM_004134 | T2019/HSPA9B.r1 | AGCAGCTGTGGGCTCATT | 488 |
| HSPA9B | NM_004134 | T2020/HSPA9B.p1 | ATCACCCGAAGCACATTCAGTCCA | 489 |
| HSPB1 | NM_001540 | S6720/HSPB1.f1 | CCGACTGGAGGAGCATAAA | 490 |
| HSPB1 | NM_001540 | S6721/HSPB1.r1 | ATGCTGGCTGACTCTGCTC | 491 |
| HSPB1 | NM_001540 | S6722/HSPB1.p1 | CGCACTTTTCTGAGCAGACGTCCA | 492 |
| HSPCA | NM_005348 | S7097/HSPCA.f1 | CAAAAGGCAGAGGCTGATAA | 493 |
| HSPCA | NM_005348 | S7098/HSPCA.r1 | AGCGCAGTTTCATAAAGCAA | 494 |
| HSPCA | NM_005348 | S7099/HSPCA.p1 | TGACCAGATCCTTCACAGACTTGTCGT | 495 |
| ID1 | NM_002165 | S0820/ID1.f1 | AGAACCGCAAGGTGAGCAA | 496 |
| ID1 | NM_002165 | S0821/ID1.r1 | TCCAACTGAAGGTCCCTGATG | 497 |
| ID1 | NM_002165 | S4832/ID1.p1 | TGGAGATTCTCCAGCACGTCATCGAC | 498 |
| IFITM1 | NM_003641 | S7768/IFITM1.f1 | CACGCAGAAAACCACACTTC | 499 |
| IFITM1 | NM_003641 | S7769/IFITM1.r1 | CATGTTCCTCCTTGTGCATC | 500 |
| IFITM1 | NM_003641 | S7770/IFITM1.p1 | CAACACTTCCTTCCCCAAAGCCAG | 501 |
| IGF1R | NM_000875 | S1249/IGF1R.f3 | GCATGGTAGCCGAAGATTTCA | 502 |
| IGF1R | NM_000875 | S1250/IGF1R.r3 | TTTCCGGTAATAGTCTGTCTCATAGATATC | 503 |
| IGF1R | NM_000875 | S4895/IGF1R.p3 | CGCGTCATACCAAAATCTCCGATTTTGA | 504 |
| IGFBP2 | NM_000597 | S1128/IGFBP2.f1 | GTGGACAGCACCATGAACA | 505 |
| IGFBP2 | NM_000597 | S1129/IGFBP2.r1 | CCTTCATACCCGACTTGAGG | 506 |
| IGFBP2 | NM_000597 | S4837/IGFBP2.p1 | CTTCCGGCCAGCACTGCCTC | 507 |
| IGFBP3 | NM_000598 | S0157/IGFBP3.f3 | ACGCACCGGGTGTCTGA | 508 |
| IGFBP3 | NM_000598 | S0159/IGFBP3.r3 | TGCCCTTTCTTGATGATGATTATC | 509 |
| IGFBP3 | NM_000598 | S5011/IGFBP3.p3 | CCCAAGTTCCACCCCCTCCATTCA | 510 |
| IGFBP5 | NM_000599 | S1644/IGFBP5.f1 | TGGACAAGTACGGGATGAAGCT | 511 |
| IGFBP5 | NM_000599 | S1645/IGFBP5.r1 | CGAAGGTGTGGCACTGAAAGT | 512 |
| IGFBP5 | NM_000599 | S4908/IGFBP5.p1 | CCCGTCAACGTACTCCATGCCTGG | 513 |
| IL-7 | NM_000880 | S5781/IL-7.f1 | GCGGTGATTCGGAAATTCG | 514 |
| IL-7 | NM_000880 | S5782/IL-7.r1 | CTCTCCTGGGCACCTGCTT | 515 |
| IL-7 | NM_000880 | S5783/IL-7.p1 | CTCTGGTCCTCATCCAGGTGCGC | 516 |
| IL-8 | NM_000584 | S5790/IL-8.f1 | AAGGAACCATCTCACTGTGTGTAAAC | 517 |
| IL-8 | NM_000584 | S5791/IL-8.r1 | ATCAGGAAGGCTGCCAAGAG | 518 |
| IL-8 | NM_000584 | S5792/IL-8.p1 | TGACTTCCAAGCTGGCCGTGGC | 519 |
| IL2RA | NM_000417 | T2147/IL2RA.f1 | TCTGCGTGGTTCCTTTCTCA | 520 |
| IL2RA | NM_000417 | T2148/IL2RA.r1 | TTGAAGGATGTTTATTAGGCAACGT | 521 |
| IL2RA | NM_000417 | T2149/IL2RA.p1 | CGCTTCTGACTGCTGATTCTCCCGTT | 522 |
| IL6 | NM_000600 | S0760/IL6.f3 | CCTGAACCTTCCAAAGATGG | 523 |
| IL6 | NM_000600 | S0761/IL6.r3 | ACCAGGCAAGTCTCCTCATT | 524 |
| IL6 | NM_000600 | S4800/IL6.p3 | CCAGATTGGAAGCATCCATCTTTTTCA | 525 |
| IL8RB | NM_001557 | T2168/IL8RB.f1 | CCGCTCCGTCACTGATGTCT | 526 |
| IL8RB | NM_001557 | T2169/IL8RB.r1 | GCAAGGTCAGGGCAAAGAGTA | 527 |
| IL8RB | NM_001557 | T2170/IL8RB.p1 | CCTGCTGAACCTAGCCTTGGCCGA | 528 |
| ILK | NM_001014794 | T0618/ILK.f1 | CTCAGGATTTTCTCGCATCC | 529 |
| ILK | NM_001014794 | T0619/ILK.r1 | AGGAGCAGGTGGAGACTGG | 530 |
| ILK | NM_001014794 | T0620/ILK.p1 | ATGTGCTCCCAGTGCTAGGTGCCT | 531 |
| ILT-2 | NM_006669 | S1611/ILT-2.f2 | AGCCATCACTCTCAGTGCAG | 532 |
| ILT-2 | NM_006669 | S1612/ILT-2.r2 | ACTGCAGAGTCAGGGTCTCC | 533 |
| ILT-2 | NM_006669 | S4904/ILT-2.p2 | CAGGTCCTATCGTGGCCCCTGA | 534 |
| INCENP | NM_020238 | T2024/INCENP.f1 | GCCAGGATACTGGAGTCCATC | 535 |
| INCENP | NM_020238 | T2025/INCENP.r1 | CTTGACCCTTGGGGTCCT | 536 |
| INCENP | NM_020238 | T2026/INCENP.p1 | TGAGCTCCCTGATGGCTACACCC | 537 |
| IRAK2 | NM_001570 | T2027/IRAK2.f1 | GGATGGAGTTCGCCTCCT | 538 |
| IRAK2 | NM_001570 | T2028/IRAK2.r1 | CGCTCCATGGACTTGATCTT | 539 |
| IRAK2 | NM_001570 | T2029/IRAK2.p1 | CGTGATCACAGACCTGACCCAGCT | 540 |
| IRS1 | NM_005544 | S1943/IRS1.f3 | CCACAGCTCACCTTCTGTCA | 541 |
| IRS1 | NM_005544 | S1944/IRS1.r3 | CCTCAGTGCCAGTCTCTTCC | 542 |
| IRS1 | NM_005544 | S5050/1RS1.p3 | TCCATCCCAGCTCCAGCCAG | 543 |
| ITGB1 | NM_002211 | S7497/ITGB1.f1 | TCAGAATTGGATTTGGCTCA | 544 |
| ITGB1 | NM_002211 | S7498/ITGB1.r1 | CCTGAGCTTAGCTGGTGTTG | 545 |
| ITGB1 | NM_002211 | S7499/ITGB1.p1 | TGCTAATGTAAGGCATCACAGTCTTTTCCA | 546 |
| K-Alpha-1 | NM_006082 | S8706/K-Alph.f2 | TGAGGAAGAAGGAGAGGAATACTAAT | 547 |
| K-Alpha-1 | NM_006082 | S8707/K-Alph.r2 | CTGAAATTCTGGGAGCATGAC | 548 |
| K-Alpha-1 | NM_006082 | S8708/K-Alph.p2 | TATCCATTCCTTTTGGCCCTGCAG | 549 |
| KDR | NM_002253 | S1343/KDR.f6 | GAGGACGAAGGCCTCTACAC | 550 |
| KDR | NM_002253 | S1344/KDR.r6 | AAAAATGCCTCCACTTTTGC | 551 |
| KDR | NM_002253 | S4903/KDR.p6 | CAGGCATGCAGTGTTCTTGGCTGT | 552 |
| Ki-67 | NM_002417 | S0436/Ki-67.f2 | CGGACTTTGGGTGCGACTT | 553 |
| Ki-67 | NM_002417 | S0437/Ki-67.r2 | TTACAACTCTTCCACTGGGACGAT | 554 |
| Ki-67 | NM_002417 | S4741/Ki-67.p2 | CCACTTGTCGAACCACCGCTCGT | 555 |
| KIF11 | NM_004523 | T2409/KIF11.f2 | TGGAGGTTGTAAGCCAATGT | 556 |
| KIF11 | NM_004523 | T2410/KIF11.r2 | TGCCTTACGTCCATCTGATT | 557 |
| KIF11 | NM_004523 | T2411/KIF11.p2 | CAGTGATGTCTGAACTTGAAGCCTCACA | 558 |
| KIF22 | NM_007317 | S8505/KIF22.f1 | CTAAGGCACTTGCTGGAAGG | 559 |
| KIF22 | NM_007317 | S8506/KIF22.r1 | TCTTCCCAGCTCCTGTGG | 560 |
| KIF22 | NM_007317 | S8507/KIF22.p1 | TCCATAGGCAAGCACACTGGCATT | 561 |
| KIF2C | NM_006845 | S7262/KIF2C.f1 | AATTCCTGCTCCAAAAGAAAGTCTT | 562 |
| KIF2C | NM_006845 | S7263/KIF2C.r1 | CGTGATGCGAAGCTCTGAGA | 563 |
| KIF2C | NM_006845 | S7264/KIF2C.p1 | AAGCCGCTCCACTCGCATGTCC | 564 |
| KIFC1 | NM_002263 | S8517/KIFC1.f1 | CCACAGGGTTGAAGAACCAG | 565 |
| KIFC1 | NM_002263 | S8519/KIFC1.r1 | CACCTGATGTGCCAGACTTC | 566 |
| KIFC1 | NM_002263 | S8520/KIFC1.p1 | AGCCAGTTCCTGCTGTTCCTGTCC | 567 |
| KLK10 | NM_002776 | S2624/KLK10.f3 | GCCCAGAGGCTCCATCGT | 568 |
| KLK10 | NM_002776 | S2625/KLK10.r3 | CAGAGGTTTGAACAGTGCAGACA | 569 |
| KLK10 | NM_002776 | S4978/KLK10.p3 | CCTCTTCCTCCCCAGTCGGCTGA | 570 |
| KNS2 | NM_005552 | T2030/KNS2.f1 | CAAACAGAGGGTGGCAGAAG | 571 |
| KNS2 | NM_005552 | T2031/KNS2.r1 | GAGGCTCTCACGGCTCCT | 572 |
| KNS2 | NM_005552 | T2032/KNS2.p1 | CGCTTCTCCATGTTCTCAGGGTCA | 573 |
| KNTC1 | NM_014708 | T2126/KNTC1.f1 | AGCCGAGGCTTTGTTGAA | 574 |
| KNTC1 | NM_014708 | T2127/KNTC1.r1 | TGGGCTATGAGCACAGCTT | 575 |
| KNTC1 | NM_014708 | T2128/KNTC1.p1 | TTCATATCCAGTACCGGCGATCGG | 576 |
| KNTC2 | NM_006101 | S7296/KNTC2.f1 | ATGTGCCAGTGAGCTTGAGT | 577 |
| KNTC2 | NM_006101 | S7297/KNTC2.r1 | TGAGCCCCTGGTTAACAGTA | 578 |
| KNTC2 | NM_006101 | S7298/KNTC2.p1 | CCTTGGAGAAACACAAGCACCTGC | 579 |
| KRT14 | NM_000526 | S1853/KRT14.f1 | GGCCTGCTGAGATCAAAGAC | 580 |
| KRT14 | NM_000526 | S1854/KRT14.r1 | GTCCACTGTGGCTGTGAGAA | 581 |
| KRT14 | NM_000526 | S5037/KRT14.p1 | TGTTCCTCAGGTCCTCAATGGTCTTG | 582 |
| KRT17 | NM_000422 | S0172/KRT17.f2 | CGAGGATTGGTTCTTCAGCAA | 583 |
| KRT17 | NM_000422 | S0173/KRT17.p2 | CACCTCGCGGTTCAGTTCCTCTGT | 584 |
| KRT17 | NM_000422 | S0174/KRT17.r2 | ACTCTGCACCAGCTCACTGTTG | 585 |
| KRT19 | NM_002276 | S1515/KRT19.f3 | TGAGCGGCAGAATCAGGAGTA | 586 |
| KRT19 | NM_002276 | S1516/KRT19.r3 | TGCGGTAGGTGGCAATCTC | 587 |
| KRT19 | NM_002276 | S4866/KRT19.p3 | CTCATGGACATCAAGTCGCGGCTG | 588 |
| KRT5 | NM_000424 | S0175/KRT5.f3 | TCAGTGGAGAAGGAGTTGGA | 589 |
| KRT5 | NM_000424 | S0177/KRT5.r3 | TGCCATATCCAGAGGAAACA | 590 |
| KRT5 | NM_000424 | S5015/KRT5.p3 | CCAGTCAACATCTCTGTTGTCACAAGCA | 591 |
| L1CAM | NM_000425 | T1341/L1CAM.f1 | CTTGCTGGCCAATGCCTA | 592 |
| L1CAM | NM_000425 | T1342/L1CAM.r1 | TGATTGTCCGCAGTCAGG | 593 |
| L1CAM | NM_000425 | T1343/L1CAM.p1 | ATCTACGTTGTCCAGCTGCCAGCC | 594 |
| LAMC2 | NM_005562 | S2826/LAMC2.f2 | ACTCAAGCGGAAATTGAAGCA | 595 |
| LAMC2 | NM_005562 | S2827/LAMC2.r2 | ACTCCCTGAAGCCGAGACACT | 596 |
| LAMC2 | NM_005562 | S4969/LAMC2.p2 | AGGTCTTATCAGCACAGTCTCCGCCTCC | 597 |
| LAPTM4B | NM_018407 | T2063/LAPTM4.f1 | AGCGATGAAGATGGTCGC | 598 |
| LAPTM4B | NM_018407 | T2064/LAPTM4.r1 | GACATGGCAGCACAAGCA | 599 |
| LAPTM4B | NM_018407 | T2065/LAPTM4.p1 | CTGGACGCGGTTCTACTCCAACAG | 600 |
| LIMK1 | NM_016735 | T0759/LIMK1.f1 | GCTTCAGGTGTTGTGACTGC | 601 |
| LIMK1 | NM_016735 | T0760/LIMK1.r1 | AAGAGCTGCCCATCCTTCTC | 602 |
| LIMK1 | NM_016735 | T0761/LIMK1.p1 | TGCCTCCCTGTCGCACCAGTACTA | 603 |
| LIMK2 | NM_005569 | T2033/LIMK2.f1 | CTTTGGGCCAGGAGGAAT | 604 |
| LIMK2 | NM_005569 | T2034/LIMK2.r1 | CTCCCACAATCCACTGCC | 605 |
| LIMK2 | NM_005569 | T2035/LIMK2.p1 | ACTCGAATCCACCCAGGAACTCCC | 606 |
| MAD1L1 | NM_003550 | S7299/MAD1L1.f1 | AGAAGCTGTCCCTGCAAGAG | 607 |
| MAD1L1 | NM_003550 | S7300/MAD1L1.r1 | AGCCGTACCAGCTCAGACTT | 608 |
| MAD1L1 | NM_003550 | S7301/MAD1L1.p1 | CATGTTCTTCACAATCGCTGCATCC | 609 |
| MAD2L1 | NM_002358 | S7302/MAD2L1.f1 | CCGGGAGCAGGGAATCAC | 610 |
| MAD2L1 | NM_002358 | S7303/MAD2L1.r1 | ATGCTGTTGATGCCGAATGA | 611 |
| MAD2L1 | NM_002358 | S7304/MAD2L1.p1 | CGGCCACGATTTCGGCGCT | 612 |
| MAD2L1BP | NM_014628 | T2123/MAD2L1.f1 | CTGTCATGTGGCAGACCTTC | 613 |
| MAD2L1BP | NM_014628 | T2124/MAD2L1.r1 | TAAATGTCACTGGTGCCTGG | 614 |
| MAD2L1BP | NM_014628 | T2125/MAD2L1.p1 | CGAACCACGGCTTGGGAAGACTAC | 615 |
| MAD2L2 | NM_006341 | T1125/MAD2L2.f1 | GCCCAGTGGAGAAATTCGT | 616 |
| MAD2L2 | NM_006341 | T1126/MAD2L2.r1 | GCGAGTCTGAGCTGATGGA | 617 |
| MAD2L2 | NM_006341 | T1127/MAD2L2.p1 | TTTGAGATCACCCAGCCTCCACTG | 618 |
| MAGE2 | NM_005361 | S5623/MAGE2.f1 | CCTCAGAAATTGCCAGGACT | 619 |
| MAGE2 | NM_005361 | S5625/MAGE2.p1 | TTCCCGTGATCTTCAGCAAAGCCT | 620 |
| MAGE2 | NM_005361 | S5626/MAGE2.r1 | CCAAAGACCAGCTGCAAGTA | 621 |
| MAGE6 | NM_005363 | S5639/MAGE6.f3 | AGGACTCCAGCAACCAAGAA | 622 |
| MAGE6 | NM_005363 | S5640/MAGE6.r3 | GAGTGCTGCTTGGAACTCAG | 623 |
| MAGE6 | NM_005363 | S5641/MAGE6.p3 | CAAGCACCTTCCCTGACCTGGAGT | 624 |
| MAP2 | NM_031846 | S8493/MAP2.f1 | CGGACCACCAGGTCAGAG | 625 |
| MAP2 | NM_031846 | S8494/MAP2.r1 | CAGGGGTAGTGGGTGTTGAG | 626 |
| MAP2 | NM_031846 | S8495/MAP2.p1 | CCACTCTTCCCTGCTCTGCGAATT | 627 |
| MAP2K3 | NM_002756 | T2090/MAP2K3.f1 | GCCCTCCAATGTCCTTATCA | 628 |
| MAP2K3 | NM_002756 | T2091/MAP2K3.r1 | GTAGCCACTGATGCCAAAGTC | 629 |
| MAP2K3 | NM_002756 | T2092/MAP2K3.p1 | CACATCTTCACATGGCCCTCCTTG | 630 |
| MAP4 | NM_002375 | S5724/MAP4.f1 | GCCGGTCAGGCACACAAG | 631 |
| MAP4 | NM_002375 | S5725/MAP4.r1 | GCAGCATACACACAACAAAATGG | 632 |
| MAP4 | NM_002375 | S5726/MAP4.p1 | ACCAACCAGTCCACGCTCCAAGGG | 633 |
| MAP6 | NM_033063 | T2341/MAP6.f2 | CCCTCAACCGGCAAATCC | 634 |
| MAP6 | NM_033063 | T2342/MAP6.r2 | CGTCCATGCCCTGAATTCA | 635 |
| MAP6 | NM_033063 | T2343/MAP6.p2 | TGGCGAGTGCAGTGAGCAGCTCC | 636 |
| MAPK14 | NM_139012 | S5557/MAPK14.f2 | TGAGTGGAAAAGCCTGACCTATG | 637 |
| MAPK14 | NM_139012 | S5558/MAPK14.r2 | GGACTCCATCTCTTCTTGGTCAA | 638 |
| MAPK14 | NM_139012 | S5559/MAPK14.p2 | TGAAGTCATCAGCTTTGTGCCACCACC | 639 |
| MAPK8 | NM_002750 | T2087/MAPK8.f1 | CAACACCCGTACATCAATGTCT | 640 |
| MAPK8 | NM_002750 | T2088/MAPK8.r1 | TCATCTAACTGCTTGTCAGGGA | 641 |
| MAPK8 | NM_002750 | T2089/MAPK8.p1 | CTGAAGCAGAAGCTCCACCACCAA | 642 |
| MAPRE1 | NM_012325 | T2180/MAPRE1.f1 | GACCTTGGAACCTTTGGAAC | 643 |
| MAPRE1 | NM_012325 | T2181/MAPRE1.r1 | CCTAGGCCTATGAGGGTTCA | 644 |
| MAPRE1 | NM_012325 | T2182/MAPRE1.p1 | CAGCCCTGTAAGACCTGTTGACAGCA | 645 |
| MAPT | NM_016835 | S8502/MAPT.f1 | CACAAGCTGACCTTCCGC | 646 |
| MAPT | NM_016835 | S8503/MAPT.r1 | ACTTGTACACGATCTCCGCC | 647 |
| MAPT | NM_016835 | S8504/MAPT.p1 | AGAACGCCAAAGCCAAGACAGACC | 648 |
| Maspin | NM_002639 | S0836/Maspin.f2 | CAGATGGCCACTTTGAGAACATT | 649 |
| Maspin | NM_002639 | S0837/Maspin.r2 | GGCAGCATTAACCACAAGGATT | 650 |
| Maspin | NM_002639 | S4835/Maspin.p2 | AGCTGACAACAGTGTGAACGACCAGACC | 651 |
| MCL1 | NM_021960 | S5545/MCL1.f1 | CTTCGGAAACTGGACATCAA | 652 |
| MCL1 | NM_021960 | S5546/MCL1.r1 | GTCGCTGAAAACATGGATCA | 653 |
| MCL1 | NM_021960 | S5547/MCL1.p1 | TCACTCGAGACAACGATTTCACATCG | 654 |
| MCM2 | NM_004526 | S1602/MCM2.f2 | GACTTTTGCCCGCTACCTTTC | 655 |
| MCM2 | NM_004526 | S1603/MCM2.r2 | GCCACTAACTGCTTCAGTATGAAGAG | 656 |
| MCM2 | NM_004526 | S4900/MCM2.p2 | ACAGCTCATTGTTGTCACGCCGGA | 657 |
| MCM6 | NM_005915 | S1704/MCM6.f3 | TGATGGTCCTATGTGTCACATTCA | 658 |
| MCM6 | NM_005915 | S1705/MCM6.r3 | TGGGACAGGAAACACACCAA | 659 |
| MCM6 | NM_005915 | S4919/MCM6.p3 | CAGGTTTCATACCAACACAGGCTTCAGCAC | 660 |
| MCP1 | NM_002982 | S1955/MCP1.f1 | CGCTCAGCCAGATGCAATC | 661 |
| MCP1 | NM_002982 | S1956/MCP1.r1 | GCACTGAGATCTTCCTATTGGTGAA | 662 |
| MCP1 | NM_002982 | S5052/MCP1.p1 | TGCCCCAGTCACCTGCTGTTA | 663 |
| MGMT | NM_002412 | S1922/MGMT.f1 | GTGAAATGAAACGCACCACA | 664 |
| MGMT | NM_002412 | S1923/MGMT.r1 | GACCCTGCTCACAACCAGAC | 665 |
| MGMT | NM_002412 | S5045/MGMT.p1 | CAGCCCTTTGGGGAAGCTGG | 666 |
| MMP12 | NM_002426 | S4381/MMP12.f2 | CCAACGCTTGCCAAATCCT | 667 |
| MMP12 | NM_002426 | S4382/MMP12.r2 | ACGGTAGTGACAGCATCAAAACTC | 668 |
| MMP12 | NM_002426 | S4383/MMP12.p2 | AACCAGCTCTCTGTGACCCCAATT | 669 |
| MMP2 | NM_004530 | S1874/MMP2.f2 | CCATGATGGAGAGGCAGACA | 670 |
| MMP2 | NM_004530 | S1875/MMP2.r2 | GGAGTCCGTCCTTACCGTCAA | 671 |
| MMP2 | NM_004530 | S5039/MMP2.p2 | CTGGGAGCATGGCGATGGATACCC | 672 |
| MMP9 | NM_004994 | S0656/MMP9.f1 | GAGAACCAATCTCACCGACA | 673 |
| MMP9 | NM_004994 | S0657/MMP9.r1 | CACCCGAGTGTAACCATAGC | 674 |
| MMP9 | NM_004994 | S4760/MMP9.p1 | ACAGGTATTCCTCTGCCAGCTGCC | 675 |
| MRE11A | NM_005590 | T2039/MRE11A.f1 | GCCATGCTGGCTCAGTCT | 676 |
| MRE11A | NM_005590 | T2040/MRE11A.r1 | CACCCAGACCCACCTAACTG | 677 |
| MRE11A | NM_005590 | T2041/MRE11A.p1 | CACTAGCTGATGTGGCCCACAGCT | 678 |
| MRP1 | NM_004996 | S0181/MRP1.f1 | TCATGGTGCCCGTCAATG | 679 |
| MRP1 | NM_004996 | S0183/MRP1.r1 | CGATTGTCTTTGCTCTTCATGTG | 680 |
| MRP1 | NM_004996 | S5019/MRP1.p1 | ACCTGATACGTCTTGGTCTTCATCGCCAT | 681 |
| MRP2 | NM_000392 | S0184/MRP2.f3 | AGGGGATGACTTGGACACAT | 682 |
| MRP2 | NM_000392 | S0186/MRP2.r3 | AAAACTGCATGGCTTTGTCA | 683 |
| MRP2 | NM_000392 | S5021/MRP2.p3 | CTGCCATTCGACATGACTGCAATTT | 684 |
| MRP3 | NM_003786 | S0187/MRP3.f1 | TCATCCTGGCGATCTACTTCCT | 685 |
| MRP3 | NM_003786 | S0189/MRP3.r1 | CCGTTGAGTGGAATCAGCAA | 686 |
| MRP3 | NM_003786 | S5023/MRP3.p1 | TCTGTCCTGGCTGGAGTCGCTTTCAT | 687 |
| MSH3 | NM_002439 | S5940/MSH3.f2 | TGATTACCATCATGGCTCAGA | 688 |
| MSH3 | NM_002439 | S5941/MSH3.r2 | CTTGTGAAAATGCCATCCAC | 689 |
| MSH3 | NM_002439 | S5942/MSH3.p2 | TCCCAATTGTCGCTTCTTCTGCAG | 690 |
| MUC1 | NM_002456 | S0782/MUC1.f2 | GGCCAGGATCTGTGGTGGTA | 691 |
| MUC1 | NM_002456 | S0783/MUC1.r2 | CTCCACGTCGTGGACATTGA | 692 |
| MUC1 | NM_002456 | S4807/MUC1.p2 | CTCTGGCCTTCCGAGAAGGTACC | 693 |
| MX1 | NM_002462 | S7611/MX1.f1 | GAAGGAATGGGAATCAGTCATGA | 694 |
| MX1 | NM_002462 | S7612/MX1.r1 | GTCTATTAGAGTCAGATCCGGGACAT | 695 |
| MX1 | NM_002462 | S7613/MX1.p1 | TCACCCTGGAGATCAGCTCCCGA | 696 |
| MYBL2 | NM_002466 | S3270/MYBL2.f1 | GCCGAGATCGCCAAGATG | 697 |
| MYBL2 | NM_002466 | S3271/MYBL2.r1 | CTTTTGATGGTAGAGTTCCAGTGATTC | 698 |
| MYBL2 | NM_002466 | S4742/MYBL2.p1 | CAGCATTGTCTGTCCTCCCTGGCA | 699 |
| MYH11 | NM_002474 | S4555/MYH11.f1 | CGGTACTTCTCAGGGCTAATATATACG | 700 |
| MYH11 | NM_002474 | S4556/MYH11.r1 | CCGAGTAGATGGGCAGGTGTT | 701 |
| MYH11 | NM_002474 | S4557/MYH11.p1 | CTCTTCTGCGTGGTGGTCAACCCCTA | 702 |
| NEK2 | NM_002497 | S4327/NEK2.f1 | GTGAGGCAGCGCGACTCT | 703 |
| NEK2 | NM_002497 | S4328/NEK2.r1 | TGCCAATGGTGTACAACACTTCA | 704 |
| NEK2 | NM_002497 | S4329/NEK2.p1 | TGCCTTCCCGGGCTGAGGACT | 705 |
| NFKBp50 | NM_003998 | S9661/NFKBp5.f3 | CAGACCAAGGAGATGGACCT | 706 |
| NFKBp50 | NM_003998 | S9662/NFKBp5.r3 | AGCTGCCAGTGCTATCCG | 707 |
| NFKBp50 | NM_003998 | S9663/NFKBp5.p3 | AAGCTGTAAACATGAGCCGCACCA | 708 |
| NFKBp65 | NM_021975 | S0196/NFKBp6.f3 | CTGCCGGGATGGCTTCTAT | 709 |
| NFKBp65 | NM_021975 | S0198/NFKBp6.r3 | CCAGGTTCTGGAAACTGTGGAT | 710 |
| NFKBp65 | NM_021975 | S5030/NFKBp6.p3 | CTGAGCTCTGCCCGGACCGCT | 711 |
| NME6 | NM_005793 | T2129/NME6.f1 | CACTGACACCCGCAACAC | 712 |
| NME6 | NM_005793 | T2130/NME6.r1 | GGCTGCAATCTCTCTGCTG | 713 |
| NME6 | NM_005793 | T2131/NME6.p1 | AACCACAGAGTCCGAACCATGGGT | 714 |
| NPC2 | NM_006432 | T2141/NPC2.f1 | CTGCTTCTTTCCCGAGCTT | 715 |
| NPC2 | NM_006432 | T2142/NPC2.r1 | AGCAGGAATGTAGCTGCCA | 716 |
| NPC2 | NM_006432 | T2143/NPC2.p1 | ACTTCGTTATCCGCGATGCGTTTC | 717 |
| NPD009 (ABAT | NM_020686 | S4474/NPD009.f3 | GGCTGTGGCTGAGGCTGTAG | 718 |
| official) | | | | |
| NPD009 (ABAT | NM_020686 | S4475/NPD009.r3 | GGAGCATTCGAGGTCAAATCA | 719 |
| official) | | | | |
| NPD009 (ABAT | NM_020686 | S4476/NPD009.p3 | TTCCCAGAGTGTCTCACCTCCAGCAGAG | 720 |
| official) | | | | |
| NTSR2 | NM_012344 | T2332/NTSR2.f2 | CGGACCTGAATGTAATGCAA | 721 |
| NTSR2 | NM_012344 | T2333/NTSR2.r2 | CTTTGCCAGGTGACTAAGCA | 722 |
| NTSR2 | NM_012344 | T2334/NTSR2.p2 | AATGAACAGAACAAGCAAAATGACCAGC | 723 |
| NUSAP1 | NM_016359 | S7106/NUSAP1.f1 | CAAAGGAAGAGCAACGGAAG | 724 |
| NUSAP1 | NM_016359 | S7107/NUSAP1.r1 | ATTCCCAAAACCTTTGCTT | 725 |
| NUSAP1 | NM_016359 | S7108/NUSAP1.p1 | TTCTCCTTTCGTTCTTGCTCGCGT | 726 |
| p21 | NM_000389 | S0202/p21.f3 | TGGAGACTCTCAGGGTCGAAA | 727 |
| p21 | NM_000389 | S0204/p21.r3 | GGCGTTTGGAGTGGTAGAAATC | 728 |
| p21 | NM_000389 | S5047/p21.p3 | CGGCGGCAGACCAGCATGAC | 729 |
| p27 | NM_004064 | S0205/p27.f3 | CGGTGGACCACGAAGAGTTAA | 730 |
| p27 | NM_004064 | S0207/p27.r3 | GGCTCGCCTCTTCCATGTC | 731 |
| p27 | NM_004064 | S4750/p27.p3 | CCGGGACTTGGAGAAGCACTGCA | 732 |
| PCTK1 | NM_006201 | T2075/PCTK1.f1 | TCACTACCAGCTGACATCCG | 733 |
| PCTK1 | NM_006201 | T2076/PCTK1.r1 | AGATGGGGCTATTGAGGGTC | 734 |
| PCTK1 | NM_006201 | T2077/PCTK1.p1 | CTTCTCCAGGTAGCCCTCAGGCAG | 735 |
| PDGFRb | NM_002609 | S1346/PDGFRb.f3 | CCAGCTCTCCTTCCAGCTAC | 736 |
| PDGFRb | NM_002609 | S1347/PDGFRb.r3 | GGGTGGCTCTCACTTAGCTC | 737 |
| PDGFRb | NM_002609 | S4931/PDGFRb.p3 | ATCAATGTCCCTGTCCGAGTGCTG | 738 |
| PFDN5 | NM_145897 | T2078/PFDN5.f1 | GAGAAGCACGCCATGAAAC | 739 |
| PFDN5 | NM_145897 | T2079/PFDN5.r1 | GGCTGTGAGCTGCTGAATCT | 740 |
| PFDN5 | NM_145897 | T2080/PFDN5.p1 | TGACTCATCATTTCCATGACGGCC | 741 |
| PGK1 | NM_000291 | S0232/PGK1.f1 | AGAGCCAGTTGCTGTAGAACTCAA | 742 |
| PGK1 | NM_000291 | S0234/PGK1.r1 | CTGGGCCTACACAGTCCTTCA | 743 |
| PGK1 | NM_000291 | S5022/PGK1.p1 | TCTCTGCTGGGCAAGGATGTTCTGTTC | 744 |
| PHB | NM_002634 | T2171/PHB.f1 | GACATTGTGGTAGGGGAAGG | 745 |
| PHB | NM_002634 | T2172/PHB.r1 | CGGCAGTCAAAGATAATTGG | 746 |
| PHB | NM_002634 | T2173/PHB.p1 | TCATTTTCTCATCCCGTGGGTACAGA | 747 |
| PI3KC2A | NM_002645 | S2020/PI3KC2.r1 | CACACTAGCATTTTCTCCGCATA | 748 |
| PI3KC2A | NM_002645 | S2021/PI3KC2.f1 | ATACCAATCACCGCACAAACC | 749 |
| PI3KC2A | NM_002645 | S5062/PI3KC2.p1 | TGCGCTGTGACTGGACTTAACAAATAGCCT | 750 |
| PIM1 | NM_002648 | S7858/PIM1.f3 | CTGCTCAAGGACACCGTCTA | 751 |
| PIM1 | NM_002648 | S7859/PIM1.r3 | GGATCCACTCTGGAGGGC | 752 |
| PIM1 | NM_002648 | S7860/PIM1.p3 | TACACTCGGGTCCCATCGAAGTCC | 753 |
| PIM2 | NM_006875 | T2144/PIM2.f1 | TGGGGACATTCCCTTTGAG | 754 |
| PIM2 | NM_006875 | T2145/PIM2.r1 | GACATGGGCTGGGAAGTG | 755 |
| PIM2 | NM_006875 | T2146/PIM2.p1 | CAGCTTCCAGAATCTCCTGGTCCC | 756 |
| PLAUR | NM_002659 | S1976/PLAUR.f3 | CCCATGGATGCTCCTCTGAA | 757 |
| PLAUR | NM_002659 | S1977/PLAUR.r3 | CCGGTGGCTACCAGACATTG | 758 |
| PLAUR | NM_002659 | S5054/PLAUR.p3 | CATTGACTGCCGAGGCCCCATG | 759 |
| PLD3 | NM_012268 | S8645/PLD3.f1 | CCAAGTTCTGGGTGGTGG | 760 |
| PLD3 | NM_012268 | S8646/PLD3.r1 | GTGAACGCCAGTCCATGTT | 761 |
| PLD3 | NM_012268 | S8647/PLD3.p1 | CCAGACCCACTTCTACCTGGGCAG | 762 |
| PLK | NM_005030 | S3099/PLK.f3 | AATGAATACAGTATTCCCAAGCACAT | 763 |
| PLK | NM_005030 | S3100/PLK.r3 | TGTCTGAAGCATCTTCTGGATGA | 764 |
| PLK | NM_005030 | S4825/PLK.p3 | AACCCCGTGGCCGCCTCC | 765 |
| PMS1 | NM_000534 | S5894/PMS1.f2 | CTTACGGTTTTCGTGGAGAAG | 766 |
| PMS1 | NM_000534 | S5895/PMS1.r2 | AGCAGCCGTTCTTGTTGTAA | 767 |
| PMS1 | NM_000534 | S5896/PMS1.p2 | CCTCAGCTATACAACAAATTGACCCCAAG | 768 |
| PMS2 | NM_000535 | S5878/PMS2.f3 | GATGTGGACTGCCATTCAAA | 769 |
| PMS2 | NM_000535 | S5879/PMS2.r3 | TGCGAGATTAGTTGGCTGAG | 770 |
| PMS2 | NM_000535 | S5880/PMS2.p3 | TCGAAATTTACATCCGGTATCTTCCTGG | 771 |
| PP591 | NM_025207 | S8657/PP591.f1 | CCACATACCGTCCAGCCTA | 772 |
| PP591 | NM_025207 | S8658/PP591.r1 | GAGGTCATGTGCGGGAGT | 773 |
| PP591 | NM_025207 | S8659/PP591.p1 | CCGCTCCTCTTCTTCGTTCTCCAG | 774 |
| PPP2CA | NM_002715 | T0732/PPP2CA.f1 | GCAATCATGGAACTTGACGA | 775 |
| PPP2CA | NM_002715 | T0733/PPP2CA.r1 | ATGTGGCTCGCCTCTACG | 776 |
| PPP2CA | NM_002715 | T0734/PPP2CA.p1 | TTTCTTGCAGTTTGACCCAGCACC | 777 |
| PR | NM_000926 | S1336/PR.f6 | GCATCAGGCTGTCATTATGG | 778 |
| PR | NM_000926 | S1337/PR.r6 | AGTAGTTGTGCTGCCCTTCC | 779 |
| PR | NM_000926 | S4743/PR.p6 | TGTCCTTACCTGTGGGAGCTGTAAGGTC | 780 |
| PRDX1 | NM_002574 | T1241/PRDX1.f1 | AGGACTGGGACCCATGAAC | 781 |
| PRDX1 | NM_002574 | T1242/PRDX1.r1 | CCCATAATCCTGAGCAATGG | 782 |
| PRDX1 | NM_002574 | T1243/PRDX1.p1 | TCCTTTGGTATCAGACCCGAAGCG | 783 |
| PRDX2 | NM_005809 | S8761/PRDX2.f1 | GGTGTCCTTCGCCAGATCAC | 784 |
| PRDX2 | NM_005809 | S8762/PRDX2.r1 | CAGCCGCAGAGCCTCATC | 785 |
| PRDX2 | NM_005809 | S8763/PRDX2.p1 | TTAATGATTTGCCTGTGGGACGCTCC | 786 |
| PRKCA | NM_002737 | S7369/PRKCA.f1 | CAAGCAATGCGTCATCAATGT | 787 |
| PRKCA | NM_002737 | S7370/PRKCA.r1 | GTAAATCCGCCCCCTCTTCT | 788 |
| PRKCA | NM_002737 | S7371/PRKCA.p1 | CAGCCTCTGCGGAATGGATCACACT | 789 |
| PRKCD | NM_006254 | S1738/PRKCD.f2 | CTGACACTTGCCGCAGAGAA | 790 |
| PRKCD | NM_006254 | S1739/PRKCD.r2 | AGGTGGTCCTTGGTCTGGAA | 791 |
| PRKCD | NM_006254 | S4923/PRKCD.p2 | CCCTTTCTCACCCACCTCATCTGCAC | 792 |
| PRKCG | NM_002739 | T2081/PRKCG.f1 | GGGTTCTAGACGCCCCTC | 793 |
| PRKCG | NM_002739 | T2082/PRKCG.r1 | GGACGGCTGTAGAGGCTGTAT | 794 |
| PRKCG | NM_002739 | T2083/PRKCG.p1 | CAAGCGTTCCTGGCCTTCTGAACT | 795 |
| PRKCH | NM_006255 | T2084/PRKCH.f1 | CTCCACCTATGAGCGTCTGTC | 796 |
| PRKCH | NM_006255 | T2085/PRKCH.r1 | CACACTTTCCCTCCTTTTGG | 797 |
| PRKCH | NM_006255 | T2086/PRKCH.p1 | TCCTGTTAACATCCCAAGCCCACA | 798 |
| pS2 | NM_003225 | S0241/p52.f2 | GCCCTCCCAGTGTGCAAAT | 799 |
| pS2 | NM_003225 | S0243/p52.r2 | CGTCGATGGTATTAGGATAGAAGCA | 800 |
| pS2 | NM_003225 | S5026/p52.p2 | TGCTGTTTCGACGACACCGTTCG | 801 |
| PTEN | NM_000314 | S0244/PTEN.f2 | TGGCTAAGTGAAGATGACAATCATG | 802 |
| PTEN | NM_000314 | S0246/PTEN.r2 | TGCACATATCATTACACCAGTTCGT | 803 |
| PTEN | NM_000314 | S5027/PTEN.p2 | CCTTTCCAGCTTTACAGTGAATTGCTGCA | 804 |
| PTPD1 | NM_007039 | S3069/PTPD1.f2 | CGCTTGCCTAACTCATACTTTCC | 805 |
| PTPD1 | NM_007039 | S3070/PTPD1.r2 | CCATTCAGACTGCGCCACTT | 806 |
| PTPD1 | NM_007039 | S4822/PTPD1.p2 | TCCACGCAGCGTGGCACTG | 807 |
| PTTG1 | NM_004219 | S4525/PTTG1.f2 | GGCTACTCTGATCTATGTTGATAAGGAA | 808 |
| PTTG1 | NM_004219 | S4526/PTTG1.r2 | GCTTCAGCCCATCCTTAGCA | 809 |
| PTTG1 | NM_004219 | S4527/PTTG1.p2 | CACACGGGTGCCTGGTTCTCCA | 810 |
| RAB27B | NM_004163 | S4336/RAB27B.f1 | GGGACACTGCGGGACAAG | 811 |
| RAB27B | NM_004163 | S4337/RAB27B.r1 | GCCCATGGCGTCTCTGAA | 812 |
| RAB27B | NM_004163 | S4338/RAB27B.p1 | CGGTTCCGGAGTCTCACCACTGCAT | 813 |
| RAB31 | NM_006868 | S9306/RAB31.f1 | CTGAAGGACCCTACGCTCG | 814 |
| RAB31 | NM_006868 | S9307/RAB31.r1 | ATGCAAAGCCAGTGTGCTC | 815 |
| RAB31 | NM_006868 | S9308/RAB31.p1 | CTTCTCAAAGTGAGGTGCCAGGCC | 816 |
| RAB6C | NM_032144 | S5535/RAB6C.f1 | GCGACAGCTCCTCTAGTTCCA | 817 |
| RAB6C | NM_032144 | S5537/RAB6C.p1 | TTCCCGAAGTCTCCGCCCG | 818 |
| RAB6C | NM_032144 | S5538/RAB6C.r1 | GGAACACCAGCTTGAATTTCCT | 819 |
| RAD1 | NM_002853 | T2174/RAD1.f1 | GAGGAGTGGTGACAGTCTGC | 820 |
| RAD1 | NM_002853 | T2175/RAD1.r1 | GCTGCAGAAATCAAAGTCCA | 821 |
| RAD1 | NM_002853 | T2176/RAD1.p1 | TCAATACACAGGAACCTGAGGAGACCC | 822 |
| RAD54L | NM_003579 | S4369/RAD54L.f1 | AGCTAGCCTCAGTGACACACATG | 823 |
| RAD54L | NM_003579 | S4370/RAD54L.r1 | CCGGATCTGACGGCTGTT | 824 |
| RAD54L | NM_003579 | S4371/RAD54L.p1 | ACACAACGTCGGCAGTGCAACCTG | 825 |
| RAF1 | NM_002880 | S5933/RAF1.f3 | CGTCGTATGCGAGAGTCTGT | 826 |
| RAF1 | NM_002880 | S5934/RAF1.r3 | TGAAGGCGTGAGGTGTAGAA | 827 |
| RAF1 | NM_002880 | S5935/RAF1.p3 | TCCAGGATGCCTGTTAGTTCTCAGCA | 828 |
| RALBP1 | NM_006788 | S5853/RALBP1.f1 | GGTGTCAGATATAAATGTGCAAATGC | 829 |
| RALBP1 | NM_006788 | S5854/RALBP1.r1 | TTCGATATTGCCAGCAGCTATAAA | 830 |
| RALBP1 | NM_006788 | S5855/RALBP1.p1 | TGCTGTCCTGTCGGTCTCAGTACGTTCA | 831 |
| RAP1GDS1 | NM_021159 | S5306/RAP1GD.f2 | TGTGGATGCTGGATTGATTT | 832 |
| RAP1GDS1 | NM_021159 | S5307/RAP1GD.r2 | AAGCAGCACTTCCTGGTCTT | 833 |
| RAP1GDS1 | NM_021159 | S5308/RAP1GD.p2 | CCACTGGTGCAGCTGCTAAATAGCA | 834 |
| RASSF1 | NM_007182 | S2393/RASSF1.f3 | AGTGGGAGACACCTGACCTT | 835 |
| RASSF1 | NM_007182 | S2394/RASSF1.r3 | TGATCTGGGCATTGTACTCC | 836 |
| RASSF1 | NM_007182 | S4909/RASSF1.p3 | TTGATCTTCTGCTCAATCTCAGCTTGAGA | 837 |
| RB1 | NM_000321 | S2700/RB1.f1 | CGAAGCCCTTACAAGTTTCC | 838 |
| RB1 | NM_000321 | S2701/RB1.r1 | GGACTCTTCAGGGGTGAAAT | 839 |
| RB1 | NM_000321 | S4765/RB1.p1 | CCCTTACGGATTCCTGGAGGGAAC | 840 |
| RBM17 | NM_032905 | T2186/RBM17.f1 | CCCAGTGTACGAGGAACAAG | 841 |
| RBM17 | NM_032905 | T2187/RBM17.r1 | TTAGCGAGGAAGGAGTTGCT | 842 |
| RBM17 | NM_032905 | T2188/RBM17.p1 | ACAGACCGAGATCTCCAACCGGAC | 843 |
| RCC1 | NM_001269 | S8854/RCC1.f1 | GGGCTGGGTGAGAATGTG | 844 |
| RCC1 | NM_001269 | S8855/RCC1.r1 | CACAACATCCTCCGGAATG | 845 |
| RCC1 | NM_001269 | S8856/RCC1.p1 | ATACCAGGGCCGGCTTCTTCCTCT | 846 |
| REG1A | NM_002909 | T2093/REG1A.f1 | CCTACAAGTCCTGGGGCA | 847 |
| REG1A | NM_002909 | T2094/REG1A.r1 | TGAGGTCAGGCTCACACAGT | 848 |
| REG1A | NM_002909 | T2095/REG1A.p1 | TGGAGCCCCAAGCAGTGTTAATCC | 849 |
| RELB | NM_006509 | T2096/RELB.f1 | GCGAGGAGCTCTACTTGCTC | 850 |
| RELB | NM_006509 | T2097/RELB.r1 | GCCCTGCTGAACACCACT | 851 |
| RELB | NM_006509 | T2098/RELB.p1 | TGTCCTCTTTCTGCACCTTGTCGC | 852 |
| RhoB | NM_004040 | S8284/RhoB.f1 | AAGCATGAACAGGACTTGACC | 853 |
| RhoB | NM_004040 | S8285/RhoB.r1 | CCTCCCCAAGTCAGTTGC | 854 |
| RhoB | NM_004040 | S8286/RhoB.p1 | CTTTCCAACCCCTGGGGAAGACAT | 855 |
| rhoC | NM_175744 | S2162/rhoC.f1 | CCCGTTCGGTCTGAGGAA | 856 |
| rhoC | NM_175744 | S2163/rhoC.r1 | GAGCACTCAAGGTAGCCAAAGG | 857 |
| rhoC | NM_175744 | S5042/rhoC.p1 | TCCGGTTCGCCATGTCCCG | 858 |
| RIZ1 | NM_012231 | S1320/RIZ1.f2 | CCAGACGAGCGATTAGAAGC | 859 |
| RIZ1 | NM_012231 | S1321/RIZ1.r2 | TCCTCCTCTTCCTCCTCCTC | 860 |
| RIZ1 | NM_012231 | S4761/RIZ1.p2 | TGTGAGGTGAATGATTTGGGGGA | 861 |
| ROCK1 | NM_005406 | S8305/ROCK1.f1 | TGTGCACATAGGAATGAGCTTC | 862 |
| ROCK1 | NM_005406 | S8306/ROCK1.r1 | GTTTAGCACGCAATTGCTCA | 863 |
| ROCK1 | NM_005406 | S8307/ROCK1.p1 | TCACTCTCTTTGCTGGCCAACTGC | 864 |
| RPL37A | NM_000998 | T2418/RPL37A.f2 | GATCTGGCACTGTGGTTCC | 865 |
| RPL37A | NM_000998 | T2419/RPL37A.r2 | TGACAGCGGAAGTGGTATTG | 866 |
| RPL37A | NM_000998 | T2420/RPL37A.p2 | CACCGCCAGCCACTGTCTTCAT | 867 |
| RPLPO | NM_001002 | S0256/RPLPO.f2 | CCATTCTATCATCAACGGGTACAA | 868 |
| RPLPO | NM_001002 | S0258/RPLPO.r2 | TCAGCAAGTGGGAAGGTGTAATC | 869 |
| RPLPO | NM_001002 | S4744/RPLPO.p2 | TCTCCACAGACAAGGCCAGGACTCG | 870 |
| RPN2 | NM_002951 | T1158/RPN2.f1 | CTGTCTTCCTGTTGGCCCT | 871 |
| RPN2 | NM_002951 | T1159/RPN2.r1 | GTGAGGTAGTGAGTGGGCGT | 872 |
| RPN2 | NM_002951 | T1160/RPN2.p1 | ACAATCATAGCCAGCACCTGGGCT | 873 |
| RPS6KB1 | NM_003161 | S2615/RPS6KB.f3 | GCTCATTATGAAAAACATCCCAAAC | 874 |
| RPS6KB1 | NM_003161 | S2616/RPS6KB.r3 | AAGAAACAGAAGTTGTCTGGCTTTCT | 875 |
| RPS6KB1 | NM_003161 | S4759/RPS6KB.p3 | CACACCAACCAATAATTTCGCATT | 876 |
| RXRA | NM_002957 | S8463/RXRA.f1 | GCTCTGTTGTGTCCTGTTGC | 877 |
| RXRA | NM_002957 | S8464/RXRA.r1 | GTACGGAGAAGCCACTTCACA | 878 |
| RXRA | NM_002957 | S8465/RXRA.p1 | TCAGTCACAGGAAGGCCAGAGCC | 879 |
| RXRB | NM_021976 | S8490/RXRB.f1 | CGAGGAGATGCCTGTGGA | 880 |
| RXRB | NM_021976 | S8491/RXRB.r1 | CAACGCCCTGGTCACTCT | 881 |
| RXRB | NM_021976 | S8492/RXRB.p1 | CTGTTCCACAGCAAGCTCTGCCTC | 882 |
| S100A10 | NM_002966 | S9950/S100A1.f1 | ACACCAAAATGCCATCTCAA | 883 |
| S100A10 | NM_002966 | S9951/S100A1.r1 | TTTATCCCCAGCGAATTTGT | 884 |
| S100A10 | NM_002966 | S9952/S100A1.p1 | CACGCCATGGAAACCATGATGTTT | 885 |
| SEC61A | NM_013336 | S8648/SEC61A.f1 | CTTCTGAGCCCGTCTCCC | 886 |
| SEC61A | NM_013336 | S8649/SEC61A.r1 | GAGAGCTCCCCTTCCGAG | 887 |
| SEC61A | NM_013336 | S8650/SEC61A.p1 | CGCTTCTGGAGCAGCTTCCTCAAC | 888 |
| SEMA3F | NM_004186 | S2857/SEMA3F.f3 | CGCGAGCCCCTCATTATACA | 889 |
| SEMA3F | NM_004186 | S2858/SEMA3F.r3 | CACTCGCCGTTGACATCCT | 890 |
| SEMA3F | NM_004186 | S4972/SEMA3F.p3 | CTCCCCACAGCGCATCGAGGAA | 891 |
| SFN | NM_006142 | S9953/SFN.f1 | GAGAGAGCCAGTCTGATCCA | 892 |
| SFN | NM_006142 | S9954/SFN.r1 | AGGCTGCCATGTCCTCATA | 893 |
| SFN | NM_006142 | S9955/SFN.p1 | CTGCTCTGCCAGCTTGGCCTTC | 894 |
| SGCB | NM_000232 | S5752/SGCB.f1 | CAGTGGAGACCAGTTGGGTAGTG | 895 |
| SGCB | NM_000232 | S5753/SGCB.r1 | CCTTGAAGAGCGTCCCATCA | 896 |
| SGCB | NM_000232 | S5754/SGCB.p1 | CACACATGCAGAGCTTGTAGCGTACCCA | 897 |
| SGK | NM_005627 | S8308/SGK.f1 | TCCGCAAGACACCTCCTG | 898 |
| SGK | NM_005627 | S8309/SGK.r1 | TGAAGTCATCCTTGGCCC | 899 |
| SGK | NM_005627 | S8310/SGK.p1 | TGTCCTGTCCTTCTGCAGGAGGC | 900 |
| SGKL | NM_170709 | T2183/SGKL.f1 | TGCATTCGTTGGTTTCTCTT | 901 |
| SGKL | NM_170709 | T2184/SGKL.r1 | TTTCTGAATGGCAAACTGCT | 902 |
| SGKL | NM_170709 | T2185/SGKL.p1 | TGCACCTCCTTCAGAAGACTTATTTTTGTG | 903 |
| SHC1 | NM_003029 | S6456/SHC1.f1 | CCAACACCTTCTTGGCTTCT | 904 |
| SHC1 | NM_003029 | S6457/SHC1.r1 | CTGTTATCCCAACCCAAACC | 905 |
| SHC1 | NM_003029 | S6458/SHC1.p1 | CCTGTGTTCTTGCTGAGCACCCTC | 906 |
| SIR2 | NM_012238 | S1575/SIR2.f2 | AGCTGGGGTGTCTGTTTCAT | 907 |
| SIR2 | NM_012238 | S1576/SIR2.r2 | ACAGCAAGGCGAGCATAAAT | 908 |
| SIR2 | NM_012238 | S4885/SIR2.p2 | CCTGACTTCAGGTCAAGGGATGG | 909 |
| SLC1A3 | NM_004172 | S8469/SLC1A3.f1 | GTGGGGAGCCCATCATCT | 910 |
| SLC1A3 | NM_004172 | S8470/SLC1A3.r1 | CCAGTCCACACTGAGTGCAT | 911 |
| SLC1A3 | NM_004172 | S8471/SLC1A3.p1 | CCAAGCCATCACAGGCTCTGCATA | 912 |
| SLC25A3 | NM_213611 | T0278/SLC25A.f2 | TCTGCCAGTGCTGAATTCTT | 913 |
| SLC25A3 | NM_213611 | T0279/SLC25A.r2 | TTCGAACCTTAGCAGCTTCC | 914 |
| SLC25A3 | NM_213611 | T0280/SLC25A.p2 | TGCTGACATTGCCCTGGCTCCTAT | 915 |
| SLC35B1 | NM_005827 | S8642/SLC35B.f1 | CCCAACTCAGGTCCTTGGTA | 916 |
| SLC35B1 | NM_005827 | S8643/SLC35B.r1 | CAAGAGGGTCACCCCAAG | 917 |
| SLC35B1 | NM_005827 | S8644/SLC35B.p1 | ATCCTGCAAGCCAATCCCAGTCAT | 918 |
| SLC7A11 | NM_014331 | T2045/SLC7A1.f1 | AGATGCATACTTGGAAGCACAG | 919 |
| SLC7A11 | NM_014331 | T2046/SLC7A1.r1 | AACCTAGGACCAGGTAACCACA | 920 |
| SLC7A11 | NM_014331 | T2047/SLC7A1.p1 | CATATCACACTGGGAGGCAATGCA | 921 |
| SLC7A5 | NM_003486 | S9244/SLC7A5.f2 | GCGCAGAGGCCAGTTAAA | 922 |
| SLC7A5 | NM_003486 | S9245/SLC7A5.r2 | AGCTGAGCTGTGGGTTGC | 923 |
| SLC7A5 | NM_003486 | S9246/SLC7A5.p2 | AGATCACCTCCTCGAACCCACTCC | 924 |
| SNAI2 | NM_003068 | S7824/SNAI2.f1 | GGCTGGCCAAACATAAGCA | 925 |
| SNAI2 | NM_003068 | S7825/SNAI2.r1 | TCCTTGTCACAGTATTTACAGCTGAA | 926 |
| SNAI2 | NM_003068 | S7826/SNAI2.p1 | CTGCACTGCGATGCCCAGTCTAGAAAATC | 927 |
| SNCA | NM_007308 | T2320/SNCA.f1 | AGTGACAAATGTTGGAGGAGC | 928 |
| SNCA | NM_007308 | T2321/SNCA.r1 | CCCTCCACTGTCTTCTGGG | 929 |
| SNCA | NM_007308 | T2322/SNCA.p1 | TACTGCTGTCACACCCGTCACCAC | 930 |
| SNCG | NM_003087 | T1704/SNCG.f1 | ACCCACCATGGATGTCTTC | 931 |
| SNCG | NM_003087 | T1705/SNCG.r1 | CCTGCTTGGTCTTTTCCAC | 932 |
| SNCG | NM_003087 | T1706/SNCG.p1 | AAGAAGGGCTTCTCCATCGCCAAG | 933 |
| SOD1 | NM_000454 | S7683/SOD1.f1 | TGAAGAGAGGCATGTTGGAG | 934 |
| SOD1 | NM_000454 | S7684/SOD1.r1 | AATAGACACATCGGCCACAC | 935 |
| SOD1 | NM_000454 | S7685/SOD1.p1 | TTTGTCAGCAGTCACATTGCCCAA | 936 |
| SRI | NM_003130 | T2177/SRI.f1 | ATACAGCACCAATGGAAAGATCAC | 937 |
| SRI | NM_003130 | T2178/SRI.r1 | TGTCTGTAAGAGCCCTCAGTTTGA | 938 |
| SRI | NM_003130 | T2179/SRI.p1 | TTCGACGACTACATCGCCTGCTGC | 939 |
| STAT1 | NM_007315 | S1542/STAT1.f3 | GGGCTCAGCTTTCAGAAGTG | 940 |
| STAT1 | NM_007315 | S1543/STAT1.r3 | ACATGTTCAGCTGGTCCACA | 941 |
| STAT1 | NM_007315 | S4878/STAT1.p3 | TGGCAGTTTTCTTCTGTCACCAAAA | 942 |
| STAT3 | NM_003150 | S1545/STAT3.f1 | TCACATGCCACTTTGGTGTT | 943 |
| STAT3 | NM_003150 | S1546/STAT3.r1 | CTTGCAGGAAGCGGCTATAC | 944 |
| STAT3 | NM_003150 | S4881/STAT3.p1 | TCCTGGGAGAGATTGACCAGCA | 945 |
| STK10 | NM_005990 | T2099/STK10.f1 | CAAGAGGGACTCGGACTGC | 946 |
| STK10 | NM_005990 | T2100/STK10.r1 | CAGGTCAGTGGAGAGATTGGT | 947 |
| STK10 | NM_005990 | T2101/STK10.p1 | CCTCTGCACCTCTGAGAGCATGGA | 948 |
| STK11 | NM_000455 | S9454/STK11.f1 | GGACTCGGAGACGCTGTG | 949 |
| STK11 | NM_000455 | S9455/STK11.r1 | GGGATCCTTCGCAACTTCTT | 950 |
| STK11 | NM_000455 | S9456/STK11.p1 | TTCTTGAGGATCTTGACGGCCCTC | 951 |
| STK15 | NM_003600 | S0794/STK15.f2 | CATCTTCCAGGAGGACCACT | 952 |
| STK15 | NM_003600 | S0795/STK15.r2 | TCCGACCTTCAATCATTTCA | 953 |
| STK15 | NM_003600 | S4745/STK15.p2 | CTCTGTGGCACCCTGGACTACCTG | 954 |
| STMN1 | NM_005563 | S5838/STMN1.f1 | AATACCCAACGCACAAATGA | 955 |
| STMN1 | NM_005563 | S5839/STMN1.r1 | GGAGACAATGCAAACCACAC | 956 |
| STMN1 | NM_005563 | S5840/STMN1.p1 | CACGTTCTCTGCCCCGTTTCTTG | 957 |
| STMY3 | NM_005940 | S2067/STMY3.f3 | CCTGGAGGCTGCAACATACC | 958 |
| STMY3 | NM_005940 | S2068/STMY3.r3 | TACAATGGCTTTGGAGGATAGCA | 959 |
| STMY3 | NM_005940 | S4746/STMY3.p3 | ATCCTCCTGAAGCCCTTTTCGCAGC | 960 |
| SURV | NM_001168 | S0259/SURV.f2 | TGTTTTGATTCCCGGGCTTA | 961 |
| SURV | NM_001168 | S0261/SURV.r2 | CAAAGCTGTCAGCTCTAGCAAAAG | 962 |
| SURV | NM_001168 | S4747/SURV.p2 | TGCCTTCTTCCTCCCTCACTTCTCACCT | 963 |
| TACC3 | NM_006342 | S7124/TACC3.f1 | CACCCTTGGACTGGAAAACT | 964 |
| TACC3 | NM_006342 | S7125/TACC3.r1 | CCTTGATGAGCTGTTGGTTC | 965 |
| TACC3 | NM_006342 | S7126/TACC3.p1 | CACACCCGGTCTGGACACAGAAAG | 966 |
| TBCA | NM_004607 | T2284/TBCA.f1 | GATCCTCGCGTGAGACAGA | 967 |
| TBCA | NM_004607 | T2285/TBCA.r1 | CACTTTTTCTTTGACCAACCG | 968 |
| TBCA | NM_004607 | T2286/TBCA.p1 | TTCACCACGCCGGTCTTGATCTT | 969 |
| TBCC | NM_003192 | T2302/TBCC.f1 | CTGTTTTCCTGGAGGACTGC | 970 |
| TBCC | NM_003192 | T2303/TBCC.r1 | ACTGTGTATGCGGAGCTGTT | 971 |
| TBCC | NM_003192 | T2304/TBCC.p1 | CCACTGCCAGCACGCAGTCAC | 972 |
| TBCD | NM_005993 | T2287/TBCD.f1 | CAGCCAGGTGTACGAGACATT | 973 |
| TBCD | NM_005993 | T2288/TBCD.r1 | ACCTCGTCCAGCACATCC | 974 |
| TBCD | NM_005993 | T2289/TBCD.p1 | CTCACCTACAGTGACGTCGTGGGC | 975 |
| TBCE | NM_003193 | T2290/TBCE.f1 | TCCCGAGAGAGGAAAGCAT | 976 |
| TBCE | NM_003193 | T2291/TBCE.r1 | GTCGGGTGCCTGCATTTA | 977 |
| TBCE | NM_003193 | T2292/TBCE.p1 | ATACACAGTCCCTTCGTGGCTCCC | 978 |
| TBD | NM_016261 | S3347/TBD.f2 | CCTGGTTGAAGCCTGTTAATGC | 979 |
| TBD | NM_016261 | S3348/TBD.r2 | TGCAGACTTCTCATATTTGCTAAAGG | 980 |
| TBD | NM_016261 | S4864/TBD.p2 | CCGCTGGGTTTTCCACACGTTGA | 981 |
| TCP1 | NM_030752 | T2296/TCP1.f1 | CCAGTGTGTGTAACAGGGTCAC | 982 |
| TCP1 | NM_030752 | T2297/TCP1.r1 | TATAGCCTTGGGCCACCC | 983 |
| TCP1 | NM_030752 | T2298/TCP1.p1 | AGAATTCGACAGCCAGATGCTCCA | 984 |
| TFRC | NM_003234 | S1352/TFRC.f3 | GCCAACTGCTTTCATTTGTG | 985 |
| TFRC | NM_003234 | S1353/TFRC.r3 | ACTCAGGCCCATTTCCTTTA | 986 |
| TFRC | NM_003234 | S4748/TFRC.p3 | AGGGATCTGAACCAATACAGAGCAGACA | 987 |
| THBS1 | NM_003246 | S6474/THBS1.f1 | CATCCGCAAAGTGACTGAAGAG | 988 |
| THBS1 | NM_003246 | S6475/THBS1.r1 | GTACTGAACTCCGTTGTGATAGCATAG | 989 |
| THBS1 | NM_003246 | S6476/THBS1.p1 | CCAATGAGCTGAGGCGGCCTCC | 990 |
| TK1 | NM_003258 | S0866/TK1.f2 | GCCGGGAAGACCGTAATTGT | 991 |
| TK1 | NM_003258 | S0927/TK1.r2 | CAGCGGCACCAGGTTCAG | 992 |
| TK1 | NM_003258 | S4798/TK1.p2 | CAAATGGCTTCCTCTGGAAGGTCCCA | 993 |
| TOP2A | NM_001067 | S0271/TOP2A.f4 | AATCCAAGGGGGAGAGTGAT | 994 |
| TOP2A | NM_001067 | S0273/TOP2A.r4 | GTACAGATTTTGCCCGAGGA | 995 |
| TOP2A | NM_001067 | S4777/TOP2A.p4 | CATATGGACTTTGACTCAGCTGTGGC | 996 |
| TOP3B | NM_003935 | T2114/TOP3B.f1 | GTGATGCCTTCCCTGTGG | 997 |
| TOP3B | NM_003935 | T2115/TOP3B.r1 | TCAGGTAGTCGGGTGGGTT | 998 |
| TOP3B | NM_003935 | T2116/TOP3B.p1 | TGCTTCTCCAGCATCTTCACCTCG | 999 |
| TP | NM_001953 | S0277/TP.f3 | CTATATGCAGCCAGAGATGTGACA | 1000 |
| TP | NM_001953 | S0279/TP.r3 | CCACGAGTTTCTTACTGAGAATGG | 1001 |
| TP | NM_001953 | S4779/TP.p3 | ACAGCCTGCCACTCATCACAGCC | 1002 |
| TP53BP1 | NM_005657 | S1747/TP53BP.f2 | TGCTGTTGCTGAGTCTGTTG | 1003 |
| TP53BP1 | NM_005657 | S1748/TP53BP.r2 | CTTGCCTGGCTTCACAGATA | 1004 |
| TP53BP1 | NM_005657 | S4924/TP53BP.p2 | CCAGTCCCCAGAAGACCATGTCTG | 1005 |
| TPT1 | NM_003295 | S9098/TPT1.f1 | GGTGTCGATATTGTCATGAACC | 1006 |
| TPT1 | NM_003295 | S9099/TPT1.r1 | GTAATCTTTGATGTACTTCTTGTAGGC | 1007 |
| TPT1 | NM_003295 | S9100/TPT1.p1 | TCACCTGCAGGAAACAAGTTTCACAAA | 1008 |
| TRAG3 | NM_004909 | S5881/TRAG3.f1 | GACGCTGGTCTGGTGAAGATG | 1009 |
| TRAG3 | NM_004909 | S5882/TRAG3.r1 | TGGGTGGTTGTTGGACAATG | 1010 |
| TRAG3 | NM_004909 | S5883/TRAG3.p1 | CCAGGAAACCACGAGCCTCCAGC | 1011 |
| TRAIL | NM_003810 | S2539/TRAIL.f1 | CTTCACAGTGCTCCTGCAGTCT | 1012 |
| TRAIL | NM_003810 | S2540/TRAIL.r1 | CATCTGCTTCAGCTCGTTGGT | 1013 |
| TRAIL | NM_003810 | S4980/TRAIL.p1 | AAGTACACGTAAGTTACAGCCACACA | 1014 |
| TS | NM_001071 | S0280/TS.f1 | GCCTCGGTGTGCCTTTCA | 1015 |
| TS | NM_001071 | S0282/TS.r1 | CGTGATGTGCGCAATCATG | 1016 |
| TS | NM_001071 | S4780/TS.p1 | CATCGCCAGCTACGCCCTGCTC | 1017 |
| TSPAN4 | NM_003271 | T2102/TSPAN4.f1 | CTGGTCAGCCTTCAGGGAC | 1018 |
| TSPAN4 | NM_003271 | T2103/TSPAN4.r1 | CTTCAGTTCTGGGCTGGC | 1019 |
| TSPAN4 | NM_003271 | T2104/TSPAN4.p1 | CTGAGCACCGCCTGGTCTCTTTC | 1020 |
| TTK | NM_003318 | S7247/TTK.f1 | TGCTTGTCAGTTGTCAACACCTT | 1021 |
| TTK | NM_003318 | S7248/TTK.r1 | TGGAGTGGCAAGTATTTGATGCT | 1022 |
| TTK | NM_003318 | S7249/TTK.p1 | TGGCCAACCTGCCTGTTTCCAGC | 1023 |
| TUBA1 | NM_006000 | S8578/TUBA1.f1 | TGTCACCCCGACTCAACGT | 1024 |
| TUBA1 | NM_006000 | S8579/TUBA1.r1 | ACGTGGACTGAGATGCATTCAC | 1025 |
| TUBA1 | NM_006000 | S8580/TUBA1.p1 | AGACGCACCGCCCGGACTCAC | 1026 |
| TUBA2 | NM_006001 | S8581/TUBA2.f1 | AGCTCAACATGCGTGAGTGT | 1027 |
| TUBA2 | NM_006001 | S8582/TUBA2.r1 | ATTGCCGATCTGGACTCCT | 1028 |
| TUBA2 | NM_006001 | S8583/TUBA2.p1 | ATCTCTATCCACGTGGGGCAGGC | 1029 |
| TUBA3 | NM_006009 | S8584/TUBA3.f1 | CTCTTACATCGACCGCCTAAGAG | 1030 |
| TUBA3 | NM_006009 | S8585/TUBA3.r1 | GCTGATGGCGGAGACGAA | 1031 |
| TUBA3 | NM_006009 | S8586/TUBA3.p1 | CGCGCTGTAAGAAGCAACAACCTCTCC | 1032 |
| TUBA4 | NM_025019 | T2415/TUBA4.f3 | GAGGAGGGTGAGTTCTCCAA | 1033 |
| TUBA4 | NM_025019 | T2416/TUBA4.r3 | ATGCCCACCTCCTTGTAATC | 1034 |
| TUBA4 | NM_025019 | T2417/TUBA4.p3 | CCATGAGGATATGACTGCCCTGGA | 1035 |
| TUBA6 | NM_032704 | S8590/TUBA6.f1 | GTCCCTTCGCCTCCTTCAC | 1036 |
| TUBA6 | NM_032704 | S8591/TUBA6.r1 | CGTGGATGGAGATGCACTCA | 1037 |
| TUBA6 | NM_032704 | S8592/TUBA6.p1 | CCGCAGACCCCTTCAAGTTCTAGTCATG | 1038 |
| TUBA8 | NM_018943 | T2412/TUBA8.f2 | CGCCCTACCTATACCAACCT | 1039 |
| TUBA8 | NM_018943 | T2413/TUBA8.r2 | CGGAGAGAAGCAGTGATTGA | 1040 |
| TUBA8 | NM_018943 | T2414/TUBA8.p2 | CAACCGCCTCATCAGTCAGATTGTG | 1041 |
| TUBB | NM_001069 | S5820/TUBB.f1 | CGAGGACGAGGCTTAAAAAC | 1042 |
| TUBB | NM_001069 | S5821/TUBB.r1 | ACCATGCTTGAGGACAACAG | 1043 |
| TUBB | NM_001069 | S5822/TUBB.p1 | TCTCAGATCAATCGTGCATCCTTAGTGAA | 1044 |
| TUBB classIII | NM_006086 | S8090/TUBB c.f3 | CGCCCTCCTGCAGTATTTATG | 1045 |
| TUBB classIII | NM_006086 | S8091/TUBB c.r3 | ACAGAGACAGGAGCAGCTCACA | 1046 |
| TUBB classIII | NM_006086 | S8092/TUBB c.p3 | CCTCGTCCTCCCCACCTAGGCCA | 1047 |
| TUBB1 | NM_030773 | S8093/TUBB1.f1 | ACACTGACTGGCATCCTGCTT | 1048 |
| TUBB1 | NM_030773 | S8094/TUBB1.r1 | GCTCTGTAGCTCCCCATGTACTAGT | 1049 |
| TUBB1 | NM_030773 | S8095/TUBB1.p1 | AGCCTCCAGAAGAGCCAGGTGCCT | 1050 |
| TUBB2 | NM_006088 | S8096/TUBB2.f1 | GTGGCCTAGAGCCTTCAGTC | 1051 |
| TUBB2 | NM_006088 | S8097/TUBB2.r1 | CAGGCTGGGAGTGAATAAAGA | 1052 |
| TUBB2 | NM_006088 | S8098/TUBB2.p1 | TTCACACTGCTTCCCTGCTTTCCC | 1053 |
| TUBB5 | NM_006087 | S8102/TUBB5.f1 | ACAGGCCCCATGCATCCT | 1054 |
| TUBB5 | NM_006087 | S8103/TUBB5.r1 | TGTTTCTCTCCCAGATAAGCTAAGG | 1055 |
| TUBB5 | NM_006087 | S8104/TUBB5.p1 | TGCCTCACTCCCCTCAGCCCC | 1056 |
| TUBBM | NM_032525 | S8105/TUBBM.f1 | CCCTATGGCCCTGAATGGT | 1057 |
| TUBBM | NM_032525 | S8106/TUBBM.r1 | ACTAATTACATGACTTGGCTGCATTT | 1058 |
| TUBBM | NM_032525 | S8107/TUBBM.p1 | TGAGGGGCCGACACCAACACAAT | 1059 |
| TUBBOK | NM_178014 | S8108/TUBBOK.f1 | AGTGGAATCCTTCCCTTTCC | 1060 |
| TUBBOK | NM_178014 | S8109/TUBBOK.r1 | CCCTTGATCCCTTTCTCTGA | 1061 |
| TUBBOK | NM_178014 | S8110/TUBBOK.p1 | CCTCACTCAGCTCCTTTCCCCTGA | 1062 |
| TUBBP | NM_178012 | S8111/TUBBP.f1 | GGAAGGAAAGAAGCATGGTCTACT | 1063 |
| TUBBP | NM_178012 | S8112/TUBBP.r1 | AAAAAGTGACAGGCAACAGTGAAG | 1064 |
| TUBBP | NM_178012 | S8113/TUBBP.p1 | CACCAGAGACCCAGCGCACACCTA | 1065 |
| TUBG1 | NM_001070 | T2299/TUBG1.f1 | GATGCCGAGGGAAATCATC | 1066 |
| TUBG1 | NM_001070 | T2300/TUBG1.r1 | CCAGAACTCGAACCCAATCT | 1067 |
| TUBG1 | NM_001070 | T2301/TUBG1.p1 | ATTGCCGCACTGGCCCAACTGTAG | 1068 |
| TWIST1 | NM_000474 | S7929/TWIST1.f1 | GCGCTGCGGAAGATCATC | 1069 |
| TWIST1 | NM_000474 | S7930/TWIST1.r1 | GCTTGAGGGTCTGAATCTTGCT | 1070 |
| TWIST1 | NM_000474 | S7931/TWIST1.p1 | CCACGCTGCCCTCGGACAAGC | 1071 |
| TYRO3 | NM_006293 | T2105/TYRO3.f1 | CAGTGTGGAGGGGATGGA | 1072 |
| TYRO3 | NM_006293 | T2106/TYRO3.r1 | CAAGTTCTGGACCACAGCC | 1073 |
| TYRO3 | NM_006293 | T2107/TYRO3.p1 | CTTCACCCACTGGATGTCAGGCTC | 1074 |
| UFM1 | NM_016617 | T1284/UFM1.f2 | AGTTGTCGTGTGTTCTGGATTCA | 1075 |
| UFM1 | NM_016617 | T1285/UFM1.r2 | CGTCAGCGTGATCTTAAAGGAA | 1076 |
| UFM1 | NM_016617 | T1286/UFM1.p2 | TCCGGCACCACCATGTCGAAGG | 1077 |
| upa | NM_002658 | S0283/upa.f3 | GTGGATGTGCCCTGAAGGA | 1078 |
| upa | NM_002658 | S0285/upa.r3 | CTGCGGATCCAGGGTAAGAA | 1079 |
| upa | NM_002658 | S4769/upa.p3 | AAGCCAGGCGTCTACACGAGAGTCTCAC | 1080 |
| V-RAF | NM_001654 | S5763/V-RAF.f1 | GGTTGTGCTCTACGAGCTTATGAC | 1081 |
| V-RAF | NM_001654 | S5764/V-RAF.r1 | CGGCCCACCATAAAGATAATCT | 1082 |
| V-RAF | NM_001654 | S5765/V-RAF.p1 | TGCCTTACAGCCACATTGGCTGCC | 1083 |
| VCAM1 | NM_001078 | S3505/VCAM1.f1 | TGGCTTCAGGAGCTGAATACC | 1084 |
| VCAM1 | NM_001078 | S3506/VCAM1.r1 | TGCTGTCGTGATGAGAAAATAGTG | 1085 |
| VCAM1 | NM_001078 | S3507/VCAM1.p1 | CAGGCACACACAGGTGGGACACAAAT | 1086 |
| VEGF | NM_003376 | S0286/VEGF.f1 | CTGCTGTCTTGGGTGCATTG | 1087 |
| VEGF | NM_003376 | S0288/VEGF.r1 | GCAGCCTGGGACCACTTG | 1088 |
| VEGF | NM_003376 | S4782/VEGF.p1 | TTGCCTTGCTGCTCTACCTCCACCA | 1089 |
| VEGFB | NM_003377 | S2724/VEGFB.f1 | TGACGATGGCCTGGAGTGT | 1090 |
| VEGFB | NM_003377 | S2725/VEGFB.r1 | GGTACCGGATCATGAGGATCTG | 1091 |
| VEGFB | NM_003377 | S4960/VEGFB.p1 | CTGGGCAGCACCAAGTCCGGA | 1092 |
| VEGFC | NM_005429 | S2251/VEGFC.f1 | CCTCAGCAAGACGTTATTTGAAATT | 1093 |
| VEGFC | NM_005429 | S2252/VEGFC.r1 | AAGTGTGATTGGCAAAACTGATTG | 1094 |
| VEGFC | NM_005429 | S4758/VEGFC.p1 | CCTCTCTCTCAAGGCCCCAAACCAGT | 1095 |
| VHL | NM_000551 | T1359/VHL.f1 | CGGTTGGTGACTTGTCTGC | 1096 |
| VHL | NM_000551 | T1360/VHL.r1 | AAGACTTGTCCCTGCCTCAC | 1097 |
| VHL | NM_000551 | T1361/VHL.p1 | ATGCCTCAGTCTTCCCAAAGCAGG | 1098 |
| VIM | NM_003380 | S0790/VIM.f3 | TGCCCTTAAAGGAACCAATGA | 1099 |
| VIM | NM_003380 | S0791/VIM.r3 | GCTTCAACGGCAAAGTTCTCTT | 1100 |
| VIM | NM_003380 | S4810/VIM.p3 | ATTTCACGCATCTGGCGTTCCA | 1101 |
| WAVE3 | NM_006646 | T2640/WAVE3.f1 | CTCTCCAGTGTGGGCACC | 1102 |
| WAVE3 | NM_006646 | T2641/WAVE3.r1 | GCGGTGTAGCTCCCAGAGT | 1103 |
| WAVE3 | NM_006646 | T2642/WAVE3.p1 | CCAGAACAGATGCGAGCAGTCCAT | 1104 |
| Wnt-5a | NM_003392 | S6183/Wnt-5a.f1 | GTATCAGGACCACATGCAGTACATC | 1105 |
| Wnt-5a | NM_003392 | S6184/Wnt-5a.r1 | TGTCGGAATTGATACTGGCATT | 1106 |
| Wnt-5a | NM_003392 | S6185/Wnt-5a.p1 | TTGATGCCTGTCTTCGCGCCTTCT | 1107 |
| XIAP | NM_001167 | S0289/XIAP.f1 | GCAGTTGGAAGACACAGGAAAGT | 1108 |
| XIAP | NM_001167 | S0291/XIAP.r1 | TGCGTGGCACTATTTTCAAGA | 1109 |
| XIAP | NM_001167 | S4752/XIAP.p1 | TCCCCAAATTGCAGATTTATCAACGGC | 1110 |
| XIST | M97168 | S1844/XIST.f1 | CAGGTCAGGCAGAGGAAGTC | 1111 |
| XIST | M97168 | S1845/XIST.r1 | CCTAACAAGCCCCAAATCAA | 1112 |
| XIST | M97168 | S8271/XIST.p1 | TGCATTGCATGAGCTAAACCTATCTGA | 1113 |
| ZW10 | NM_004724 | T2117/ZW10.f1 | TGGTCAGATGCTGCTGAAGT | 1114 |
| ZW10 | NM_004724 | T2118/ZW10.r1 | ATCACAGCATGAAGGGATGG | 1115 |
| ZW10 | NM_004724 | T2119/ZW10.p1 | TATCCTTAGGCCGCTGGCATCTTG | 1116 |
| ZWILCH | NM_017975 | T2057/ZWILCH.f1 | GAGGGAGCAGACAGTGGGT | 1117 |
| ZWILCH | NM_017975 | T2058/ZWILCH.r1 | TCAGAGCCCTTGCTAAGTCAC | 1118 |
| ZWILCH | NM_017975 | T2059/ZWILCH.p1 | CCACGATCTCCGTAACCATTTGCA | 1119 |
| ZWINT | NM_007057 | S8920/ZWINT.f1 | TAGAGGCCATCAAAATTGGC | 1120 |
| ZWINT | NM_007057 | S8921/ZWINT.r1 | TCCGTTTCCTCTGGGCTT | 1121 |
| ZWINT | NM_007057 | S8922/ZWINT.p1 | ACCAAGGCCCTGACTCAGATGGAG | 1122 |

TABLE 3

| | Access | | SEQ ID |
|---|---|---|---|
| Gene Name | ion # | Amplicon Sequence | NO: |
| ABCA9 | NM_080283 | TTACCCGTGGGAACTGTCTCCAAATACATACTTCCTCTCACCAGGA | 1123 |
| | | CAACAACCACAGGATCCTCTGACCCATTTACTGGTC | |
| ABCB1 | NM_000927 | AAACACCACTGGAGCATTGACTACCAGGCTCGCCAATGATGCTGCT | 1124 |
| | | CAAGTTAAAGGGGCTATAGGTTCCAGGCTTG | |
| ABCB5 | NM_178559 | AGACAGTCGCCTTGGTCGGTCTCAATGGCAGTGGGAAGAGTACGG | 1125 |
| | | TAGTCCAGCTTCTGCAGAGGTT | |
| ABCC10 | NM_033450 | ACCAGTGCCACAATGCAGTGGCTGGACATTCGGCTACAGCTCATG | 1126 |
| | | GGGGCGGCAGTGGTCAGCGCTAT | |
| ABCC11 | NM_032583 | AAGCCACAGCCTCCATTGACATGGAGACAGACACCCTGATCCAGC | 1127 |
| | | GCACAATCCGTGAAGCCTTCC | |
| ABCC5 | NM_005688 | TGCAGACTGTACCATGCTGACCATTGCCCATCGCCTGCACACGGTT | 1128 |
| | | CTAGGCTCCGATAGGATTATGGTGCTGGCC | |
| ABCD1 | NM_000033 | TCTGTGGCCCACCTCTACTCCAACCTGACCAAGCCACTCCTGGAC | 1129 |
| | | GTGGCTGTGACTTCCTACACCC | |
| ACTG2 | NM_001615 | ATGTACGTCGCCATTCAAGCTGTGCTCTCCCTCTATGCCTCTGGCC | 1130 |
| | | GCACGACAGGCATCGTCCTGGATTCAGGTGATGGCGT | |
| ACTR2 | NM_005722 | ATCCGCATTGAAGACCCACCCCGCAGAAAGCACATGGTATTCCTG | 1131 |
| | | GGTGGTGCAGTTCTAGCGGAT | |
| ACTR3 | NM_005721 | CAACTGCTGAGAGACCGAGAAGTAGGAATCCCTCCAGAACAATCCT | 1132 |
| | | TGGAAACTGCTAAGGCAGTAAAGGAGCG | |
| AK055699 | NM_194317 | CTGCATGTGATTGAATAAGAAACAAGAAAGTGACCACACCAAAGCC | 1133 |
| | | TCCCTGGCTGGTGTACAGGGATCAGGTCCACA | |
| AKT1 | NM_005163 | CGCTTCTATGGCGCTGAGATTGTGTCAGCCCTGGACTACCTGCACT | 1134 |
| | | CGGAGAAGAACGTGGTGTACCGGGA | |
| AKT2 | NM_001626 | TCCTGCCACCCTTCAAACCTCAGGTCACGTCCGAGGTCGACACAA | 1135 |
| | | GGTACTTCGATGATGAATTTACCGCC | |
| AKT3 | NM_005465 | TTGTCTCTGCCTTGGACTATCTACATTCCGGAAAGATTGTGTACCGT | 1136 |
| | | GATCTCAAGTTGGAGAATCTAATGCTGG | |
| ANXA4 | NM_001153 | TGGGAGGGATGAAGGAAATTATCTGGACGATGCTCTCGTGAGACA | 1137 |
| | | GGATGCCCAGGACCTGTATGAG | |
| APC | NM_000038 | GGACAGCAGGAATGTGTTTCTCCATACAGGTCACGGGGAGCCAAT | 1138 |
| | | GGTTCAGAAACAAATCGAGTGGGT | |
| APEX-1 | NM_001641 | GATGAAGCCTTTCGCAAGTTCCTGAAGGGCCTGGCTTCCCGAAAG | 1139 |
| | | CCCCTTGTGCTGTGTGGAGACCT | |
| APOC1 | NM_001645 | GGAAACACACTGGAGGACAAGGCTCGGGAACTCATCAGCCGCATC | 1140 |
| | | AAACAGAGTGAACTTTCTGCCAAGATGCG | |
| APOD | NM_001647 | GTTTATGCCATCGGCACCGTACTGGATCCTGGCCACCGACTATGA | 1141 |
| | | GAACTATGCCCTCGTGTATTCC | |
| APOE | NM_000041 | GCCTCAAGAGCTGGTTCGAGCCCCTGGTGGAAGACATGCAGCGCC | 1142 |
| | | AGTGGGCCGGGCTGGTGGAGAAGGTGCAGG | |
| APRT | NM_000485 | GAGGTCCTGGAGTGCGTGAGCCTGGTGGAGCTGACCTCGCTTAAG | 1143 |
| | | GGCAGGGAGAAGCTGGCACCT | |
| ARHA | NM_001664 | GGTCCTCCGTCGGTTCTCTCATTAGTCCACGGTCTGGTCTTCAGCT | 1144 |
| | | ACCCGCCTTCGTCTCCGAGTTTGCGAC | |
| AURKB | NM_004217 | AGCTGCAGAAGAGCTGCACATTTGACGAGCAGCGAACAGCCACGA | 1145 |
| | | TCATGGAGGAGTTGGCAGATGC | |
| B-actin | NM_001101 | CAGCAGATGTGGATCAGCAAGCAGGAGTATGACGAGTCCGGCCCC | 1146 |
| | | TCCATCGTCCACCGCAAATGC | |
| BAD | NM_032989 | GGGTCAGGTGCCTCGAGATCGGGCTTGGGCCCAGAGCATGTTCCA | 1147 |
| | | GATCCCAGAGTTTGAGCCGAGTGAGCAG | |
| BAG1 | NM_004323 | CGTTGTCAGCACTTGGAATACAAGATGGTTGCCGGGTCATGTTAAT | 1148 |
| | | TGGGAAAAAGAACAGTCCACAGGAAGAGGTTGAAC | |
| Bak | NM_001188 | CCATTCCCACCATTCTACCTGAGGCCAGGACGTCTGGGGTGTGGG | 1149 |
| | | GATTGGTGGGTCTATGTTCCC | |
| Bax | NM_004324 | CCGCCGTGGACACAGACTCCCCCCGAGAGGTCTTTTTCCGAGTGG | 1150 |
| | | CAGCTGACATGTTTTCTGACGGCAA | |
| BBC3 | NM_014417 | CCTGGAGGGTCCTGTACAATCTCATCATGGGACTCCTGCCCTTACC | 1151 |
| | | CAGGGGCCACAGAGCCCCCGAGATGGAGCCCAATTAG | |
| B-Catenin | NM_001904 | GGCTCTTGTGCGTACTGTCCTTCGGGCTGGTGACAGGGAAGACAT | 1152 |
| | | CACTGAGCCTGCCATCTGTGCTCTTCGTCATCTGA | |
| Bcl2 | NM_000633 | CAGATGGACCTAGTACCCACTGAGATTTCCACGCCGAAGGACAGC | 1153 |
| | | GATGGGAAAAATGCCCTTAAATCATAGG | |
| BCL2L11 | NM_138621 | AATTACCAAGCAGCCGAAGACCACCCACGAATGGTTATCTTACGAC | 1154 |
| | | TGTTACGTTACATTGTCCGCCTG | |
| BCL2L13 | NM_015367 | CAGCGACAACTCTGGACAAGTCAGTCCCCCAGAGTCTCCAACTGT | 1155 |
| | | GACCACTTCCTGGCAGTCTGAGAGC | |
| Bclx | NM_001191 | CTTTTGTGGAACTCTATGGGAACAATGCAGCAGCCGAGAGCCGAA | 1156 |
| | | AGGGCCAGGAACGCTTCAACCGCTG | |
| BCRP | NM_004827 | TGTACTGGCGAAGAATATTTGGTAAAGCAGGGCATCGATCTCTCAC | 1157 |
| | | CCTGGGGCTTGTGGAAGAATCACGTGGC | |
| BID | NM_001196 | GGACTGTGAGGTCAACAACGGTTCCAGCCTCAGGGATGAGTGCAT | 1158 |
| | | CACAAACCTACTGGTGTTTGGCTTCC | |
| BIN1 | NM_004305 | CCTGCAAAAGGGAACAAGAGCCCTTCGCCTCCAGATGGCTCCCCT | 1159 |
| | | GCCGCCACCCCCGAGATCAGAGTCAACCACG | |
| BRCA1 | NM_007295 | TCAGGGGGCTAGAAATCTGTTGCTATGGGCCCTTCACCAACATGCC | 1160 |
| | | CACAGATCAACTGGAATGG | |
| BRCA2 | NM_000059 | AGTTCGTGCTTTGCAAGATGGTGCAGAGCTTTATGAAGCAGTGAAG | 1161 |
| | | AATGCAGCAGACCCAGCTTACCTT | |
| BUB1 | NM_004336 | CCGAGGTTAATCCAGCACGTATGGGGCCAAGTGTAGGCTCCCAGC | 1162 |
| | | AGGAACTGAGAGCGCCATGTCTT | |
| BUB1B | NM_001211 | TCAACAGAAGGCTGAACCACTAGAAAGACTACAGTCCCAGCACCG | 1163 |
| | | ACAATTCCAAGCTCGAGTGTCTCGGCAAACTCTGTTG | |
| BUB3 | NM_004725 | CTGAAGCAGATGGTTCATCATTTCCTGGGCTGTTAAACAAAGCGAG | 1164 |
| | | GTTAAGGTTAGACTCTTGGGAATCAGC | |
| C14orf10 | NM_017917 | GTCAGCGTGGTAGCGGTATTCTCCGCGGCAGTGACAGTAATTGTTT | 1165 |
| | | TTGCCTCTTTAGCCAAGACTTCC | |
| C20_orf1 | NM_012112 | TCAGCTGTGAGCTGCGGATACCGCCCGGCAATGGGACCTGCTCTT | 1166 |
| | | AACCTCAAACCTAGGACCGT | |
| CA9 | NM_001216 | ATCCTAGCCCTGGTTTTTGGCCTCCTTTTTGCTGTCACCAGCGTCG | 1167 |
| | | CGTTCCTTGTGCAGATGAGAAGGCAG | |
| CALD1 | NM_004342 | CACTAAGGTTTGAGACAGTTCCAGAAAGAACCCAAGCTCAAGACGC | 1168 |
| | | AGGACGAGCTCAGTTGTAGAGGGCTAATTCGC | |
| CAPZA1 | NM_006135 | TCGTTGGAGATCAGAGTGGAAGTTCACCATCACACCACCTACAGCC | 1169 |
| | | CAGGTGGTTGGCGTGCTTAA | |
| CAV1 | NM_001753 | GTGGCTCAACATTGTGTTCCCATTTCAGCTGATCAGTGGGCCTCCA | 1170 |
| | | AGGAGGGGCTGTAAAATGGAGGCCATTG | |
| CCNB1 | NM_031966 | TTCAGGTTGTTGCAGGAGACCATGTACATGACTGTCTCCATTATTG | 1171 |
| | | ATCGGTTCATGCAGAATAATTGTGTGCCCAAGAAGATG | |
| CCND1 | NM_053056 | GCATGTTCGTGGCCTCTAAGATGAAGGAGACCATCCCCCTGACGG | 1172 |
| | | CCGAGAAGCTGTGCATCTACACCG | |
| CCNE2 | NM_057749 | ATGCTGTGGCTCCTTCCTAACTGGGGCTTTCTTGACATGTAGGTTG | 1173 |
| | | CTTGGTAATAACCTTTTTGTATATCACAATTTGGGT | |
| CCT3 | NM_001008800 | ATCCAAGGCCATGACTGGTGTGGAACAATGGCCATACAGGGCTGT | 1174 |
| | | TGCCCAGGCCCTAGAGGTCATTCC | |
| CD14 | NM_000591 | GTGTGCTAGCGTACTCCCGCCTCAAGGAACTGACGCTCGAGGACC | 1175 |
| | | TAAAGATAACCGGCACCATGC | |
| CD31 | NM_000442 | TGTATTTCAAGACCTCTGTGCACTTATTTATGAACCTGCCCTGCTCC | 1176 |
| | | CACAGAACACAGCAATTCCTCAGGCTAA | |
| CD3z | NM_000734 | AGATGAAGTGGAAGGCGCTTTTCACCGCGGCCATCCTGCAGGCAC | 1177 |
| | | AGTTGCCGATTACAGAGGCA | |
| CD63 | NM_001780 | AGTGGGACTGATTGCCGTGGGTGTCGGGGCACAGCTTGTCCTGAG | 1178 |
| | | TCAGACCATAATCCAGGGGGCTACCC | |
| CD68 | NM_001251 | TGGTTCCCAGCCCTGTGTCCACCTCCAAGCCCAGATTCAGATTCGA | 1179 |
| | | GTCATGTACACAACCCAGGGTGGAGGAG | |
| CDC2 | NM_001786 | GAGAGCGACGCGGTTGTTGTAGCTGCCGCTGCGGCCGCCGCGGA | 1180 |
| | | ATAATAAGCCGGGATCTACCATAC | |
| CDC20 | NM_001255 | TGGATTGGAGTTCTGGGAATGTACTGGCCGTGGCACTGGACAACA | 1181 |
| | | GTGTGTACCTGTGGAGTGCAAGC | |
| CDC25B | NM_021873 | AAACGAGCAGTTTGCCATCAGACGCTTCCAGTCTATGCCGGTGAG | 1182 |
| | | GCTGCTGGGCCACAGCCCCGTGCTTCGGAACATCACCAAC | |
| CDCA8 | NM_018101 | GAGGCACAGTATTGCCCAGCTGGATCCAGAGGCCTTGGGAAACAT | 1183 |
| | | TAAGAAGCTCTCCAACCGTCTC | |
| CDH1 | NM_004360 | TGAGTGTCCCCCGGTATCTTCCCCGCCCTGCCAATCCCGATGAAAT | 1184 |
| | | TGGAAATTTTATTGATGAAAATCTGAAAGCGGCTG | |
| CDK5 | NM_004935 | AAGCCCTATCCGATGTACCCGGCCACAACATCCCTGGTGAACGTC | 1185 |
| | | GTGCCCAAACTCAATGCCACAG | |
| CDKN1C | NM_000076 | CGGCGATCAAGAAGCTGTCCGGGCCTCTGATCTCCGATTTCTTCG | 1186 |
| | | CCAAGCGCAAGAGATCAGCGCCTG | |
| CEGP1 | NM_020974 | TGACAATCAGCACACCTGCATTCACCGCTCGGAAGAGGGCCTGAG | 1187 |
| | | CTGCATGAATAAGGATCACGGCTGTAGTCACA | |
| CENPA | NM_001809 | TAAATTCACTCGTGGTGTGGACTTCAATTGGCAAGCCCAGGCCCTA | 1188 |
| | | TTGGCCCTACAAGAGGC | |
| CENPE | NM_001813 | GGATGCTGGTGACCTCTTCTTCCCTCACGTTGCAACAGGAATTAAA | 1189 |
| | | GGCTAAAAGAAAACGAAGAGTTACTTGGTGCCTTGGC | |
| CENPF | NM_016343 | CTCCCGTCAACAGCGTTCTTTCCAAACACTGGACCAGGAGTGCATC | 1190 |
| | | CAGATGAAGGCCAGACTCACCC | |
| CGA (CHGA | NM_001275 | CTGAAGGAGCTCCAAGACCTCGCTCTCCAAGGCGCCAAGGAGAGG | 1191 |
| official) | | GCACATCAGCAGAAGAAACACAGCGGTTTTG | |
| CHFR | NM_018223 | AAGGAAGTGGTCCCTCTGTGGCAAGTGATGAAGTCTCCAGCTTTGC | 1192 |
| | | CTCAGCTCTCCCAGACAGAAAGACTGCGTC | |
| Chk1 | NM_001274 | GATAAATTGGTACAAGGGATCAGCTTTTCCCAGCCCACATGTCCTG | 1193 |
| | | ATCATATGCTTTTGAATAGTCAGTTACTTGGCACCC | |
| Chk2 | NM_007194 | ATGTGGAACCCCCACCTACTTGGCGCCTGAAGTTCTTGTTTCTGTT | 1194 |
| | | GGGACTGCTGGGTATAACCGTGCTGTGGACTG | |
| cIAP2 | NM_001165 | GGATATTTCCGTGGCTCTTATTCAAACTCTCCATCAAATCCTGTAAA | 1195 |
| | | CTCCAGAGCAAATCAAGATTTTTCTGCCTTGATGAGAAG | |
| CKAP1 | NM_001281 | TCATTGACCACAGTGGCGCCCGCCTTGGTGAGTATGAGGACGTGT | 1196 |
| | | CCCGGGTGGAGAAGTACACGA | |
| CLU | NM_001831 | CCCCAGGATACCTACCACTACCTGCCCTTCAGCCTGCCCCACCGG | 1197 |
| | | AGGCCTCACTTCTTCTTTCCCAAGTCCCGCA | |
| cMet | NM_000245 | GACATTTCCAGTCCTGCAGTCAATGCCTCTCTGCCCCACCCTTTGT | 1198 |
| | | TCAGTGTGGCTGGTGCCACGACAAATGTGTGCGATCGGAG | |
| cMYC | NM—002467 | TCCCTCCACTCGGAAGGACTATCCTGCTGCCAAGAGGGTCAAGTT | 1199 |
| | | GGACAGTGTCAGAGTCCTGAGACAGATCAGCAACAACCG | |
| CNN | NM_001299 | TCCACCCTCCTGGCTTTGGCCAGCATGGCGAAGACGAAAGGAAAC | 1200 |
| | | AAGGTGAACGTGGGAGTGA | |
| COL1A1 | NM_000088 | GTGGCCATCCAGCTGACCTTCCTGCGCCTGATGTCCACCGAGGCC | 1201 |
| | | TCCCAGAACATCACCTACCACTG | |
| COL1A2 | NM_000089 | CAGCCAAGAACTGGTATAGGAGCTCCAAGGACAAGAAACACGTCT | 1202 |
| | | GGCTAGGAGAAACTATCAATGCTGGCAGCCAGTTT | |
| COL6A3 | NM_004369 | GAGAGCAAGCGAGACATTCTGTTCCTCTTTGACGGCTCAGCCAATC | 1203 |
| | | TTGTGGGCCAGTTCCCTGTT | |
| Contig | NM_198477 | CGACAGTTGCGATGAAAGTTCTAATCTCTTCCCTCCTCCTGTTGCT | 1204 |
| 51037 | | GCCACTAATGCTGATGTCCATGGTCTCTAGCAGCC | |
| COX2 | NM_000963 | TCTGCAGAGTTGGAAGCACTCTATGGTGACATCGATGCTGTGGAG | 1205 |
| | | CTGTATCCTGCCCTTCTGGTAGAAAAGCCTCGGC | |
| COX7C | NM_001867 | ACCTCTGTGGTCCGTAGGAGCCACTATGAGGAGGGCCCTGGGAAG | 1206 |
| | | AATTTGCCATTTTCAGTGGAAAACAAGTGGTCG | |
| CRABP1 | NM_004378 | AACTTCAAGGTCGGAGAAGGCTTTGAGGAGGAGACCGTGGACGGA | 1207 |
| | | CGCAAGTGCAGGAGTTTAGCCA | |
| CRIP2 | NM_001312 | GTGCTACGCCACCCTGTTCGGACCCAAAGGCGTGAACATCGGGGG | 1208 |
| | | CGCGGGCTCCTACATCTACGAGAAGCCCCTG | |
| CRYAB | NM_001885 | GATGTGATTGAGGTGCATGGAAAACATGAAGAGCGCCAGGATGAA | 1209 |
| | | CATGGTTTCATCTCCAGGGAGTTC | |
| CSF1 | NM_000757 | TGCAGCGGCTGATTGACAGTCAGATGGAGACCTCGTGCCAAATTA | 1210 |
| | | CATTTGAGTTTGTAGACCAGGAACAGTTG | |
| CSNK1D | NM_001893 | AGCTTTTCCGGAATCTGTTCCATCGCCAGGGCTTCTCCTATGACTA | 1211 |
| | | CGTGTTCGACTGGAACATGCTCAAAT | |
| CST7 | NM_003650 | TGGCAGAACTACCTGCAAGAAAAACCAGCACCTGCGTCTGGATGA | 1212 |
| | | CTGTGACTTCCAAACCAACCACACCTTGAAGCA | |
| CTSD | NM_001909 | GTACATGATCCCCTGTGAGAAGGTGTCCACCCTGCCCGCGATCAC | 1213 |
| | | ACTGAAGCTGGGAGGCAAAGGCTACAAGCTGTCCC | |
| CTSL | NM_001912 | GGGAGGCTTATCTCACTGAGTGAGCAGAATCTGGTAGACTGCTCT | 1214 |
| | | GGGCCTCAAGGCAATGAAGGCTGCAATGG | |
| CTSL2 | NM_001333 | TGTCTCACTGAGCGAGCAGAATCTGGTGGACTGTTCGCGTCCTCAA | 1215 |
| | | GGCAATCAGGGCTGCAATGGT | |
| CXCR4 | NM_003467 | TGACCGCTTCTACCCCAATGACTTGTGGGTGGTTGTGTTCCAGTTT | 1216 |
| | | CAGCACATCATGGTTGGCCTTATCCT | |
| CYBA | NM_000101 | GGTGCCTACTCCATTGTGGCGGGCGTGTTTGTGTGCCTGCTGGAG | 1217 |
| | | TACCCCCGGGGGAAGAGGAAGAAGGGCTCCAC | |
| CYP1B1 | NM_000104 | CCAGCTTTGTGCCTGTCACTATTCCTCATGCCACCACTGCCAACAC | 1218 |
| | | CTCTGTCTTGGGCTACCACATTCCC | |
| CYP2C8 | NM_000770 | CCGTGTTCAAGAGGAAGCTCACTGCCTTGTGGAGGAGTTGAGAAA | 1219 |
| | | AACCAAGGCTTCACCCTGTGATCCCACT | |
| CYP3A4 | NM_017460 | AGAACAAGGACAACATAGATCCTTACATATACACACCCTTTGGAAG | 1220 |
| | | TGGACCCAGAAACTGCATTGGCATGAGGTTTGC | |
| DDR1 | NM_001954 | CCGTGTGGCTCGCTTTCTGCAGTGCCGCTTCCTCTTTGCGGGGCC | 1221 |
| | | CTGGTTACTCTTCAGCGAAATCTCC | |
| DIABLO | NM_019887 | CACAATGGCGGCTCTGAAGAGTTGGCTGTCGCGCAGCGTAACTTC | 1222 |
| | | ATTCTTCAGGTACAGACAGTGTTTGTGT | |
| DIAPH1 | NM_005219 | CAAGCAGTCAAGGAGAACCAGAAGCGGCGGGAGACAGAAGAAAA | 1223 |
| | | GATGAGGCGAGCAAAACT | |
| DICER1 | NM_177438 | TCCAATTCCAGCATCACTGTGGAGAAAAGCTGTTTGTCTCCCCAGC | 1224 |
| | | ATACTTTATCGCCTTCACTGCC | |
| DKFZp564D0462; | NM_198569 | CAGTGCTTCCATGGACAAGTCCTTGTCAAAACTGGCCCATGCTGAT | 1225 |
| | | GGAGATCAAACATCAATCATCCCTGTCCA | |
| DR4 | NM_003844 | TGCACAGAGGGTGTGGGTTACACCAATGCTTCCAACAATTTGTTTG | 1226 |
| | | CTTGCCTCCCATGTACAGCTTGTAAATCAGATGAAGA | |
| DR5 | NM_003842 | CTCTGAGACAGTGCTTCGATGACTTTGCAGACTTGGTGCCCTTTGA | 1227 |
| | | CTCCTGGGAGCCGCTCATGAGGAAGTTGGGCCTCATGG | |
| DUSP1 | NM_004417 | AGACATCAGCTCCTGGTTCAACGAGGCCATTGACTTCATAGACTCC | 1228 |
| | | ATCAAGAATGCTGGAGGAAGGGTGTTTGTC | |
| EEF1D | NM_001960 | CAGAGGATGACGAGGATGATGACATTGACCTGTTTGGCAGTGACA | 1229 |
| | | ATGAGGAGGAGGACAAGGAGGCGGCACAG | |
| EGFR | NM_005228 | TGTCGATGGACTTCCAGAACCACCTGGGCAGCTGCCAAAAGTGTG | 1230 |
| | | ATCCAAGCTGTCCCAAT | |
| EIF4E | NM_001968 | GATCTAAGATGGCGACTGTCGAACCGGAAACCACCCCTACTCCTAA | 1231 |
| | | TCCCCCGACTACAGAAGAGGAGAAAACGGAATCTAA | |
| EIF4EL3 | NM_004846 | AAGCCGCGGTTGAATGTGCCATGACCCTCTCCCTCTCTGGATGGC | 1232 |
| | | ACCATCATTGAAGCTGGCGTCA | |
| ELP3 | NM_018091 | CTCGGATCCTAGCCCTCGTGCCTCCATGGACTCGAGTGTACCGAG | 1233 |
| | | TACAGAGGGATATTCCAATGCC | |
| ER2 | NM_001437 | TGGTCCATCGCCAGTTATCACATCTGTATGCGGAACCTCAAAAGAG | 1234 |
| | | TCCCTGGTGTGAAGCAAGATCGCTAGAACA | |
| ErbB3 | NM_001982 | CGGTTATGTCATGCCAGATACACACCTCAAAGGTACTCCCTCCTCC | 1235 |
| | | CGGGAAGGCACCCTTTCTTCAGTGGGTCTCAGTTC | |
| ERBB4 | NM_005235 | TGGCTCTTAATCAGTTTCGTTACCTGCCTCTGGAGAATTTACGCATT | 1236 |
| | | ATTCGTGGGACAAAACTTTATGAGGATCGATATGCCTTG | |
| ERCC1 | NM_001983 | GTCCAGGTGGATGTGAAAGATCCCCAGCAGGCCCTCAAGGAGCTG | 1237 |
| | | GCTAAGATGTGTATCCTGGCCG | |
| ERK1 | NM_002746 | ACGGATCACAGTGGAGGAAGCGCTGGCTCACCCCTACCTGGAGCA | 1238 |
| | | GTACTATGACCCGACGGATGAG | |
| ESPL1 | NM_012291 | ACCCCCAGACCGGATCAGGCAAGCTGGCCCTCATGTCCCCTTCAC | 1239 |
| | | GGTGTTTGAGGAAGTCTGCCCTACA | |
| EstR1 | NM_000125 | CGTGGTGCCCCTCTATGACCTGCTGCTGGAGATGCTGGACGCCCA | 1240 |
| | | CCGCCTACATGCGCCCACTAGCC | |
| fas | NM_000043 | GGATTGCTCAACAACCATGCTGGGCATCTGGACCCTCCTACCTCTG | 1241 |
| | | GTTCTTACGTCTGTTGCTAGATTATCGTCCAAAAGTGTTAATGCC | |
| fasl | NM_000639 | GCACTTTGGGATTCTTTCCATTATGATTCTTTGTTACAGGCACCGAG | 1242 |
| | | AATGTTGTATTCAGTGAGGGTCTTCTTACATGC | |
| FASN | NM_004104 | GCCTCTTCCTGTTCGACGGCTCGCCCACCTACGTACTGGCCTACA | 1243 |
| | | CCCAGAGCTACCGGGCAAAGC | |
| FBXO5 | NM_012177 | GGCTATTCCTCATTTTCTCTACAAAGTGGCCTCAGTGAACATGAAG | 1244 |
| | | AAGGTAGCCTCCTGGAGGAGAATTTCGGTGACAGTCTACAATCC | |
| FDFT1 | NM_004462 | AAGGAAAGGGTGCCTCATCCCAGCAACCTGTCCTTGTGGGTGATG | 1245 |
| | | ATCACTGTGCTGCTTGTGGCTC | |
| FGFR1 | NM_023109 | CACGGGACATTCACCACATCGACTACTATAAAAAGACAACCAACGG | 1246 |
| | | CCGACTGCCTGTGAAGTGGATGGCACCC | |
| FHIT | NM_002012 | CCAGTGGAGCGCTTCCATGACCTGCGTCCTGATGAAGTGGCCGAT | 1247 |
| | | TTGTTTCAGACGACCCAGAGAG | |
| FIGF | NM_004469 | GGTTCCAGCTTTCTGTAGCTGTAAGCATTGGTGGCCACACCACCTC | 1248 |
| | | CTTACAAAGCAACTAGAACCTGCGGC | |
| FLJ20354 | NM_017779 | GCGTATGATTTCCCGAATGAGTCAAAATGTTGATATGCCCAAACTTC | 1249 |
| (DEPDC1 | | ATGATGCAATGGGTACGAGGTCACTG | |
| official) | | | |
| FOS | NM_005252 | CGAGCCCTTTGATGACTTCCTGTTCCCAGCATCATCCAGGCCCAGT | 1250 |
| | | GGCTCTGAGACAGCCCGCTCC | |
| FOXM1 | NM_021953 | CCACCCCGAGCAAATCTGTCCTCCCCAGAACCCCTGAATCCTGGA | 1251 |
| | | GGCTCACGCCCCCAGCCAAAGTAGGGGGACTGGATTT | |
| FUS | NM_004960 | GGATAATTCAGACAACAACACCATCTTTGTGCAAGGCCTGGGTGAG | 1252 |
| | | AATGTTACAATTGAGTCTGTGGCTGATTACTTCA | |
| FYN | NM_002037 | GAAGCGCAGATCATGAAGAAGCTGAAGCACGACAAGCTGGTCCAG | 1253 |
| | | CTCTATGCAGTGGTGTCTGAGGAG | |
| G1P3 | NM_002038 | CCTCCAACTCCTAGCCTCAAGTGATCCTCCTGTCTCAACCTCCCAA | 1254 |
| | | GTAGGATTACAAGCATGCGCC | |
| GADD45 | NM_001924 | GTGCTGGTGACGAATCCACATTCATCTCAATGGAAGGATCCTGCCT | 1255 |
| | | TAAGTCAACTTATTTGTTTTTGCCGGG | |
| GADD45B | NM_015675 | ACCCTCGACAAGACCACACTTTGGGACTTGGGAGCTGGGGCTGAA | 1256 |
| | | GTTGCTCTGTACCCATGAACTCCCA | |
| GAGE1 | NM_001468 | AAGGGCAATCACAGTGTTAAAAGAAGACATGCTGAAATGTTGCAGG | 1257 |
| | | CTGCTCCTATGTTGGAAAATTCTTCATTGAAGTTCTCC | |
| GAPDH | NM_002046 | ATTCCACCCATGGCAAATTCCATGGCACCGTCAAGGCTGAGAACG | 1258 |
| | | GGAAGCTTGTCATCAATGGAAATCCCATC | |
| GATA3 | NM_002051 | CAAAGGAGCTCACTGTGGTGTCTGTGTTCCAACCACTGAATCTGGA | 1259 |
| | | CCCCATCTGTGAATAAGCCATTCTGACTC | |
| GBP1 | NM_002053 | TTGGGAAATATTTGGGCATTGGTCTGGCCAAGTCTACAATGTCCCA | 1260 |
| | | ATATCAAGGACAACCACCCTAGCTTCT | |
| GBP2 | NM_004120 | GCATGGGAACCATCAACCAGCAGGCCATGGACCAACTTCACTATGT | 1261 |
| | | GACAGAGCTGACAGATCGAATCAAGGCAAACTCCTCA | |
| GCLC | NM_001498 | CTGTTGCAGGAAGGCATTGATCATCTCCTGGCCCAGCATGTTGCTC | 1262 |
| | | ATCTCTTTATTAGAGACCCACTGAC | |
| GDF15 | NM_004864 | CGCTCCAGACCTATGATGACTTGTTAGCCAAAGACTGCCACTGCAT | 1263 |
| | | ATGAGCAGTCCTGGTCCTTCCACTGT | |
| GGPS1 | NM_004837 | CTCCGACGTGGCTTTCCAGTGGCCCACAGCATCTATGGAATCCCAT | 1264 |
| | | CTGTCATCAATTCTGCCAATTACG | |
| GLRX | NM_002064 | GGAGCTCTGCAGTAACCACAGAACAGGCCCCATGCTGACGTCCCT | 1265 |
| | | CCTCAAGAGCTGGATGGCATTG | |
| GNS | NM_002076 | GGTGAAGGTTGTCTCTTCCGAGGGCCTTCTGAAGACAGGGCTCTT | 1266 |
| | | GAACAGACAAGTGGAAGGGCTG | |
| GPR56 | NM_005682 | TACCCTTCCATGTGCTGGATCCGGGACTCCCTGGTCAGCTACATCA | 1267 |
| | | CCAACCTGGGCCTCTTCAGC | |
| GPX1 | NM_000581 | GCTTATGACCGACCCCAAGCTCATCACCTGGTCTCCGGTGTGTCG | 1268 |
| | | CAACGATGTTGCCTGGAACTTT | |
| GRB7 | NM_005310 | CCATCTGCATCCATCTTGTTTGGGCTCCCCACCCTTGAGAAGTGCC | 1269 |
| | | TCAGATAATACCCTGGTGGCC | |
| GSK3B | NM_002093 | GACAAGGACGGCAGCAAGGTGACAACAGTGGTGGCAACTCCTGG | 1270 |
| | | GCAGGGTCCAGACAGGCCACAA | |
| GSR | NM_000637 | GTGATCCCAAGCCCACAATAGAGGTCAGTGGGAAAAAGTACACCG | 1271 |
| | | CCCCACACATCCTGATCGCCACA | |
| GSTM1 | NM_000561 | AAGCTATGAGGAAAAGAAGTACACGATGGGGGACGCTCCTGATTAT | 1272 |
| | | GACAGAAGCCAGTGGCTGAATGAAAAATTCAAGCTGGGCC | |
| GSTp | NM_000852 | GAGACCCTGCTGTCCCAGAACCAGGGAGGCAAGACCTTCATTGTG | 1273 |
| | | GGAGACCAGATCTCCTTCGCTGACTACAACC | |
| GUS | NM_000181 | CCCACTCAGTAGCCAAGTCACAATGTTTGGAAAACAGCCCGTTTAC | 1274 |
| | | TTGAGCAAGACTGATACCACCTGCGTG | |
| HDAC6 | NM_006044 | TCCTGTGCTCTGGAAGCCCTTGAGCCCTTCTGGGAGGTTCTTGTGA | 1275 |
| | | GATCAACTGAGACCGTGGAG | |
| HER2 | NM_004448 | CGGTGTGAGAAGTGCAGCAAGCCCTGTGCCCGAGTGTGCTATGGT | 1276 |
| | | CTGGGCATGGAGCACTTGCGAGAGG | |
| HIF1A | NM_001530 | TGAACATAAAGTCTGCAACATGGAAGGTATTGCACTGCACAGGCCA | 1277 |
| | | CATTCACGTATATGATACCAACAGTAACCAACCTCA | |
| HNF3A | NM_004496 | TCCAGGATGTTAGGAACTGTGAAGATGGAAGGGCATGAAACCAGC | 1278 |
| | | GACTGGAACAGCTACTACGCAGACACGC | |
| HRAS | NM_005343 | GGACGAATACGACCCCACTATAGAGGATTCCTACCGGAAGCAGGT | 1279 |
| | | GGTCATTGATGGGGAGACGTGC | |
| HSPA1A | NM_005345 | CTGCTGCGACAGTCCACTACCTTTTTCGAGAGTGACTCCCGTTGTC | 1280 |
| | | CCAAGGCTTCCCAGAGCGAACCTG | |
| HSPA1B | NM_005346 | GGTCCGCTTCGTCTTTCGAGAGTGACTCCCGCGGTCCCAAGGCTT | 1281 |
| | | TCCAGAGCGAACCTGTGC | |
| HSPA1L | NM_005527 | GCAGGTGTGATTGCTGGACTTAATGTGCTAAGAATCATCAATGAGC | 1282 |
| | | CCACGGCTGCTGCCATTGCCTATGGT | |
| HSPA5 | NM_005347 | GGCTAGTAGAACTGGATCCCAACACCAAACTCTTAATTAGACCTAG | 1283 |
| | | GCCTCAGCTGCACTGCCCGAAAAGCATTTGGGCAGACC | |
| HSPA9B | NM_004134 | GGCCACTAAAGATGCTGGCCAGATATCTGGACTGAATGTGCTTCG | 1284 |
| | | GGTGATTAATGAGCCCACAGCTGCT | |
| HSPB1 | NM_001540 | CCGACTGGAGGAGCATAAAAGCGCAGCCGAGCCCAGCGCCCCGC | 1285 |
| | | ACTTTTCTGAGCAGACGTCCAGAGCAGAGTCAGCCAGCAT | |
| HSPCA | NM_005348 | CAAAAGGCAGAGGCTGATAAGAACGACAAGTCTGTGAAGGATCTG | 1286 |
| | | GTCATCTTGCTTTATGAAACTGCGCT | |
| ID1 | NM_002165 | AGAACCGCAAGGTGAGCAAGGTGGAGATTCTCCAGCACGTCATCG | 1287 |
| | | ACTACATCAGGGACCTTCAGTTGGA | |
| IFITM1 | NM_003641 | CACGCAGAAAACCACACTTCTCAAACCTTCACTCAACACTTCCTTCC | 1288 |
| | | CCAAAGCCAGAAGATGCACAAGGAGGAACATG | |
| IGF1R | NM_000875 | GCATGGTAGCCGAAGATTTCACAGTCAAAATCGGAGATTTTGGTAT | 1289 |
| | | GACGCGAGATATCTATGAGACAGACTATTACCGGAAA | |
| IGFBP2 | NM_000597 | GTGGACAGCACCATGAACATGTTGGGCGGGGGAGGCAGTGCTGG | 1290 |
| | | CCGGAAGCCCCTCAAGTCGGGTATGAAGG | |
| IGFBP3 | NM_000598 | ACGCACCGGGTGTCTGATCCCAAGTTCCACCCCCTCCATTCAAAGA | 1291 |
| | | TAATCATCATCAAGAAAGGGCA | |
| IGFBP5 | NM_000599 | TGGACAAGTACGGGATGAAGCTGCCAGGCATGGAGTACGTTGACG | 1292 |
| | | GGGACTTTCAGTGCCACACCTTCG | |
| IL2RA | NM_000417 | TCTGCGTGGTTCCTTTCTCAGCCGCTTCTGACTGCTGATTCTCCCG | 1293 |
| | | TTCACGTTGCCTAATAAACATCCTTCAA | |
| IL6 | NM_000600 | CCTGAACCTTCCAAAGATGGCTGAAAAAGATGGATGCTTCCAATCT | 1294 |
| | | GGATTCAATGAGGAGACTTGCCTGGT | |
| IL-7 | NM_000880 | GCGGTGATTCGGAAATTCGCGAATTCCTCTGGTCCTCATCCAGGTG | 1295 |
| | | CGCGGGAAGCAGGTGCCCAGGAGAG | |
| IL-8 | NM_000584 | AAGGAACCATCTCACTGTGTGTAAACATGACTTCCAAGCTGGCCGT | 1296 |
| | | GGCTCTCTTGGCAGCCTTCCTGAT | |
| IL8RB | NM_001557 | CCGCTCCGTCACTGATGTCTACCTGCTGAACCTAGCCTTGGCCGA | 1297 |
| | | CCTACTCTTTGCCCTGACCTTGC | |
| ILK | NM_001014794 | CTCAGGATTTTCTCGCATCCAAATGTGCTCCCAGTGCTAGGTGCCT | 1298 |
| | | GCCAGTCTCCACCTGCTCCT | |
| ILT-2 | NM_006669 | AGCCATCACTCTCAGTGCAGCCAGGTCCTATCGTGGCCCCTGAGG | 1299 |
| | | AGACCCTGACTCTGCAGT | |
| INCENP | NM_020238 | GCCAGGATACTGGAGTCCATCACAGTGAGCTCCCTGATGGCTACA | 1300 |
| | | CCCCAGGACCCCAAGGGTCAAG | |
| IRAK2 | NM_001570 | GGATGGAGTTCGCCTCCTACGTGATCACAGACCTGACCCAGCTGC | 1301 |
| | | GGAAGATCAAGTCCATGGAGCG | |
| IRS1 | NM_005544 | CCACAGCTCACCTTCTGTCAGGTGTCCATCCCAGCTCCAGCCAGCT | 1302 |
| | | CCCAGAGAGGAAGAGACTGGCACTGAGG | |
| ITGB1 | NM_002211 | TCAGAATTGGATTTGGCTCATTTGTGGAAAAGACTGTGATGCCTTA | 1303 |
| | | CATTAGCACAACACCAGCTAAGCTCAGG | |
| K-Alpha-1 | NM_006082 | TGAGGAAGAAGGAGAGGAATACTAATTATCCATTCCTTTTGGCCCT | 1304 |
| | | GCAGCATGTCATGCTCCCAGAATTTCAG | |
| KDR | NM_002253 | GAGGACGAAGGCCTCTACACCTGCCAGGCATGCAGTGTTCTTGGC | 1305 |
| | | TGTGCAAAAGTGGAGGCATTTTT | |
| Ki-67 | NM_002417 | CGGACTTTGGGTGCGACTTGACGAGCGGTGGTTCGACAAGTGGCC | 1306 |
| | | TTGCGGGCCGGATCGTCCCAGTGGAAGAGTTGTAA | |
| KIF11 | NM_004523 | TGGAGGTTGTAAGCCAATGTTGTGAGGCTTCAAGTTCAGACATCAC | 1307 |
| | | TGAGAAATCAGATGGACGTAAGGCA | |
| KIF22 | NM_007317 | CTAAGGCACTTGCTGGAAGGGCAGAATGCCAGTGTGCTTGCCTAT | 1308 |
| | | GGACCCACAGGAGCTGGGAAGA | |
| KIF2C | NM_006845 | AATTCCTGCTCCAAAAGAAAGTCTTCGAAGCCGCTCCACTCGCATG | 1309 |
| | | TCCACTGTCTCAGAGCTTCGCATCACG | |
| KIFC1 | NM_002263 | CCACAGGGTTGAAGAACCAGAAGCCAGTTCCTGCTGTTCCTGTCCA | 1310 |
| | | GAAGTCTGGCACATCAGGTG | |
| KLK10 | NM_002776 | GCCCAGAGGCTCCATCGTCCATCCTCTTCCTCCCCAGTCGGCTGA | 1311 |
| | | ACTCTCCCCTTGTCTGCACTGTTCAAACCTCTG | |
| KNS2 | NM_005552 | CAAACAGAGGGTGGCAGAAGTGCTCAATGACCCTGAGAACATGGA | 1312 |
| | | GAAGCGCAGGAGCCGTGAGAGCCTC | |
| KNTC1 | NM_014708 | AGCCGAGGCTTTGTTGAAGAAGCTTCATATCCAGTACCGGCGATCG | 1313 |
| | | GGCACAGAAGCTGTGCTCATAGCCCA | |
| KNTC2 | NM_006101 | ATGTGCCAGTGAGCTTGAGTCCTTGGAGAAACACAAGCACCTGCTA | 1314 |
| | | GAAAGTACTGTTAACCAGGGGCTCA | |
| KRT14 | NM_000526 | GGCCTGCTGAGATCAAAGACTACAGTCCCTACTTCAAGACCATTGA | 1315 |
| | | GGACCTGAGGAACAAGATTCTCACAGCCACAGTGGAC | |
| KRT17 | NM_000422 | CGAGGATTGGTTCTTCAGCAAGACAGAGGAACTGAACCGCGAGGT | 1316 |
| | | GGCCACCAACAGTGAGCTGGTGCAGAGT | |
| KRT19 | NM_002276 | TGAGCGGCAGAATCAGGAGTACCAGCGGCTCATGGACATCAAGTC | 1317 |
| | | GCGGCTGGAGCAGGAGATTGCCACCTACCGCA | |
| KRT5 | NM_000424 | TCAGTGGAGAAGGAGTTGGACCAGTCAACATCTCTGTTGTCACAAG | 1318 |
| | | CAGTGTTTCCTCTGGATATGGCA | |
| L1CAM | NM_000425 | CTTGCTGGCCAATGCCTACATCTACGTTGTCCAGCTGCCAGCCAAG | 1319 |
| | | ATCCTGACTGCGGACAATCA | |
| LAMC2 | NM_005562 | ACTCAAGCGGAAATTGAAGCAGATAGGTCTTATCAGCACAGTCTCC | 1320 |
| | | GCCTCCTGGATTCAGTGTCTCGGCTTCAGGGAGT | |
| LAPTM4B | NM_018407 | AGCGATGAAGATGGTCGCGCCCTGGACGCGGTTCTACTCCAACAG | 1321 |
| | | CTGCTGCTTGTGCTGCCATGTC | |
| LIMK1 | NM_016735 | GCTTCAGGTGTTGTGACTGCAGTGCCTCCCTGTCGCACCAGTACTA | 1322 |
| | | TGAGAAGGATGGGCAGCTCTT | |
| LIMK2 | NM_005569 | CTTTGGGCCAGGAGGAATCTGTTACTCGAATCCACCCAGGAACTCC | 1323 |
| | | CTGGCAGTGGATTGTGGGAG | |
| MAD1L1 | NM_003550 | AGAAGCTGTCCCTGCAAGAGCAGGATGCAGCGATTGTGAAGAACA | 1324 |
| | | TGAAGTCTGAGCTGGTACGGCT | |
| MAD2L1 | NM_002358 | CCGGGAGCAGGGAATCACCCTGCGCGGGAGCGCCGAAATCGTGG | 1325 |
| | | CCGAGTTCTTCTCATTCGGCATCAACAGCAT | |
| MAD2L1BP | NM_014628 | CTGTCATGTGGCAGACCTTCCATCCGAACCACGGCTTGGGAAGAC | 1326 |
| | | TACATTTGGTTCCAGGCACCAGTGACATTTA | |
| MAD2L2 | NM_006341 | GCCCAGTGGAGAAATTCGTCTTTGAGATCACCCAGCCTCCACTGCT | 1327 |
| | | GTCCATCAGCTCAGACTCGC | |
| MAGE2 | NM_005361 | CCTCAGAAATTGCCAGGACTTCTTTCCCGTGATCTTCAGCAAAGCC | 1328 |
| | | TCCGAGTACTTGCAGCTGGTCTTTGG | |
| MAGE6 | NM_005363 | AGGACTCCAGCAACCAAGAAGAGGAGGGGCCAAGCACCTTCCCTG | 1329 |
| | | ACCTGGAGTCTGAGTTCCAAGCAGCACTC | |
| MAP2 | NM_002374 | CGGACCACCAGGTCAGAGCCAATTCGCAGAGCAGGGAAGAGTGGT | 1330 |
| | | ACCTCAACACCCACTACCCCTG | |
| MAP2K3 | NM_002756 | GCCCTCCAATGTCCTTATCAACAAGGAGGGCCATGTGAAGATGTGT | 1331 |
| | | GACTTTGGCATCAGTGGCTAC | |
| MAP4 | NM_002375 | GCCGGTCAGGCACACAAGGGGCCCTTGGAGCGTGGACTGGTTGG | 1332 |
| | | TTTTGCCATTTTGTTGTGTGTATGCTGC | |
| MAP6 | NM_033063 | CCCTCAACCGGCAAATCCGCGAGGAGGTGGCGAGTGCAGTGAGC | 1333 |
| | | AGCTCCTACAGGAATGAATTCAGGGCATGGACG | |
| MAPK14 | NM_139012 | TGAGTGGAAAAGCCTGACCTATGATGAAGTCATCAGCTTTGTGCCA | 1334 |
| | | CCACCCCTTGACCAAGAAGAGATGGAGTCC | |
| MAPK8 | NM_002750 | CAACACCCGTACATCAATGTCTGGTATGATCCTTCTGAAGCAGAAG | 1335 |
| | | CTCCACCACCAAAGATCCCTGACAAGCAGTTAGATGA | |
| MAPRE1 | NM_012325 | GACCTTGGAACCTTTGGAACCTGCTGTCAACAGGTCTTACAGGGCT | 1336 |
| | | GCTTGAACCCTCATAGGCCTAGG | |
| MAPT | NM_016835 | CACAAGCTGACCTTCCGCGAGAACGCCAAAGCCAAGACAGACCAC | 1337 |
| | | GGGGCGGAGATCGTGTACAAGT | |
| Maspin | NM_002639 | CAGATGGCCACTTTGAGAACATTTTAGCTGACAACAGTGTGAACGA | 1338 |
| | | CCAGACCAAAATCCTTGTGGTTAATGCTGCC | |
| MCL1 | NM_021960 | CTTCGGAAACTGGACATCAAAAACGAAGACGATGTGAAATCGTTGT | 1339 |
| | | CTCGAGTGATGATCCATGTTTTCAGCGAC | |
| MCM2 | NM_004526 | GACTTTTGCCCGCTACCTTTCATTCCGGCGTGACAACAATGAGCTG | 1340 |
| | | TTGCTCTTCATACTGAAGCAGTTAGTGGC | |
| MCM6 | NM_005915 | TGATGGTCCTATGTGTCACATTCATCACAGGTTTCATACCAACACAG | 1341 |
| | | GCTTCAGCACTTCCTTTGGTGTGTTTCCTGTCCCA | |
| MCP1 | NM_002982 | CGCTCAGCCAGATGCAATCAATGCCCCAGTCACCTGCTGTTATAAC | 1342 |
| | | TTCACCAATAGGAAGATCTCAGTGC | |
| MGMT | NM_002412 | GTGAAATGAAACGCACCACACTGGACAGCCCTTTGGGGAAGCTGG | 1343 |
| | | AGCTGTCTGGTTGTGAGCAGGGTC | |
| MMP12 | NM_002426 | CCAACGCTTGCCAAATCCTGACAATTCAGAACCAGCTCTCTGTGAC | 1344 |
| | | CCCAATTTGAGTTTTGATGCTGTCACTACCGT | |
| MMP2 | NM_004530 | CCATGATGGAGAGGCAGACATCATGATCAACTTTGGCCGCTGGGA | 1345 |
| | | GCATGGCGATGGATACCCCTTTGACGGTAAGGACGGACTCC | |
| MMP9 | NM_004994 | GAGAACCAATCTCACCGACAGGCAGCTGGCAGAGGAATACCTGTA | 1346 |
| | | CCGCTATGGTTACACTCGGGTG | |
| MRE11A | NM_005590 | GCCATGCTGGCTCAGTCTGAGCTGTGGGCCACATCAGCTAGTGGC | 1347 |
| | | TCTTCTCATGCATCAGTTAGGTGGGTCTGGGTG | |
| MRP1 | NM_004996 | TCATGGTGCCCGTCAATGCTGTGATGGCGATGAAGACCAAGACGT | 1348 |
| | | ATCAGGTGGCCCACATGAAGAGCAAAGACAATCG | |
| MRP2 | NM_000392 | AGGGGATGACTTGGACACATCTGCCATTCGACATGACTGCAATTTT | 1349 |
| | | GACAAAGCCATGCAGTTTT | |
| MRP3 | NM_003786 | TCATCCTGGCGATCTACTTCCTCTGGCAGAACCTAGGTCCCTCTGT | 1350 |
| | | CCTGGCTGGAGTCGCTTTCATGGTCTTGCTGATTCCACTCAACGG | |
| MSH3 | NM_002439 | TGATTACCATCATGGCTCAGATTGGCTCCTATGTTCCTGCAGAAGA | 1351 |
| | | AGCGACAATTGGGATTGTGGATGGCATTTTCACAAG | |
| MUC1 | NM_002456 | GGCCAGGATCTGTGGTGGTACAATTGACTCTGGCCTTCCGAGAAG | 1352 |
| | | GTACCATCAATGTCCACGACGTGGAG | |
| MX1 | NM_002462 | GAAGGAATGGGAATCAGTCATGAGCTAATCACCCTGGAGATCAGCT | 1353 |
| | | CCCGAGATGTCCCGGATCTGACTCTAATAGAC | |
| MYBL2 | NM_002466 | GCCGAGATCGCCAAGATGTTGCCAGGGAGGACAGACAATGCTGTG | 1354 |
| | | AAGAATCACTGGAACTCTACCATCAAAAG | |
| MYH11 | NM_002474 | CGGTACTTCTCAGGGCTAATATATACGTACTCTGGCCTCTTCTGCG | 1355 |
| | | TGGTGGTCAACCCCTATAAACACCTGCCCATCTACTCGG | |
| NEK2 | NM_002497 | GTGAGGCAGCGCGACTCTGGCGACTGGCCGGCCATGCCTTCCCG | 1356 |
| | | GGCTGAGGACTATGAAGTGTTGTACACCATTGGCA | |
| NFKBp50 | NM_003998 | CAGACCAAGGAGATGGACCTCAGCGTGGTGCGGCTCATGTTTACA | 1357 |
| | | GCTTTTCTTCCGGATAGCACTGGCAGCT | |
| NFKBp65 | NM_021975 | CTGCCGGGATGGCTTCTATGAGGCTGAGCTCTGCCCGGACCGCTG | 1358 |
| | | CATCCACAGTTTCCAGAACCTGG | |
| NME6 | NM_005793 | CACTGACACCCGCAACACCACCCATGGTTCGGACTCTGTGGTTTCA | 1359 |
| | | GCCAGCAGAGAGATTGCAGCC | |
| NPC2 | NM_006432 | CTGCTTCTTTCCCGAGCTTGGAACTTCGTTATCCGCGATGCGTTTC | 1360 |
| | | CTGGCAGCTACATTCCTGCT | |
| NPD009 | NM_020686 | GGCTGTGGCTGAGGCTGTAGCATCTCTGCTGGAGGTGAGACACTC | 1361 |
| (ABAT | | TGGGAACTGATTTGACCTCGAATGCTCC | |
| official) | | | |
| NTSR2 | NM_012344 | CGGACCTGAATGTAATGCAAGAATGAACAGAACAAGCAAAATGACC | 1362 |
| | | AGCTGCTTAGTCACCTGGCAAAG | |
| NUSAP1 | NM_016359 | CAAAGGAAGAGCAACGGAAGAAACGCGAGCAAGAACGAAAGGAGA | 1363 |
| | | AGAAAGCAAAGGTTTTGGGAAT | |
| p21 | NM_000389 | TGGAGACTCTCAGGGTCGAAAACGGCGGCAGACCAGCATGACAGA | 1364 |
| | | TTTCTACCACTCCAAACGCC | |
| p27 | NM_004064 | CGGTGGACCACGAAGAGTTAACCCGGGACTTGGAGAAGCACTGCA | 1365 |
| | | GAGACATGGAAGAGGCGAGCC | |
| PCTK1 | NM_006201 | TCACTACCAGCTGACATCCGGCTGCCTGAGGGCTACCTGGAGAAG | 1366 |
| | | CTGACCCTCAATAGCCCCATCT | |
| PDGFRb | NM_002609 | CCAGCTCTCCTTCCAGCTACAGATCAATGTCCCTGTCCGAGTGCTG | 1367 |
| | | GAGCTAAGTGAGAGCCACCC | |
| PFDN5 | NM_145897 | GAGAAGCACGCCATGAAACAGGCCGTCATGGAAATGATGAGTCAG | 1368 |
| | | AAGATTCAGCAGCTCACAGCC | |
| PGK1 | NM_000291 | AGAGCCAGTTGCTGTAGAACTCAAATCTCTGCTGGGCAAGGATGTT | 1369 |
| | | CTGTTCTTGAAGGACTGTGTAGGCCCAG | |
| PHB | NM_002634 | GACATTGTGGTAGGGGAAGGGACTCATTTTCTCATCCCGTGGGTAC | 1370 |
| | | AGAAACCAATTATCTTTGACTGCCG | |
| PI3KC2A | NM_002645 | ATACCAATCACCGCACAAACCCAGGCTATTTGTTAAGTCCAGTCAC | 1371 |
| | | AGCGCAAAGAAACATATGCGGAGAAAATGCTAGTGTG | |
| PIM1 | NM_002648 | CTGCTCAAGGACACCGTCTACACGGACTTCGATGGGACCCGAGTG | 1372 |
| | | TATAGCCCTCCAGAGTGGATCC | |
| PIM2 | NM_006875 | TGGGGACATTCCCTTTGAGAGGGACCAGGAGATTCTGGAAGCTGA | 1373 |
| | | GCTCCACTTCCCAGCCCATGTC | |
| PLAUR | NM_002659 | CCCATGGATGCTCCTCTGAAGAGACTTTCCTCATTGACTGCCGAGG | 1374 |
| | | CCCCATGAATCAATGTCTGGTAGCCACCGG | |
| PLD3 | NM_012268 | CCAAGTTCTGGGTGGTGGACCAGACCCACTTCTACCTGGGCAGTG | 1375 |
| | | CCAACATGGACTGGCGTTCAC | |
| PLK | NM_005030 | AATGAATACAGTATTCCCAAGCACATCAACCCCGTGGCCGCCTCCC | 1376 |
| | | TCATCCAGAAGATGCTTCAGACA | |
| PMS1 | NM_000534 | CTTACGGTTTTCGTGGAGAAGCCTTGGGGTCAATTTGTTGTATAGC | 1377 |
| | | TGAGGTTTTAATTACAACAAGAACGGCTGCT | |
| PMS2 | NM_000535 | GATGTGGACTGCCATTCAAACCAGGAAGATACCGGATGTAAATTTC | 1378 |
| | | GAGTTTTGCCTCAGCCAACTAATCTCGCA | |
| PP591 | NM_025207 | CCACATACCGTCCAGCCTATCTACTGGAGAACGAAGAAGAGGAGC | 1379 |
| | | GGAACTCCCGCACATGACCTC | |
| PPP2CA | NM_002715 | GCAATCATGGAACTTGACGATACTCTAAAATACTCTTTCTTGCAGTT | 1380 |
| | | TGACCCAGCACCTCGTAGAGGCGAGCCACAT | |
| PR | NM_000926 | GCATCAGGCTGTCATTATGGTGTCCTTACCTGTGGGAGCTGTAAGG | 1381 |
| | | TCTTCTTTAAGAGGGCAATGGAAGGGCAGCACAACTACT | |
| PRDX1 | NM_002574 | AGGACTGGGACCCATGAACATTCCTTTGGTATCAGACCCGAAGCG | 1382 |
| | | CACCATTGCTCAGGATTATGGG | |
| PRDX2 | NM_005809 | GGTGTCCTTCGCCAGATCACTGTTAATGATTTGCCTGTGGGACGCT | 1383 |
| | | CCGTGGATGAGGCTCTGCGGCTG | |
| PRKCA | NM_002737 | CAAGCAATGCGTCATCAATGTCCCCAGCCTCTGCGGAATGGATCAC | 1384 |
| | | ACTGAGAAGAGGGGGCGGATTTAC | |
| PRKCD | NM_006254 | CTGACACTTGCCGCAGAGAATCCCTTTCTCACCCACCTCATCTGCA | 1385 |
| | | CCTTCCAGACCAAGGACCACCT | |
| PRKCG | NM_002739 | GGGTTCTAGACGCCCCTCCCAAGCGTTCCTGGCCTTCTGAACTCC | 1386 |
| | | ATACAGCCTCTACAGCCGTCC | |
| PRKCH | NM_006255 | CTCCACCTATGAGCGTCTGTCTCTGTGGGCTTGGGATGTTAACAGG | 1387 |
| | | AGCCAAAAGGAGGGAAAGTGTG | |
| pS2 | NM_003225 | GCCCTCCCAGTGTGCAAATAAGGGCTGCTGTTTCGACGACACCGT | 1388 |
| | | TCGTGGGGTCCCCTGGTGCTTCTATCCTAATACCATCGACG | |
| PTEN | NM_000314 | TGGCTAAGTGAAGATGACAATCATGTTGCAGCAATTCACTGTAAAG | 1389 |
| | | CTGGAAAGGGACGAACTGGTGTAATGATATGTGCA | |
| PTPD1 | NM_007039 | CGCTTGCCTAACTCATACTTTCCCGTTGACACTTGATCCACGCAGC | 1390 |
| | | GTGGCACTGGGACGTAAGTGGCGCAGTCTGAATGG | |
| PTTG1 | NM_004219 | GGCTACTCTGATCTATGTTGATAAGGAAAATGGAGAACCAGGCACC | 1391 |
| | | CGTGTGGTTGCTAAGGATGGGCTGAAGC | |
| RAB27B | NM_004163 | GGGACACTGCGGGACAAGAGCGGTTCCGGAGTCTCACCACTGCAT | 1392 |
| | | TTTTCAGAGACGCCATGGGC | |
| RAB31 | NM_006868 | CTGAAGGACCCTACGCTCGGTGGCCTGGCACCTCACTTTGAGAAG | 1393 |
| | | AGTGAGCACACTGGCTTTGCAT | |
| RAB6C | NM_032144 | GCGACAGCTCCTCTAGTTCCACCATGTCCGCGGGCGGAGACTTCG | 1394 |
| | | GGAATCCGCTGAGGAAATTCAAGCTGGTGTTCC | |
| RAD1 | NM_002853 | GAGGAGTGGTGACAGTCTGCAAAATCAATACACAGGAACCTGAGG | 1395 |
| | | AGACCCTGGACTTTGATTTCTGCAGC | |
| RAD54L | NM_003579 | AGCTAGCCTCAGTGACACACATGACAGGTTGCACTGCCGACGTTG | 1396 |
| | | TGTCAACAGCCGTCAGATCCGG | |
| RAF1 | NM_002880 | CGTCGTATGCGAGAGTCTGTTTCCAGGATGCCTGTTAGTTCTCAGC | 1397 |
| | | ACAGATATTCTACACCTCACGCCTTCA | |
| RALBP1 | NM_006788 | GGTGTCAGATATAAATGTGCAAATGCCTTCTTGCTGTCCTGTCGGT | 1398 |
| | | CTCAGTACGTTCACTTTATAGCTGCTGGCAATATCGAA | |
| RAP1GDS | NM_021159 | TGTGGATGCTGGATTGATTTCACCACTGGTGCAGCTGCTAAATAGC | 1399 |
| | | AAAGACCAGGAAGTGCTGCTT | |
| RASSF1 | NM_007182 | AGTGGGAGACACCTGACCTTTCTCAAGCTGAGATTGAGCAGAAGAT | 1400 |
| | | CAAGGAGTACAATGCCCAGATCA | |
| RB1 | NM_000321 | CGAAGCCCTTACAAGTTTCCTAGTTCACCCTTACGGATTCCTGGAG | 1401 |
| | | GGAACATCTATATTTCACCCCTGAAGAGTCC | |
| RBM17 | NM_032905 | CCCAGTGTACGAGGAACAAGACAGACCGAGATCTCCAACCGGACC | 1402 |
| | | TAGCAACTCCTTCCTCGCTAA | |
| RCC1 | NM_001269 | GGGCTGGGTGAGAATGTGATGGAGAGGAAGAAGCCGGCCCTGGT | 1403 |
| | | ATCCATTCCGGAGGATGTTGTG | |
| REG1A | NM_002909 | CCTACAAGTCCTGGGGCATTGGAGCCCCAAGCAGTGTTAATCCTG | 1404 |
| | | GCTACTGTGTGAGCCTGACCTCA | |
| RELB | NM_006509 | GCGAGGAGCTCTACTTGCTCTGCGACAAGGTGCAGAAAGAGGACA | 1405 |
| | | TATCAGTGGTGTTCAGCAGGGC | |
| RhoB | NM_004040 | AAGCATGAACAGGACTTGACCATCTTTCCAACCCCTGGGGAAGACA | 1406 |
| | | TTTGCAACTGACTTGGGGAGG | |
| rhoC | NM_175744 | CCCGTTCGGTCTGAGGAAGGCCGGGACATGGCGAACCGGATCAG | 1407 |
| | | TGCCTTTGGCTACCTTGAGTGCTC | |
| RIZ1 | NM_012231 | CCAGACGAGCGATTAGAAGCGGCAGCTTGTGAGGTGAATGATTTG | 1408 |
| | | GGGGAAGAGGAGGAGGAGGAAGAGGAGGA | |
| ROCK1 | NM_005406 | TGTGCACATAGGAATGAGCTTCAGATGCAGTTGGCCAGCAAAGAG | 1409 |
| | | AGTGATATTGAGCAATTGCGTGCTAAAC | |
| RPL37A | NM_000998 | GATCTGGCACTGTGGTTCCTGCATGAAGACAGTGGCTGGCGGTGC | 1410 |
| | | CTGGACGTACAATACCACTTCCGCTGTCA | |
| RPLPO | NM_001002 | CCATTCTATCATCAACGGGTACAAACGAGTCCTGGCCTTGTCTGTG | 1411 |
| | | GAGACGGATTACACCTTCCCACTTGCTGA | |
| RPN2 | NM_002951 | CTGTCTTCCTGTTGGCCCTGACAATCATAGCCAGCACCTGGGCTCT | 1412 |
| | | GACGCCCACTCACTACCTCAC | |
| RPS6KB1 | NM_003161 | GCTCATTATGAAAAACATCCCAAACTTTAAAATGCGAAATTATTGGT | 1413 |
| | | TGGTGTGAAGAAAGCCAGACAACTTCTGTTTCTT | |
| RXRA | NM_002957 | GCTCTGTTGTGTCCTGTTGCCGGCTCTGGCCTTCCTGTGACTGACT | 1414 |
| | | GTGAAGTGGCTTCTCCGTAC | |
| RXRB | NM_021976 | CGAGGAGATGCCTGTGGACAGGATCCTGGAGGCAGAGCTTGCTGT | 1415 |
| | | GGAACAGAAGAGTGACCAGGGCGTTG | |
| S100A10 | NM_002966 | ACACCAAAATGCCATCTCAAATGGAACACGCCATGGAAACCATGAT | 1416 |
| | | GTTTACATTTCACAAATTCGCTGGGGATAAA | |
| SEC61A | NM_013336 | CTTCTGAGCCCGTCTCCCGGACAGGTTGAGGAAGCTGCTCCAGAA | 1417 |
| | | GCGCCTCGGAAGGGGAGCTCTC | |
| SEMA3F | NM_004186 | CGCGAGCCCCTCATTATACACTGGGCAGCCTCCCCACAGCGCATC | 1418 |
| | | GAGGAATGCGTGCTCTCAGGCAAGGATGTCAACGGCGAGTG | |
| SFN | NM_006142 | GAGAGAGCCAGTCTGATCCAGAAGGCCAAGCTGGCAGAGCAGGC | 1419 |
| | | CGAACGCTATGAGGACATGGCAGCCT | |
| SGCB | NM_000232 | CAGTGGAGACCAGTTGGGTAGTGGTGACTGGGTACGCTACAAGCT | 1420 |
| | | CTGCATGTGTGCTGATGGGACGCTCTTCAAGG | |
| SGK | NM_005627 | TCCGCAAGACACCTCCTGGAGGGCCTCCTGCAGAAGGACAGGACA | 1421 |
| | | AAGCGGCTCGGGGCCAAGGATGACTTCA | |
| SGKL | NM_170709 | TGCATTCGTTGGTTTCTCTTATGCACCTCCTTCAGAAGACTTATTTT | 1422 |
| | | TGTGAGCAGTTTGCCATTCAGAAA | |
| SHC1 | NM_003029 | CCAACACCTTCTTGGCTTCTGGGACCTGTGTTCTTGCTGAGCACCC | 1423 |
| | | TCTCCGGTTTGGGTTGGGATAACAG | |
| SIR2 | NM_012238 | AGCTGGGGTGTCTGTTTCATGTGGAATACCTGACTTCAGGTCAAGG | 1424 |
| | | GATGGTATTTATGCTCGCCTTGCTGT | |
| SLC1A3 | NM_004172 | GTGGGGAGCCCATCATCTCGCCAAGCCATCACAGGCTCTGCATAC | 1425 |
| | | ACATGCACTCAGTGTGGACTGG | |
| SLC25A3 | NM_213611 | TCTGCCAGTGCTGAATTCTTTGCTGACATTGCCCTGGCTCCTATGG | 1426 |
| | | AAGCTGCTAAGGTTCGAA | |
| SLC35B1 | NM_005827 | CCCAACTCAGGTCCTTGGTAAATCCTGCAAGCCAATCCCAGTCATG | 1427 |
| | | CTCCTTGGGGTGACCCTCTTG | |
| SLC7A11 | NM_014331 | AGATGCATACTTGGAAGCACAGTCATATCACACTGGGAGGCAATGC | 1428 |
| | | AATGTGGTTACCTGGTCCTAGGTT | |
| SLC7A5 | NM_003486 | GCGCAGAGGCCAGTTAAAGTAGATCACCTCCTCGAACCCACTCCG | 1429 |
| | | GTTCCCCGCAACCCACAGCTCAGCT | |
| SNAI2 | NM_003068 | GGCTGGCCAAACATAAGCAGCTGCACTGCGATGCCCAGTCTAGAA | 1430 |
| | | AATCTTTCAGCTGTAAATACTGTGACAAGGA | |
| SNCA | NM_007308 | AGTGACAAATGTTGGAGGAGCAGTGGTGACGGGTGTGACAGCAGT | 1431 |
| | | AGCCCAGAAGACAGTGGAGGG | |
| SNCG | NM_003087 | ACCCACCATGGATGTCTTCAAGAAGGGCTTCTCCATCGCCAAGGA | 1432 |
| | | GGGCGTGGTGGGTGCGGTGGAAAAGACCAAGCAGG | |
| SOD1 | NM_000454 | TGAAGAGAGGCATGTTGGAGACTTGGGCAATGTGACTGCTGACAA | 1433 |
| | | AGATGGTGTGGCCGATGTGTCTATT | |
| SRC | NM_005417 | TGAGGAGTGGTATTTTGGCAAGATCACCAGACGGGAGTCAGAGCG | 1434 |
| | | GTTACTGCTCAATGCAGAGAACCCGAGAG | |
| SRI | NM_003130 | ATACAGCACCAATGGAAAGATCACCTTCGACGACTACATCGCCTGC | 1435 |
| | | TGCGTCAAACTGAGGGCTCTTACAGACA | |
| STAT1 | NM_007315 | GGGCTCAGCTTTCAGAAGTGCTGAGTTGGCAGTTTTCTTCTGTCAC | 1436 |
| | | CAAAAGAGGTCTCAATGTGGACCAGCTGAACATGT | |
| STAT3 | NM_003150 | TCACATGCCACTTTGGTGTTTCATAATCTCCTGGGAGAGATTGACC | 1437 |
| | | AGCAGTATAGCCGCTTCCTGCAAG | |
| STK10 | NM_005990 | CAAGAGGGACTCGGACTGCAGCAGCCTCTGCACCTCTGAGAGCAT | 1438 |
| | | GGACTATGGTACCAATCTCTCCACTGACCTG | |
| STK11 | NM_000455 | GGACTCGGAGACGCTGTGCAGGAGGGCCGTCAAGATCCTCAAGAA | 1439 |
| | | GAAGAAGTTGCGAAGGATCCC | |
| STK15 | NM_003600 | CATCTTCCAGGAGGACCACTCTCTGTGGCACCCTGGACTACCTGC | 1440 |
| | | CCCCTGAAATGATTGAAGGTCGGA | |
| STMN1 | NM_005563 | AATACCCAACGCACAAATGACCGCACGTTCTCTGCCCCGTTTCTTG | 1441 |
| | | CCCCAGTGTGGTTTGCATTGTCTCC | |
| STMY3 | NM_005940 | CCTGGAGGCTGCAACATACCTCAATCCTGTCCCAGGCCGGATCCT | 1442 |
| | | CCTGAAGCCCTTTTCGCAGCACTGCTATCCTCCAAAGCCATTGTA | |
| SURV | NM_001168 | TGTTTTGATTCCCGGGCTTACCAGGTGAGAAGTGAGGGAGGAAGA | 1443 |
| | | AGGCAGTGTCCCTTTTGCTAGAGCTGACAGCTTTG | |
| TACC3 | NM_006342 | CACCCTTGGACTGGAAAACTCACACCCGGTCTGGACACAGAAAGA | 1444 |
| | | GAACCAACAGCTCATCAAGG | |
| TBCA | NM_004607 | GATCCTCGCGTGAGACAGATCAAGATCAAGACCGGCGTGGTGAAG | 1445 |
| | | CGGTTGGTCAAAGAAAAAGTG | |
| TBCC | NM_003192 | CTGTTTTCCTGGAGGACTGCAGTGACTGCGTGCTGGCAGTGGCCT | 1446 |
| | | GCCAACAGCTCCGCATACACAGT | |
| TBCD | NM_005993 | CAGCCAGGTGTACGAGACATTGCTCACCTACAGTGACGTCGTGGG | 1447 |
| | | CGCGGATGTGCTGGACGAGGT | |
| TBCE | NM_003193 | TCCCGAGAGAGGAAAGCATGATGGGAGCCACGAAGGGACTGTGTA | 1448 |
| | | TTTTAAATGCAGGCACCCGAC | |
| TBD | NM_016261 | CCTGGTTGAAGCCTGTTAATGCTTTCAACGTGTGGAAAACCCAGCG | 1449 |
| | | GGCCTTTAGCAAATATGAGAAGTCTGCA | |
| TCP1 | NM_030752 | CCAGTGTGTGTAACAGGGTCACAAGAATTCGACAGCCAGATGCTC | 1450 |
| | | CAAGAGGGTGGCCCAAGGCTATA | |
| TFRC | NM_003234 | GCCAACTGCTTTCATTTGTGAGGGATCTGAACCAATACAGAGCAGA | 1451 |
| | | CATAAAGGAAATGGGCCTGAGT | |
| THBS1 | NM_003246 | CATCCGCAAAGTGACTGAAGAGAACAAAGAGTTGGCCAATGAGCT | 1452 |
| | | GAGGCGGCCTCCCCTATGCTATCACAACGGAGTTCAGTAC | |
| TK1 | NM_003258 | GCCGGGAAGACCGTAATTGTGGCTGCACTGGATGGGACCTTCCAG | 1453 |
| | | AGGAAGCCATTTGGGGCCATCCTGAACCTGGTGCCGCTG | |
| TOP2A | NM_001067 | AATCCAAGGGGGAGAGTGATGACTTCCATATGGACTTTGACTCAGC | 1454 |
| | | TGTGGCTCCTCGGGCAAAATCTGTAC | |
| TOP3B | NM_003935 | GTGATGCCTTCCCTGTGGGCGAGGTGAAGATGCTGGAGAAGCAGA | 1455 |
| | | CGAACCCACCCGACTACCTGA | |
| TP | NM_001953 | CTATATGCAGCCAGAGATGTGACAGCCACCGTGGACAGCCTGCCA | 1456 |
| | | CTCATCACAGCCTCCATTCTCAGTAAGAAACTCGTGG | |
| TP53BP1 | NM_005657 | TGCTGTTGCTGAGTCTGTTGCCAGTCCCCAGAAGACCATGTCTGTG | 1457 |
| | | TTGAGCTGTATCTGTGAAGCCAGGCAAG | |
| TPT1 | NM_003295 | GGTGTCGATATTGTCATGAACCATCACCTGCAGGAAACAAGTTTCA | 1458 |
| | | CAAAAGAAGCCTACAAGAAGTACATCAAAGATTAC | |
| TRAG3 | NM_004909 | GACGCTGGTCTGGTGAAGATGTCCAGGAAACCACGAGCCTCCAGC | 1459 |
| | | CCATTGTCCAACAACCACCCA | |
| TRAIL | NM_003810 | CTTCACAGTGCTCCTGCAGTCTCTCTGTGTGGCTGTAACTTACGTG | 1460 |
| | | TACTTTACCAACGAGCTGAAGCAGATG | |
| TS | NM_001071 | GCCTCGGTGTGCCTTTCAACATCGCCAGCTACGCCCTGCTCACGT | 1461 |
| | | ACATGATTGCGCACATCACG | |
| TSPAN4 | NM_003271 | CTGGTCAGCCTTCAGGGACCCTGAGCACCGCCTGGTCTCTTTCCT | 1462 |
| | | GTGGCCAGCCCAGAACTGAAG | |
| TTK | NM_003318 | TGCTTGTCAGTTGTCAACACCTTATGGCCAACCTGCCTGTTTCCAG | 1463 |
| | | CAGCAACAGCATCAAATACTTGCCACTCCA | |
| TUBA1 | NM_006000 | TGTCACCCCGACTCAACGTGAGACGCACCGCCCGGACTCACCATG | 1464 |
| | | CGTGAATGCATCTCAGTCCACGT | |
| TUBA2 | NM_006001 | AGCTCAACATGCGTGAGTGTATCTCTATCCACGTGGGGCAGGCAG | 1465 |
| | | GAGTCCAGATCGGCAAT | |
| TUBA3 | NM_006009 | CTCTTACATCGACCGCCTAAGAGTCGCGCTGTAAGAAGCAACAACC | 1466 |
| | | TCTCCTCTTCGTCTCCGCCATCAGC | |
| TUBA4 | NM_025019 | GAGGAGGGTGAGTTCTCCAAGGCCCATGAGGATATGACTGCCCTG | 1467 |
| | | GAGAAGGATTACAAGGAGGTGGGCAT | |
| TUBA6 | NM_032704 | GTCCCTTCGCCTCCTTCACCGCCGCAGACCCCTTCAAGTTCTAGTC | 1468 |
| | | ATGCGTGAGTGCATCTCCATCCACG | |
| TUBA8 | NM_018943 | CGCCCTACCTATACCAACCTCAACCGCCTCATCAGTCAGATTGTGT | 1469 |
| | | CCTCAATCACTGCTTCTCTCCG | |
| TUBB | NM_001069 | CGAGGACGAGGCTTAAAAACTTCTCAGATCAATCGTGCATCCTTAG | 1470 |
| | | TGAACTTCTGTTGTCCTCAAGCATGGT | |
| TUBB | NM_006086 | CGCCCTCCTGCAGTATTTATGGCCTCGTCCTCCCCCACCTAGGCCA | 1471 |
| classIII | | CGTGTGAGCTGCTCCTGTCTCTGT | |
| TUBB1 | NM_030773 | ACACTGACTGGCATCCTGCTTTCCAGTGCCTGCCAGCCTCCAGAA | 1472 |
| | | GAGCCAGGTGCCTGACTAGTACATGGGGAGCTACAGAGC | |
| TUBB2 | NM_006088 | GTGGCCTAGAGCCTTCAGTCACTGGGGAAAGCAGGGAAGCAGTGT | 1473 |
| | | GAACTCTTTATTCACTCCCAGCCTG | |
| TUBB5 | NM_006087 | ACAGGCCCCATGCATCCTCCCTGCCTCACTCCCCTCAGCCCCTGC | 1474 |
| | | CGACCTTAGCTTATCTGGGAGAGAAACA | |
| TUBBM | NM_032525 | CCCTATGGCCCTGAATGGTGCACTGGTTTAATTGTGTTGGTGTCGG | 1475 |
| | | CCCCTCACAAATGCAGCCAAGTCATGTAATTAGT | |
| TUBBOK | NM_178014 | AGTGGAATCCTTCCCTTTCCAACTCTACCTCCCTCACTCAGCTCCTT | 1476 |
| | | TCCCCTGATCAGAGAAAGGGATCAAGGG | |
| TUBBP | NM_178012 | GGAAGGAAAGAAGCATGGTCTACTTTAGGTGTGCGCTGGGTCTCT | 1477 |
| | | GGTGCTCTTCACTGTTGCCTGTCACTTTTT | |
| TUBG1 | NM_001070 | GATGCCGAGGGAAATCATCACCCTACAGTTGGGCCAGTGCGGCAA | 1478 |
| | | TCAGATTGGGTTCGAGTTCTGG | |
| TWIST1 | NM_000474 | GCGCTGCGGAAGATCATCCCCACGCTGCCCTCGGACAAGCTGAGC | 1479 |
| | | AAGATTCAGACCCTCAAGC | |
| TYRO3 | NM_006293 | CAGTGTGGAGGGGATGGAGGAGCCTGACATCCAGTGGGTGAAGG | 1480 |
| | | ATGGGGCTGTGGTCCAGAACTTG | |
| UFM1 | NM_016617 | AGTTGTCGTGTGTTCTGGATTCATTCCGGCACCACCATGTCGAAGG | 1481 |
| | | TTTCCTTTAAGATCACGCTGACG | |
| upa | NM_002658 | GTGGATGTGCCCTGAAGGACAAGCCAGGCGTCTACACGAGAGTCT | 1482 |
| | | CACACTTCTTACCCTGGATCCGCAG | |
| VCAM1 | NM_001078 | TGGCTTCAGGAGCTGAATACCCTCCCAGGCACACACAGGTGGGAC | 1483 |
| | | ACAAATAAGGGTTTTGGAACCACTATTTTCTCATCACGACAGCA | |
| VEGF | NM_003376 | CTGCTGTCTTGGGTGCATTGGAGCCTTGCCTTGCTGCTCTACCTCC | 1484 |
| | | ACCATGCCAAGTGGTCCCAGGCTGC | |
| VEGFB | NM_003377 | TGACGATGGCCTGGAGTGTGTGCCCACTGGGCAGCACCAAGTCCG | 1485 |
| | | GATGCAGATCCTCATGATCCGGTACC | |
| VEGFC | NM_005429 | CCTCAGCAAGACGTTATTTGAAATTACAGTGCCTCTCTCTCAAGGC | 1486 |
| | | CCCAAACCAGTAACAATCAGTTTTGCCAATCACACTT | |
| VHL | NM_000551 | CGGTTGGTGACTTGTCTGCCTCCTGCTTTGGGAAGACTGAGGCAT | 1487 |
| | | CCGTGAGGCAGGGACAAGTCTT | |
| VIM | NM_003380 | TGCCCTTAAAGGAACCAATGAGTCCCTGGAACGCCAGATGCGTGA | 1488 |
| | | AATGGAAGAGAACTTTGCCGTTGAAGC | |
| V-RAF | NM_001654 | GGTTGTGCTCTACGAGCTTATGACTGGCTCACTGCCTTACAGCCAC | 1489 |
| | | ATTGGCTGCCGTGACCAGATTATCTTTATGGTGGGCCG | |
| WAVE3 | NM_006646 | CTCTCCAGTGTGGGCACCAGCCGGCCAGAACAGATGCGAGCAGTC | 1490 |
| | | CATGACTCTGGGAGCTACACCGC | |
| Wnt-5a | NM_003392 | GTATCAGGACCACATGCAGTACATCGGAGAAGGCGCGAAGACAGG | 1491 |
| | | CATCAAAGAATGCCAGTATCAATTCCGACA | |
| XIAP | NM_001167 | GCAGTTGGAAGACACAGGAAAGTATCCCCAAATTGCAGATTTATCA | 1492 |
| | | ACGGCTTTTATCTTGAAAATAGTGCCACGCA | |
| XIST | NR_001564 | CAGGTCAGGCAGAGGAAGTCATGTGCATTGCATGAGCTAAACCTAT | 1493 |
| | | CTGAATGAATTGATTTGGGGCTTGTTAGG | |
| ZW10 | NM_004724 | TGGTCAGATGCTGCTGAAGTATATCCTTAGGCCGCTGGCATCTTGC | 1494 |
| | | CCATCCCTTCATGCTGTGAT | |
| ZWILCH | NM_017975 | GAGGGAGCAGACAGTGGGTACCACGATCTCCGTAACCATTTGCAT | 1495 |
| | | GTGACTTAGCAAGGGCTCTGA | |
| ZWINT | NM_007057 | TAGAGGCCATCAAAATTGGCCTCACCAAGGCCCTGACTCAGATGG | 1496 |
| | | AGGAAGCCCAGAGGAAACGGA | |

1. A method of predicting the clinical outcome for a human patient receiving adjuvant anthracycline-based chemotherapy and having breast cancer, the method comprising:
assaying an expression level of at least one RNA transcript listed in Tables 4A-B, or 5A-B, or its expression product, in a biological sample comprising cancer cells obtained from the patient, wherein
if the patient has hormone receptor positive (HR+) breast cancer, the at least one RNA transcript, or its expression product, is selected from Tables 4A-B,
if the patient has hormone receptor negative (HR−) breast cancer, the at least one RNA transcript, or its expression product, is selected from Tables 5A-B; and
determining a normalized expression level of the at least one RNA transcript, or its expression product,
wherein the normalized expression level of the at least one RNA transcript listed in Table 4A, or its expression product, correlates with a decreased likelihood of a positive clinical outcome in a patient having HR+ breast cancer;
wherein the normalized expression level of the at least one RNA transcript listed in Table 4B, or its expression product, correlates with an increased likelihood of a positive clinical outcome in a patient having HR+ breast cancer;
wherein the normalized expression level of the at least one RNA transcript listed in Table 5A, or its expression product, correlates with a decreased likelihood of a positive clinical outcome in a patient having HR− breast cancer; and
wherein the normalized expression level of the at least one RNA transcript listed in Table 5B, or its expression product, correlates with an increased likelihood of a positive clinical outcome in a patient having HR− breast cancer.

2. (canceled)

3. The method of claim 1, wherein the expression level is obtained by gene expression profiling.

4. The method of claim 3, wherein gene expression profiling comprises a reverse transcription-polymerase chain reaction (RT-PCR)-based method.

5. The method of claim 3, wherein gene expression profiling comprises digital gene expression.

6. The method of claim 1, further comprising creating a report based on the normalized expression level.

7. The method of claim 6, wherein, if the patient has a decreased likelihood of a positive clinical outcome, the report provides information to support a decision to use an adjuvant treatment.

8. The method of claim 7, wherein the adjuvant treatment is at least one from the list consisting of a non-anthracycline chemotherapy and a radiation therapy.

9.-16. (canceled)

17. A method of predicting the clinical outcome for a human patient receiving adjuvant anthracycline-based chemotherapy and having breast cancer, the method comprising:
assaying an expression level of at least one RNA transcript listed in Tables 6A-B, 7A-B, 8A-B or 9A-B, or its expression product, in a biological sample comprising cancer cells obtained from the patient, wherein
if the patient has hormone receptor positive (HR+), human epidermal growth factor receptor 2 negative (HER2−) breast cancer, the at least one RNA transcript, or its expression product, is selected from Tables 6A-B,
if the patient has hormone receptor negative (HR−), HER2− breast cancer, the at least one RNA transcript, or its expression product, is selected from Tables 7A-B;
if the patient has HR+, human epidermal growth factor receptor 2 positive (HER2+) breast cancer, the at least one RNA transcript, or its expression product, is selected from Tables 8A-B,
if the patient has HR−, HER2+ breast cancer, the at least one RNA transcript, or its expression product, is selected from Tables 9A-B; and
determining a normalized expression level of the at least one RNA transcript, or its expression product,
wherein the normalized expression level of the at least one RNA transcript listed in Table 6A, or its expression product, correlates with a decreased likelihood of a positive clinical outcome in a HR+, HER2− breast cancer patient;
wherein the normalized expression level of the at least one RNA transcript listed in Table 6B, or its expression product, correlates with an increased likelihood of a positive clinical outcome in a HR+, HER2− breast cancer patient;
wherein the normalized expression level of the at least one RNA transcript listed in Table 7A, or its expression product, correlates with a decreased likelihood of a positive clinical outcome in a HR−, HER2− breast cancer patient;
wherein the normalized expression level of the at least one RNA transcript listed in Table 7B, or its expression product, correlates with an increased likelihood of a positive clinical outcome in a HR−, HER2− breast cancer patient;
wherein the normalized expression level of the at least one RNA transcript listed in Table 8A, or its expression product, correlates with a decreased likelihood of a positive clinical outcome in a HR+, HER2+ breast cancer patient;
wherein the normalized expression level of the at least one RNA transcript listed in Table 8B, or its expression product, correlates with an increased likelihood of a positive clinical outcome in a HR+, HER2+ breast cancer patient.
wherein the normalized expression level of the at least one RNA transcript listed in Table 9A, or its expression product, correlates with a decreased likelihood of a positive clinical outcome in a HR−, HER2+ breast cancer patient; and
wherein the normalized expression level of the at least one RNA transcript listed in Table 9B, or its expression product, correlates with an increased likelihood of a positive clinical outcome in a HR−, HER2+ breast cancer patient.

18. The method of claim 17, further comprising creating a report summarizing the normalized expression level.

19. The method of claim 18, wherein, if the patient has a decreased likelihood of a positive clinical outcome, the report contains information to support the use of at least one adjuvant treatment from the list consisting of a non-anthracycline chemotherapy and a radiation therapy.

20.-30. (canceled)

31. A method of predicting the likelihood that a patient having breast cancer will exhibit a clinical benefit in response to adjuvant treatment with an anthracycline-based chemotherapy, the method comprising:
assaying a biological sample obtained from a cancer tumor of the patient for an expression level of at least one RNA transcript listed in Tables 4A-B, 5A-B, 6A-B, 7A-B, 8A-B, or 9A-B, or its expression product, wherein
if the patient has hormone receptor positive (HR+) breast cancer, the at least one RNA transcript, or its expression product, is selected from Tables 4A-B, 6A-6B and/or 8A-B,
if the patient has hormone receptor negative (HR−) breast cancer, the at least one RNA transcript, or its expression product, is selected from Tables 5A-B, 7A-B, and/or 9A-B; and
determining a normalized expression level of the at least one RNA transcript, or its expression product,
wherein the normalized expression level of the at least one RNA transcript listed in Table 4A, 6A, and/or 8A, or its expression product, positively correlates with a clinical benefit in response to treatment with an anthracycline-based chemotherapy in a patient having HR+ breast cancer; and
wherein the normalized expression level of the at least one RNA transcript listed in Table 4B, 6B, and/or 8B, or its expression product, negatively correlates with a clinical benefit in response to treatment with an anthracycline-based chemotherapy in a patient having HR+ breast cancer;
wherein the normalized expression level of the at least one RNA transcript listed in Table 5A, 7A, and/or 9A, or its expression product, positively correlates with a clinical benefit in response to treatment with an anthracycline-based chemotherapy in a patient having HR− breast cancer; and
wherein the normalized expression level of the at least one RNA transcript listed in Table 5B, 7B, and/or 9B, or its expression product, negatively correlates with a clinical benefit in response to treatment with an anthracycline-based chemotherapy in a patient having HR− breast cancer.

32. The method of claim 31, further comprising: creating a report summarizing the normalized expression level.

33.-43. (canceled)

44. The method of claim 17, wherein the expression level is obtained by gene expression profiling.

45. The method of claim 44, wherein gene expression profiling comprises a reverse transcription-polymerase chain reaction (RT-PCR)-based method.

46. The method of claim 18, wherein, if the patient has a decreased likelihood of a positive clinical outcome, the report provides information to support a decision to use an adjuvant treatment.

47. The method of claim 31, wherein the expression level is obtained by gene expression profiling.

48. The method of claim 47, wherein gene expression profiling comprises a reverse transcription-polymerase chain reaction (RT-PCR)-based method.

49. The method of claim 32, wherein if the patient has a decreased likelihood of a clinical benefit, the report provides information to support a treatment decision.

50. The method of claim 49, wherein, if the patient has a decreased likelihood of a clinical benefit, the report provides information to support a decision to use an adjuvant treatment.


SEQUENCE LISTING (1496 sequences)

SEQ ID NO: 1 - DNA, 20 bp, Artificial Sequence
  source: /note=“Description of Artificial Sequence Synthetic primer”
ttacccgtgggaactgtctc

SEQ ID NO: 2 - DNA, 22 bp, Artificial Sequence
  source: /note=“Description of Artificial Sequence Synthetic primer”
gaccagtaaatgggtcagagga

SEQ ID NO: 3 - DNA, 25 bp, Artificial Sequence
  source: /note=“Description of Artificial Sequence Synthetic primer”
tcctctcaccaggacaacaaccaca

SEQ ID NO: 4 - DNA, 20 bp, Artificial Sequence
  source: /note=“Description of Artificial Sequence Synthetic primer”
aaacaccactggagcattga

SEQ ID NO: 5 - DNA, 20 bp, Artificial Sequence
  source: /note=“Description of Artificial Sequence Synthetic primer”
caagcctggaacctatagcc

SEQ ID NO: 6 - DNA, 24 bp, Artificial Sequence
  source: /note=“Description of Artificial Sequence Synthetic primer”
ctcgccaatgatgctgctcaagtt

SEQ ID NO: 7 - DNA, 18 bp, Artificial Sequence
  source: /note=“Description of Artificial Sequence Synthetic primer”
agacagtcgccttggtcg

SEQ ID NO: 8 - DNA, 20 bp, Artificial Sequence
  source: /note=“Description of Artificial Sequence Synthetic primer”
aacctctgcagaagctggac

SEQ ID NO: 9 - DNA, 24 bp, Artificial Sequence
  source: /note=“Description of Artificial Sequence Synthetic primer”
ccgtactcttcccactgccattga

SEQ ID NO: 10 - DNA, 18 bp, Artificial Sequence
  source: /note=“Description of Artificial Sequence Synthetic primer”
accagtgccacaatgcag

SEQ ID NO: 11 - DNA, 18 bp, Artificial Sequence
  source: /note=“Description of Artificial Sequence Synthetic primer”
atagcgctgaccactgcc

SEQ ID NO: 12 - DNA, 24 bp, Artificial Sequence
  source: /note=“Description of Artificial Sequence Synthetic primer”
ccatgagctgtagccgaatgtcca

SEQ ID NO: 13 - DNA, 18 bp, Artificial Sequence
  source: /note=“Description of Artificial Sequence Synthetic primer”
aagccacagcctccattg

SEQ ID NO: 14 - DNA, 19 bp, Artificial Sequence
  source: /note=“Description of Artificial Sequence Synthetic primer”
ggaaggcttcacggattgt

SEQ ID NO: 15 - DNA, 24 bp, Artificial Sequence
  source: /note=“Description of Artificial Sequence Synthetic primer”
tggagacagacaccctgatccagc

SEQ ID NO: 16 - DNA, 20 bp, Artificial Sequence
  source: /note=“Description of Artificial Sequence Synthetic primer”
tgcagactgtaccatgctga

SEQ ID NO: 17 - DNA, 20 bp, Artificial Sequence
  source: /note=“Description of Artificial Sequence Synthetic primer”
ggccagcaccataatcctat

SEQ ID NO: 18 - DNA, 22 bp, Artificial Sequence
  source: /note=“Description of Artificial Sequence Synthetic primer”
ctgcacacggttctaggctccg

SEQ ID NO: 19 - DNA, 20 bp, Artificial Sequence
  source: /note=“Description of Artificial Sequence Synthetic primer”
tctgtggcccacctctactc

SEQ ID NO: 20 - DNA, 20 bp, Artificial Sequence
  source: /note=“Description of Artificial Sequence Synthetic primer”
gggtgtaggaagtcacagcc

SEQ ID NO: 21 - DNA, 24 bp, Artificial Sequence
  source: /note=“Description of Artificial Sequence Synthetic primer”
aacctgaccaagccactcctggac

SEQ ID NO: 22 - DNA, 21 bp, Artificial Sequence
  source: /note=“Description of Artificial Sequence Synthetic primer”
atgtacgtcgccattcaagct

SEQ ID NO: 23 - DNA, 19 bp, Artificial Sequence
  source: /note=“Description of Artificial Sequence Synthetic primer”
acgccatcacctgaatcca

SEQ ID NO: 24 - DNA, 20 bp, Artificial Sequence
  source: /note=“Description of Artificial Sequence Synthetic primer”
ctggccgcacgacaggcatc

SEQ ID NO: 25 - DNA, 18 bp, Artificial Sequence
  source: /note=“Description of Artificial Sequence Synthetic primer”
atccgcattgaagaccca

SEQ ID NO: 26 - DNA, 20 bp, Artificial Sequence
  source: /note=“Description of Artificial Sequence Synthetic primer”
atccgctagaactgcaccac

SEQ ID NO: 27 - DNA, 24 bp, Artificial Sequence
  source: /note=“Description of Artificial Sequence Synthetic primer”
cccgcagaaagcacatggtattcc

SEQ ID NO: 28 - DNA, 20 bp, Artificial Sequence
  source: /note=“Description of Artificial Sequence Synthetic primer”
caactgctgagagaccgaga

SEQ ID NO: 29 - DNA, 20 bp, Artificial Sequence
  source: /note=“Description of Artificial Sequence Synthetic primer”
cgctcctttactgccttagc

SEQ ID NO: 30 - DNA, 26 bp, Artificial Sequence
  source: /note=“Description of Artificial Sequence Synthetic primer”
aggaatccctccagaacaatccttgg

SEQ ID NO: 31 - DNA, 27 bp, Artificial Sequence
  source: /note=“Description of Artificial Sequence Synthetic primer”
ctgcatgtgattgaataagaaacaaga

SEQ ID NO: 32 - DNA, 22 bp, Artificial Sequence
  source: /note=“Description of Artificial Sequence Synthetic primer”
tgtggacctgatccctgtacac

SEQ ID NO: 33 - DNA, 23 bp, Artificial Sequence
  source: /note=“Description of Artificial Sequence Synthetic primer”
tgaccacaccaaagcctccctgg

SEQ ID NO: 34 - DNA, 20 bp, Artificial Sequence
  source: /note=“Description of Artificial Sequence Synthetic primer”
cgcttctatggcgctgagat

SEQ ID NO: 35 - DNA, 20 bp, Artificial Sequence
  source: /note=“Description of Artificial Sequence Synthetic primer”
tcccggtacaccacgttctt

SEQ ID NO: 36 - DNA, 24 bp, Artificial Sequence
  source: /note=“Description of Artificial Sequence Synthetic primer”
cagccctggactacctgcactcgg

SEQ ID NO: 37 - DNA, 19 bp, Artificial Sequence
  source: /note=“Description of Artificial Sequence Synthetic primer”
tcctgccacccttcaaacc

SEQ ID NO: 38 - DNA, 21 bp, Artificial Sequence
  source: /note=“Description of Artificial Sequence Synthetic primer”
ggcggtaaattcatcatcgaa

SEQ ID NO: 39 - DNA, 24 bp, Artificial Sequence
  source: /note=“Description of Artificial Sequence Synthetic primer”
caggtcacgtccgaggtcgacaca

SEQ ID NO: 40 - DNA, 25 bp, Artificial Sequence
  source: /note=“Description of Artificial Sequence Synthetic primer”
ttgtctctgccttggactatctaca

SEQ ID NO: 41 - DNA, 24 bp, Artificial Sequence
  source: /note=“Description of Artificial Sequence Synthetic primer”
ccagcattagattctccaacttga

SEQ ID NO: 42 - DNA, 23 bp, Artificial Sequence
  source: /note=“Description of Artificial Sequence Synthetic primer”
tcacggtacacaatctttccgga

SEQ ID NO: 43 - DNA, 19 bp, Artificial Sequence
  source: /note=“Description of Artificial Sequence Synthetic primer”
tgggagggatgaaggaaat

SEQ ID NO: 44 - DNA, 19 bp, Artificial Sequence
  source: /note=“Description of Artificial Sequence Synthetic primer”
ctcatacaggtcctgggca

SEQ ID NO: 45 - DNA, 24 bp, Artificial Sequence
  source: /note=“Description of Artificial Sequence Synthetic primer”
tgtctcacgagagcatcgtccaga

SEQ ID NO: 46 - DNA, 20 bp, Artificial Sequence
  source: /note=“Description of Artificial Sequence Synthetic primer”
ggacagcaggaatgtgtttc

SEQ ID NO: 47 - DNA, 20 bp, Artificial Sequence
  source: /note=“Description of Artificial Sequence Synthetic primer”
acccactcgatttgtttctg

SEQ ID NO: 48 - DNA, 22 bp, Artificial Sequence
  source: /note=“Description of Artificial Sequence Synthetic primer”
cattggctccccgtgacctgta

SEQ ID NO: 49 - DNA, 20 bp, Artificial Sequence
  source: /note=“Description of Artificial Sequence Synthetic primer”
gatgaagcctttcgcaagtt

SEQ ID NO: 50 - DNA, 20 bp, Artificial Sequence
  source: /note=“Description of Artificial Sequence Synthetic primer”
aggtctccacacagcacaag

SEQ ID NO: 51 - DNA, 21 bp, Artificial Sequence
  source: /note=“Description of Artificial Sequence Synthetic primer”
ctttcgggaagccaggccctt

SEQ ID NO: 52 - DNA, 21 bp, Artificial Sequence
  source: /note=“Description of Artificial Sequence Synthetic primer”
ggaaacacactggaggacaag

SEQ ID NO: 53 - DNA, 19 bp, Artificial Sequence
  source: /note=“Description of Artificial Sequence Synthetic primer”
cgcatcttggcagaaagtt

SEQ ID NO: 54 - DNA, 24 bp, Artificial Sequence
  source: /note=“Description of Artificial Sequence Synthetic primer”
tcatcagccgcatcaaacagagtg

SEQ ID NO: 55 - DNA, 18 bp, Artificial Sequence
  source: /note=“Description of Artificial Sequence Synthetic primer”
gtttatgccatcggcacc

SEQ ID NO: 56 - DNA, 22 bp, Artificial Sequence
  source: /note=“Description of Artificial Sequence Synthetic primer”
ggaatacacgagggcatagttc

SEQ ID NO: 57 - DNA, 24 bp, Artificial Sequence
  source: /note=“Description of Artificial Sequence Synthetic primer”
actggatcctggccaccgactatg

SEQ ID NO: 58 - DNA, 18 bp, Artificial Sequence
  source: /note=“Description of Artificial Sequence Synthetic primer”
gcctcaagagctggttcg

SEQ ID NO: 59 - DNA, 18 bp, Artificial Sequence
  source: /note=“Description of Artificial Sequence Synthetic primer”
cctgcaccttctccacca

SEQ ID NO: 60 - DNA, 22 bp, Artificial Sequence
  source: /note=“Description of Artificial Sequence Synthetic primer”
actggcgctgcatgtcttccac

SEQ ID NO: 61 - DNA, 18 bp, Artificial Sequence
  source: /note=“Description of Artificial Sequence Synthetic primer”
gaggtcctggagtgcgtg

SEQ ID NO: 62 - DNA, 18 bp, Artificial Sequence
  source: /note=“Description of Artificial Sequence Synthetic primer”
aggtgccagcttctccct

SEQ ID NO: 63 - DNA, 24 bp, Artificial Sequence
  source: /note=“Description of Artificial Sequence Synthetic primer”
ccttaagcgaggtcagctccacca

SEQ ID NO: 64 - DNA, 18 bp, Artificial Sequence
  source: /note=“Description of Artificial Sequence Synthetic primer”
ggtcctccgtcggttctc

SEQ ID NO: 65 - DNA, 18 bp, Artificial Sequence
  source: /note=“Description of Artificial Sequence Synthetic primer”
gtcgcaaactcggagacg

SEQ ID NO: 66 - DNA, 24 bp, Artificial Sequence
  source: /note=“Description of Artificial Sequence Synthetic primer”
ccacggtctggtcttcagctaccc

SEQ ID NO: 67 - DNA, 21 bp, Artificial Sequence
  source: /note=“Description of Artificial Sequence Synthetic primer”
agctgcagaagagctgcacat

SEQ ID NO: 68 - DNA, 20 bp, Artificial Sequence
  source: /note=“Description of Artificial Sequence Synthetic primer”
gcatctgccaactcctccat

SEQ ID NO: 69 - DNA, 22 bp, Artificial Sequence
  source: /note=“Description of Artificial Sequence Synthetic primer”
tgacgagcagcgaacagccacg

SEQ ID NO: 70 - DNA, 21 bp, Artificial Sequence
  source: /note=“Description of Artificial Sequence Synthetic primer”
cagcagatgtggatcagcaag

SEQ ID NO: 71 - DNA, 18 bp, Artificial Sequence
  source: /note=“Description of Artificial Sequence Synthetic primer”
gcatttgcggtggacgat

SEQ ID NO: 72 - DNA, 23 bp, Artificial Sequence
  source: /note=“Description of Artificial Sequence Synthetic primer”
aggagtatgacgagtccggcccc

SEQ ID NO: 73 - DNA, 22 bp, Artificial Sequence
  source: /note=“Description of Artificial Sequence Synthetic primer”
ggctcttgtgcgtactgtcctt

SEQ ID NO: 74 - DNA, 23 bp, Artificial Sequence
  source: /note=“Description of Artificial Sequence Synthetic primer”
tcagatgacgaagagcacagatg

SEQ ID NO: 75 - DNA, 29 bp, Artificial Sequence
  source: /note=“Description of Artificial Sequence Synthetic primer”
aggctcagtgatgtcttccctgtcaccag

SEQ ID NO: 76 - DNA, 19 bp, Artificial Sequence
  source: /note=“Description of Artificial Sequence Synthetic primer”
gggtcaggtgcctcgagat

SEQ ID NO: 77 - DNA, 21 bp, Artificial Sequence
  source: /note=“Description of Artificial Sequence Synthetic primer”
ctgctcactcggctcaaactc

SEQ ID NO: 78 - DNA, 24 bp, Artificial Sequence
  source: /note=“Description of Artificial Sequence Synthetic primer”
tgggcccagagcatgttccagatc

SEQ ID NO: 79 - DNA, 23 bp, Artificial Sequence
  source: /note=“Description of Artificial Sequence Synthetic primer”
cgttgtcagcacttggaatacaa

SEQ ID NO: 80 - DNA, 24 bp, Artificial Sequence
  source: /note=“Description of Artificial Sequence Synthetic primer”
gttcaacctcttcctgtggactgt

SEQ ID NO: 81 - DNA, 26 bp, Artificial Sequence
  source: /note=“Description of Artificial Sequence Synthetic primer”
cccaattaacatgacccggcaaccat

SEQ ID NO: 82 - DNA, 20 bp, Artificial Sequence
  source: /note=“Description of Artificial Sequence Synthetic primer”
ccattcccaccattctacct

SEQ ID NO: 83 - DNA, 20 bp, Artificial Sequence
  source: /note=“Description of Artificial Sequence Synthetic primer”
gggaacatagacccaccaat

SEQ ID NO: 84 - DNA, 21 bp, Artificial Sequence
  source: /note=“Description of Artificial Sequence Synthetic primer”
acaccccagacgtcctggcct

SEQ ID NO: 85 - DNA, 18 bp, Artificial Sequence
  source: /note=“Description of Artificial Sequence Synthetic primer”
ccgccgtggacacagact

SEQ ID NO: 86 - DNA, 21 bp, Artificial Sequence
  source: /note=“Description of Artificial Sequence Synthetic primer”
ttgccgtcagaaaacatgtca

SEQ ID NO: 87 - DNA, 25 bp, Artificial Sequence
  source: /note=“Description of Artificial Sequence Synthetic primer”
tgccactcggaaaaagacctctcgg

SEQ ID NO: 88 - DNA, 20 bp, Artificial Sequence
  source: /note=“Description of Artificial Sequence Synthetic primer”
cctggagggtcctgtacaat

SEQ ID NO: 89 - DNA, 19 bp, Artificial Sequence
  source: /note=“Description of Artificial Sequence Synthetic primer”
ctaattgggctccatctcg

SEQ ID NO: 90 - DNA, 24 bp, Artificial Sequence
  source: /note=“Description of Artificial Sequence Synthetic primer”
catcatgggactcctgcccttacc

SEQ ID NO: 91 - DNA, 25 bp, Artificial Sequence
  source: /note=“Description of Artificial Sequence Synthetic primer”
cagatggacctagtacccactgaga

SEQ ID NO: 92 - DNA, 24 bp, Artificial Sequence
  source: /note=“Description of Artificial Sequence Synthetic primer”
cctatgatttaagggcatttttcc

SEQ ID NO: 93 - DNA, 22 bp, Artificial Sequence
  source: /note=“Description of Artificial Sequence Synthetic primer”
ttccacgccgaaggacagcgat

SEQ ID NO: 94 - DNA, 20 bp, Artificial Sequence
  source: /note=“Description of Artificial Sequence Synthetic primer”
aattaccaagcagccgaaga

SEQ ID NO: 95 - DNA, 20 bp, Artificial Sequence
  source: /note=“Description of Artificial Sequence Synthetic primer”
caggcggacaatgtaacgta

SEQ ID NO: 96 - DNA, 28 bp, Artificial Sequence
  source: /note=“Description of Artificial Sequence Synthetic primer”
ccacccacgaatggttatcttacgactg

SEQ ID NO: 97 - DNA, 20 bp, Artificial Sequence
  source: /note=“Description of Artificial Sequence Synthetic primer”
cagcgacaactctggacaag

SEQ ID NO: 98 - DNA, 19 bp, Artificial Sequence
  source: /note=“Description of Artificial Sequence Synthetic primer”
gctctcagactgccaggaa

SEQ ID NO: 99 - DNA, 24 bp, Artificial Sequence
  source: /note=“Description of Artificial Sequence Synthetic primer”
ccccagagtctccaactgtgacca

SEQ ID NO: 100 - DNA, 24 bp, Artificial Sequence
  source: /note=“Description of Artificial Sequence Synthetic primer”
cttttgtggaactctatgggaaca

SEQ ID NO: 101 - DNA, 19 bp, Artificial Sequence
  source: /note=“Description of Artificial Sequence Synthetic primer”
cagcggttgaagcgttcct

SEQ ID NO: 102 - DNA, 20 bp, Artificial Sequence
  source: /note=“Description of Artificial Sequence Synthetic primer”
ttcggctctcggctgctgca

SEQ ID NO: 103 - DNA, 26 bp, Artificial Sequence
  source: /note=“Description of Artificial Sequence Synthetic primer”
tgtactggcgaagaatatttggtaaa

SEQ ID NO: 104 - DNA, 20 bp, Artificial Sequence
  source: /note=“Description of Artificial Sequence Synthetic primer”
gccacgtgattcttccacaa

SEQ ID NO: 105 - DNA, 24 bp, Artificial Sequence
  source: /note=“Description of Artificial Sequence Synthetic primer”
cagggcatcgatctctcaccctgg

SEQ ID NO: 106 - DNA, 20 bp, Artificial Sequence
  source: /note=“Description of Artificial Sequence Synthetic primer”
ggactgtgaggtcaacaacg

SEQ ID NO: 107 - DNA, 20 bp, Artificial Sequence
  source: /note=“Description of Artificial Sequence Synthetic primer”
ggaagccaaacaccagtagg

SEQ ID NO: 108 - DNA, 24 bp, Artificial Sequence
  source: /note=“Description of Artificial Sequence Synthetic primer”
tgtgatgcactcatccctgaggct

SEQ ID NO: 109 - DNA, 20 bp, Artificial Sequence
  source: /note=“Description of Artificial Sequence Synthetic primer”
cctgcaaaagggaacaagag

SEQ ID NO: 110 - DNA, 20 bp, Artificial Sequence
  source: /note=“Description of Artificial Sequence Synthetic primer”
cgtggttgactctgatctcg

SEQ ID NO: 111 - DNA, 21 bp, Artificial Sequence
  source: /note=“Description of Artificial Sequence Synthetic primer”
cttcgcctccagatggctccc

SEQ ID NO: 112 - DNA, 20 bp, Artificial Sequence
  source: /note=“Description of Artificial Sequence Synthetic primer”
tcagggggctagaaatctgt

SEQ ID NO: 113 - DNA, 20 bp, Artificial Sequence
  source: /note=“Description of Artificial Sequence Synthetic primer”
ccattccagttgatctgtgg

SEQ ID NO: 114 - DNA, 23 bp, Artificial Sequence
  source: /note=“Description of Artificial Sequence Synthetic primer”
ctatgggcccttcaccaacatgc

SEQ ID NO: 115 - DNA, 20 bp, Artificial Sequence
  source: /note=“Description of Artificial Sequence Synthetic primer”
agttcgtgctttgcaagatg

SEQ ID NO: 116 - DNA, 20 bp, Artificial Sequence
  source: /note=“Description of Artificial Sequence Synthetic primer”
aaggtaagctgggtctgctg

SEQ ID NO: 117 - DNA, 29 bp, Artificial Sequence
  source: /note=“Description of Artificial Sequence Synthetic primer”
cattcttcactgcttcataaagctctgca

SEQ ID NO: 118 - DNA, 21 bp, Artificial Sequence
  source: /note=“Description of Artificial Sequence Synthetic primer”
ccgaggttaatccagcacgta

SEQ ID NO: 119 - DNA, 21 bp, Artificial Sequence
  source: /note=“Description of Artificial Sequence Synthetic primer”
aagacatggcgctctcagttc

SEQ ID NO: 120 - DNA, 23 bp, Artificial Sequence
  source: /note=“Description of Artificial Sequence Synthetic primer”
tgctgggagcctacacttggccc

SEQ ID NO: 121 - DNA, 24 bp, Artificial Sequence
  source: /note=“Description of Artificial Sequence Synthetic primer”
tcaacagaaggctgaaccactaga

SEQ ID NO: 122 - DNA, 22 bp, Artificial Sequence
  source: /note=“Description of Artificial Sequence Synthetic primer”
caacagagtttgccgagacact

SEQ ID NO: 123 - DNA, 24 bp, Artificial Sequence
  source: /note=“Description of Artificial Sequence Synthetic primer”
tacagtcccagcaccgacaattcc

SEQ ID NO: 124 - DNA, 22 bp, Artificial Sequence
  source: /note=“Description of Artificial Sequence Synthetic primer”
ctgaagcagatggttcatcatt

SEQ ID NO: 125 - DNA, 22 bp, Artificial Sequence
  source: /note=“Description of Artificial Sequence Synthetic primer”
gctgattcccaagagtctaacc

SEQ ID NO: 126 - DNA, 24 bp, Artificial Sequence
  source: /note=“Description of Artificial Sequence Synthetic primer”
cctcgctttgtttaacagcccagg

SEQ ID NO: 127 - DNA, 23 bp, Artificial Sequence
  source: /note=“Description of Artificial Sequence Synthetic primer”
tgaggagtggtattttggcaaga

SEQ ID NO: 128 - DNA, 21 bp, Artificial Sequence
  source: /note=“Description of Artificial Sequence Synthetic primer”
ctctcgggttctctgcattga

SEQ ID NO: 129 - DNA, 24 bp, Artificial Sequence
  source: /note=“Description of Artificial Sequence Synthetic primer”
aaccgctctgactcccgtctggtg

SEQ ID NO: 130 - DNA, 20 bp, Artificial Sequence
  source: /note=“Description of Artificial Sequence Synthetic primer”
gtcagcgtggtagcggtatt

SEQ ID NO: 131 - DNA, 21 bp, Artificial Sequence
  source: /note=“Description of Artificial Sequence Synthetic primer”
ggaagtcttggctaaagaggc

SEQ ID NO: 132 - DNA, 24 bp, Artificial Sequence
  source: /note=“Description of Artificial Sequence Synthetic primer”
aacaattactgtcactgccgcgga

SEQ ID NO: 133 - DNA, 20 bp, Artificial Sequence
  source: /note=“Description of Artificial Sequence Synthetic primer”
tcagctgtgagctgcggata

SEQ ID NO: 134 - DNA, 24 bp, Artificial Sequence
  source: /note=“Description of Artificial Sequence Synthetic primer”
acggtcctaggtttgaggttaaga

SEQ ID NO: 135 - DNA, 19 bp, Artificial Sequence
  source: /note=“Description of Artificial Sequence Synthetic primer”
caggtcccattgccgggcg

SEQ ID NO: 136 - DNA, 20 bp, Artificial Sequence
  source: /note=“Description of Artificial Sequence Synthetic primer”
atcctagccctggtttttgg

SEQ ID NO: 137 - DNA, 20 bp, Artificial Sequence
  source: /note=“Description of Artificial Sequence Synthetic primer”
ctgccttctcatctgcacaa

SEQ ID NO: 138 - DNA, 20 bp, Artificial Sequence
  source: /note=“Description of Artificial Sequence Synthetic primer”
tttgctgtcaccagcgtcgc

SEQ ID NO: 139 - DNA, 26 bp, Artificial Sequence
  source: /note=“Description of Artificial Sequence Synthetic primer”
cactaaggtttgagacagttccagaa

SEQ ID NO: 140 - DNA, 23 bp, Artificial Sequence
  source: /note=“Description of Artificial Sequence Synthetic primer”
gcgaattagccctctacaactga

SEQ ID NO: 141 - DNA, 26 bp, Artificial Sequence
  source: /note=“Description of Artificial Sequence Synthetic primer”
aacccaagctcaagacgcaggacgag

SEQ ID NO: 142 - DNA, 20 bp, Artificial Sequence
  source: /note=“Description of Artificial Sequence Synthetic primer”
tcgttggagatcagagtgga

SEQ ID NO: 143 - DNA, 18 bp, Artificial Sequence
  source: /note=“Description of Artificial Sequence Synthetic primer”
ttaagcacgccaaccacc

SEQ ID NO: 144 - DNA, 24 bp, Artificial Sequence
  source: /note=“Description of Artificial Sequence Synthetic primer”
tcaccatcacaccacctacagccc

SEQ ID NO: 145 - DNA, 20 bp, Artificial Sequence
  source: /note=“Description of Artificial Sequence Synthetic primer”
gtggctcaacattgtgttcc

SEQ ID NO: 146 - DNA, 20 bp, Artificial Sequence
  source: /note=“Description of Artificial Sequence Synthetic primer”
caatggcctccattttacag

SEQ ID NO: 147 - DNA, 24 bp, Artificial Sequence
  source: /note=“Description of Artificial Sequence Synthetic primer”
atttcagctgatcagtgggcctcc

SEQ ID NO: 148 - DNA, 20 bp, Artificial Sequence
  source: /note=“Description of Artificial Sequence Synthetic primer”
ttcaggttgttgcaggagac

SEQ ID NO: 149 - DNA, 20 bp, Artificial Sequence
  source: /note=“Description of Artificial Sequence Synthetic primer”
catcttcttgggcacacaat

SEQ ID NO: 150 - DNA, 27 bp, Artificial Sequence
  source: /note=“Description of Artificial Sequence Synthetic primer”
tgtctccattattgatcggttcatgca

SEQ ID NO: 151 - DNA, 21 bp, Artificial Sequence
  source: /note=“Description of Artificial Sequence Synthetic primer”
gcatgttcgtggcctctaaga

SEQ ID NO: 152 - DNA, 22 bp, Artificial Sequence
  source: /note=“Description of Artificial Sequence Synthetic primer”
cggtgtagatgcacagcttctc

SEQ ID NO: 153 - DNA, 23 bp, Artificial Sequence
  source: /note=“Description of Artificial Sequence Synthetic primer”
aaggagaccatccccctgacggc

SEQ ID NO: 154 - DNA, 22 bp, Artificial Sequence
  source: /note=“Description of Artificial Sequence Synthetic primer”
atgctgtggctccttcctaact

SEQ ID NO: 155 - DNA, 27 bp, Artificial Sequence
  source: /note=“Description of Artificial Sequence Synthetic primer”
acccaaattgtgatatacaaaaaggtt

SEQ ID NO: 156 - DNA, 30 bp, Artificial Sequence
  source: /note=“Description of Artificial Sequence Synthetic primer”
taccaagcaacctacatgtcaagaaagccc

SEQ ID NO: 157 - DNA, 18 bp, Artificial Sequence
  source: /note=“Description of Artificial Sequence Synthetic primer”
atccaaggccatgactgg

SEQ ID NO: 158 - DNA, 20 bp, Artificial Sequence
  source: /note=“Description of Artificial Sequence Synthetic primer”
ggaatgacctctagggcctg

SEQ ID NO: 159 - DNA, 24 bp, Artificial Sequence
  source: /note=“Description of Artificial Sequence Synthetic primer”
acagccctgtatggccattgttcc

SEQ ID NO: 160 - DNA, 19 bp, Artificial Sequence
  source: /note=“Description of Artificial Sequence Synthetic primer”
gtgtgctagcgtactcccg

SEQ ID NO: 161 - DNA, 18 bp, Artificial Sequence
  source: /note=“Description of Artificial Sequence Synthetic primer”
gcatggtgccggttatct

SEQ ID NO: 162 - DNA, 24 bp, Artificial Sequence
  source: /note=“Description of Artificial Sequence Synthetic primer”
caaggaactgacgctcgaggacct

SEQ ID NO: 163 - DNA, 25 bp, Artificial Sequence
  source: /note=“Description of Artificial Sequence Synthetic primer”
tgtatttcaagacctctgtgcactt

SEQ ID NO: 164 - DNA, 23 bp, Artificial Sequence
  source: /note=“Description of Artificial Sequence Synthetic primer”
ttagcctgaggaattgctgtgtt

SEQ ID NO: 165 - DNA, 25 bp, Artificial Sequence
  source: /note=“Description of Artificial Sequence Synthetic primer”
tttatgaacctgccctgctcccaca

SEQ ID NO: 166 - DNA, 20 bp, Artificial Sequence
  source: /note=“Description of Artificial Sequence Synthetic primer”
agatgaagtggaaggcgctt

SEQ ID NO: 167 - DNA, 21 bp, Artificial Sequence
  source: /note=“Description of Artificial Sequence Synthetic primer”
tgcctctgtaatcggcaactg

SEQ ID NO: 168 - DNA, 18 bp, Artificial Sequence
  source: /note=“Description of Artificial Sequence Synthetic primer”
caccgcggccatcctgca

SEQ ID NO: 169 - DNA, 18 bp, Artificial Sequence
  source: /note=“Description of Artificial Sequence Synthetic primer”
agtgggactgattgccgt

SEQ ID NO: 170 - DNA, 19 bp, Artificial Sequence
  source: /note=“Description of Artificial Sequence Synthetic primer”
gggtagccccctggattat

SEQ ID NO: 171 - DNA, 24 bp, Artificial Sequence
  source: /note=“Description of Artificial Sequence Synthetic primer”
tctgactcaggacaagctgtgccc

SEQ ID NO: 172 - DNA, 18 bp, Artificial Sequence
  source: /note=“Description of Artificial Sequence Synthetic primer”
tggttcccagccctgtgt

SEQ ID NO: 173 - DNA, 19 bp, Artificial Sequence
  source: /note=“Description of Artificial Sequence Synthetic primer”
ctcctccaccctgggttgt

SEQ ID NO: 174 - DNA, 28 bp, Artificial Sequence
  source: /note=“Description of Artificial Sequence Synthetic primer”
ctccaagcccagattcagattcgagtca

SEQ ID NO: 175 - DNA, 18 bp, Artificial Sequence
  source: /note=“Description of Artificial Sequence Synthetic primer”
gagagcgacgcggttgtt

SEQ ID NO: 176 - DNA, 26 bp, Artificial Sequence
  source: /note=“Description of Artificial Sequence Synthetic primer”
gtatggtagatcccggcttattattc

SEQ ID NO: 177 - DNA, 18 bp, Artificial Sequence
  source: /note=“Description of Artificial Sequence Synthetic primer”
tagctgccgctgcggccg

SEQ ID NO: 178 - DNA, 21 bp, Artificial Sequence
  source: /note=“Description of Artificial Sequence Synthetic primer”
tggattggagttctgggaatg

SEQ ID NO: 179 - DNA, 22 bp, Artificial Sequence
  source: /note=“Description of Artificial Sequence Synthetic primer”
gcttgcactccacaggtacaca

SEQ ID NO: 180 - DNA, 23 bp, Artificial Sequence
  source: /note=“Description of Artificial Sequence Synthetic primer”
actggccgtggcactggacaaca

SEQ ID NO: 181 - DNA, 21 bp, Artificial Sequence
  source: /note=“Description of Artificial Sequence Synthetic primer”
aaacgagcagtttgccatcag

SEQ ID NO: 182 - DNA, 20 bp, Artificial Sequence
  source: /note=“Description of Artificial Sequence Synthetic primer”
gttggtgatgttccgaagca

SEQ ID NO: 183 - DNA, 24 bp, Artificial Sequence
  source: /note=“Description of Artificial Sequence Synthetic primer”
cctcaccggcatagactggaagcg

SEQ ID NO: 184 - DNA, 19 bp, Artificial Sequence
  source: /note=“Description of Artificial Sequence Synthetic primer”
gaggcacagtattgcccag

SEQ ID NO: 185 - DNA, 21 bp, Artificial Sequence
  source: /note=“Description of Artificial Sequence Synthetic primer”
gagacggttggagagcttctt

SEQ ID NO: 186 - DNA, 24 bp, Artificial Sequence
  source: /note=“Description of Artificial Sequence Synthetic primer”
atgtttcccaaggcctctggatcc

SEQ ID NO: 187 - DNA, 21 bp, Artificial Sequence
  source: /note=“Description of Artificial Sequence Synthetic primer”
tgagtgtcccccggtatcttc

SEQ ID NO: 188 - DNA, 21 bp, Artificial Sequence
  source: /note=“Description of Artificial Sequence Synthetic primer”
cagccgctttcagattttcat

SEQ ID NO: 189 - DNA, 27 bp, Artificial Sequence
  source: /note=“Description of Artificial Sequence Synthetic primer”
tgccaatcccgatgaaattggaaattt

SEQ ID NO: 190 - DNA, 20 bp, Artificial Sequence
  source: /note=“Description of Artificial Sequence Synthetic primer”
aagccctatccgatgtaccc

SEQ ID NO: 191 - DNA, 19 bp, Artificial Sequence
  source: /note=“Description of Artificial Sequence Synthetic primer”
ctgtggcattgagtttggg

SEQ ID NO: 192 - DNA, 24 bp, Artificial Sequence
  source: /note=“Description of Artificial Sequence Synthetic primer”
cacaacatccctggtgaacgtcgt

SEQ ID NO: 193 - DNA, 18 bp, Artificial Sequence
  source: /note=“Description of Artificial Sequence Synthetic primer”
cggcgatcaagaagctgt

SEQ ID NO: 194 - DNA, 18 bp, Artificial Sequence
  source: /note=“Description of Artificial Sequence Synthetic primer”
caggcgctgatctcttgc

SEQ ID NO: 195 - DNA, 24 bp, Artificial Sequence
  source: /note=“Description of Artificial Sequence Synthetic primer”
cgggcctctgatctccgatttctt

SEQ ID NO: 196 - DNA, 21 bp, Artificial Sequence
  source: /note=“Description of Artificial Sequence Synthetic primer”
tgacaatcagcacacctgcat

SEQ ID NO: 197 - DNA, 23 bp, Artificial Sequence
  source: /note=“Description of Artificial Sequence Synthetic primer”
tgtgactacagccgtgatcctta

SEQ ID NO: 198 - DNA, 20 bp, Artificial Sequence
  source: /note=“Description of Artificial Sequence Synthetic primer”
caggccctcttccgagcggt

SEQ ID NO: 199 - DNA, 21 bp, Artificial Sequence
  source: /note=“Description of Artificial Sequence Synthetic primer”
taaattcactcgtggtgtgga

SEQ ID NO: 200 - DNA, 20 bp, Artificial Sequence
  source: /note=“Description of Artificial Sequence Synthetic primer”
gcctcttgtagggccaatag

SEQ ID NO: 201 - DNA, 21 bp, Artificial Sequence
  source: /note=“Description of Artificial Sequence Synthetic primer”
cttcaattggcaagcccaggc

SEQ ID NO: 202 - DNA, 20 bp, Artificial Sequence
  source: /note=“Description of Artificial Sequence Synthetic primer”
ggatgctggtgacctcttct

SEQ ID NO: 203 - DNA, 20 bp, Artificial Sequence
  source: /note=“Description of Artificial Sequence Synthetic primer”
gccaaggcaccaagtaactc

SEQ ID NO: 204 - DNA, 25 bp, Artificial Sequence
  source: /note=“Description of Artificial Sequence Synthetic primer”
tccctcacgttgcaacaggaattaa

SEQ ID NO: 205 - DNA, 18 bp, Artificial Sequence
  source: /note=“Description of Artificial Sequence Synthetic primer”
ctcccgtcaacagcgttc

SEQ ID NO: 206 - DNA, 18 bp, Artificial Sequence
  source: /note=“Description of Artificial Sequence Synthetic primer”
gggtgagtctggccttca

SEQ ID NO: 207 - DNA, 24 bp, Artificial Sequence
  source: /note=“Description of Artificial Sequence Synthetic primer”
acactggaccaggagtgcatccag

SEQ ID NO: 208 - DNA, 20 bp, Artificial Sequence
  source: /note=“Description of Artificial Sequence Synthetic primer”
ctgaaggagctccaagacct

SEQ ID NO: 209 - DNA, 20 bp, Artificial Sequence
  source: /note=“Description of Artificial Sequence Synthetic primer”
caaaaccgctgtgtttcttc

SEQ ID NO: 210 - DNA, 22 bp, Artificial Sequence
  source: /note=“Description of Artificial Sequence Synthetic primer”
tgctgatgtgccctctccttgg

SEQ ID NO: 211 - DNA, 20 bp, Artificial Sequence
  source: /note=“Description of Artificial Sequence Synthetic primer”
aaggaagtggtccctctgtg

SEQ ID NO: 212 - DNA, 20 bp, Artificial Sequence
  source: /note=“Description of Artificial Sequence Synthetic primer”
gacgcagtctttctgtctgg

SEQ ID NO: 213 - DNA, 24 bp, Artificial Sequence
  source: /note=“Description of Artificial Sequence Synthetic primer”
tgaagtctccagctttgcctcagc

SEQ ID NO: 214 - DNA, 26 bp, Artificial Sequence
  source: /note=“Description of Artificial Sequence Synthetic primer”
gataaattggtacaagggatcagctt

SEQ ID NO: 215 - DNA, 24 bp, Artificial Sequence
  source: /note=“Description of Artificial Sequence Synthetic primer”
gggtgccaagtaactgactattca

SEQ ID NO: 216 - DNA, 26 bp, Artificial Sequence
  source: /note=“Description of Artificial Sequence Synthetic primer”
ccagcccacatgtcctgatcatatgc

SEQ ID NO: 217 - DNA, 21 bp, Artificial Sequence
  source: /note=“Description of Artificial Sequence Synthetic primer”
atgtggaacccccacctactt

SEQ ID NO: 218 - DNA, 22 bp, Artificial Sequence
  source: /note=“Description of Artificial Sequence Synthetic primer”
cagtccacagcacggttatacc

SEQ ID NO: 219 - DNA, 29 bp, Artificial Sequence
  source: /note=“Description of Artificial Sequence Synthetic primer”
agtcccaacagaaacaagaacttcaggcg

SEQ ID NO: 220 - DNA, 24 bp, Artificial Sequence
  source: /note=“Description of Artificial Sequence Synthetic primer”
ggatatttccgtggctcttattca

SEQ ID NO: 221 - DNA, 25 bp, Artificial Sequence
  source: /note=“Description of Artificial Sequence Synthetic primer”
cttctcatcaaggcagaaaaatctt

SEQ ID NO: 222 - DNA, 30 bp, Artificial Sequence
  source: /note=“Description of Artificial Sequence Synthetic primer”
tctccatcaaatcctgtaaactccagagca

SEQ ID NO: 223 - DNA, 18 bp, Artificial Sequence
  source: /note=“Description of Artificial Sequence Synthetic primer”
tcattgaccacagtggcg

SEQ ID NO: 224 - DNA, 19 bp, Artificial Sequence
  source: /note=“Description of Artificial Sequence Synthetic primer”
tcgtgtacttctccacccg

SEQ ID NO: 225 - DNA, 24 bp, Artificial Sequence
  source: /note=“Description of Artificial Sequence Synthetic primer”
cacgtcctcatactcaccaaggcg

SEQ ID NO: 226 - DNA, 23 bp, Artificial Sequence
  source: /note=“Description of Artificial Sequence Synthetic primer”
ccccaggatacctaccactacct

SEQ ID NO: 227 - DNA, 18 bp, Artificial Sequence
  source: /note=“Description of Artificial Sequence Synthetic primer”
tgcgggacttgggaaaga

SEQ ID NO: 228 - DNA, 20 bp, Artificial Sequence
  source: /note=“Description of Artificial Sequence Synthetic primer”
cccttcagcctgccccaccg

SEQ ID NO: 229 - DNA, 22 bp, Artificial Sequence
  source: /note=“Description of Artificial Sequence Synthetic primer”
gacatttccagtcctgcagtca

SEQ ID NO: 230 - DNA, 20 bp, Artificial Sequence
  source: /note=“Description of Artificial Sequence Synthetic primer”
ctccgatcgcacacatttgt

SEQ ID NO: 231 - DNA, 23 bp, Artificial Sequence
  source: /note=“Description of Artificial Sequence Synthetic primer”
tgcctctctgccccaccctttgt

SEQ ID NO: 232 - DNA, 21 bp, Artificial Sequence
  source: /note=“Description of Artificial Sequence Synthetic primer”
tccctccactcggaaggacta

SEQ ID NO: 233 - DNA, 22 bp, Artificial Sequence
  source: /note=“Description of Artificial Sequence Synthetic primer”
cggttgttgctgatctgtctca

SEQ ID NO: 234 - DNA, 27 bp, Artificial Sequence
  source: /note=“Description of Artificial Sequence Synthetic primer”
tctgacactgtccaacttgaccctctt

SEQ ID NO: 235 - DNA, 18 bp, Artificial Sequence
  source: /note=“Description of Artificial Sequence Synthetic primer”
tccaccctcctggctttg

SEQ ID NO: 236 - DNA, 21 bp, Artificial Sequence
  source: /note=“Description of Artificial Sequence Synthetic primer”
tcactcccacgttcaccttgt

SEQ ID NO: 237 - DNA, 23 bp, Artificial Sequence
  source: /note=“Description of Artificial Sequence Synthetic primer”
tcctttcgtcttcgccatgctgg

SEQ ID NO: 238 - DNA, 18 bp, Artificial Sequence
  source: /note=“Description of Artificial Sequence Synthetic primer”
gtggccatccagctgacc

SEQ ID NO: 239 - DNA, 23 bp, Artificial Sequence
  source: /note=“Description of Artificial Sequence Synthetic primer”
cagtggtaggtgatgttctggga

SEQ ID NO: 240 - DNA, 21 bp, Artificial Sequence
  source: /note=“Description of Artificial Sequence Synthetic primer”
tcctgcgcctgatgtccaccg

SEQ ID NO: 241 - DNA, 24 bp, Artificial Sequence
  source: /note=“Description of Artificial Sequence Synthetic primer”
cagccaagaactggtataggagct

SEQ ID NO: 242 - DNA, 19 bp, Artificial Sequence
  source: /note=“Description of Artificial Sequence Synthetic primer”
aaactggctgccagcattg

SEQ ID NO: 243 - DNA, 30 bp, Artificial Sequence
  source: /note=“Description of Artificial Sequence Synthetic primer”
tctcctagccagacgtgtttcttgtccttg

SEQ ID NO: 244 - DNA, 21 bp, Artificial Sequence
  source: /note=“Description of Artificial Sequence Synthetic primer”
gagagcaagcgagacattctg

SEQ ID NO: 245 - DNA, 18 bp, Artificial Sequence
  source: /note=“Description of Artificial Sequence Synthetic primer”
aacagggaactggcccac

SEQ ID NO: 246 - DNA, 24 bp, Artificial Sequence
  source: /note=“Description of Artificial Sequence Synthetic primer”
cctctttgacggctcagccaatct

SEQ ID NO: 247 - DNA, 24 bp, Artificial Sequence
  source: /note=“Description of Artificial Sequence Synthetic primer”
cgacagttgcgatgaaagttctaa

SEQ ID NO: 248 - DNA, 22 bp, Artificial Sequence
  source: /note=“Description of Artificial Sequence Synthetic primer”
ggctgctagagaccatggacat

SEQ ID NO: 249 - DNA, 27 bp, Artificial Sequence
  source: /note=“Description of Artificial Sequence Synthetic primer”
cctcctcctgttgctgccactaatgct

SEQ ID NO: 250 - DNA, 23 bp, Artificial Sequence
  source: /note=“Description of Artificial Sequence Synthetic primer”
tctgcagagttggaagcactcta

SEQ ID NO: 251 - DNA, 21 bp, Artificial Sequence
  source: /note=“Description of Artificial Sequence Synthetic primer”
gccgaggcttttctaccagaa

SEQ ID NO: 252 - DNA, 28 bp, Artificial Sequence
  source: /note=“Description of Artificial Sequence Synthetic primer”
caggatacagctccacagcatcgatgtc

SEQ ID NO: 253 - DNA, 20 bp, Artificial Sequence
  source: /note=“Description of Artificial Sequence Synthetic primer”
acctctgtggtccgtaggag

SEQ ID NO: 254 - DNA, 20 bp, Artificial Sequence
  source: /note=“Description of Artificial Sequence Synthetic primer”
cgaccacttgttttccactg

SEQ ID NO: 255 - DNA, 24 bp, Artificial Sequence
  source: /note=“Description of Artificial Sequence Synthetic primer”
tcttcccagggccctcctcatagt

SEQ ID NO: 256 - DNA, 20 bp, Artificial Sequence
  source: /note=“Description of Artificial Sequence Synthetic primer”
aacttcaaggtcggagaagg

SEQ ID NO: 257 - DNA, 20 bp, Artificial Sequence
  source: /note=“Description of Artificial Sequence Synthetic primer”
tggctaaactcctgcacttg

SEQ ID NO: 258 - DNA, 21 bp, Artificial Sequence
  source: /note=“Description of Artificial Sequence Synthetic primer”
ccgtccacggtctcctcctca

SEQ ID NO: 259 - DNA, 18 bp, Artificial Sequence
  source: /note=“Description of Artificial Sequence Synthetic primer”
gtgctacgccaccctgtt

SEQ ID NO: 260 - DNA, 20 bp, Artificial Sequence
  source: /note=“Description of Artificial Sequence Synthetic primer”
caggggcttctcgtagatgt

SEQ ID NO: 261 - DNA, 22 bp, Artificial Sequence
  source: /note=“Description of Artificial Sequence Synthetic primer”
ccgatgttcacgcctttgggtc

SEQ ID NO: 262 - DNA, 20 bp, Artificial Sequence
  source: /note=“Description of Artificial Sequence Synthetic primer”
gatgtgattgaggtgcatgg

SEQ ID NO: 263 - DNA, 21 bp, Artificial Sequence
  source: /note=“Description of Artificial Sequence Synthetic primer”
gaactccctggagatgaaacc

SEQ ID NO: 264 - DNA, 24 bp, Artificial Sequence
  source: /note=“Description of Artificial Sequence Synthetic primer”
tgttcatcctggcgctcttcatgt

SEQ ID NO: 265 - DNA, 18 bp, Artificial Sequence
  source: /note=“Description of Artificial Sequence Synthetic primer”
tgcagcggctgattgaca

SEQ ID NO: 266 - DNA, 25 bp, Artificial Sequence
  source: /note=“Description of Artificial Sequence Synthetic primer”
caactgttcctggtctacaaactca

SEQ ID NO: 267 - DNA, 28 bp, Artificial Sequence
  source: /note=“Description of Artificial Sequence Synthetic primer”
tcagatggagacctcgtgccaaattaca

SEQ ID NO: 268 - DNA, 20 bp, Artificial Sequence
  source: /note=“Description of Artificial Sequence Synthetic primer”
agcttttccggaatctgttc

SEQ ID NO: 269 - DNA, 20 bp, Artificial Sequence
  source: /note=“Description of Artificial Sequence Synthetic primer”
atttgagcatgttccagtcg

SEQ ID NO: 270 - DNA, 24 bp, Artificial Sequence
  source: /note=“Description of Artificial Sequence Synthetic primer”
catcgccagggcttctcctatgac

SEQ ID NO: 271 - DNA, 20 bp, Artificial Sequence
  source: /note=“Description of Artificial Sequence Synthetic primer”
tggcagaactacctgcaaga

SEQ ID NO: 272 - DNA, 19 bp, Artificial Sequence
  source: /note=“Description of Artificial Sequence Synthetic primer”
tgcttcaaggtgtggttgg

SEQ ID NO: 273 - DNA, 24 bp, Artificial Sequence
  source: /note=“Description of Artificial Sequence Synthetic primer”
cacctgcgtctggatgactgtgac

SEQ ID NO: 274 - DNA, 24 bp, Artificial Sequence
  source: /note=“Description of Artificial Sequence Synthetic primer”
gtacatgatcccctgtgagaaggt

SEQ ID NO: 275 - DNA, 21 bp, Artificial Sequence
  source: /note=“Description of Artificial Sequence Synthetic primer”
gggacagcttgtagcctttgc

SEQ ID NO: 276 - DNA, 22 bp, Artificial Sequence
  source: /note=“Description of Artificial Sequence Synthetic primer”
accctgcccgcgatcacactga

SEQ ID NO: 277 - DNA, 23 bp, Artificial Sequence
  source: /note=“Description of Artificial Sequence Synthetic primer”
gggaggcttatctcactgagtga

SEQ ID NO: 278 - DNA, 19 bp, Artificial Sequence
  source: /note=“Description of Artificial Sequence Synthetic primer”
ccattgcagccttcattgc

SEQ ID NO: 279 - DNA, 29 bp, Artificial Sequence
  source: /note=“Description of Artificial Sequence Synthetic primer”
ttgaggcccagagcagtctaccagattct

SEQ ID NO: 280 - DNA, 21 bp, Artificial Sequence
  source: /note=“Description of Artificial Sequence Synthetic primer”
tgtctcactgagcgagcagaa

SEQ ID NO: 281 - DNA, 19 bp, Artificial Sequence
  source: /note=“Description of Artificial Sequence Synthetic primer”
accattgcagccctgattg

SEQ ID NO: 282 - DNA, 24 bp, Artificial Sequence
  source: /note=“Description of Artificial Sequence Synthetic primer”
cttgaggacgcgaacagtccacca

SEQ ID NO: 283 - DNA, 20 bp, Artificial Sequence
  source: /note=“Description of Artificial Sequence Synthetic primer”
tgaccgcttctaccccaatg

SEQ ID NO: 284 - DNA, 22 bp, Artificial Sequence
  source: /note=“Description of Artificial Sequence Synthetic primer”
aggataaggccaaccatgatgt

SEQ ID NO: 285 - DNA, 28 bp, Artificial Sequence
  source: /note=“Description of Artificial Sequence Synthetic primer”
ctgaaactggaacacaaccacccacaag

SEQ ID NO: 286 - DNA, 19 bp, Artificial Sequence
  source: /note=“Description of Artificial Sequence Synthetic primer”
ggtgcctactccattgtgg

SEQ ID NO: 287 - DNA, 20 bp, Artificial Sequence
  source: /note=“Description of Artificial Sequence Synthetic primer”
gtggagcccttcttcctctt

SEQ ID NO: 288 - DNA, 24 bp, Artificial Sequence
  source: /note=“Description of Artificial Sequence Synthetic primer”
tactccagcaggcacacaaacacg

SEQ ID NO: 289 - DNA, 22 bp, Artificial Sequence
  source: /note=“Description of Artificial Sequence Synthetic primer”
ccagctttgtgcctgtcactat

SEQ ID NO: 290 - DNA, 20 bp, Artificial Sequence
  source: /note=“Description of Artificial Sequence Synthetic primer”
gggaatgtggtagcccaaga

SEQ ID NO: 291 - DNA, 25 bp, Artificial Sequence
  source: /note=“Description of Artificial Sequence Synthetic primer”
ctcatgccaccactgccaacacctc

SEQ ID NO: 292 - DNA, 20 bp, Artificial Sequence
  source: /note=“Description of Artificial Sequence Synthetic primer”
ccgtgttcaagaggaagctc

SEQ ID NO: 293 - DNA, 20 bp, Artificial Sequence
  source: /note=“Description of Artificial Sequence Synthetic primer”
agtgggatcacagggtgaag

SEQ ID NO: 294 - DNA, 24 bp, Artificial Sequence
  source: /note=“Description of Artificial Sequence Synthetic primer”
ttttctcaactcctccacaaggca

SEQ ID NO: 295 - DNA, 30 bp, Artificial Sequence
  source: /note=“Description of Artificial Sequence Synthetic primer”
agaacaaggacaacatagatccttacatat

SEQ ID NO: 296 - DNA, 19 bp, Artificial Sequence
  source: /note=“Description of Artificial Sequence Synthetic primer”
gcaaacctcatgccaatgc

SEQ ID NO: 297 - DNA, 26 bp, Artificial Sequence
  source: /note=“Description of Artificial Sequence Synthetic primer”
cacaccctttggaagtggacccagaa

SEQ ID NO: 298 - DNA, 18 bp, Artificial Sequence
  source: /note=“Description of Artificial Sequence Synthetic primer”
ccgtgtggctcgctttct

SEQ ID NO: 299 - DNA, 24 bp, Artificial Sequence
  source: /note=“Description of Artificial Sequence Synthetic primer”
ggagatttcgctgaagagtaacca

SEQ ID NO: 300 - DNA, 20 bp, Artificial Sequence
  source: /note=“Description of Artificial Sequence Synthetic primer”
tgccgcttcctctttgcggg

SEQ ID NO: 301 - DNA, 19 bp, Artificial Sequence
  source: /note=“Description of Artificial Sequence Synthetic primer”
cacaatggcggctctgaag

SEQ ID NO: 302 - DNA, 26 bp, Artificial Sequence
  source: /note=“Description of Artificial Sequence Synthetic primer”
acacaaacactgtctgtacctgaaga

SEQ ID NO: 303 - DNA, 23 bp, Artificial Sequence
  source: /note=“Description of Artificial Sequence Synthetic primer”
aagttacgctgcgcgacagccaa

SEQ ID NO: 304 - DNA, 20 bp, Artificial Sequence
  source: /note=“Description of Artificial Sequence Synthetic primer”
caagcagtcaaggagaacca

SEQ ID NO: 305 - DNA, 20 bp, Artificial Sequence
  source: /note=“Description of Artificial Sequence Synthetic primer”
agttttgctcgcctcatctt

SEQ ID NO: 306 - DNA, 22 bp, Artificial Sequence
  source: /note=“Description of Artificial Sequence Synthetic primer”
ttcttctgtctcccgccgcttc

SEQ ID NO: 307 - DNA, 20 bp, Artificial Sequence
  source: /note=“Description of Artificial Sequence Synthetic primer”
tccaattccagcatcactgt

SEQ ID NO: 308 - DNA, 20 bp, Artificial Sequence
  source: /note=“Description of Artificial Sequence Synthetic primer”
ggcagtgaaggcgataaagt

SEQ ID NO: 309 - DNA, 25 bp, Artificial Sequence
  source: /note=“Description of Artificial Sequence Synthetic primer”
agaaaagctgtttgtctccccagca

SEQ ID NO: 310 - DNA, 20 bp, Artificial Sequence
  source: /note=“Description of Artificial Sequence Synthetic primer”
cagtgcttccatggacaagt

SEQ ID NO: 311 - DNA, 20 bp, Artificial Sequence
  source: /note=“Description of Artificial Sequence Synthetic primer”
tggacagggatgattgatgt

SEQ ID NO: 312 - DNA, 24 bp, Artificial Sequence
  source: /note=“Description of Artificial Sequence Synthetic primer”
atctccatcagcatgggccagttt

SEQ ID NO: 313 - DNA, 21 bp, Artificial Sequence
  source: /note=“Description of Artificial Sequence Synthetic primer”
tgcacagagggtgtgggttac

SEQ ID NO: 314 - DNA, 28 bp, Artificial Sequence
  source: /note=“Description of Artificial Sequence Synthetic primer”
tcttcatctgatttacaagctgtacatg

SEQ ID NO: 315 - DNA, 29 bp, Artificial Sequence
  source: /note=“Description of Artificial Sequence Synthetic primer”
caatgcttccaacaatttgtttgcttgcc

SEQ ID NO: 316 - DNA, 24 bp, Artificial Sequence
  source: /note=“Description of Artificial Sequence Synthetic primer”
ctctgagacagtgcttcgatgact

SEQ ID NO: 317 - DNA, 19 bp, Artificial Sequence
  source: /note=“Description of Artificial Sequence Synthetic primer”
ccatgaggcccaacttcct

SEQ ID NO: 318 - DNA, 23 bp, Artificial Sequence
  source: /note=“Description of Artificial Sequence Synthetic primer”
cagacttggtgccctttgactcc

SEQ ID NO: 319 - DNA, 20 bp, Artificial Sequence
  source: /note=“Description of Artificial Sequence Synthetic primer”
agacatcagctcctggttca

SEQ ID NO: 320 - DNA, 20 bp, Artificial Sequence
  source: /note=“Description of Artificial Sequence Synthetic primer”
gacaaacacccttcctccag

SEQ ID NO: 321 - DNA, 26 bp, Artificial Sequence
  source: /note=“Description of Artificial Sequence Synthetic primer”
cgaggccattgacttcatagactcca

SEQ ID NO: 322 - DNA, 22 bp, Artificial Sequence
  source: /note=“Description of Artificial Sequence Synthetic primer”
cagaggatgacgaggatgatga

SEQ ID NO: 323 - DNA, 18 bp, Artificial Sequence
  source: /note=“Description of Artificial Sequence Synthetic primer”
ctgtgccgcctccttgtc

SEQ ID NO: 324 - DNA, 28 bp, Artificial Sequence
  source: /note=“Description of Artificial Sequence Synthetic primer”
ctcctcattgtcactgccaaacaggtca

SEQ ID NO: 325 - DNA, 20 bp, Artificial Sequence
  source: /note=“Description of Artificial Sequence Synthetic primer”
tgtcgatggacttccagaac

SEQ ID NO: 326 - DNA, 19 bp, Artificial Sequence
  source: /note=“Description of Artificial Sequence Synthetic primer”
attgggacagcttggatca

SEQ ID NO: 327 - DNA, 18 bp, Artificial Sequence
  source: /note=“Description of Artificial Sequence Synthetic primer”
cacctgggcagctgccaa

SEQ ID NO: 328 - DNA, 23 bp, Artificial Sequence
  source: /note=“Description of Artificial Sequence Synthetic primer”
gatctaagatggcgactgtcgaa

SEQ ID NO: 329 - DNA, 25 bp, Artificial Sequence
  source: /note=“Description of Artificial Sequence Synthetic primer”
ttagattccgttttctcctcttctg

SEQ ID NO: 330 - DNA, 27 bp, Artificial Sequence
  source: /note=“Description of Artificial Sequence Synthetic primer”
accacccctactcctaatcccccgact

SEQ ID NO: 331 - DNA, 18 bp, Artificial Sequence
  source: /note=“Description of Artificial Sequence Synthetic primer”
aagccgcggttgaatgtg

SEQ ID NO: 332 - DNA, 20 bp, Artificial Sequence
  source: /note=“Description of Artificial Sequence Synthetic primer”
tgacgccagcttcaatgatg

SEQ ID NO: 333 - DNA, 25 bp, Artificial Sequence
  source: /note=“Description of Artificial Sequence Synthetic primer”
tgaccctctccctctctggatggca

SEQ ID NO: 334 - DNA, 18 bp, Artificial Sequence
  source: /note=“Description of Artificial Sequence Synthetic primer”
ctcggatcctagccctcg

SEQ ID NO: 335 - DNA, 22 bp, Artificial Sequence
  source: /note=“Description of Artificial Sequence Synthetic primer”
ggcattggaatatccctctgta

SEQ ID NO: 336 - DNA, 24 bp, Artificial Sequence
  source: /note=“Description of Artificial Sequence Synthetic primer”
cctccatggactcgagtgtaccga

SEQ ID NO: 337 - DNA, 20 bp, Artificial Sequence
  source: /note=“Description of Artificial Sequence Synthetic primer”
tggtccatcgccagttatca

SEQ ID NO: 338 - DNA, 23 bp, Artificial Sequence
  source: /note=“Description of Artificial Sequence Synthetic primer”
tgttctagcgatcttgcttcaca

SEQ ID NO: 339 - DNA, 30 bp, Artificial Sequence
  source: /note=“Description of Artificial Sequence Synthetic primer”
atctgtatgcggaacctcaaaagagtccct

SEQ ID NO: 340 - DNA, 23 bp, Artificial Sequence
  source: /note=“Description of Artificial Sequence Synthetic primer”
cggttatgtcatgccagatacac

SEQ ID NO: 341 - DNA, 24 bp, Artificial Sequence
  source: /note=“Description of Artificial Sequence Synthetic primer”
gaactgagacccactgaagaaagg

SEQ ID NO: 342 - DNA, 25 bp, Artificial Sequence
  source: /note=“Description of Artificial Sequence Synthetic primer”
cctcaaaggtactccctcctcccgg

SEQ ID NO: 343 - DNA, 25 bp, Artificial Sequence
  source: /note=“Description of Artificial Sequence Synthetic primer”
tggctcttaatcagtttcgttacct

SEQ ID NO: 344 - DNA, 25 bp, Artificial Sequence
  source: /note=“Description of Artificial Sequence Synthetic primer”
caaggcatatcgatcctcataaagt

SEQ ID NO: 345 - DNA, 30 bp, Artificial Sequence
  source: /note=“Description of Artificial Sequence Synthetic primer”
tgtcccacgaataatgcgtaaattctccag

SEQ ID NO: 346 - DNA, 20 bp, Artificial Sequence
  source: /note=“Description of Artificial Sequence Synthetic primer”
gtccaggtggatgtgaaaga

SEQ ID NO: 347 - DNA, 20 bp, Artificial Sequence
  source: /note=“Description of Artificial Sequence Synthetic primer”
cggccaggatacacatctta

SEQ ID NO: 348 - DNA, 21 bp, Artificial Sequence
  source: /note=“Description of Artificial Sequence Synthetic primer”
cagcaggccctcaaggagctg

SEQ ID NO: 349 - DNA, 20 bp, Artificial Sequence
  source: /note=“Description of Artificial Sequence Synthetic primer”
acggatcacagtggaggaag

SEQ ID NO: 350 - DNA, 20 bp, Artificial Sequence
  source: /note=“Description of Artificial Sequence Synthetic primer”
ctcatccgtcgggtcatagt

SEQ ID NO: 351 - DNA, 20 bp, Artificial Sequence
  source: /note=“Description of Artificial Sequence Synthetic primer”
cgctggctcacccctacctg

SEQ ID NO: 352 - DNA, 18 bp, Artificial Sequence
  source: /note=“Description of Artificial Sequence Synthetic primer”
acccccagaccggatcag

SEQ ID NO: 353 - DNA, 23 bp, Artificial Sequence
  source: /note=“Description of Artificial Sequence Synthetic primer”
tgtagggcagacttcctcaaaca

SEQ ID NO: 354 - DNA, 23 bp, Artificial Sequence
  source: /note=“Description of Artificial Sequence Synthetic primer”
ctggccctcatgtccccttcacg

SEQ ID NO: 355 - DNA, 19 bp, Artificial Sequence
  source: /note=“Description of Artificial Sequence Synthetic primer”
cgtggtgcccctctatgac

SEQ ID NO: 356 - DNA, 19 bp, Artificial Sequence
  source: /note=“Description of Artificial Sequence Synthetic primer”
ggctagtgggcgcatgtag

SEQ ID NO: 357 - DNA, 19 bp, Artificial Sequence
  source: /note=“Description of Artificial Sequence Synthetic primer”
ctggagatgctggacgccc

SEQ ID NO: 358 - DNA, 21 bp, Artificial Sequence
  source: /note=“Description of Artificial Sequence Synthetic primer”
ggattgctcaacaaccatgct

SEQ ID NO: 359 - DNA, 24 bp, Artificial Sequence
  source: /note=“Description of Artificial Sequence Synthetic primer”
ggcattaacacttttggacgataa

SEQ ID NO: 360 - DNA, 30 bp, Artificial Sequence
  source: /note=“Description of Artificial Sequence Synthetic primer”
tctggaccctcctacctctggttcttacgt

SEQ ID NO: 361 - DNA, 24 bp, Artificial Sequence
  source: /note=“Description of Artificial Sequence Synthetic primer”
gcactttgggattctttccattat

SEQ ID NO: 362 - DNA, 24 bp, Artificial Sequence
  source: /note=“Description of Artificial Sequence Synthetic primer”
gcatgtaagaagaccctcactgaa

SEQ ID NO: 363 - DNA, 29 bp, Artificial Sequence
  source: /note=“Description of Artificial Sequence Synthetic primer”
acaacattctcggtgcctgtaacaaagaa

SEQ ID NO: 364 - DNA, 18 bp, Artificial Sequence
  source: /note=“Description of Artificial Sequence Synthetic primer”
gcctcttcctgttcgacg

SEQ ID NO: 365 - DNA, 18 bp, Artificial Sequence
  source: /note=“Description of Artificial Sequence Synthetic primer”
gctttgcccggtagctct

SEQ ID NO: 366 - DNA, 24 bp, Artificial Sequence
  source: /note=“Description of Artificial Sequence Synthetic primer”
tcgcccacctacgtactggcctac

SEQ ID NO: 367 - DNA, 25 bp, Artificial Sequence
  source: /note=“Description of Artificial Sequence Synthetic primer”
ggattgtagactgtcaccgaaattc

SEQ ID NO: 368 - DNA, 28 bp, Artificial Sequence
  source: /note=“Description of Artificial Sequence Synthetic primer”
ggctattcctcattttctctacaaagtg

SEQ ID NO: 369 - DNA, 30 bp, Artificial Sequence
  source: /note=“Description of Artificial Sequence Synthetic primer”
cctccaggaggctaccttcttcatgttcac

SEQ ID NO: 370 - DNA, 19 bp, Artificial Sequence
  source: /note=“Description of Artificial Sequence Synthetic primer”
aaggaaagggtgcctcatc

SEQ ID NO: 371 - DNA, 19 bp, Artificial Sequence
  source: /note=“Description of Artificial Sequence Synthetic primer”
gagccacaagcagcacagt

SEQ ID NO: 372 - DNA, 24 bp, Artificial Sequence
  source: /note=“Description of Artificial Sequence Synthetic primer”
catcacccacaaggacaggttgct

SEQ ID NO: 373 - DNA, 20 bp, Artificial Sequence
  source: /note=“Description of Artificial Sequence Synthetic primer”
cacgggacattcaccacatc

SEQ ID NO: 374 - DNA, 19 bp, Artificial Sequence
  source: /note=“Description of Artificial Sequence Synthetic primer”
gggtgccatccacttcaca

SEQ ID NO: 375 - DNA, 27 bp, Artificial Sequence
  source: /note=“Description of Artificial Sequence Synthetic primer”
ataaaaagacaaccaacggccgactgc

SEQ ID NO: 376 - DNA, 18 bp, Artificial Sequence
  source: /note=“Description of Artificial Sequence Synthetic primer”
ccagtggagcgcttccat

SEQ ID NO: 377 - DNA, 22 bp, Artificial Sequence
  source: /note=“Description of Artificial Sequence Synthetic primer”
ctctctgggtcgtctgaaacaa

SEQ ID NO: 378 - DNA, 23 bp, Artificial Sequence
  source: /note=“Description of Artificial Sequence Synthetic primer”
tcggccacttcatcaggacgcag

SEQ ID NO: 379 - DNA, 22 bp, Artificial Sequence
  source: /note=“Description of Artificial Sequence Synthetic primer”
ggttccagctttctgtagctgt

SEQ ID NO: 380 - DNA, 19 bp, Artificial Sequence
  source: /note=“Description of Artificial Sequence Synthetic primer”
gccgcaggttctagttgct

SEQ ID NO: 381 - DNA, 24 bp, Artificial Sequence
  source: /note=“Description of Artificial Sequence Synthetic primer”
attggtggccacaccacctcctta

SEQ ID NO: 382 - DNA, 21 bp, Artificial Sequence
  source: /note=“Description of Artificial Sequence Synthetic primer”
gcgtatgatttcccgaatgag

SEQ ID NO: 383 - DNA, 21 bp, Artificial Sequence
  source: /note=“Description of Artificial Sequence Synthetic primer”
cagtgacctcgtacccattgc

SEQ ID NO: 384 - DNA, 25 bp, Artificial Sequence
  source: /note=“Description of Artificial Sequence Synthetic primer”
atgttgatatgcccaaacttcatga

SEQ ID NO: 385 - DNA, 21 bp, Artificial Sequence
  source: /note=“Description of Artificial Sequence Synthetic primer”
cgagccctttgatgacttcct

SEQ ID NO: 386 - DNA, 18 bp, Artificial Sequence
  source: /note=“Description of Artificial Sequence Synthetic primer”
ggagcgggctgtctcaga

SEQ ID NO: 387 - DNA, 22 bp, Artificial Sequence
  source: /note=“Description of Artificial Sequence Synthetic primer”
tcccagcatcatccaggcccag

SEQ ID NO: 388 - DNA, 19 bp, Artificial Sequence
  source: /note=“Description of Artificial Sequence Synthetic primer”
ccaccccgagcaaatctgt

SEQ ID NO: 389 - DNA, 22 bp, Artificial Sequence
  source: /note=“Description of Artificial Sequence Synthetic primer”
aaatccagtccccctactttgg

SEQ ID NO: 390 - DNA, 23 bp, Artificial Sequence
  source: /note=“Description of Artificial Sequence Synthetic primer”
cctgaatcctggaggctcacgcc

SEQ ID NO: 391 - DNA, 26 bp, Artificial Sequence
  source: /note=“Description of Artificial Sequence Synthetic primer”
ggataattcagacaacaacaccatct

SEQ ID NO: 392 - DNA, 25 bp, Artificial Sequence
  source: /note=“Description of Artificial Sequence Synthetic primer”
tgaagtaatcagccacagactcaat

SEQ ID NO: 393 - DNA, 29 bp, Artificial Sequence
  source: /note=“Description of Artificial Sequence Synthetic primer”
tcaattgtaacattctcacccaggccttg

SEQ ID NO: 394 - DNA, 20 bp, Artificial Sequence
  source: /note=“Description of Artificial Sequence Synthetic primer”
gaagcgcagatcatgaagaa

SEQ ID NO: 395 - DNA, 20 bp, Artificial Sequence
  source: /note=“Description of Artificial Sequence Synthetic primer”
ctcctcagacaccactgcat

SEQ ID NO: 396 - DNA, 24 bp, Artificial Sequence
  source: /note=“Description of Artificial Sequence Synthetic primer”
ctgaagcacgacaagctggtccag

SEQ ID NO: 397 - DNA, 20 bp, Artificial Sequence
  source: /note=“Description of Artificial Sequence Synthetic primer”
cctccaactcctagcctcaa

SEQ ID NO: 398 - DNA, 18 bp, Artificial Sequence
  source: /note=“Description of Artificial Sequence Synthetic primer”
ggcgcatgcttgtaatcc

SEQ ID NO: 399 - DNA, 24 bp, Artificial Sequence
  source: /note=“Description of Artificial Sequence Synthetic primer”
tgatcctcctgtctcaacctccca

SEQ ID NO: 400 - DNA, 18 bp, Artificial Sequence
  source: /note=“Description of Artificial Sequence Synthetic primer”
gtgctggtgacgaatcca

SEQ ID NO: 401 - DNA, 20 bp, Artificial Sequence
  source: /note=“Description of Artificial Sequence Synthetic primer”
cccggcaaaaacaaataagt

SEQ ID NO: 402 - DNA, 25 bp, Artificial Sequence
  source: /note=“Description of Artificial Sequence Synthetic primer”
ttcatctcaatggaaggatcctgcc

SEQ ID NO: 403 - DNA, 20 bp, Artificial Sequence
  source: /note=“Description of Artificial Sequence Synthetic primer”
accctcgacaagaccacact

SEQ ID NO: 404 - DNA, 20 bp, Artificial Sequence
  source: /note=“Description of Artificial Sequence Synthetic primer”
tgggagttcatgggtacaga

SEQ ID NO: 405 - DNA, 24 bp, Artificial Sequence
  source: /note=“Description of Artificial Sequence Synthetic primer”
aacttcagccccagctcccaagtc

SEQ ID NO: 406 - DNA, 25 bp, Artificial Sequence
  source: /note=“Description of Artificial Sequence Synthetic primer”
aagggcaatcacagtgttaaaagaa

SEQ ID NO: 407 - DNA, 26 bp, Artificial Sequence
  source: /note=“Description of Artificial Sequence Synthetic primer”
ggagaacttcaatgaagaattttcca

SEQ ID NO: 408 - DNA, 29 bp, Artificial Sequence
  source: /note=“Description of Artificial Sequence Synthetic primer”
cataggagcagcctgcaacatttcagcat

SEQ ID NO: 409 - DNA, 20 bp, Artificial Sequence
  source: /note=“Description of Artificial Sequence Synthetic primer”
attccacccatggcaaattc

SEQ ID NO: 410 - DNA, 22 bp, Artificial Sequence
  source: /note=“Description of Artificial Sequence Synthetic primer”
gatgggatttccattgatgaca

SEQ ID NO: 411 - DNA, 22 bp, Artificial Sequence
  source: /note=“Description of Artificial Sequence Synthetic primer”
ccgttctcagccttgacggtgc

SEQ ID NO: 412 - DNA, 23 bp, Artificial Sequence
  source: /note=“Description of Artificial Sequence Synthetic primer”
caaaggagctcactgtggtgtct

SEQ ID NO: 413 - DNA, 26 bp, Artificial Sequence
  source: /note=“Description of Artificial Sequence Synthetic primer”
gagtcagaatggcttattcacagatg

SEQ ID NO: 414 - DNA, 24 bp, Artificial Sequence
  source: /note=“Description of Artificial Sequence Synthetic primer”
tgttccaaccactgaatctggacc

SEQ ID NO: 415 - DNA, 20 bp, Artificial Sequence
  source: /note=“Description of Artificial Sequence Synthetic primer”
ttgggaaatatttgggcatt

SEQ ID NO: 416 - DNA, 20 bp, Artificial Sequence
  source: /note=“Description of Artificial Sequence Synthetic primer”
agaagctagggtggttgtcc

SEQ ID NO: 417 - DNA, 26 bp, Artificial Sequence
  source: /note=“Description of Artificial Sequence Synthetic primer”
ttgggacattgtagacttggccagac

SEQ ID NO: 418 - DNA, 19 bp, Artificial Sequence
  source: /note=“Description of Artificial Sequence Synthetic primer”
gcatgggaaccatcaacca

SEQ ID NO: 419 - DNA, 21 bp, Artificial Sequence
  source: /note=“Description of Artificial Sequence Synthetic primer”
tgaggagtttgccttgattcg

SEQ ID NO: 420 - DNA, 30 bp, Artificial Sequence
  source: /note=“Description of Artificial Sequence Synthetic primer”
ccatggaccaacttcactatgtgacagagc

SEQ ID NO: 421 - DNA, 20 bp, Artificial Sequence
  source: /note=“Description of Artificial Sequence Synthetic primer”
ctgttgcaggaaggcattga

SEQ ID NO: 422 - DNA, 28 bp, Artificial Sequence
  source: /note=“Description of Artificial Sequence Synthetic primer”
gtcagtgggtctctaataaagagatgag

SEQ ID NO: 423 - DNA, 21 bp, Artificial Sequence
  source: /note=“Description of Artificial Sequence Synthetic primer”
catctcctggcccagcatgtt

SEQ ID NO: 424 - DNA, 21 bp, Artificial Sequence
  source: /note=“Description of Artificial Sequence Synthetic primer”
cgctccagacctatgatgact

SEQ ID NO: 425 - DNA, 20 bp, Artificial Sequence
  source: /note=“Description of Artificial Sequence Synthetic primer”
acagtggaaggaccaggact

SEQ ID NO: 426 - DNA, 24 bp, Artificial Sequence
  source: /note=“Description of Artificial Sequence Synthetic primer”
tgttagccaaagactgccactgca

SEQ ID NO: 427 - DNA, 18 bp, Artificial Sequence
  source: /note=“Description of Artificial Sequence Synthetic primer”
ctccgacgtggctttcca

SEQ ID NO: 428 - DNA, 23 bp, Artificial Sequence
  source: /note=“Description of Artificial Sequence Synthetic primer”
cgtaattggcagaattgatgaca

SEQ ID NO: 429 - DNA, 25 bp, Artificial Sequence
  source: /note=“Description of Artificial Sequence Synthetic primer”
tggcccacagcatctatggaatccc

SEQ ID NO: 430 - DNA, 23 bp, Artificial Sequence
  source: /note=“Description of Artificial Sequence Synthetic primer”
ggagctctgcagtaaccacagaa

SEQ ID NO: 431 - DNA, 20 bp, Artificial Sequence
  source: /note=“Description of Artificial Sequence Synthetic primer”
caatgccatccagctcttga

SEQ ID NO: 432 - DNA, 22 bp, Artificial Sequence
  source: /note=“Description of Artificial Sequence Synthetic primer”
aggccccatgctgacgtccctc

SEQ ID NO: 433 - DNA, 20 bp, Artificial Sequence
  source: /note=“Description of Artificial Sequence Synthetic primer”
ggtgaaggttgtctcttccg

SEQ ID NO: 434 - DNA, 19 bp, Artificial Sequence
  source: /note=“Description of Artificial Sequence Synthetic primer”
cagcccttccacttgtctg

SEQ ID NO: 435 - DNA, 24 bp, Artificial Sequence
  source: /note=“Description of Artificial Sequence Synthetic primer”
aagagccctgtcttcagaaggccc

SEQ ID NO: 436 - DNA, 20 bp, Artificial Sequence
  source: /note=“Description of Artificial Sequence Synthetic primer”
tacccttccatgtgctggat

SEQ ID NO: 437 - DNA, 18 bp, Artificial Sequence
  source: /note=“Description of Artificial Sequence Synthetic primer”
gctgaagaggcccaggtt

SEQ ID NO: 438 - DNA, 24 bp, Artificial Sequence
  source: /note=“Description of Artificial Sequence Synthetic primer”
cgggactccctggtcagctacatc

SEQ ID NO: 439 - DNA, 18 bp, Artificial Sequence
  source: /note=“Description of Artificial Sequence Synthetic primer”
gcttatgaccgaccccaa

SEQ ID NO: 440 - DNA, 20 bp, Artificial Sequence
  source: /note=“Description of Artificial Sequence Synthetic primer”
aaagttccaggcaacatcgt

SEQ ID NO: 441 - DNA, 24 bp, Artificial Sequence
  source: /note=“Description of Artificial Sequence Synthetic primer”
ctcatcacctggtctccggtgtgt

SEQ ID NO: 442 - DNA, 20 bp, Artificial Sequence
  source: /note=“Description of Artificial Sequence Synthetic primer”
ccatctgcatccatcttgtt

SEQ ID NO: 443 - DNA, 20 bp, Artificial Sequence
  source: /note=“Description of Artificial Sequence Synthetic primer”
ggccaccagggtattatctg

SEQ ID NO: 444 - DNA, 23 bp, Artificial Sequence
  source: /note=“Description of Artificial Sequence Synthetic primer”
ctccccacccttgagaagtgcct

SEQ ID NO: 445 - DNA, 18 bp, Artificial Sequence
  source: /note=“Description of Artificial Sequence Synthetic primer”
gacaaggacggcagcaag

SEQ ID NO: 446 - DNA, 18 bp, Artificial Sequence
  source: /note=“Description of Artificial Sequence Synthetic primer”
ttgtggcctgtctggacc

SEQ ID NO: 447 - DNA, 24 bp, Artificial Sequence
  source: /note=“Description of Artificial Sequence Synthetic primer”
ccaggagttgccaccactgttgtc

SEQ ID NO: 448 - DNA, 20 bp, Artificial Sequence
  source: /note=“Description of Artificial Sequence Synthetic primer”
gtgatcccaagcccacaata

SEQ ID NO: 449 - DNA, 18 bp, Artificial Sequence
  source: /note=“Description of Artificial Sequence Synthetic primer”
tgtggcgatcaggatgtg

SEQ ID NO: 450 - DNA, 24 bp, Artificial Sequence
  source: /note=“Description of Artificial Sequence Synthetic primer”
tcagtgggaaaaagtacaccgccc

SEQ ID NO: 451 - DNA, 20 bp, Artificial Sequence
  source: /note=“Description of Artificial Sequence Synthetic primer”
ggcccagcttgaatttttca

SEQ ID NO: 452 - DNA, 27 bp, Artificial Sequence
  source: /note=“Description of Artificial Sequence Synthetic primer”
aagctatgaggaaaagaagtacacgat

SEQ ID NO: 453 - DNA, 30 bp, Artificial Sequence
  source: /note=“Description of Artificial Sequence Synthetic primer”
tcagccactggcttctgtcataatcaggag

SEQ ID NO: 454 - DNA, 20 bp, Artificial Sequence
  source: /note=“Description of Artificial Sequence Synthetic primer”
gagaccctgctgtcccagaa

SEQ ID NO: 455 - DNA, 23 bp, Artificial Sequence
  source: /note=“Description of Artificial Sequence Synthetic primer”
ggttgtagtcagcgaaggagatc

SEQ ID NO: 456 - DNA, 26 bp, Artificial Sequence
  source: /note=“Description of Artificial Sequence Synthetic primer”
tcccacaatgaaggtcttgcctccct

SEQ ID NO: 457 - DNA, 20 bp, Artificial Sequence
  source: /note=“Description of Artificial Sequence Synthetic primer”
cccactcagtagccaagtca

SEQ ID NO: 458 - DNA, 20 bp, Artificial Sequence
  source: /note=“Description of Artificial Sequence Synthetic primer”
cacgcaggtggtatcagtct

SEQ ID NO: 459 - DNA, 27 bp, Artificial Sequence
  source: /note=“Description of Artificial Sequence Synthetic primer”
tcaagtaaacgggctgttttccaaaca

SEQ ID NO: 460 - DNA, 18 bp, Artificial Sequence
  source: /note=“Description of Artificial Sequence Synthetic primer”
tcctgtgctctggaagcc

SEQ ID NO: 461 - DNA, 21 bp, Artificial Sequence
  source: /note=“Description of Artificial Sequence Synthetic primer”
ctccacggtctcagttgatct

SEQ ID NO: 462 - DNA, 24 bp, Artificial Sequence
  source: /note=“Description of Artificial Sequence Synthetic primer”
caagaacctcccagaagggctcaa

SEQ ID NO: 463 - DNA, 20 bp, Artificial Sequence
  source: /note=“Description of Artificial Sequence Synthetic primer”
cggtgtgagaagtgcagcaa

SEQ ID NO: 464 - DNA, 19 bp, Artificial Sequence
  source: /note=“Description of Artificial Sequence Synthetic primer”
cctctcgcaagtgctccat

SEQ ID NO: 465 - DNA, 24 bp, Artificial Sequence
  source: /note=“Description of Artificial Sequence Synthetic primer”
ccagaccatagcacactcgggcac

SEQ ID NO: 466 - DNA, 24 bp, Artificial Sequence
  source: /note=“Description of Artificial Sequence Synthetic primer”
tgaacataaagtctgcaacatgga

SEQ ID NO: 467 - DNA, 28 bp, Artificial Sequence
  source: /note=“Description of Artificial Sequence Synthetic primer”
tgaggttggttactgttggtatcatata

SEQ ID NO: 468 - DNA, 24 bp, Artificial Sequence
  source: /note=“Description of Artificial Sequence Synthetic primer”
ttgcactgcacaggccacattcac

SEQ ID NO: 469 - DNA, 24 bp, Artificial Sequence
  source: /note=“Description of Artificial Sequence Synthetic primer”
tccaggatgttaggaactgtgaag

SEQ ID NO: 470 - DNA, 22 bp, Artificial Sequence
  source: /note=“Description of Artificial Sequence Synthetic primer”
gcgtgtctgcgtagtagctgtt

SEQ ID NO: 471 - DNA, 24 bp, Artificial Sequence
  source: /note=“Description of Artificial Sequence Synthetic primer”
agtcgctggtttcatgcccttcca

SEQ ID NO: 472 - DNA, 19 bp, Artificial Sequence
  source: /note=“Description of Artificial Sequence Synthetic primer”
ggacgaatacgaccccact

SEQ ID NO: 473 - DNA, 18 bp, Artificial Sequence
  source: /note=“Description of Artificial Sequence Synthetic primer”
gcacgtctccccatcaat

SEQ ID NO: 474 - DNA, 24 bp, Artificial Sequence
  source: /note=“Description of Artificial Sequence Synthetic primer”
accacctgcttccggtaggaatcc

SEQ ID NO: 475 - DNA, 19 bp, Artificial Sequence
  source: /note=“Description of Artificial Sequence Synthetic primer”
ctgctgcgacagtccacta

SEQ ID NO: 476 - DNA, 18 bp, Artificial Sequence
  source: /note=“Description of Artificial Sequence Synthetic primer”
caggttcgctctgggaag

SEQ ID NO: 477 - DNA, 24 bp, Artificial Sequence
  source: /note=“Description of Artificial Sequence Synthetic primer”
agagtgactcccgttgtcccaagg

SEQ ID NO: 478 - DNA, 19 bp, Artificial Sequence
  source: /note=“Description of Artificial Sequence Synthetic primer”
ggtccgcttcgtctttcga

SEQ ID NO: 479 - DNA, 19 bp, Artificial Sequence
  source: /note=“Description of Artificial Sequence Synthetic primer”
gcacaggttcgctctggaa

SEQ ID NO: 480 - DNA, 20 bp, Artificial Sequence
  source: /note=“Description of Artificial Sequence Synthetic primer”
tgactcccgcggtcccaagg

SEQ ID NO: 481 - DNA, 19 bp, Artificial Sequence
  source: /note=“Description of Artificial Sequence Synthetic primer”
gcaggtgtgattgctggac

SEQ ID NO: 482 - DNA, 18 bp, Artificial Sequence
  source: /note=“Description of Artificial Sequence Synthetic primer”
accataggcaatggcagc

SEQ ID NO: 483 - DNA, 24 bp, Artificial Sequence
  source: /note=“Description of Artificial Sequence Synthetic primer”
aagaatcatcaatgagcccacggc

SEQ ID NO: 484 - DNA, 24 bp, Artificial Sequence
  source: /note=“Description of Artificial Sequence Synthetic primer”
ggctagtagaactggatcccaaca

SEQ ID NO: 485 - DNA, 20 bp, Artificial Sequence
  source: /note=“Description of Artificial Sequence Synthetic primer”
ggtctgcccaaatgcttttc

SEQ ID NO: 486 - DNA, 30 bp, Artificial Sequence
  source: /note=“Description of Artificial Sequence Synthetic primer”
taattagacctaggcctcagctgcactgcc

SEQ ID NO: 487 - DNA, 19 bp, Artificial Sequence
  source: /note=“Description of Artificial Sequence Synthetic primer”
ggccactaaagatgctggc

SEQ ID NO: 488 - DNA, 18 bp, Artificial Sequence
  source: /note=“Description of Artificial Sequence Synthetic primer”
agcagctgtgggctcatt

SEQ ID NO: 489 - DNA, 24 bp, Artificial Sequence
  source: /note=“Description of Artificial Sequence Synthetic primer”
atcacccgaagcacattcagtcca

SEQ ID NO: 490 - DNA, 19 bp, Artificial Sequence
  source: /note=“Description of Artificial Sequence Synthetic primer”
ccgactggaggagcataaa

SEQ ID NO: 491 - DNA, 19 bp, Artificial Sequence
  source: /note=“Description of Artificial Sequence Synthetic primer”
atgctggctgactctgctc

SEQ ID NO: 492 - DNA, 24 bp, Artificial Sequence
  source: /note=“Description of Artificial Sequence Synthetic primer”
cgcacttttctgagcagacgtcca

SEQ ID NO: 493 - DNA, 20 bp, Artificial Sequence
  source: /note=“Description of Artificial Sequence Synthetic primer”
caaaaggcagaggctgataa

SEQ ID NO: 494 - DNA, 20 bp, Artificial Sequence
  source: /note=“Description of Artificial Sequence Synthetic primer”
agcgcagtttcataaagcaa

SEQ ID NO: 495 - DNA, 27 bp, Artificial Sequence
  source: /note=“Description of Artificial Sequence Synthetic primer”
tgaccagatccttcacagacttgtcgt

SEQ ID NO: 496 - DNA, 19 bp, Artificial Sequence
  source: /note=“Description of Artificial Sequence Synthetic primer”
agaaccgcaaggtgagcaa

SEQ ID NO: 497 - DNA, 21 bp, Artificial Sequence
  source: /note=“Description of Artificial Sequence Synthetic primer”
tccaactgaaggtccctgatg

SEQ ID NO: 498 - DNA, 26 bp, Artificial Sequence
  source: /note=“Description of Artificial Sequence Synthetic primer”
tggagattctccagcacgtcatcgac

SEQ ID NO: 499 - DNA, 20 bp, Artificial Sequence
  source: /note=“Description of Artificial Sequence Synthetic primer”
cacgcagaaaaccacacttc

SEQ ID NO: 500 - DNA, 20 bp, Artificial Sequence
  source: /note=“Description of Artificial Sequence Synthetic primer”
catgttcctccttgtgcatc

SEQ ID NO: 501 - DNA, 24 bp, Artificial Sequence
  source: /note=“Description of Artificial Sequence Synthetic primer”
caacacttccttccccaaagccag

SEQ ID NO: 502 - DNA, 21 bp, Artificial Sequence
  source: /note=“Description of Artificial Sequence Synthetic primer”
gcatggtagccgaagatttca

SEQ ID NO: 503 - DNA, 30 bp, Artificial Sequence
  source: /note=“Description of Artificial Sequence Synthetic primer”
tttccggtaatagtctgtctcatagatatc

SEQ ID NO: 504 - DNA, 28 bp, Artificial Sequence
  source: /note=“Description of Artificial Sequence Synthetic primer”
cgcgtcataccaaaatctccgattttga

SEQ ID NO: 505 - DNA, 19 bp, Artificial Sequence
  source: /note=“Description of Artificial Sequence Synthetic primer”
gtggacagcaccatgaaca

SEQ ID NO: 506 - DNA, 20 bp, Artificial Sequence
  source: /note=“Description of Artificial Sequence Synthetic primer”
ccttcatacccgacttgagg

SEQ ID NO: 507 - DNA, 20 bp, Artificial Sequence
  source: /note=“Description of Artificial Sequence Synthetic primer”
cttccggccagcactgcctc

SEQ ID NO: 508 - DNA, 17 bp, Artificial Sequence
  source: /note=“Description of Artificial Sequence Synthetic primer”
acgcaccgggtgtctga

SEQ ID NO: 509 - DNA, 24 bp, Artificial Sequence
  source: /note=“Description of Artificial Sequence Synthetic primer”
tgccctttcttgatgatgattatc

SEQ ID NO: 510 - DNA, 24 bp, Artificial Sequence
  source: /note=“Description of Artificial Sequence Synthetic primer”
cccaagttccaccccctccattca

SEQ ID NO: 511 - DNA, 22 bp, Artificial Sequence
  source: /note=“Description of Artificial Sequence Synthetic primer”
tggacaagtacgggatgaagct

SEQ ID NO: 512 - DNA, 21 bp, Artificial Sequence
  source: /note=“Description of Artificial Sequence Synthetic primer”
cgaaggtgtggcactgaaagt

SEQ ID NO: 513 - DNA, 24 bp, Artificial Sequence
  source: /note=“Description of Artificial Sequence Synthetic primer”
cccgtcaacgtactccatgcctgg

SEQ ID NO: 514 - DNA, 19 bp, Artificial Sequence
  source: /note=“Description of Artificial Sequence Synthetic primer”
gcggtgattcggaaattcg

SEQ ID NO: 515 - DNA, 19 bp, Artificial Sequence
  source: /note=“Description of Artificial Sequence Synthetic primer”
ctctcctgggcacctgctt

SEQ ID NO: 516 - DNA, 23 bp, Artificial Sequence
  source: /note=“Description of Artificial Sequence Synthetic primer”
ctctggtcctcatccaggtgcgc

SEQ ID NO: 517 - DNA, 26 bp, Artificial Sequence
  source: /note=“Description of Artificial Sequence Synthetic primer”
aaggaaccatctcactgtgtgtaaac

SEQ ID NO: 518 - DNA, 20 bp, Artificial Sequence
  source: /note=“Description of Artificial Sequence Synthetic primer”
atcaggaaggctgccaagag

SEQ ID NO: 519 - DNA, 22 bp, Artificial Sequence
  source: /note=“Description of Artificial Sequence Synthetic primer”
tgacttccaagctggccgtggc

SEQ ID NO: 520 - DNA, 20 bp, Artificial Sequence
  source: /note=“Description of Artificial Sequence Synthetic primer”
tctgcgtggttcctttctca

SEQ ID NO: 521 - DNA, 25 bp, Artificial Sequence
  source: /note=“Description of Artificial Sequence Synthetic primer”
ttgaaggatgtttattaggcaacgt

SEQ ID NO: 522 - DNA, 26 bp, Artificial Sequence
  source: /note=“Description of Artificial Sequence Synthetic primer”
cgcttctgactgctgattctcccgtt

SEQ ID NO: 523 - DNA, 20 bp, Artificial Sequence
  source: /note=“Description of Artificial Sequence Synthetic primer”
cctgaaccttccaaagatgg

SEQ ID NO: 524 - DNA, 20 bp, Artificial Sequence
  source: /note=“Description of Artificial Sequence Synthetic primer”
accaggcaagtctcctcatt

SEQ ID NO: 525 - DNA, 27 bp, Artificial Sequence
  source: /note=“Description of Artificial Sequence Synthetic primer”
ccagattggaagcatccatctttttca

SEQ ID NO: 526 - DNA, 20 bp, Artificial Sequence
  source: /note=“Description of Artificial Sequence Synthetic primer”
ccgctccgtcactgatgtct

SEQ ID NO: 527 - DNA, 21 bp, Artificial Sequence
  source: /note=“Description of Artificial Sequence Synthetic primer”
gcaaggtcagggcaaagagta

SEQ ID NO: 528 - DNA, 24 bp, Artificial Sequence
  source: /note=“Description of Artificial Sequence Synthetic primer”
cctgctgaacctagccttggccga

SEQ ID NO: 529 - DNA, 20 bp, Artificial Sequence
  source: /note=“Description of Artificial Sequence Synthetic primer”
ctcaggattttctcgcatcc

SEQ ID NO: 530 - DNA, 19 bp, Artificial Sequence
  source: /note=“Description of Artificial Sequence Synthetic primer”
aggagcaggtggagactgg

SEQ ID NO: 531 - DNA, 24 bp, Artificial Sequence
  source: /note=“Description of Artificial Sequence Synthetic primer”
atgtgctcccagtgctaggtgcct

SEQ ID NO: 532 - DNA, 20 bp, Artificial Sequence
  source: /note=“Description of Artificial Sequence Synthetic primer”
agccatcactctcagtgcag

SEQ ID NO: 533 - DNA, 20 bp, Artificial Sequence
  source: /note=“Description of Artificial Sequence Synthetic primer”
actgcagagtcagggtctcc

SEQ ID NO: 534 - DNA, 22 bp, Artificial Sequence
  source: /note=“Description of Artificial Sequence Synthetic primer”
caggtcctatcgtggcccctga

SEQ ID NO: 535 - DNA, 21 bp, Artificial Sequence
  source: /note=“Description of Artificial Sequence Synthetic primer”
gccaggatactggagtccatc

SEQ ID NO: 536 - DNA, 18 bp, Artificial Sequence
  source: /note=“Description of Artificial Sequence Synthetic primer”
cttgacccttggggtcct

SEQ ID NO: 537 - DNA, 23 bp, Artificial Sequence
  source: /note=“Description of Artificial Sequence Synthetic primer”
tgagctccctgatggctacaccc

SEQ ID NO: 538 - DNA, 18 bp, Artificial Sequence
  source: /note=“Description of Artificial Sequence Synthetic primer”
ggatggagttcgcctcct

SEQ ID NO: 539 - DNA, 20 bp, Artificial Sequence
  source: /note=“Description of Artificial Sequence Synthetic primer”
cgctccatggacttgatctt

SEQ ID NO: 540 - DNA, 24 bp, Artificial Sequence
  source: /note=“Description of Artificial Sequence Synthetic primer”
cgtgatcacagacctgacccagct

SEQ ID NO: 541 - DNA, 20 bp, Artificial Sequence
  source: /note=“Description of Artificial Sequence Synthetic primer”
ccacagctcaccttctgtca

SEQ ID NO: 542 - DNA, 20 bp, Artificial Sequence
  source: /note=“Description of Artificial Sequence Synthetic primer”
cctcagtgccagtctcttcc

SEQ ID NO: 543 - DNA, 20 bp, Artificial Sequence
  source: /note=“Description of Artificial Sequence Synthetic primer”
tccatcccagctccagccag

SEQ ID NO: 544 - DNA, 20 bp, Artificial Sequence
  source: /note=“Description of Artificial Sequence Synthetic primer”
tcagaattggatttggctca

SEQ ID NO: 545 - DNA, 20 bp, Artificial Sequence
  source: /note=“Description of Artificial Sequence Synthetic primer”
cctgagcttagctggtgttg

SEQ ID NO: 546 - DNA, 30 bp, Artificial Sequence
  source: /note=“Description of Artificial Sequence Synthetic primer”
tgctaatgtaaggcatcacagtcttttcca

SEQ ID NO: 547 - DNA, 26 bp, Artificial Sequence
  source: /note=“Description of Artificial Sequence Synthetic primer”
tgaggaagaaggagaggaatactaat

SEQ ID NO: 548 - DNA, 21 bp, Artificial Sequence
  source: /note=“Description of Artificial Sequence Synthetic primer”
ctgaaattctgggagcatgac

SEQ ID NO: 549 - DNA, 24 bp, Artificial Sequence
  source: /note=“Description of Artificial Sequence Synthetic primer”
tatccattccttttggccctgcag

SEQ ID NO: 550 - DNA, 20 bp, Artificial Sequence
  source: /note=“Description of Artificial Sequence Synthetic primer”
gaggacgaaggcctctacac

SEQ ID NO: 551 - DNA, 20 bp, Artificial Sequence
  source: /note=“Description of Artificial Sequence Synthetic primer”
aaaaatgcctccacttttgc

SEQ ID NO: 552 - DNA, 24 bp, Artificial Sequence
  source: /note=“Description of Artificial Sequence Synthetic primer”
caggcatgcagtgttcttggctgt

SEQ ID NO: 553 - DNA, 19 bp, Artificial Sequence
  source: /note=“Description of Artificial Sequence Synthetic primer”
cggactttgggtgcgactt

SEQ ID NO: 554 - DNA, 24 bp, Artificial Sequence
  source: /note=“Description of Artificial Sequence Synthetic primer”
ttacaactcttccactgggacgat

SEQ ID NO: 555 - DNA, 23 bp, Artificial Sequence
  source: /note=“Description of Artificial Sequence Synthetic primer”
ccacttgtcgaaccaccgctcgt

SEQ ID NO: 556 - DNA, 20 bp, Artificial Sequence
  source: /note=“Description of Artificial Sequence Synthetic primer”
tggaggttgtaagccaatgt

SEQ ID NO: 557 - DNA, 20 bp, Artificial Sequence
  source: /note=“Description of Artificial Sequence Synthetic primer”
tgccttacgtccatctgatt

SEQ ID NO: 558 - DNA, 28 bp, Artificial Sequence
  source: /note=“Description of Artificial Sequence Synthetic primer”
cagtgatgtctgaacttgaagcctcaca

SEQ ID NO: 559 - DNA, 20 bp, Artificial Sequence
  source: /note=“Description of Artificial Sequence Synthetic primer”
ctaaggcacttgctggaagg

SEQ ID NO: 560 - DNA, 18 bp, Artificial Sequence
  source: /note=“Description of Artificial Sequence Synthetic primer”
tcttcccagctcctgtgg

SEQ ID NO: 561 - DNA, 24 bp, Artificial Sequence
  source: /note=“Description of Artificial Sequence Synthetic primer”
tccataggcaagcacactggcatt

SEQ ID NO: 562 - DNA, 25 bp, Artificial Sequence
  source: /note=“Description of Artificial Sequence Synthetic primer”
aattcctgctccaaaagaaagtctt

SEQ ID NO: 563 - DNA, 20 bp, Artificial Sequence
  source: /note=“Description of Artificial Sequence Synthetic primer”
cgtgatgcgaagctctgaga

SEQ ID NO: 564 - DNA, 22 bp, Artificial Sequence
  source: /note=“Description of Artificial Sequence Synthetic primer”
aagccgctccactcgcatgtcc

SEQ ID NO: 565 - DNA, 20 bp, Artificial Sequence
  source: /note=“Description of Artificial Sequence Synthetic primer”
ccacagggttgaagaaccag

SEQ ID NO: 566 - DNA, 20 bp, Artificial Sequence
  source: /note=“Description of Artificial Sequence Synthetic primer”
cacctgatgtgccagacttc

SEQ ID NO: 567 - DNA, 24 bp, Artificial Sequence
  source: /note=“Description of Artificial Sequence Synthetic primer”
agccagttcctgctgttcctgtcc

SEQ ID NO: 568 - DNA, 18 bp, Artificial Sequence
  source: /note=“Description of Artificial Sequence Synthetic primer”
gcccagaggctccatcgt

SEQ ID NO: 569 - DNA, 23 bp, Artificial Sequence
  source: /note=“Description of Artificial Sequence Synthetic primer”
cagaggtttgaacagtgcagaca

SEQ ID NO: 570 - DNA, 23 bp, Artificial Sequence
  source: /note=“Description of Artificial Sequence Synthetic primer”
cctcttcctccccagtcggctga

SEQ ID NO: 571 - DNA, 20 bp, Artificial Sequence
  source: /note=“Description of Artificial Sequence Synthetic primer”
caaacagagggtggcagaag

SEQ ID NO: 572 - DNA, 18 bp, Artificial Sequence
  source: /note=“Description of Artificial Sequence Synthetic primer”
gaggctctcacggctcct

SEQ ID NO: 573 - DNA, 24 bp, Artificial Sequence
  source: /note=“Description of Artificial Sequence Synthetic primer”
cgcttctccatgttctcagggtca

SEQ ID NO: 574 - DNA, 18 bp, Artificial Sequence
  source: /note=“Description of Artificial Sequence Synthetic primer”
agccgaggctttgttgaa

SEQ ID NO: 575 - DNA, 19 bp, Artificial Sequence
  source: /note=“Description of Artificial Sequence Synthetic primer”
tgggctatgagcacagctt

SEQ ID NO: 576 - DNA, 24 bp, Artificial Sequence
  source: /note=“Description of Artificial Sequence Synthetic primer”
ttcatatccagtaccggcgatcgg

SEQ ID NO: 577 - DNA, 20 bp, Artificial Sequence
  source: /note=“Description of Artificial Sequence Synthetic primer”
atgtgccagtgagcttgagt

SEQ ID NO: 578 - DNA, 20 bp, Artificial Sequence
  source: /note=“Description of Artificial Sequence Synthetic primer”
tgagcccctggttaacagta

SEQ ID NO: 579 - DNA, 24 bp, Artificial Sequence
  source: /note=“Description of Artificial Sequence Synthetic primer”
ccttggagaaacacaagcacctgc

SEQ ID NO: 580 - DNA, 20 bp, Artificial Sequence
  source: /note=“Description of Artificial Sequence Synthetic primer”
ggcctgctgagatcaaagac

SEQ ID NO: 581 - DNA, 20 bp, Artificial Sequence
  source: /note=“Description of Artificial Sequence Synthetic primer”
gtccactgtggctgtgagaa

SEQ ID NO: 582 - DNA, 26 bp, Artificial Sequence
  source: /note=“Description of Artificial Sequence Synthetic primer”
tgttcctcaggtcctcaatggtcttg

SEQ ID NO: 583 - DNA, 21 bp, Artificial Sequence
  source: /note=“Description of Artificial Sequence Synthetic primer”
cgaggattggttcttcagcaa

SEQ ID NO: 584 - DNA, 24 bp, Artificial Sequence
  source: /note=“Description of Artificial Sequence Synthetic primer”
cacctcgcggttcagttcctctgt

SEQ ID NO: 585 - DNA, 22 bp, Artificial Sequence
  source: /note=“Description of Artificial Sequence Synthetic primer”
actctgcaccagctcactgttg

SEQ ID NO: 586 - DNA, 21 bp, Artificial Sequence
  source: /note=“Description of Artificial Sequence Synthetic primer”
tgagcggcagaatcaggagta

SEQ ID NO: 587 - DNA, 19 bp, Artificial Sequence
  source: /note=“Description of Artificial Sequence Synthetic primer”
tgcggtaggtggcaatctc

SEQ ID NO: 588 - DNA, 24 bp, Artificial Sequence
  source: /note=“Description of Artificial Sequence Synthetic primer”
ctcatggacatcaagtcgcggctg

SEQ ID NO: 589 - DNA, 20 bp, Artificial Sequence
  source: /note=“Description of Artificial Sequence Synthetic primer”
tcagtggagaaggagttgga

SEQ ID NO: 590 - DNA, 20 bp, Artificial Sequence
  source: /note=“Description of Artificial Sequence Synthetic primer”
tgccatatccagaggaaaca

SEQ ID NO: 591 - DNA, 28 bp, Artificial Sequence
  source: /note=“Description of Artificial Sequence Synthetic primer”
ccagtcaacatctctgttgtcacaagca

SEQ ID NO: 592 - DNA, 18 bp, Artificial Sequence
  source: /note=“Description of Artificial Sequence Synthetic primer”
cttgctggccaatgccta

SEQ ID NO: 593 - DNA, 18 bp, Artificial Sequence
  source: /note=“Description of Artificial Sequence Synthetic primer”
tgattgtccgcagtcagg

SEQ ID NO: 594 - DNA, 24 bp, Artificial Sequence
  source: /note=“Description of Artificial Sequence Synthetic primer”
atctacgttgtccagctgccagcc

SEQ ID NO: 595 - DNA, 21 bp, Artificial Sequence
  source: /note=“Description of Artificial Sequence Synthetic primer”
actcaagcggaaattgaagca

SEQ ID NO: 596 - DNA, 21 bp, Artificial Sequence
  source: /note=“Description of Artificial Sequence Synthetic primer”
actccctgaagccgagacact

SEQ ID NO: 597 - DNA, 28 bp, Artificial Sequence
  source: /note=“Description of Artificial Sequence Synthetic primer”
aggtcttatcagcacagtctccgcctcc

SEQ ID NO: 598 - DNA, 18 bp, Artificial Sequence
  source: /note=“Description of Artificial Sequence Synthetic primer”
agcgatgaagatggtcgc

SEQ ID NO: 599 - DNA, 18 bp, Artificial Sequence
  source: /note=“Description of Artificial Sequence Synthetic primer”
gacatggcagcacaagca

SEQ ID NO: 600 - DNA, 24 bp, Artificial Sequence
  source: /note=“Description of Artificial Sequence Synthetic primer”
ctggacgcggttctactccaacag

SEQ ID NO: 601 - DNA, 20 bp, Artificial Sequence
  source: /note=“Description of Artificial Sequence Synthetic primer”
gcttcaggtgttgtgactgc

SEQ ID NO: 602 - DNA, 20 bp, Artificial Sequence
  source: /note=“Description of Artificial Sequence Synthetic primer”
aagagctgcccatccttctc

SEQ ID NO: 603 - DNA, 24 bp, Artificial Sequence
  source: /note=“Description of Artificial Sequence Synthetic primer”
tgcctccctgtcgcaccagtacta

SEQ ID NO: 604 - DNA, 18 bp, Artificial Sequence
  source: /note=“Description of Artificial Sequence Synthetic primer”
ctttgggccaggaggaat

SEQ ID NO: 605 - DNA, 18 bp, Artificial Sequence
  source: /note=“Description of Artificial Sequence Synthetic primer”
ctcccacaatccactgcc

SEQ ID NO: 606 - DNA, 24 bp, Artificial Sequence
  source: /note=“Description of Artificial Sequence Synthetic primer”
actcgaatccacccaggaactccc

SEQ ID NO: 607 - DNA, 20 bp, Artificial Sequence
  source: /note=“Description of Artificial Sequence Synthetic primer”
agaagctgtccctgcaagag

SEQ ID NO: 608 - DNA, 20 bp, Artificial Sequence
  source: /note=“Description of Artificial Sequence Synthetic primer”
agccgtaccagctcagactt

SEQ ID NO: 609 - DNA, 25 bp, Artificial Sequence
  source: /note=“Description of Artificial Sequence Synthetic primer”
catgttcttcacaatcgctgcatcc

SEQ ID NO: 610 - DNA, 18 bp, Artificial Sequence
  source: /note=“Description of Artificial Sequence Synthetic primer”
ccgggagcagggaatcac

SEQ ID NO: 611 - DNA, 20 bp, Artificial Sequence
  source: /note=“Description of Artificial Sequence Synthetic primer”
atgctgttgatgccgaatga

SEQ ID NO: 612 - DNA, 19 bp, Artificial Sequence
  source: /note=“Description of Artificial Sequence Synthetic primer”
cggccacgatttcggcgct

SEQ ID NO: 613 - DNA, 20 bp, Artificial Sequence
  source: /note=“Description of Artificial Sequence Synthetic primer”
ctgtcatgtggcagaccttc

SEQ ID NO: 614 - DNA, 20 bp, Artificial Sequence
  source: /note=“Description of Artificial Sequence Synthetic primer”
taaatgtcactggtgcctgg

SEQ ID NO: 615 - DNA, 24 bp, Artificial Sequence
  source: /note=“Description of Artificial Sequence Synthetic primer”
cgaaccacggcttgggaagactac

SEQ ID NO: 616 - DNA, 19 bp, Artificial Sequence
  source: /note=“Description of Artificial Sequence Synthetic primer”
gcccagtggagaaattcgt

SEQ ID NO: 617 - DNA, 19 bp, Artificial Sequence
  source: /note=“Description of Artificial Sequence Synthetic primer”
gcgagtctgagctgatgga

SEQ ID NO: 618 - DNA, 24 bp, Artificial Sequence
  source: /note=“Description of Artificial Sequence Synthetic primer”
tttgagatcacccagcctccactg

SEQ ID NO: 619 - DNA, 20 bp, Artificial Sequence
  source: /note=“Description of Artificial Sequence Synthetic primer”
cctcagaaattgccaggact

SEQ ID NO: 620 - DNA, 24 bp, Artificial Sequence
  source: /note=“Description of Artificial Sequence Synthetic primer”
ttcccgtgatcttcagcaaagcct

SEQ ID NO: 621 - DNA, 20 bp, Artificial Sequence
  source: /note=“Description of Artificial Sequence Synthetic primer”
ccaaagaccagctgcaagta

SEQ ID NO: 622 - DNA, 20 bp, Artificial Sequence
  source: /note=“Description of Artificial Sequence Synthetic primer”
aggactccagcaaccaagaa

SEQ ID NO: 623 - DNA, 20 bp, Artificial Sequence
  source: /note=“Description of Artificial Sequence Synthetic primer”
gagtgctgcttggaactcag

SEQ ID NO: 624 - DNA, 24 bp, Artificial Sequence
  source: /note=“Description of Artificial Sequence Synthetic primer”
caagcaccttccctgacctggagt

SEQ ID NO: 625 - DNA, 18 bp, Artificial Sequence
  source: /note=“Description of Artificial Sequence Synthetic primer”
cggaccaccaggtcagag

SEQ ID NO: 626 - DNA, 20 bp, Artificial Sequence
  source: /note=“Description of Artificial Sequence Synthetic primer”
caggggtagtgggtgttgag

SEQ ID NO: 627 - DNA, 24 bp, Artificial Sequence
  source: /note=“Description of Artificial Sequence Synthetic primer”
ccactcttccctgctctgcgaatt

SEQ ID NO: 628 - DNA, 20 bp, Artificial Sequence
  source: /note=“Description of Artificial Sequence Synthetic primer”
gccctccaatgtccttatca

SEQ ID NO: 629 - DNA, 21 bp, Artificial Sequence
  source: /note=“Description of Artificial Sequence Synthetic primer”
gtagccactgatgccaaagtc

SEQ ID NO: 630 - DNA, 24 bp, Artificial Sequence
  source: /note=“Description of Artificial Sequence Synthetic primer”
cacatcttcacatggccctccttg

SEQ ID NO: 631 - DNA, 18 bp, Artificial Sequence
  source: /note=“Description of Artificial Sequence Synthetic primer”
gccggtcaggcacacaag

SEQ ID NO: 632 - DNA, 23 bp, Artificial Sequence
  source: /note=“Description of Artificial Sequence Synthetic primer”
gcagcatacacacaacaaaatgg

SEQ ID NO: 633 - DNA, 24 bp, Artificial Sequence
  source: /note=“Description of Artificial Sequence Synthetic primer”
accaaccagtccacgctccaaggg

SEQ ID NO: 634 - DNA, 18 bp, Artificial Sequence
  source: /note=“Description of Artificial Sequence Synthetic primer”
ccctcaaccggcaaatcc

SEQ ID NO: 635 - DNA, 19 bp, Artificial Sequence
  source: /note=“Description of Artificial Sequence Synthetic primer”
cgtccatgccctgaattca

SEQ ID NO: 636 - DNA, 23 bp, Artificial Sequence
  source: /note=“Description of Artificial Sequence Synthetic primer”
tggcgagtgcagtgagcagctcc

SEQ ID NO: 637 - DNA, 23 bp, Artificial Sequence
  source: /note=“Description of Artificial Sequence Synthetic primer”
tgagtggaaaagcctgacctatg

SEQ ID NO: 638 - DNA, 23 bp, Artificial Sequence
  source: /note=“Description of Artificial Sequence Synthetic primer”
ggactccatctcttcttggtcaa

SEQ ID NO: 639 - DNA, 27 bp, Artificial Sequence
  source: /note=“Description of Artificial Sequence Synthetic primer”
tgaagtcatcagctttgtgccaccacc

SEQ ID NO: 640 - DNA, 22 bp, Artificial Sequence
  source: /note=“Description of Artificial Sequence Synthetic primer”
caacacccgtacatcaatgtct

SEQ ID NO: 641 - DNA, 22 bp, Artificial Sequence
  source: /note=“Description of Artificial Sequence Synthetic primer”
tcatctaactgcttgtcaggga

SEQ ID NO: 642 - DNA, 24 bp, Artificial Sequence
  source: /note=“Description of Artificial Sequence Synthetic primer”
ctgaagcagaagctccaccaccaa

SEQ ID NO: 643 - DNA, 20 bp, Artificial Sequence
  source: /note=“Description of Artificial Sequence Synthetic primer”
gaccttggaacctttggaac

SEQ ID NO: 644 - DNA, 20 bp, Artificial Sequence
  source: /note=“Description of Artificial Sequence Synthetic primer”
cctaggcctatgagggttca

SEQ ID NO: 645 - DNA, 26 bp, Artificial Sequence
  source: /note=“Description of Artificial Sequence Synthetic primer”
cagccctgtaagacctgttgacagca

SEQ ID NO: 646 - DNA, 18 bp, Artificial Sequence
  source: /note=“Description of Artificial Sequence Synthetic primer”
cacaagctgaccttccgc

SEQ ID NO: 647 - DNA, 20 bp, Artificial Sequence
  source: /note=“Description of Artificial Sequence Synthetic primer”
acttgtacacgatctccgcc

SEQ ID NO: 648 - DNA, 24 bp, Artificial Sequence
  source: /note=“Description of Artificial Sequence Synthetic primer”
agaacgccaaagccaagacagacc

SEQ ID NO: 649 - DNA, 23 bp, Artificial Sequence
  source: /note=“Description of Artificial Sequence Synthetic primer”
cagatggccactttgagaacatt

SEQ ID NO: 650 - DNA, 22 bp, Artificial Sequence
  source: /note=“Description of Artificial Sequence Synthetic primer”
ggcagcattaaccacaaggatt

SEQ ID NO: 651 - DNA, 28 bp, Artificial Sequence
  source: /note=“Description of Artificial Sequence Synthetic primer”
agctgacaacagtgtgaacgaccagacc

SEQ ID NO: 652 - DNA, 20 bp, Artificial Sequence
  source: /note=“Description of Artificial Sequence Synthetic primer”
cttcggaaactggacatcaa

SEQ ID NO: 653 - DNA, 20 bp, Artificial Sequence
  source: /note=“Description of Artificial Sequence Synthetic primer”
gtcgctgaaaacatggatca

SEQ ID NO: 654 - DNA, 26 bp, Artificial Sequence
  source: /note=“Description of Artificial Sequence Synthetic primer”
tcactcgagacaacgatttcacatcg

SEQ ID NO: 655 - DNA, 21 bp, Artificial Sequence
  source: /note=“Description of Artificial Sequence Synthetic primer”
gacttttgcccgctacctttc

SEQ ID NO: 656 - DNA, 26 bp, Artificial Sequence
  source: /note=“Description of Artificial Sequence Synthetic primer”
gccactaactgcttcagtatgaagag

SEQ ID NO: 657 - DNA, 24 bp, Artificial Sequence
  source: /note=“Description of Artificial Sequence Synthetic primer”
acagctcattgttgtcacgccgga

SEQ ID NO: 658 - DNA, 24 bp, Artificial Sequence
  source: /note=“Description of Artificial Sequence Synthetic primer”
tgatggtcctatgtgtcacattca

SEQ ID NO: 659 - DNA, 20 bp, Artificial Sequence
  source: /note=“Description of Artificial Sequence Synthetic primer”
tgggacaggaaacacaccaa

SEQ ID NO: 660 - DNA, 30 bp, Artificial Sequence
  source: /note=“Description of Artificial Sequence Synthetic primer”
caggtttcataccaacacaggcttcagcac

SEQ ID NO: 661 - DNA, 19 bp, Artificial Sequence
  source: /note=“Description of Artificial Sequence Synthetic primer”
cgctcagccagatgcaatc

SEQ ID NO: 662 - DNA, 25 bp, Artificial Sequence
  source: /note=“Description of Artificial Sequence Synthetic primer”
gcactgagatcttcctattggtgaa

SEQ ID NO: 663 - DNA, 21 bp, Artificial Sequence
  source: /note=“Description of Artificial Sequence Synthetic primer”
tgccccagtcacctgctgtta

SEQ ID NO: 664 - DNA, 20 bp, Artificial Sequence
  source: /note=“Description of Artificial Sequence Synthetic primer”
gtgaaatgaaacgcaccaca

SEQ ID NO: 665 - DNA, 20 bp, Artificial Sequence
  source: /note=“Description of Artificial Sequence Synthetic primer”
gaccctgctcacaaccagac

SEQ ID NO: 666 - DNA, 20 bp, Artificial Sequence
  source: /note=“Description of Artificial Sequence Synthetic primer”
cagccctttggggaagctgg

SEQ ID NO: 667 - DNA, 19 bp, Artificial Sequence
  source: /note=“Description of Artificial Sequence Synthetic primer”
ccaacgcttgccaaatcct

SEQ ID NO: 668 - DNA, 24 bp, Artificial Sequence
  source: /note=“Description of Artificial Sequence Synthetic primer”
acggtagtgacagcatcaaaactc

SEQ ID NO: 669 - DNA, 24 bp, Artificial Sequence
  source: /note=“Description of Artificial Sequence Synthetic primer”
aaccagctctctgtgaccccaatt

SEQ ID NO: 670 - DNA, 20 bp, Artificial Sequence
  source: /note=“Description of Artificial Sequence Synthetic primer”
ccatgatggagaggcagaca

SEQ ID NO: 671 - DNA, 21 bp, Artificial Sequence
  source: /note=“Description of Artificial Sequence Synthetic primer”
ggagtccgtccttaccgtcaa

SEQ ID NO: 672 - DNA, 24 bp, Artificial Sequence
  source: /note=“Description of Artificial Sequence Synthetic primer”
ctgggagcatggcgatggataccc

SEQ ID NO: 673 - DNA, 20 bp, Artificial Sequence
  source: /note=“Description of Artificial Sequence Synthetic primer”
gagaaccaatctcaccgaca

SEQ ID NO: 674 - DNA, 20 bp, Artificial Sequence
  source: /note=“Description of Artificial Sequence Synthetic primer”
cacccgagtgtaaccatagc

SEQ ID NO: 675 - DNA, 24 bp, Artificial Sequence
  source: /note=“Description of Artificial Sequence Synthetic primer”
acaggtattcctctgccagctgcc

SEQ ID NO: 676 - DNA, 18 bp, Artificial Sequence
  source: /note=“Description of Artificial Sequence Synthetic primer”
gccatgctggctcagtct

SEQ ID NO: 677 - DNA, 20 bp, Artificial Sequence
  source: /note=“Description of Artificial Sequence Synthetic primer”
cacccagacccacctaactg

SEQ ID NO: 678 - DNA, 24 bp, Artificial Sequence
  source: /note=“Description of Artificial Sequence Synthetic primer”
cactagctgatgtggcccacagct

SEQ ID NO: 679 - DNA, 18 bp, Artificial Sequence
  source: /note=“Description of Artificial Sequence Synthetic primer”
tcatggtgcccgtcaatg

SEQ ID NO: 680 - DNA, 23 bp, Artificial Sequence
  source: /note=“Description of Artificial Sequence Synthetic primer”
cgattgtctttgctcttcatgtg

SEQ ID NO: 681 - DNA, 29 bp, Artificial Sequence
  source: /note=“Description of Artificial Sequence Synthetic primer”
acctgatacgtcttggtcttcatcgccat

SEQ ID NO: 682 - DNA, 20 bp, Artificial Sequence
  source: /note=“Description of Artificial Sequence Synthetic primer”
aggggatgacttggacacat

SEQ ID NO: 683 - DNA, 20 bp, Artificial Sequence
  source: /note=“Description of Artificial Sequence Synthetic primer”
aaaactgcatggctttgtca

SEQ ID NO: 684 - DNA, 25 bp, Artificial Sequence
  source: /note=“Description of Artificial Sequence Synthetic primer”
ctgccattcgacatgactgcaattt

SEQ ID NO: 685 - DNA, 22 bp, Artificial Sequence
  source: /note=“Description of Artificial Sequence Synthetic primer”
tcatcctggcgatctacttcct

SEQ ID NO: 686 - DNA, 20 bp, Artificial Sequence
  source: /note=“Description of Artificial Sequence Synthetic primer”
ccgttgagtggaatcagcaa

SEQ ID NO: 687 - DNA, 26 bp, Artificial Sequence
  source: /note=“Description of Artificial Sequence Synthetic primer”
tctgtcctggctggagtcgctttcat

SEQ ID NO: 688 - DNA, 21 bp, Artificial Sequence
  source: /note=“Description of Artificial Sequence Synthetic primer”
tgattaccatcatggctcaga

SEQ ID NO: 689 - DNA, 20 bp, Artificial Sequence
  source: /note=“Description of Artificial Sequence Synthetic primer”
cttgtgaaaatgccatccac

SEQ ID NO: 690 - DNA, 24 bp, Artificial Sequence
  source: /note=“Description of Artificial Sequence Synthetic primer”
tcccaattgtcgcttcttctgcag

SEQ ID NO: 691 - DNA, 20 bp, Artificial Sequence
  source: /note=“Description of Artificial Sequence Synthetic primer”
ggccaggatctgtggtggta

SEQ ID NO: 692 - DNA, 20 bp, Artificial Sequence
  source: /note=“Description of Artificial Sequence Synthetic primer”
ctccacgtcgtggacattga

SEQ ID NO: 693 - DNA, 23 bp, Artificial Sequence
  source: /note=“Description of Artificial Sequence Synthetic primer”
ctctggccttccgagaaggtacc

SEQ ID NO: 694 - DNA, 23 bp, Artificial Sequence
  source: /note=“Description of Artificial Sequence Synthetic primer”
gaaggaatgggaatcagtcatga

SEQ ID NO: 695 - DNA, 26 bp, Artificial Sequence
  source: /note=“Description of Artificial Sequence Synthetic primer”
gtctattagagtcagatccgggacat

SEQ ID NO: 696 - DNA, 23 bp, Artificial Sequence
  source: /note=“Description of Artificial Sequence Synthetic primer”
tcaccctggagatcagctcccga

SEQ ID NO: 697 - DNA, 18 bp, Artificial Sequence
  source: /note=“Description of Artificial Sequence Synthetic primer”
gccgagatcgccaagatg

SEQ ID NO: 698 - DNA, 27 bp, Artificial Sequence
  source: /note=“Description of Artificial Sequence Synthetic primer”
cttttgatggtagagttccagtgattc

SEQ ID NO: 699 - DNA, 24 bp, Artificial Sequence
  source: /note=“Description of Artificial Sequence Synthetic primer”
cagcattgtctgtcctccctggca

SEQ ID NO: 700 - DNA, 27 bp, Artificial Sequence
  source: /note=“Description of Artificial Sequence Synthetic primer”
cggtacttctcagggctaatatatacg

SEQ ID NO: 701 - DNA, 21 bp, Artificial Sequence
  source: /note=“Description of Artificial Sequence Synthetic primer”
ccgagtagatgggcaggtgtt

SEQ ID NO: 702 - DNA, 26 bp, Artificial Sequence
  source: /note=“Description of Artificial Sequence Synthetic primer”
ctcttctgcgtggtggtcaaccccta

SEQ ID NO: 703 - DNA, 18 bp, Artificial Sequence
  source: /note=“Description of Artificial Sequence Synthetic primer”
gtgaggcagcgcgactct

SEQ ID NO: 704 - DNA, 23 bp, Artificial Sequence
  source: /note=“Description of Artificial Sequence Synthetic primer”
tgccaatggtgtacaacacttca

SEQ ID NO: 705 - DNA, 21 bp, Artificial Sequence
  source: /note=“Description of Artificial Sequence Synthetic primer”
tgccttcccgggctgaggact

SEQ ID NO: 706 - DNA, 20 bp, Artificial Sequence
  source: /note=“Description of Artificial Sequence Synthetic primer”
cagaccaaggagatggacct

SEQ ID NO: 707 - DNA, 18 bp, Artificial Sequence
  source: /note=“Description of Artificial Sequence Synthetic primer”
agctgccagtgctatccg

SEQ ID NO: 708 - DNA, 24 bp, Artificial Sequence
  source: /note=“Description of Artificial Sequence Synthetic primer”
aagctgtaaacatgagccgcacca

SEQ ID NO: 709 - DNA, 19 bp, Artificial Sequence
  source: /note=“Description of Artificial Sequence Synthetic primer”
ctgccgggatggcttctat

SEQ ID NO: 710 - DNA, 22 bp, Artificial Sequence
  source: /note=“Description of Artificial Sequence Synthetic primer”
ccaggttctggaaactgtggat

SEQ ID NO: 711 - DNA, 21 bp, Artificial Sequence
  source: /note=“Description of Artificial Sequence Synthetic primer”
ctgagctctgcccggaccgct

SEQ ID NO: 712 - DNA, 18 bp, Artificial Sequence
  source: /note=“Description of Artificial Sequence Synthetic primer”
cactgacacccgcaacac

SEQ ID NO: 713 - DNA, 19 bp, Artificial Sequence
  source: /note=“Description of Artificial Sequence Synthetic primer”
ggctgcaatctctctgctg

SEQ ID NO: 714 - DNA, 24 bp, Artificial Sequence
  source: /note=“Description of Artificial Sequence Synthetic primer”
aaccacagagtccgaaccatgggt

SEQ ID NO: 715 - DNA, 19 bp, Artificial Sequence
  source: /note=“Description of Artificial Sequence Synthetic primer”
ctgcttctttcccgagctt

SEQ ID NO: 716 - DNA, 19 bp, Artificial Sequence
  source: /note=“Description of Artificial Sequence Synthetic primer”
agcaggaatgtagctgcca

SEQ ID NO: 717 - DNA, 24 bp, Artificial Sequence
  source: /note=“Description of Artificial Sequence Synthetic primer”
acttcgttatccgcgatgcgtttc

SEQ ID NO: 718 - DNA, 20 bp, Artificial Sequence
  source: /note=“Description of Artificial Sequence Synthetic primer”
ggctgtggctgaggctgtag

SEQ ID NO: 719 - DNA, 21 bp, Artificial Sequence
  source: /note=“Description of Artificial Sequence Synthetic primer”
ggagcattcgaggtcaaatca

SEQ ID NO: 720 - DNA, 28 bp, Artificial Sequence
  source: /note=“Description of Artificial Sequence Synthetic primer”
ttcccagagtgtctcacctccagcagag

SEQ ID NO: 721 - DNA, 20 bp, Artificial Sequence
  source: /note=“Description of Artificial Sequence Synthetic primer”
cggacctgaatgtaatgcaa

SEQ ID NO: 722 - DNA, 20 bp, Artificial Sequence
  source: /note=“Description of Artificial Sequence Synthetic primer”
ctttgccaggtgactaagca

SEQ ID NO: 723 - DNA, 28 bp, Artificial Sequence
  source: /note=“Description of Artificial Sequence Synthetic primer”
aatgaacagaacaagcaaaatgaccagc

SEQ ID NO: 724 - DNA, 20 bp, Artificial Sequence
  source: /note=“Description of Artificial Sequence Synthetic primer”
caaaggaagagcaacggaag

SEQ ID NO: 725 - DNA, 19 bp, Artificial Sequence
  source: /note=“Description of Artificial Sequence Synthetic primer”
attcccaaaacctttgctt

SEQ ID NO: 726 - DNA, 24 bp, Artificial Sequence
  source: /note=“Description of Artificial Sequence Synthetic primer”
ttctcctttcgttcttgctcgcgt

SEQ ID NO: 727 - DNA, 21 bp, Artificial Sequence
  source: /note=“Description of Artificial Sequence Synthetic primer”
tggagactctcagggtcgaaa

SEQ ID NO: 728 - DNA, 22 bp, Artificial Sequence
  source: /note=“Description of Artificial Sequence Synthetic primer”
ggcgtttggagtggtagaaatc

SEQ ID NO: 729 - DNA, 20 bp, Artificial Sequence
  source: /note=“Description of Artificial Sequence Synthetic primer”
cggcggcagaccagcatgac

SEQ ID NO: 730 - DNA, 21 bp, Artificial Sequence
  source: /note=“Description of Artificial Sequence Synthetic primer”
cggtggaccacgaagagttaa

SEQ ID NO: 731 - DNA, 19 bp, Artificial Sequence
  source: /note=“Description of Artificial Sequence Synthetic primer”
ggctcgcctcttccatgtc

SEQ ID NO: 732 - DNA, 23 bp, Artificial Sequence
  source: /note=“Description of Artificial Sequence Synthetic primer”
ccgggacttggagaagcactgca

SEQ ID NO: 733 - DNA, 20 bp, Artificial Sequence
  source: /note=“Description of Artificial Sequence Synthetic primer”
tcactaccagctgacatccg

SEQ ID NO: 734 - DNA, 20 bp, Artificial Sequence
  source: /note=“Description of Artificial Sequence Synthetic primer”
agatggggctattgagggtc

SEQ ID NO: 735 - DNA, 24 bp, Artificial Sequence
  source: /note=“Description of Artificial Sequence Synthetic primer”
cttctccaggtagccctcaggcag

SEQ ID NO: 736 - DNA, 20 bp, Artificial Sequence
  source: /note=“Description of Artificial Sequence Synthetic primer”
ccagctctccttccagctac

SEQ ID NO: 737 - DNA, 20 bp, Artificial Sequence
  source: /note=“Description of Artificial Sequence Synthetic primer”
gggtggctctcacttagctc

SEQ ID NO: 738 - DNA, 24 bp, Artificial Sequence
  source: /note=“Description of Artificial Sequence Synthetic primer”
atcaatgtccctgtccgagtgctg

SEQ ID NO: 739 - DNA, 19 bp, Artificial Sequence
  source: /note=“Description of Artificial Sequence Synthetic primer”
gagaagcacgccatgaaac

SEQ ID NO: 740 - DNA, 20 bp, Artificial Sequence
  source: /note=“Description of Artificial Sequence Synthetic primer”
ggctgtgagctgctgaatct

SEQ ID NO: 741 - DNA, 24 bp, Artificial Sequence
  source: /note=“Description of Artificial Sequence Synthetic primer”
tgactcatcatttccatgacggcc

SEQ ID NO: 742 - DNA, 24 bp, Artificial Sequence
  source: /note=“Description of Artificial Sequence Synthetic primer”
agagccagttgctgtagaactcaa

SEQ ID NO: 743 - DNA, 21 bp, Artificial Sequence
  source: /note=“Description of Artificial Sequence Synthetic primer”
ctgggcctacacagtccttca

SEQ ID NO: 744 - DNA, 27 bp, Artificial Sequence
  source: /note=“Description of Artificial Sequence Synthetic primer”
tctctgctgggcaaggatgttctgttc

SEQ ID NO: 745 - DNA, 20 bp, Artificial Sequence
  source: /note=“Description of Artificial Sequence Synthetic primer”
gacattgtggtaggggaagg

SEQ ID NO: 746 - DNA, 20 bp, Artificial Sequence
  source: /note=“Description of Artificial Sequence Synthetic primer”
cggcagtcaaagataattgg

SEQ ID NO: 747 - DNA, 26 bp, Artificial Sequence
  source: /note=“Description of Artificial Sequence Synthetic primer”
tcattttctcatcccgtgggtacaga

SEQ ID NO: 748 - DNA, 23 bp, Artificial Sequence
  source: /note=“Description of Artificial Sequence Synthetic primer”
cacactagcattttctccgcata

SEQ ID NO: 749 - DNA, 21 bp, Artificial Sequence
  source: /note=“Description of Artificial Sequence Synthetic primer”
ataccaatcaccgcacaaacc

SEQ ID NO: 750 - DNA, 30 bp, Artificial Sequence
  source: /note=“Description of Artificial Sequence Synthetic primer”
tgcgctgtgactggacttaacaaatagcct

SEQ ID NO: 751 - DNA, 20 bp, Artificial Sequence
  source: /note=“Description of Artificial Sequence Synthetic primer”
ctgctcaaggacaccgtcta

SEQ ID NO: 752 - DNA, 18 bp, Artificial Sequence
  source: /note=“Description of Artificial Sequence Synthetic primer”
ggatccactctggagggc

SEQ ID NO: 753 - DNA, 24 bp, Artificial Sequence
  source: /note=“Description of Artificial Sequence Synthetic primer”
tacactcgggtcccatcgaagtcc

SEQ ID NO: 754 - DNA, 19 bp, Artificial Sequence
  source: /note=“Description of Artificial Sequence Synthetic primer”
tggggacattccctttgag

SEQ ID NO: 755 - DNA, 18 bp, Artificial Sequence
  source: /note=“Description of Artificial Sequence Synthetic primer”
gacatgggctgggaagtg

SEQ ID NO: 756 - DNA, 24 bp, Artificial Sequence
  source: /note=“Description of Artificial Sequence Synthetic primer”
cagcttccagaatctcctggtccc

SEQ ID NO: 757 - DNA, 20 bp, Artificial Sequence
  source: /note=“Description of Artificial Sequence Synthetic primer”
cccatggatgctcctctgaa

SEQ ID NO: 758 - DNA, 20 bp, Artificial Sequence
  source: /note=“Description of Artificial Sequence Synthetic primer”
ccggtggctaccagacattg

SEQ ID NO: 759 - DNA, 22 bp, Artificial Sequence
  source: /note=“Description of Artificial Sequence Synthetic primer”
cattgactgccgaggccccatg

SEQ ID NO: 760 - DNA, 18 bp, Artificial Sequence
  source: /note=“Description of Artificial Sequence Synthetic primer”
ccaagttctgggtggtgg

SEQ ID NO: 761 - DNA, 19 bp, Artificial Sequence
  source: /note=“Description of Artificial Sequence Synthetic primer”
gtgaacgccagtccatgtt

SEQ ID NO: 762 - DNA, 24 bp, Artificial Sequence
  source: /note=“Description of Artificial Sequence Synthetic primer”
ccagacccacttctacctgggcag

SEQ ID NO: 763 - DNA, 26 bp, Artificial Sequence
  source: /note=“Description of Artificial Sequence Synthetic primer”
aatgaatacagtattcccaagcacat

SEQ ID NO: 764 - DNA, 23 bp, Artificial Sequence
  source: /note=“Description of Artificial Sequence Synthetic primer”
tgtctgaagcatcttctggatga

SEQ ID NO: 765 - DNA, 18 bp, Artificial Sequence
  source: /note=“Description of Artificial Sequence Synthetic primer”
aaccccgtggccgcctcc

SEQ ID NO: 766 - DNA, 21 bp, Artificial Sequence
  source: /note=“Description of Artificial Sequence Synthetic primer”
cttacggttttcgtggagaag

SEQ ID NO: 767 - DNA, 20 bp, Artificial Sequence
  source: /note=“Description of Artificial Sequence Synthetic primer”
agcagccgttcttgttgtaa

SEQ ID NO: 768 - DNA, 29 bp, Artificial Sequence
  source: /note=“Description of Artificial Sequence Synthetic primer”
cctcagctatacaacaaattgaccccaag

SEQ ID NO: 769 - DNA, 20 bp, Artificial Sequence
  source: /note=“Description of Artificial Sequence Synthetic primer”
gatgtggactgccattcaaa

SEQ ID NO: 770 - DNA, 20 bp, Artificial Sequence
  source: /note=“Description of Artificial Sequence Synthetic primer”
tgcgagattagttggctgag

SEQ ID NO: 771 - DNA, 28 bp, Artificial Sequence
  source: /note=“Description of Artificial Sequence Synthetic primer”
tcgaaatttacatccggtatcttcctgg

SEQ ID NO: 772 - DNA, 19 bp, Artificial Sequence
  source: /note=“Description of Artificial Sequence Synthetic primer”
ccacataccgtccagccta

SEQ ID NO: 773 - DNA, 18 bp, Artificial Sequence
  source: /note=“Description of Artificial Sequence Synthetic primer”
gaggtcatgtgcgggagt

SEQ ID NO: 774 - DNA, 24 bp, Artificial Sequence
  source: /note=“Description of Artificial Sequence Synthetic primer”
ccgctcctcttcttcgttctccag

SEQ ID NO: 775 - DNA, 20 bp, Artificial Sequence
  source: /note=“Description of Artificial Sequence Synthetic primer”
gcaatcatggaacttgacga

SEQ ID NO: 776 - DNA, 18 bp, Artificial Sequence
  source: /note=“Description of Artificial Sequence Synthetic primer”
atgtggctcgcctctacg

SEQ ID NO: 777 - DNA, 24 bp, Artificial Sequence
  source: /note=“Description of Artificial Sequence Synthetic primer”
tttcttgcagtttgacccagcacc

SEQ ID NO: 778 - DNA, 20 bp, Artificial Sequence
  source: /note=“Description of Artificial Sequence Synthetic primer”
gcatcaggctgtcattatgg

SEQ ID NO: 779 - DNA, 20 bp, Artificial Sequence
  source: /note=“Description of Artificial Sequence Synthetic primer”
agtagttgtgctgcccttcc

SEQ ID NO: 780 - DNA, 28 bp, Artificial Sequence
  source: /note=“Description of Artificial Sequence Synthetic primer”
tgtccttacctgtgggagctgtaaggtc

SEQ ID NO: 781 - DNA, 19 bp, Artificial Sequence
  source: /note=“Description of Artificial Sequence Synthetic primer”
aggactgggacccatgaac

SEQ ID NO: 782 - DNA, 20 bp, Artificial Sequence
  source: /note=“Description of Artificial Sequence Synthetic primer”
cccataatcctgagcaatgg

SEQ ID NO: 783 - DNA, 24 bp, Artificial Sequence
  source: /note=“Description of Artificial Sequence Synthetic primer”
tcctttggtatcagacccgaagcg

SEQ ID NO: 784 - DNA, 20 bp, Artificial Sequence
  source: /note=“Description of Artificial Sequence Synthetic primer”
ggtgtccttcgccagatcac

SEQ ID NO: 785 - DNA, 18 bp, Artificial Sequence
  source: /note=“Description of Artificial Sequence Synthetic primer”
cagccgcagagcctcatc

SEQ ID NO: 786 - DNA, 26 bp, Artificial Sequence
  source: /note=“Description of Artificial Sequence Synthetic primer”
ttaatgatttgcctgtgggacgctcc

SEQ ID NO: 787 - DNA, 21 bp, Artificial Sequence
  source: /note=“Description of Artificial Sequence Synthetic primer”
caagcaatgcgtcatcaatgt

SEQ ID NO: 788 - DNA, 20 bp, Artificial Sequence
  source: /note=“Description of Artificial Sequence Synthetic primer”
gtaaatccgccccctcttct

SEQ ID NO: 789 - DNA, 25 bp, Artificial Sequence
  source: /note=“Description of Artificial Sequence Synthetic primer”
cagcctctgcggaatggatcacact

SEQ ID NO: 790 - DNA, 20 bp, Artificial Sequence
  source: /note=“Description of Artificial Sequence Synthetic primer”
ctgacacttgccgcagagaa

SEQ ID NO: 791 - DNA, 20 bp, Artificial Sequence
  source: /note=“Description of Artificial Sequence Synthetic primer”
aggtggtccttggtctggaa

SEQ ID NO: 792 - DNA, 26 bp, Artificial Sequence
  source: /note=“Description of Artificial Sequence Synthetic primer”
ccctttctcacccacctcatctgcac

SEQ ID NO: 793 - DNA, 18 bp, Artificial Sequence
  source: /note=“Description of Artificial Sequence Synthetic primer”
gggttctagacgcccctc

SEQ ID NO: 794 - DNA, 21 bp, Artificial Sequence
  source: /note=“Description of Artificial Sequence Synthetic primer”
ggacggctgtagaggctgtat

SEQ ID NO: 795 - DNA, 24 bp, Artificial Sequence
  source: /note=“Description of Artificial Sequence Synthetic primer”
caagcgttcctggccttctgaact

SEQ ID NO: 796 - DNA, 21 bp, Artificial Sequence
  source: /note=“Description of Artificial Sequence Synthetic primer”
ctccacctatgagcgtctgtc

SEQ ID NO: 797 - DNA, 20 bp, Artificial Sequence
  source: /note=“Description of Artificial Sequence Synthetic primer”
cacactttccctccttttgg

SEQ ID NO: 798 - DNA, 24 bp, Artificial Sequence
  source: /note=“Description of Artificial Sequence Synthetic primer”
tcctgttaacatcccaagcccaca

SEQ ID NO: 799 - DNA, 19 bp, Artificial Sequence
  source: /note=“Description of Artificial Sequence Synthetic primer”
gccctcccagtgtgcaaat

SEQ ID NO: 800 - DNA, 25 bp, Artificial Sequence
  source: /note=“Description of Artificial Sequence Synthetic primer”
cgtcgatggtattaggatagaagca

SEQ ID NO: 801 - DNA, 23 bp, Artificial Sequence
  source: /note=“Description of Artificial Sequence Synthetic primer”
tgctgtttcgacgacaccgttcg

SEQ ID NO: 802 - DNA, 25 bp, Artificial Sequence
  source: /note=“Description of Artificial Sequence Synthetic primer”
tggctaagtgaagatgacaatcatg

SEQ ID NO: 803 - DNA, 25 bp, Artificial Sequence
  source: /note=“Description of Artificial Sequence Synthetic primer”
tgcacatatcattacaccagttcgt

SEQ ID NO: 804 - DNA, 29 bp, Artificial Sequence
  source: /note=“Description of Artificial Sequence Synthetic primer”
cctttccagctttacagtgaattgctgca

SEQ ID NO: 805 - DNA, 23 bp, Artificial Sequence
  source: /note=“Description of Artificial Sequence Synthetic primer”
cgcttgcctaactcatactttcc

SEQ ID NO: 806 - DNA, 20 bp, Artificial Sequence
  source: /note=“Description of Artificial Sequence Synthetic primer”
ccattcagactgcgccactt

SEQ ID NO: 807 - DNA, 19 bp, Artificial Sequence
  source: /note=“Description of Artificial Sequence Synthetic primer”
tccacgcagcgtggcactg

SEQ ID NO: 808 - DNA, 28 bp, Artificial Sequence
  source: /note=“Description of Artificial Sequence Synthetic primer”
ggctactctgatctatgttgataaggaa

SEQ ID NO: 809 - DNA, 20 bp, Artificial Sequence
  source: /note=“Description of Artificial Sequence Synthetic primer”
gcttcagcccatccttagca

SEQ ID NO: 810 - DNA, 22 bp, Artificial Sequence
  source: /note=“Description of Artificial Sequence Synthetic primer”
cacacgggtgcctggttctcca

SEQ ID NO: 811 - DNA, 18 bp, Artificial Sequence
  source: /note=“Description of Artificial Sequence Synthetic primer”
gggacactgcgggacaag

SEQ ID NO: 812 - DNA, 18 bp, Artificial Sequence
  source: /note=“Description of Artificial Sequence Synthetic primer”
gcccatggcgtctctgaa

SEQ ID NO: 813 - DNA, 25 bp, Artificial Sequence
  source: /note=“Description of Artificial Sequence Synthetic primer”
cggttccggagtctcaccactgcat

SEQ ID NO: 814 - DNA, 19 bp, Artificial Sequence
  source: /note=“Description of Artificial Sequence Synthetic primer”
ctgaaggaccctacgctcg

SEQ ID NO: 815 - DNA, 19 bp, Artificial Sequence
  source: /note=“Description of Artificial Sequence Synthetic primer”
atgcaaagccagtgtgctc

SEQ ID NO: 816 - DNA, 24 bp, Artificial Sequence
  source: /note=“Description of Artificial Sequence Synthetic primer”
cttctcaaagtgaggtgccaggcc

SEQ ID NO: 817 - DNA, 21 bp, Artificial Sequence
  source: /note=“Description of Artificial Sequence Synthetic primer”
gcgacagctcctctagttcca

SEQ ID NO: 818 - DNA, 19 bp, Artificial Sequence
  source: /note=“Description of Artificial Sequence Synthetic primer”
ttcccgaagtctccgcccg

SEQ ID NO: 819 - DNA, 22 bp, Artificial Sequence
  source: /note=“Description of Artificial Sequence Synthetic primer”
ggaacaccagcttgaatttcct

SEQ ID NO: 820 - DNA, 20 bp, Artificial Sequence
  source: /note=“Description of Artificial Sequence Synthetic primer”
gaggagtggtgacagtctgc

SEQ ID NO: 821 - DNA, 20 bp, Artificial Sequence
  source: /note=“Description of Artificial Sequence Synthetic primer”
gctgcagaaatcaaagtcca

SEQ ID NO: 822 - DNA, 27 bp, Artificial Sequence
  source: /note=“Description of Artificial Sequence Synthetic primer”
tcaatacacaggaacctgaggagaccc

SEQ ID NO: 823 - DNA, 23 bp, Artificial Sequence
  source: /note=“Description of Artificial Sequence Synthetic primer”
agctagcctcagtgacacacatg

SEQ ID NO: 824 - DNA, 18 bp, Artificial Sequence
  source: /note=“Description of Artificial Sequence Synthetic primer”
ccggatctgacggctgtt

SEQ ID NO: 825 - DNA, 24 bp, Artificial Sequence
  source: /note=“Description of Artificial Sequence Synthetic primer”
acacaacgtcggcagtgcaacctg

SEQ ID NO: 826 - DNA, 20 bp, Artificial Sequence
  source: /note=“Description of Artificial Sequence Synthetic primer”
cgtcgtatgcgagagtctgt

SEQ ID NO: 827 - DNA, 20 bp, Artificial Sequence
  source: /note=“Description of Artificial Sequence Synthetic primer”
tgaaggcgtgaggtgtagaa

SEQ ID NO: 828 - DNA, 26 bp, Artificial Sequence
  source: /note=“Description of Artificial Sequence Synthetic primer”
tccaggatgcctgttagttctcagca

SEQ ID NO: 829 - DNA, 26 bp, Artificial Sequence
  source: /note=“Description of Artificial Sequence Synthetic primer”
ggtgtcagatataaatgtgcaaatgc

SEQ ID NO: 830 - DNA, 24 bp, Artificial Sequence
  source: /note=“Description of Artificial Sequence Synthetic primer”
ttcgatattgccagcagctataaa

SEQ ID NO: 831 - DNA, 28 bp, Artificial Sequence
  source: /note=“Description of Artificial Sequence Synthetic primer”
tgctgtcctgtcggtctcagtacgttca

SEQ ID NO: 832 - DNA, 20 bp, Artificial Sequence
  source: /note=“Description of Artificial Sequence Synthetic primer”
tgtggatgctggattgattt

SEQ ID NO: 833 - DNA, 20 bp, Artificial Sequence
  source: /note=“Description of Artificial Sequence Synthetic primer”
aagcagcacttcctggtctt

SEQ ID NO: 834 - DNA, 25 bp, Artificial Sequence
  source: /note=“Description of Artificial Sequence Synthetic primer”
ccactggtgcagctgctaaatagca

SEQ ID NO: 835 - DNA, 20 bp, Artificial Sequence
  source: /note=“Description of Artificial Sequence Synthetic primer”
agtgggagacacctgacctt

SEQ ID NO: 836 - DNA, 20 bp, Artificial Sequence
  source: /note=“Description of Artificial Sequence Synthetic primer”
tgatctgggcattgtactcc

SEQ ID NO: 837 - DNA, 29 bp, Artificial Sequence
  source: /note=“Description of Artificial Sequence Synthetic primer”
ttgatcttctgctcaatctcagcttgaga

SEQ ID NO: 838 - DNA, 20 bp, Artificial Sequence
  source: /note=“Description of Artificial Sequence Synthetic primer”
cgaagcccttacaagtttcc

SEQ ID NO: 839 - DNA, 20 bp, Artificial Sequence
  source: /note=“Description of Artificial Sequence Synthetic primer”
ggactcttcaggggtgaaat

SEQ ID NO: 840 - DNA, 24 bp, Artificial Sequence
  source: /note=“Description of Artificial Sequence Synthetic primer”
cccttacggattcctggagggaac

SEQ ID NO: 841 - DNA, 20 bp, Artificial Sequence
  source: /note=“Description of Artificial Sequence Synthetic primer”
cccagtgtacgaggaacaag

SEQ ID NO: 842 - DNA, 20 bp, Artificial Sequence
  source: /note=“Description of Artificial Sequence Synthetic primer”
ttagcgaggaaggagttgct

SEQ ID NO: 843 - DNA, 24 bp, Artificial Sequence
  source: /note=“Description of Artificial Sequence Synthetic primer”
acagaccgagatctccaaccggac

SEQ ID NO: 844 - DNA, 18 bp, Artificial Sequence
  source: /note=“Description of Artificial Sequence Synthetic primer”
gggctgggtgagaatgtg

SEQ ID NO: 845 - DNA, 19 bp, Artificial Sequence
  source: /note=“Description of Artificial Sequence Synthetic primer”
cacaacatcctccggaatg

SEQ ID NO: 846 - DNA, 24 bp, Artificial Sequence
  source: /note=“Description of Artificial Sequence Synthetic primer”
ataccagggccggcttcttcctct

SEQ ID NO: 847 - DNA, 18 bp, Artificial Sequence
  source: /note=“Description of Artificial Sequence Synthetic primer”
cctacaagtcctggggca

SEQ ID NO: 848 - DNA, 20 bp, Artificial Sequence
  source: /note=“Description of Artificial Sequence Synthetic primer”
tgaggtcaggctcacacagt

SEQ ID NO: 849 - DNA, 24 bp, Artificial Sequence
  source: /note=“Description of Artificial Sequence Synthetic primer”
tggagccccaagcagtgttaatcc

SEQ ID NO: 850 - DNA, 20 bp, Artificial Sequence
  source: /note=“Description of Artificial Sequence Synthetic primer”
gcgaggagctctacttgctc

SEQ ID NO: 851 - DNA, 18 bp, Artificial Sequence
  source: /note=“Description of Artificial Sequence Synthetic primer”
gccctgctgaacaccact

SEQ ID NO: 852 - DNA, 24 bp, Artificial Sequence
  source: /note=“Description of Artificial Sequence Synthetic primer”
tgtcctctttctgcaccttgtcgc

SEQ ID NO: 853 - DNA, 21 bp, Artificial Sequence
  source: /note=“Description of Artificial Sequence Synthetic primer”
aagcatgaacaggacttgacc

SEQ ID NO: 854 - DNA, 18 bp, Artificial Sequence
  source: /note=“Description of Artificial Sequence Synthetic primer”
cctccccaagtcagttgc

SEQ ID NO: 855 - DNA, 24 bp, Artificial Sequence
  source: /note=“Description of Artificial Sequence Synthetic primer”
ctttccaacccctggggaagacat

SEQ ID NO: 856 - DNA, 18 bp, Artificial Sequence
  source: /note=“Description of Artificial Sequence Synthetic primer”
cccgttcggtctgaggaa

SEQ ID NO: 857 - DNA, 22 bp, Artificial Sequence
  source: /note=“Description of Artificial Sequence Synthetic primer”
gagcactcaaggtagccaaagg

SEQ ID NO: 858 - DNA, 19 bp, Artificial Sequence
  source: /note=“Description of Artificial Sequence Synthetic primer”
tccggttcgccatgtcccg

SEQ ID NO: 859 - DNA, 20 bp, Artificial Sequence
  source: /note=“Description of Artificial Sequence Synthetic primer”
ccagacgagcgattagaagc

SEQ ID NO: 860 - DNA, 20 bp, Artificial Sequence
  source: /note=“Description of Artificial Sequence Synthetic primer”
tcctcctcttcctcctcctc

SEQ ID NO: 861 - DNA, 23 bp, Artificial Sequence
  source: /note=“Description of Artificial Sequence Synthetic primer”
tgtgaggtgaatgatttggggga

SEQ ID NO: 862 - DNA, 22 bp, Artificial Sequence
  source: /note=“Description of Artificial Sequence Synthetic primer”
tgtgcacataggaatgagcttc

SEQ ID NO: 863 - DNA, 20 bp, Artificial Sequence
  source: /note=“Description of Artificial Sequence Synthetic primer”
gtttagcacgcaattgctca

SEQ ID NO: 864 - DNA, 24 bp, Artificial Sequence
  source: /note=“Description of Artificial Sequence Synthetic primer”
tcactctctttgctggccaactgc

SEQ ID NO: 865 - DNA, 19 bp, Artificial Sequence
  source: /note=“Description of Artificial Sequence Synthetic primer”
gatctggcactgtggttcc

SEQ ID NO: 866 - DNA, 20 bp, Artificial Sequence
  source: /note=“Description of Artificial Sequence Synthetic primer”
tgacagcggaagtggtattg

SEQ ID NO: 867 - DNA, 22 bp, Artificial Sequence
  source: /note=“Description of Artificial Sequence Synthetic primer”
caccgccagccactgtcttcat

SEQ ID NO: 868 - DNA, 24 bp, Artificial Sequence
  source: /note=“Description of Artificial Sequence Synthetic primer”
ccattctatcatcaacgggtacaa

SEQ ID NO: 869 - DNA, 23 bp, Artificial Sequence
  source: /note=“Description of Artificial Sequence Synthetic primer”
tcagcaagtgggaaggtgtaatc

SEQ ID NO: 870 - DNA, 25 bp, Artificial Sequence
  source: /note=“Description of Artificial Sequence Synthetic primer”
tctccacagacaaggccaggactcg

SEQ ID NO: 871 - DNA, 19 bp, Artificial Sequence
  source: /note=“Description of Artificial Sequence Synthetic primer”
ctgtcttcctgttggccct

SEQ ID NO: 872 - DNA, 20 bp, Artificial Sequence
  source: /note=“Description of Artificial Sequence Synthetic primer”
gtgaggtagtgagtgggcgt

SEQ ID NO: 873 - DNA, 24 bp, Artificial Sequence
  source: /note=“Description of Artificial Sequence Synthetic primer”
acaatcatagccagcacctgggct

SEQ ID NO: 874 - DNA, 25 bp, Artificial Sequence
  source: /note=“Description of Artificial Sequence Synthetic primer”
gctcattatgaaaaacatcccaaac

SEQ ID NO: 875 - DNA, 26 bp, Artificial Sequence
  source: /note=“Description of Artificial Sequence Synthetic primer”
aagaaacagaagttgtctggctttct

SEQ ID NO: 876 - DNA, 24 bp, Artificial Sequence
  source: /note=“Description of Artificial Sequence Synthetic primer”
cacaccaaccaataatttcgcatt

SEQ ID NO: 877 - DNA, 20 bp, Artificial Sequence
  source: /note=“Description of Artificial Sequence Synthetic primer”
gctctgttgtgtcctgttgc

SEQ ID NO: 878 - DNA, 21 bp, Artificial Sequence
  source: /note=“Description of Artificial Sequence Synthetic primer”
gtacggagaagccacttcaca

SEQ ID NO: 879 - DNA, 23 bp, Artificial Sequence
  source: /note=“Description of Artificial Sequence Synthetic primer”
tcagtcacaggaaggccagagcc

SEQ ID NO: 880 - DNA, 18 bp, Artificial Sequence
  source: /note=“Description of Artificial Sequence Synthetic primer”
cgaggagatgcctgtgga

SEQ ID NO: 881 - DNA, 18 bp, Artificial Sequence
  source: /note=“Description of Artificial Sequence Synthetic primer”
caacgccctggtcactct

SEQ ID NO: 882 - DNA, 24 bp, Artificial Sequence
  source: /note=“Description of Artificial Sequence Synthetic primer”
ctgttccacagcaagctctgcctc

SEQ ID NO: 883 - DNA, 20 bp, Artificial Sequence
  source: /note=“Description of Artificial Sequence Synthetic primer”
acaccaaaatgccatctcaa

SEQ ID NO: 884 - DNA, 20 bp, Artificial Sequence
  source: /note=“Description of Artificial Sequence Synthetic primer”
tttatccccagcgaatttgt

SEQ ID NO: 885 - DNA, 24 bp, Artificial Sequence
  source: /note=“Description of Artificial Sequence Synthetic primer”
cacgccatggaaaccatgatgttt

SEQ ID NO: 886 - DNA, 18 bp, Artificial Sequence
  source: /note=“Description of Artificial Sequence Synthetic primer”
cttctgagcccgtctccc

SEQ ID NO: 887 - DNA, 18 bp, Artificial Sequence
  source: /note=“Description of Artificial Sequence Synthetic primer”
gagagctccccttccgag

SEQ ID NO: 888 - DNA, 24 bp, Artificial Sequence
  source: /note=“Description of Artificial Sequence Synthetic primer”
cgcttctggagcagcttcctcaac

SEQ ID NO: 889 - DNA, 20 bp, Artificial Sequence
  source: /note=“Description of Artificial Sequence Synthetic primer”
cgcgagcccctcattataca

SEQ ID NO: 890 - DNA, 19 bp, Artificial Sequence
  source: /note=“Description of Artificial Sequence Synthetic primer”
cactcgccgttgacatcct

SEQ ID NO: 891 - DNA, 22 bp, Artificial Sequence
  source: /note=“Description of Artificial Sequence Synthetic primer”
ctccccacagcgcatcgaggaa

SEQ ID NO: 892 - DNA, 20 bp, Artificial Sequence
  source: /note=“Description of Artificial Sequence Synthetic primer”
gagagagccagtctgatcca

SEQ ID NO: 893 - DNA, 19 bp, Artificial Sequence
  source: /note=“Description of Artificial Sequence Synthetic primer”
aggctgccatgtcctcata

SEQ ID NO: 894 - DNA, 22 bp, Artificial Sequence
  source: /note=“Description of Artificial Sequence Synthetic primer”
ctgctctgccagcttggccttc

SEQ ID NO: 895 - DNA, 23 bp, Artificial Sequence
  source: /note=“Description of Artificial Sequence Synthetic primer”
cagtggagaccagttgggtagtg

SEQ ID NO: 896 - DNA, 20 bp, Artificial Sequence
  source: /note=“Description of Artificial Sequence Synthetic primer”
ccttgaagagcgtcccatca

SEQ ID NO: 897 - DNA, 28 bp, Artificial Sequence
  source: /note=“Description of Artificial Sequence Synthetic primer”
cacacatgcagagcttgtagcgtaccca

SEQ ID NO: 898 - DNA, 18 bp, Artificial Sequence
  source: /note=“Description of Artificial Sequence Synthetic primer”
tccgcaagacacctcctg

SEQ ID NO: 899 - DNA, 18 bp, Artificial Sequence
  source: /note=“Description of Artificial Sequence Synthetic primer”
tgaagtcatccttggccc

SEQ ID NO: 900 - DNA, 23 bp, Artificial Sequence
  source: /note=“Description of Artificial Sequence Synthetic primer”
tgtcctgtccttctgcaggaggc

SEQ ID NO: 901 - DNA, 20 bp, Artificial Sequence
  source: /note=“Description of Artificial Sequence Synthetic primer”
tgcattcgttggtttctctt

SEQ ID NO: 902 - DNA, 20 bp, Artificial Sequence
  source: /note=“Description of Artificial Sequence Synthetic primer”
tttctgaatggcaaactgct

SEQ ID NO: 903 - DNA, 30 bp, Artificial Sequence
  source: /note=“Description of Artificial Sequence Synthetic primer”
tgcacctccttcagaagacttatttttgtg

SEQ ID NO: 904 - DNA, 20 bp, Artificial Sequence
  source: /note=“Description of Artificial Sequence Synthetic primer”
ccaacaccttcttggcttct

SEQ ID NO: 905 - DNA, 20 bp, Artificial Sequence
  source: /note=“Description of Artificial Sequence Synthetic primer”
ctgttatcccaacccaaacc

SEQ ID NO: 906 - DNA, 24 bp, Artificial Sequence
  source: /note=“Description of Artificial Sequence Synthetic primer”
cctgtgttcttgctgagcaccctc

SEQ ID NO: 907 - DNA, 20 bp, Artificial Sequence
  source: /note=“Description of Artificial Sequence Synthetic primer”
agctggggtgtctgtttcat

SEQ ID NO: 908 - DNA, 20 bp, Artificial Sequence
  source: /note=“Description of Artificial Sequence Synthetic primer”
acagcaaggcgagcataaat

SEQ ID NO: 909 - DNA, 23 bp, Artificial Sequence
  source: /note=“Description of Artificial Sequence Synthetic primer”
cctgacttcaggtcaagggatgg

SEQ ID NO: 910 - DNA, 18 bp, Artificial Sequence
  source: /note=“Description of Artificial Sequence Synthetic primer”
gtggggagcccatcatct

SEQ ID NO: 911 - DNA, 20 bp, Artificial Sequence
  source: /note=“Description of Artificial Sequence Synthetic primer”
ccagtccacactgagtgcat

SEQ ID NO: 912 - DNA, 24 bp, Artificial Sequence
  source: /note=“Description of Artificial Sequence Synthetic primer”
ccaagccatcacaggctctgcata

SEQ ID NO: 913 - DNA, 20 bp, Artificial Sequence
  source: /note=“Description of Artificial Sequence Synthetic primer”
tctgccagtgctgaattctt

SEQ ID NO: 914 - DNA, 20 bp, Artificial Sequence
  source: /note=“Description of Artificial Sequence Synthetic primer”
ttcgaaccttagcagcttcc

SEQ ID NO: 915 - DNA, 24 bp, Artificial Sequence
  source: /note=“Description of Artificial Sequence Synthetic primer”
tgctgacattgccctggctcctat

SEQ ID NO: 916 - DNA, 20 bp, Artificial Sequence
  source: /note=“Description of Artificial Sequence Synthetic primer”
cccaactcaggtccttggta

SEQ ID NO: 917 - DNA, 18 bp, Artificial Sequence
  source: /note=“Description of Artificial Sequence Synthetic primer”
caagagggtcaccccaag

SEQ ID NO: 918 - DNA, 24 bp, Artificial Sequence
  source: /note=“Description of Artificial Sequence Synthetic primer”
atcctgcaagccaatcccagtcat

SEQ ID NO: 919 - DNA, 22 bp, Artificial Sequence
  source: /note=“Description of Artificial Sequence Synthetic primer”
agatgcatacttggaagcacag

SEQ ID NO: 920 - DNA, 22 bp, Artificial Sequence
  source: /note=“Description of Artificial Sequence Synthetic primer”
aacctaggaccaggtaaccaca

SEQ ID NO: 921 - DNA, 24 bp, Artificial Sequence
  source: /note=“Description of Artificial Sequence Synthetic primer”
catatcacactgggaggcaatgca

SEQ ID NO: 922 - DNA, 18 bp, Artificial Sequence
  source: /note=“Description of Artificial Sequence Synthetic primer”
gcgcagaggccagttaaa

SEQ ID NO: 923 - DNA, 18 bp, Artificial Sequence
  source: /note=“Description of Artificial Sequence Synthetic primer”
agctgagctgtgggttgc

SEQ ID NO: 924 - DNA, 24 bp, Artificial Sequence
  source: /note=“Description of Artificial Sequence Synthetic primer”
agatcacctcctcgaacccactcc

SEQ ID NO: 925 - DNA, 19 bp, Artificial Sequence
  source: /note=“Description of Artificial Sequence Synthetic primer”
ggctggccaaacataagca

SEQ ID NO: 926 - DNA, 26 bp, Artificial Sequence
  source: /note=“Description of Artificial Sequence Synthetic primer”
tccttgtcacagtatttacagctgaa

SEQ ID NO: 927 - DNA, 29 bp, Artificial Sequence
  source: /note=“Description of Artificial Sequence Synthetic primer”
ctgcactgcgatgcccagtctagaaaatc

SEQ ID NO: 928 - DNA, 21 bp, Artificial Sequence
  source: /note=“Description of Artificial Sequence Synthetic primer”
agtgacaaatgttggaggagc

SEQ ID NO: 929 - DNA, 19 bp, Artificial Sequence
  source: /note=“Description of Artificial Sequence Synthetic primer”
ccctccactgtcttctggg

SEQ ID NO: 930 - DNA, 24 bp, Artificial Sequence
  source: /note=“Description of Artificial Sequence Synthetic primer”
tactgctgtcacacccgtcaccac

SEQ ID NO: 931 - DNA, 19 bp, Artificial Sequence
  source: /note=“Description of Artificial Sequence Synthetic primer”
acccaccatggatgtcttc

SEQ ID NO: 932 - DNA, 19 bp, Artificial Sequence
  source: /note=“Description of Artificial Sequence Synthetic primer”
cctgcttggtcttttccac

SEQ ID NO: 933 - DNA, 24 bp, Artificial Sequence
  source: /note=“Description of Artificial Sequence Synthetic primer”
aagaagggcttctccatcgccaag

SEQ ID NO: 934 - DNA, 20 bp, Artificial Sequence
  source: /note=“Description of Artificial Sequence Synthetic primer”
tgaagagaggcatgttggag

SEQ ID NO: 935 - DNA, 20 bp, Artificial Sequence
  source: /note=“Description of Artificial Sequence Synthetic primer”
aatagacacatcggccacac

SEQ ID NO: 936 - DNA, 24 bp, Artificial Sequence
  source: /note=“Description of Artificial Sequence Synthetic primer”
tttgtcagcagtcacattgcccaa

SEQ ID NO: 937 - DNA, 24 bp, Artificial Sequence
  source: /note=“Description of Artificial Sequence Synthetic primer”
atacagcaccaatggaaagatcac

SEQ ID NO: 938 - DNA, 24 bp, Artificial Sequence
  source: /note=“Description of Artificial Sequence Synthetic primer”
tgtctgtaagagccctcagtttga

SEQ ID NO: 939 - DNA, 24 bp, Artificial Sequence
  source: /note=“Description of Artificial Sequence Synthetic primer”
ttcgacgactacatcgcctgctgc

SEQ ID NO: 940 - DNA, 20 bp, Artificial Sequence
  source: /note=“Description of Artificial Sequence Synthetic primer”
gggctcagctttcagaagtg

SEQ ID NO: 941 - DNA, 20 bp, Artificial Sequence
  source: /note=“Description of Artificial Sequence Synthetic primer”
acatgttcagctggtccaca

SEQ ID NO: 942 - DNA, 25 bp, Artificial Sequence
  source: /note=“Description of Artificial Sequence Synthetic primer”
tggcagttttcttctgtcaccaaaa

SEQ ID NO: 943 - DNA, 20 bp, Artificial Sequence
  source: /note=“Description of Artificial Sequence Synthetic primer”
tcacatgccactttggtgtt

SEQ ID NO: 944 - DNA, 20 bp, Artificial Sequence
  source: /note=“Description of Artificial Sequence Synthetic primer”
cttgcaggaagcggctatac

SEQ ID NO: 945 - DNA, 22 bp, Artificial Sequence
  source: /note=“Description of Artificial Sequence Synthetic primer”
tcctgggagagattgaccagca

SEQ ID NO: 946 - DNA, 19 bp, Artificial Sequence
  source: /note=“Description of Artificial Sequence Synthetic primer”
caagagggactcggactgc

SEQ ID NO: 947 - DNA, 21 bp, Artificial Sequence
  source: /note=“Description of Artificial Sequence Synthetic primer”
caggtcagtggagagattggt

SEQ ID NO: 948 - DNA, 24 bp, Artificial Sequence
  source: /note=“Description of Artificial Sequence Synthetic primer”
cctctgcacctctgagagcatgga

SEQ ID NO: 949 - DNA, 18 bp, Artificial Sequence
  source: /note=“Description of Artificial Sequence Synthetic primer”
ggactcggagacgctgtg

SEQ ID NO: 950 - DNA, 20 bp, Artificial Sequence
  source: /note=“Description of Artificial Sequence Synthetic primer”
gggatccttcgcaacttctt

SEQ ID NO: 951 - DNA, 24 bp, Artificial Sequence
  source: /note=“Description of Artificial Sequence Synthetic primer”
ttcttgaggatcttgacggccctc

SEQ ID NO: 952 - DNA, 20 bp, Artificial Sequence
  source: /note=“Description of Artificial Sequence Synthetic primer”
catcttccaggaggaccact

SEQ ID NO: 953 - DNA, 20 bp, Artificial Sequence
  source: /note=“Description of Artificial Sequence Synthetic primer”
tccgaccttcaatcatttca

SEQ ID NO: 954 - DNA, 24 bp, Artificial Sequence
  source: /note=“Description of Artificial Sequence Synthetic primer”
ctctgtggcaccctggactacctg

SEQ ID NO: 955 - DNA, 20 bp, Artificial Sequence
  source: /note=“Description of Artificial Sequence Synthetic primer”
aatacccaacgcacaaatga

SEQ ID NO: 956 - DNA, 20 bp, Artificial Sequence
  source: /note=“Description of Artificial Sequence Synthetic primer”
ggagacaatgcaaaccacac

SEQ ID NO: 957 - DNA, 23 bp, Artificial Sequence
  source: /note=“Description of Artificial Sequence Synthetic primer”
cacgttctctgccccgtttcttg

SEQ ID NO: 958 - DNA, 20 bp, Artificial Sequence
  source: /note=“Description of Artificial Sequence Synthetic primer”
cctggaggctgcaacatacc

SEQ ID NO: 959 - DNA, 23 bp, Artificial Sequence
  source: /note=“Description of Artificial Sequence Synthetic primer”
tacaatggctttggaggatagca

SEQ ID NO: 960 - DNA, 25 bp, Artificial Sequence
  source: /note=“Description of Artificial Sequence Synthetic primer”
atcctcctgaagcccttttcgcagc

SEQ ID NO: 961 - DNA, 20 bp, Artificial Sequence
  source: /note=“Description of Artificial Sequence Synthetic primer”
tgttttgattcccgggctta

SEQ ID NO: 962 - DNA, 24 bp, Artificial Sequence
  source: /note=“Description of Artificial Sequence Synthetic primer”
caaagctgtcagctctagcaaaag

SEQ ID NO: 963 - DNA, 28 bp, Artificial Sequence
  source: /note=“Description of Artificial Sequence Synthetic primer”
tgccttcttcctccctcacttctcacct

SEQ ID NO: 964 - DNA, 20 bp, Artificial Sequence
  source: /note=“Description of Artificial Sequence Synthetic primer”
cacccttggactggaaaact

SEQ ID NO: 965 - DNA, 20 bp, Artificial Sequence
  source: /note=“Description of Artificial Sequence Synthetic primer”
ccttgatgagctgttggttc

SEQ ID NO: 966 - DNA, 24 bp, Artificial Sequence
  source: /note=“Description of Artificial Sequence Synthetic primer”
cacacccggtctggacacagaaag

SEQ ID NO: 967 - DNA, 19 bp, Artificial Sequence
  source: /note=“Description of Artificial Sequence Synthetic primer”
gatcctcgcgtgagacaga

SEQ ID NO: 968 - DNA, 21 bp, Artificial Sequence
  source: /note=“Description of Artificial Sequence Synthetic primer”
cactttttctttgaccaaccg

SEQ ID NO: 969 - DNA, 23 bp, Artificial Sequence
  source: /note=“Description of Artificial Sequence Synthetic primer”
ttcaccacgccggtcttgatctt

SEQ ID NO: 970 - DNA, 20 bp, Artificial Sequence
  source: /note=“Description of Artificial Sequence Synthetic primer”
ctgttttcctggaggactgc

SEQ ID NO: 971 - DNA, 20 bp, Artificial Sequence
  source: /note=“Description of Artificial Sequence Synthetic primer”
actgtgtatgcggagctgtt

SEQ ID NO: 972 - DNA, 21 bp, Artificial Sequence
  source: /note=“Description of Artificial Sequence Synthetic primer”
ccactgccagcacgcagtcac

SEQ ID NO: 973 - DNA, 21 bp, Artificial Sequence
  source: /note=“Description of Artificial Sequence Synthetic primer”
cagccaggtgtacgagacatt

SEQ ID NO: 974 - DNA, 18 bp, Artificial Sequence
  source: /note=“Description of Artificial Sequence Synthetic primer”
acctcgtccagcacatcc

SEQ ID NO: 975 - DNA, 24 bp, Artificial Sequence
  source: /note=“Description of Artificial Sequence Synthetic primer”
ctcacctacagtgacgtcgtgggc

SEQ ID NO: 976 - DNA, 19 bp, Artificial Sequence
  source: /note=“Description of Artificial Sequence Synthetic primer”
tcccgagagaggaaagcat

SEQ ID NO: 977 - DNA, 18 bp, Artificial Sequence
  source: /note=“Description of Artificial Sequence Synthetic primer”
gtcgggtgcctgcattta

SEQ ID NO: 978 - DNA, 24 bp, Artificial Sequence
  source: /note=“Description of Artificial Sequence Synthetic primer”
atacacagtcccttcgtggctccc

SEQ ID NO: 979 - DNA, 22 bp, Artificial Sequence
  source: /note=“Description of Artificial Sequence Synthetic primer”
cctggttgaagcctgttaatgc

SEQ ID NO: 980 - DNA, 26 bp, Artificial Sequence
  source: /note=“Description of Artificial Sequence Synthetic primer”
tgcagacttctcatatttgctaaagg

SEQ ID NO: 981 - DNA, 23 bp, Artificial Sequence
  source: /note=“Description of Artificial Sequence Synthetic primer”
ccgctgggttttccacacgttga

SEQ ID NO: 982 - DNA, 22 bp, Artificial Sequence
  source: /note=“Description of Artificial Sequence Synthetic primer”
ccagtgtgtgtaacagggtcac

SEQ ID NO: 983 - DNA, 18 bp, Artificial Sequence
  source: /note=“Description of Artificial Sequence Synthetic primer”
tatagccttgggccaccc

SEQ ID NO: 984 - DNA, 24 bp, Artificial Sequence
  source: /note=“Description of Artificial Sequence Synthetic primer”
agaattcgacagccagatgctcca

SEQ ID NO: 985 - DNA, 20 bp, Artificial Sequence
  source: /note=“Description of Artificial Sequence Synthetic primer”
gccaactgctttcatttgtg

SEQ ID NO: 986 - DNA, 20 bp, Artificial Sequence
  source: /note=“Description of Artificial Sequence Synthetic primer”
actcaggcccatttccttta

SEQ ID NO: 987 - DNA, 28 bp, Artificial Sequence
  source: /note=“Description of Artificial Sequence Synthetic primer”
agggatctgaaccaatacagagcagaca

SEQ ID NO: 988 - DNA, 22 bp, Artificial Sequence
  source: /note=“Description of Artificial Sequence Synthetic primer”
catccgcaaagtgactgaagag

SEQ ID NO: 989 - DNA, 27 bp, Artificial Sequence
  source: /note=“Description of Artificial Sequence Synthetic primer”
gtactgaactccgttgtgatagcatag

SEQ ID NO: 990 - DNA, 22 bp, Artificial Sequence
  source: /note=“Description of Artificial Sequence Synthetic primer”
ccaatgagctgaggcggcctcc

SEQ ID NO: 991 - DNA, 20 bp, Artificial Sequence
  source: /note=“Description of Artificial Sequence Synthetic primer”
gccgggaagaccgtaattgt

SEQ ID NO: 992 - DNA, 18 bp, Artificial Sequence
  source: /note=“Description of Artificial Sequence Synthetic primer”
cagcggcaccaggttcag

SEQ ID NO: 993 - DNA, 26 bp, Artificial Sequence
  source: /note=“Description of Artificial Sequence Synthetic primer”
caaatggcttcctctggaaggtccca

SEQ ID NO: 994 - DNA, 20 bp, Artificial Sequence
  source: /note=“Description of Artificial Sequence Synthetic primer”
aatccaagggggagagtgat

SEQ ID NO: 995 - DNA, 20 bp, Artificial Sequence
  source: /note=“Description of Artificial Sequence Synthetic primer”
gtacagattttgcccgagga

SEQ ID NO: 996 - DNA, 26 bp, Artificial Sequence
  source: /note=“Description of Artificial Sequence Synthetic primer”
catatggactttgactcagctgtggc

SEQ ID NO: 997 - DNA, 18 bp, Artificial Sequence
  source: /note=“Description of Artificial Sequence Synthetic primer”
gtgatgccttccctgtgg

SEQ ID NO: 998 - DNA, 19 bp, Artificial Sequence
  source: /note=“Description of Artificial Sequence Synthetic primer”
tcaggtagtcgggtgggtt

SEQ ID NO: 999 - DNA, 24 bp, Artificial Sequence
  source: /note=“Description of Artificial Sequence Synthetic primer”
tgcttctccagcatcttcacctcg

SEQ ID NO: 1000 - DNA, 24 bp, Artificial Sequence
  source: /note=“Description of Artificial Sequence Synthetic primer”
ctatatgcagccagagatgtgaca

SEQ ID NO: 1001 - DNA, 24 bp, Artificial Sequence
  source: /note=“Description of Artificial Sequence Synthetic primer”
ccacgagtttcttactgagaatgg

SEQ ID NO: 1002 - DNA, 23 bp, Artificial Sequence
  source: /note=“Description of Artificial Sequence Synthetic primer”
acagcctgccactcatcacagcc

SEQ ID NO: 1003 - DNA, 20 bp, Artificial Sequence
  source: /note=“Description of Artificial Sequence Synthetic primer”
tgctgttgctgagtctgttg

SEQ ID NO: 1004 - DNA, 20 bp, Artificial Sequence
  source: /note=“Description of Artificial Sequence Synthetic primer”
cttgcctggcttcacagata

SEQ ID NO: 1005 - DNA, 24 bp, Artificial Sequence
  source: /note=“Description of Artificial Sequence Synthetic primer”
ccagtccccagaagaccatgtctg

SEQ ID NO: 1006 - DNA, 22 bp, Artificial Sequence
  source: /note=“Description of Artificial Sequence Synthetic primer”
ggtgtcgatattgtcatgaacc

SEQ ID NO: 1007 - DNA, 27 bp, Artificial Sequence
  source: /note=“Description of Artificial Sequence Synthetic primer”
gtaatctttgatgtacttcttgtaggc

SEQ ID NO: 1008 - DNA, 27 bp, Artificial Sequence
  source: /note=“Description of Artificial Sequence Synthetic primer”
tcacctgcaggaaacaagtttcacaaa

SEQ ID NO: 1009 - DNA, 21 bp, Artificial Sequence
  source: /note=“Description of Artificial Sequence Synthetic primer”
gacgctggtctggtgaagatg

SEQ ID NO: 1010 - DNA, 20 bp, Artificial Sequence
  source: /note=“Description of Artificial Sequence Synthetic primer”
tgggtggttgttggacaatg

SEQ ID NO: 1011 - DNA, 23 bp, Artificial Sequence
  source: /note=“Description of Artificial Sequence Synthetic primer”
ccaggaaaccacgagcctccagc

SEQ ID NO: 1012 - DNA, 22 bp, Artificial Sequence
  source: /note=“Description of Artificial Sequence Synthetic primer”
cttcacagtgctcctgcagtct

SEQ ID NO: 1013 - DNA, 21 bp, Artificial Sequence
  source: /note=“Description of Artificial Sequence Synthetic primer”
catctgcttcagctcgttggt

SEQ ID NO: 1014 - DNA, 26 bp, Artificial Sequence
  source: /note=“Description of Artificial Sequence Synthetic primer”
aagtacacgtaagttacagccacaca

SEQ ID NO: 1015 - DNA, 18 bp, Artificial Sequence
  source: /note=“Description of Artificial Sequence Synthetic primer”
gcctcggtgtgcctttca

SEQ ID NO: 1016 - DNA, 19 bp, Artificial Sequence
  source: /note=“Description of Artificial Sequence Synthetic primer”
cgtgatgtgcgcaatcatg

SEQ ID NO: 1017 - DNA, 22 bp, Artificial Sequence
  source: /note=“Description of Artificial Sequence Synthetic primer”
catcgccagctacgccctgctc

SEQ ID NO: 1018 - DNA, 19 bp, Artificial Sequence
  source: /note=“Description of Artificial Sequence Synthetic primer”
ctggtcagccttcagggac

SEQ ID NO: 1019 - DNA, 18 bp, Artificial Sequence
  source: /note=“Description of Artificial Sequence Synthetic primer”
cttcagttctgggctggc

SEQ ID NO: 1020 - DNA, 23 bp, Artificial Sequence
  source: /note=“Description of Artificial Sequence Synthetic primer”
ctgagcaccgcctggtctctttc

SEQ ID NO: 1021 - DNA, 23 bp, Artificial Sequence
  source: /note=“Description of Artificial Sequence Synthetic primer”
tgcttgtcagttgtcaacacctt

SEQ ID NO: 1022 - DNA, 23 bp, Artificial Sequence
  source: /note=“Description of Artificial Sequence Synthetic primer”
tggagtggcaagtatttgatgct

SEQ ID NO: 1023 - DNA, 23 bp, Artificial Sequence
  source: /note=“Description of Artificial Sequence Synthetic primer”
tggccaacctgcctgtttccagc

SEQ ID NO: 1024 - DNA, 19 bp, Artificial Sequence
  source: /note=“Description of Artificial Sequence Synthetic primer”
tgtcaccccgactcaacgt

SEQ ID NO: 1025 - DNA, 22 bp, Artificial Sequence
  source: /note=“Description of Artificial Sequence Synthetic primer”
acgtggactgagatgcattcac

SEQ ID NO: 1026 - DNA, 21 bp, Artificial Sequence
  source: /note=“Description of Artificial Sequence Synthetic primer”
agacgcaccgcccggactcac

SEQ ID NO: 1027 - DNA, 20 bp, Artificial Sequence
  source: /note=“Description of Artificial Sequence Synthetic primer”
agctcaacatgcgtgagtgt

SEQ ID NO: 1028 - DNA, 19 bp, Artificial Sequence
  source: /note=“Description of Artificial Sequence Synthetic primer”
attgccgatctggactcct

SEQ ID NO: 1029 - DNA, 23 bp, Artificial Sequence
  source: /note=“Description of Artificial Sequence Synthetic primer”
atctctatccacgtggggcaggc

SEQ ID NO: 1030 - DNA, 23 bp, Artificial Sequence
  source: /note=“Description of Artificial Sequence Synthetic primer”
ctcttacatcgaccgcctaagag

SEQ ID NO: 1031 - DNA, 18 bp, Artificial Sequence
  source: /note=“Description of Artificial Sequence Synthetic primer”
gctgatggcggagacgaa

SEQ ID NO: 1032 - DNA, 27 bp, Artificial Sequence
  source: /note=“Description of Artificial Sequence Synthetic primer”
cgcgctgtaagaagcaacaacctctcc

SEQ ID NO: 1033 - DNA, 20 bp, Artificial Sequence
  source: /note=“Description of Artificial Sequence Synthetic primer”
gaggagggtgagttctccaa

SEQ ID NO: 1034 - DNA, 20 bp, Artificial Sequence
  source: /note=“Description of Artificial Sequence Synthetic primer”
atgcccacctccttgtaatc

SEQ ID NO: 1035 - DNA, 24 bp, Artificial Sequence
  source: /note=“Description of Artificial Sequence Synthetic primer”
ccatgaggatatgactgccctgga

SEQ ID NO: 1036 - DNA, 19 bp, Artificial Sequence
  source: /note=“Description of Artificial Sequence Synthetic primer”
gtcccttcgcctccttcac

SEQ ID NO: 1037 - DNA, 20 bp, Artificial Sequence
  source: /note=“Description of Artificial Sequence Synthetic primer”
cgtggatggagatgcactca

SEQ ID NO: 1038 - DNA, 28 bp, Artificial Sequence
  source: /note=“Description of Artificial Sequence Synthetic primer”
ccgcagaccccttcaagttctagtcatg

SEQ ID NO: 1039 - DNA, 20 bp, Artificial Sequence
  source: /note=“Description of Artificial Sequence Synthetic primer”
cgccctacctataccaacct

SEQ ID NO: 1040 - DNA, 20 bp, Artificial Sequence
  source: /note=“Description of Artificial Sequence Synthetic primer”
cggagagaagcagtgattga

SEQ ID NO: 1041 - DNA, 25 bp, Artificial Sequence
  source: /note=“Description of Artificial Sequence Synthetic primer”
caaccgcctcatcagtcagattgtg

SEQ ID NO: 1042 - DNA, 20 bp, Artificial Sequence
  source: /note=“Description of Artificial Sequence Synthetic primer”
cgaggacgaggcttaaaaac

SEQ ID NO: 1043 - DNA, 20 bp, Artificial Sequence
  source: /note=“Description of Artificial Sequence Synthetic primer”
accatgcttgaggacaacag

SEQ ID NO: 1044 - DNA, 29 bp, Artificial Sequence
  source: /note=“Description of Artificial Sequence Synthetic primer”
tctcagatcaatcgtgcatccttagtgaa

SEQ ID NO: 1045 - DNA, 21 bp, Artificial Sequence
  source: /note=“Description of Artificial Sequence Synthetic primer”
cgccctcctgcagtatttatg

SEQ ID NO: 1046 - DNA, 22 bp, Artificial Sequence
  source: /note=“Description of Artificial Sequence Synthetic primer”
acagagacaggagcagctcaca

SEQ ID NO: 1047 - DNA, 23 bp, Artificial Sequence
  source: /note=“Description of Artificial Sequence Synthetic primer”
cctcgtcctccccacctaggcca

SEQ ID NO: 1048 - DNA, 21 bp, Artificial Sequence
  source: /note=“Description of Artificial Sequence Synthetic primer”
acactgactggcatcctgctt

SEQ ID NO: 1049 - DNA, 25 bp, Artificial Sequence
  source: /note=“Description of Artificial Sequence Synthetic primer”
gctctgtagctccccatgtactagt

SEQ ID NO: 1050 - DNA, 24 bp, Artificial Sequence
  source: /note=“Description of Artificial Sequence Synthetic primer”
agcctccagaagagccaggtgcct

SEQ ID NO: 1051 - DNA, 20 bp, Artificial Sequence
  source: /note=“Description of Artificial Sequence Synthetic primer”
gtggcctagagccttcagtc

SEQ ID NO: 1052 - DNA, 21 bp, Artificial Sequence
  source: /note=“Description of Artificial Sequence Synthetic primer”
caggctgggagtgaataaaga

SEQ ID NO: 1053 - DNA, 24 bp, Artificial Sequence
  source: /note=“Description of Artificial Sequence Synthetic primer”
ttcacactgcttccctgctttccc

SEQ ID NO: 1054 - DNA, 18 bp, Artificial Sequence
  source: /note=“Description of Artificial Sequence Synthetic primer”
acaggccccatgcatcct

SEQ ID NO: 1055 - DNA, 25 bp, Artificial Sequence
  source: /note=“Description of Artificial Sequence Synthetic primer”
tgtttctctcccagataagctaagg

SEQ ID NO: 1056 - DNA, 21 bp, Artificial Sequence
  source: /note=“Description of Artificial Sequence Synthetic primer”
tgcctcactcccctcagcccc

SEQ ID NO: 1057 - DNA, 19 bp, Artificial Sequence
  source: /note=“Description of Artificial Sequence Synthetic primer”
ccctatggccctgaatggt

SEQ ID NO: 1058 - DNA, 26 bp, Artificial Sequence
  source: /note=“Description of Artificial Sequence Synthetic primer”
actaattacatgacttggctgcattt

SEQ ID NO: 1059 - DNA, 23 bp, Artificial Sequence
  source: /note=“Description of Artificial Sequence Synthetic primer”
tgaggggccgacaccaacacaat

SEQ ID NO: 1060 - DNA, 20 bp, Artificial Sequence
  source: /note=“Description of Artificial Sequence Synthetic primer”
agtggaatccttccctttcc

SEQ ID NO: 1061 - DNA, 20 bp, Artificial Sequence
  source: /note=“Description of Artificial Sequence Synthetic primer”
cccttgatccctttctctga

SEQ ID NO: 1062 - DNA, 24 bp, Artificial Sequence
  source: /note=“Description of Artificial Sequence Synthetic primer”
cctcactcagctcctttcccctga

SEQ ID NO: 1063 - DNA, 24 bp, Artificial Sequence
  source: /note=“Description of Artificial Sequence Synthetic primer”
ggaaggaaagaagcatggtctact

SEQ ID NO: 1064 - DNA, 24 bp, Artificial Sequence
  source: /note=“Description of Artificial Sequence Synthetic primer”
aaaaagtgacaggcaacagtgaag

SEQ ID NO: 1065 - DNA, 24 bp, Artificial Sequence
  source: /note=“Description of Artificial Sequence Synthetic primer”
caccagagacccagcgcacaccta

SEQ ID NO: 1066 - DNA, 19 bp, Artificial Sequence
  source: /note=“Description of Artificial Sequence Synthetic primer”
gatgccgagggaaatcatc

SEQ ID NO: 1067 - DNA, 20 bp, Artificial Sequence
  source: /note=“Description of Artificial Sequence Synthetic primer”
ccagaactcgaacccaatct

SEQ ID NO: 1068 - DNA, 24 bp, Artificial Sequence
  source: /note=“Description of Artificial Sequence Synthetic primer”
attgccgcactggcccaactgtag

SEQ ID NO: 1069 - DNA, 18 bp, Artificial Sequence
  source: /note=“Description of Artificial Sequence Synthetic primer”
gcgctgcggaagatcatc

SEQ ID NO: 1070 - DNA, 22 bp, Artificial Sequence
  source: /note=“Description of Artificial Sequence Synthetic primer”
gcttgagggtctgaatcttgct

SEQ ID NO: 1071 - DNA, 21 bp, Artificial Sequence
  source: /note=“Description of Artificial Sequence Synthetic primer”
ccacgctgccctcggacaagc

SEQ ID NO: 1072 - DNA, 18 bp, Artificial Sequence
  source: /note=“Description of Artificial Sequence Synthetic primer”
cagtgtggaggggatgga

SEQ ID NO: 1073 - DNA, 19 bp, Artificial Sequence
  source: /note=“Description of Artificial Sequence Synthetic primer”
caagttctggaccacagcc

SEQ ID NO: 1074 - DNA, 24 bp, Artificial Sequence
  source: /note=“Description of Artificial Sequence Synthetic primer”
cttcacccactggatgtcaggctc

SEQ ID NO: 1075 - DNA, 23 bp, Artificial Sequence
  source: /note=“Description of Artificial Sequence Synthetic primer”
agttgtcgtgtgttctggattca

SEQ ID NO: 1076 - DNA, 22 bp, Artificial Sequence
  source: /note=“Description of Artificial Sequence Synthetic primer”
cgtcagcgtgatcttaaaggaa

SEQ ID NO: 1077 - DNA, 22 bp, Artificial Sequence
  source: /note=“Description of Artificial Sequence Synthetic primer”
tccggcaccaccatgtcgaagg

SEQ ID NO: 1078 - DNA, 19 bp, Artificial Sequence
  source: /note=“Description of Artificial Sequence Synthetic primer”
gtggatgtgccctgaagga

SEQ ID NO: 1079 - DNA, 20 bp, Artificial Sequence
  source: /note=“Description of Artificial Sequence Synthetic primer”
ctgcggatccagggtaagaa

SEQ ID NO: 1080 - DNA, 28 bp, Artificial Sequence
  source: /note=“Description of Artificial Sequence Synthetic primer”
aagccaggcgtctacacgagagtctcac

SEQ ID NO: 1081 - DNA, 24 bp, Artificial Sequence
  source: /note=“Description of Artificial Sequence Synthetic primer”
ggttgtgctctacgagcttatgac

SEQ ID NO: 1082 - DNA, 22 bp, Artificial Sequence
  source: /note=“Description of Artificial Sequence Synthetic primer”
cggcccaccataaagataatct

SEQ ID NO: 1083 - DNA, 24 bp, Artificial Sequence
  source: /note=“Description of Artificial Sequence Synthetic primer”
tgccttacagccacattggctgcc

SEQ ID NO: 1084 - DNA, 21 bp, Artificial Sequence
  source: /note=“Description of Artificial Sequence Synthetic primer”
tggcttcaggagctgaatacc

SEQ ID NO: 1085 - DNA, 24 bp, Artificial Sequence
  source: /note=“Description of Artificial Sequence Synthetic primer”
tgctgtcgtgatgagaaaatagtg

SEQ ID NO: 1086 - DNA, 26 bp, Artificial Sequence
  source: /note=“Description of Artificial Sequence Synthetic primer”
caggcacacacaggtgggacacaaat

SEQ ID NO: 1087 - DNA, 20 bp, Artificial Sequence
  source: /note=“Description of Artificial Sequence Synthetic primer”
ctgctgtcttgggtgcattg

SEQ ID NO: 1088 - DNA, 18 bp, Artificial Sequence
  source: /note=“Description of Artificial Sequence Synthetic primer”
gcagcctgggaccacttg

SEQ ID NO: 1089 - DNA, 25 bp, Artificial Sequence
  source: /note=“Description of Artificial Sequence Synthetic primer”
ttgccttgctgctctacctccacca

SEQ ID NO: 1090 - DNA, 19 bp, Artificial Sequence
  source: /note=“Description of Artificial Sequence Synthetic primer”
tgacgatggcctggagtgt

SEQ ID NO: 1091 - DNA, 22 bp, Artificial Sequence
  source: /note=“Description of Artificial Sequence Synthetic primer”
ggtaccggatcatgaggatctg

SEQ ID NO: 1092 - DNA, 21 bp, Artificial Sequence
  source: /note=“Description of Artificial Sequence Synthetic primer”
ctgggcagcaccaagtccgga

SEQ ID NO: 1093 - DNA, 25 bp, Artificial Sequence
  source: /note=“Description of Artificial Sequence Synthetic primer”
cctcagcaagacgttatttgaaatt

SEQ ID NO: 1094 - DNA, 24 bp, Artificial Sequence
  source: /note=“Description of Artificial Sequence Synthetic primer”
aagtgtgattggcaaaactgattg

SEQ ID NO: 1095 - DNA, 26 bp, Artificial Sequence
  source: /note=“Description of Artificial Sequence Synthetic primer”
cctctctctcaaggccccaaaccagt

SEQ ID NO: 1096 - DNA, 19 bp, Artificial Sequence
  source: /note=“Description of Artificial Sequence Synthetic primer”
cggttggtgacttgtctgc

SEQ ID NO: 1097 - DNA, 20 bp, Artificial Sequence
  source: /note=“Description of Artificial Sequence Synthetic primer”
aagacttgtccctgcctcac

SEQ ID NO: 1098 - DNA, 24 bp, Artificial Sequence
  source: /note=“Description of Artificial Sequence Synthetic primer”
atgcctcagtcttcccaaagcagg

SEQ ID NO: 1099 - DNA, 21 bp, Artificial Sequence
  source: /note=“Description of Artificial Sequence Synthetic primer”
tgcccttaaaggaaccaatga

SEQ ID NO: 1100 - DNA, 22 bp, Artificial Sequence
  source: /note=“Description of Artificial Sequence Synthetic primer”
gcttcaacggcaaagttctctt

SEQ ID NO: 1101 - DNA, 22 bp, Artificial Sequence
  source: /note=“Description of Artificial Sequence Synthetic primer”
atttcacgcatctggcgttcca

SEQ ID NO: 1102 - DNA, 18 bp, Artificial Sequence
  source: /note=“Description of Artificial Sequence Synthetic primer”
ctctccagtgtgggcacc

SEQ ID NO: 1103 - DNA, 19 bp, Artificial Sequence
  source: /note=“Description of Artificial Sequence Synthetic primer”
gcggtgtagctcccagagt

SEQ ID NO: 1104 - DNA, 24 bp, Artificial Sequence
  source: /note=“Description of Artificial Sequence Synthetic primer”
ccagaacagatgcgagcagtccat

SEQ ID NO: 1105 - DNA, 25 bp, Artificial Sequence
  source: /note=“Description of Artificial Sequence Synthetic primer”
gtatcaggaccacatgcagtacatc

SEQ ID NO: 1106 - DNA, 22 bp, Artificial Sequence
  source: /note=“Description of Artificial Sequence Synthetic primer”
tgtcggaattgatactggcatt

SEQ ID NO: 1107 - DNA, 24 bp, Artificial Sequence
  source: /note=“Description of Artificial Sequence Synthetic primer”
ttgatgcctgtcttcgcgccttct

SEQ ID NO: 1108 - DNA, 23 bp, Artificial Sequence
  source: /note=“Description of Artificial Sequence Synthetic primer”
gcagttggaagacacaggaaagt

SEQ ID NO: 1109 - DNA, 21 bp, Artificial Sequence
  source: /note=“Description of Artificial Sequence Synthetic primer”
tgcgtggcactattttcaaga

SEQ ID NO: 1110 - DNA, 27 bp, Artificial Sequence
  source: /note=“Description of Artificial Sequence Synthetic primer”
tccccaaattgcagatttatcaacggc

SEQ ID NO: 1111 - DNA, 20 bp, Artificial Sequence
  source: /note=“Description of Artificial Sequence Synthetic primer”
caggtcaggcagaggaagtc

SEQ ID NO: 1112 - DNA, 20 bp, Artificial Sequence
  source: /note=“Description of Artificial Sequence Synthetic primer”
cctaacaagccccaaatcaa

SEQ ID NO: 1113 - DNA, 27 bp, Artificial Sequence
  source: /note=“Description of Artificial Sequence Synthetic primer”
tgcattgcatgagctaaacctatctga

SEQ ID NO: 1114 - DNA, 20 bp, Artificial Sequence
  source: /note=“Description of Artificial Sequence Synthetic primer”
tggtcagatgctgctgaagt

SEQ ID NO: 1115 - DNA, 20 bp, Artificial Sequence
  source: /note=“Description of Artificial Sequence Synthetic primer”
atcacagcatgaagggatgg

SEQ ID NO: 1116 - DNA, 24 bp, Artificial Sequence
  source: /note=“Description of Artificial Sequence Synthetic primer”
tatccttaggccgctggcatcttg

SEQ ID NO: 1117 - DNA, 19 bp, Artificial Sequence
  source: /note=“Description of Artificial Sequence Synthetic primer”
gagggagcagacagtgggt

SEQ ID NO: 1118 - DNA, 21 bp, Artificial Sequence
  source: /note=“Description of Artificial Sequence Synthetic primer”
tcagagcccttgctaagtcac

SEQ ID NO: 1119 - DNA, 24 bp, Artificial Sequence
  source: /note=“Description of Artificial Sequence Synthetic primer”
ccacgatctccgtaaccatttgca

SEQ ID NO: 1120 - DNA, 20 bp, Artificial Sequence
  source: /note=“Description of Artificial Sequence Synthetic primer”
tagaggccatcaaaattggc

SEQ ID NO: 1121 - DNA, 18 bp, Artificial Sequence
  source: /note=“Description of Artificial Sequence Synthetic primer”
tccgtttcctctgggctt

SEQ ID NO: 1122 - DNA, 24 bp, Artificial Sequence
  source: /note=“Description of Artificial Sequence Synthetic primer”
accaaggccctgactcagatggag

SEQ ID NO: 1123 - DNA, 82 bp, Artificial Sequence
  source: /note=“Description of Artificial Sequence Synthetic primer”
ttacccgtgggaactgtctccaaatacatacttcctctcaccaggacaacaaccacaggatcctctgacccatttactggtc

SEQ ID NO: 1124 - DNA, 77 bp, Artificial Sequence
  source: /note=“Description of Artificial Sequence Synthetic primer”
aaacaccactggagcattgactaccaggctcgccaatgatgctgctcaagttaaaggggctataggttccaggcttg

SEQ ID NO: 1125 - DNA, 67 bp, Artificial Sequence
  source: /note=“Description of Artificial Sequence Synthetic primer”
agacagtcgccttggtcggtctcaatggcagtgggaagagtacggtagtccagcttctgcagaggtt

SEQ ID NO: 1126 - DNA, 68 bp, Artificial Sequence
  source: /note=“Description of Artificial Sequence Synthetic primer”
accagtgccacaatgcagtggctggacattcggctacagctcatgggggcggcagtggtcagcgctat

SEQ ID NO: 1127 - DNA, 66 bp, Artificial Sequence
  source: /note=“Description of Artificial Sequence Synthetic primer”
aagccacagcctccattgacatggagacagacaccctgatccagcgcacaatccgtgaagccttcc

SEQ ID NO: 1128 - DNA, 76 bp, Artificial Sequence
  source: /note=“Description of Artificial Sequence Synthetic primer”
tgcagactgtaccatgctgaccattgcccatcgcctgcacacggttctaggctccgataggattatggtgctggcc

SEQ ID NO: 1129 - DNA, 67 bp, Artificial Sequence
  source: /note=“Description of Artificial Sequence Synthetic primer”
tctgtggcccacctctactccaacctgaccaagccactcctggacgtggctgtgacttcctacaccc

SEQ ID NO: 1130 - DNA, 83 bp, Artificial Sequence
  source: /note=“Description of Artificial Sequence Synthetic primer”
atgtacgtcgccattcaagctgtgctctccctctatgcctctggccgcacgacaggcatcgtcctggattcaggtgatggcgt

SEQ ID NO: 1131 - DNA, 66 bp, Artificial Sequence
  source: /note=“Description of Artificial Sequence Synthetic primer”
atccgcattgaagacccaccccgcagaaagcacatggtattcctgggtggtgcagttctagcggat

SEQ ID NO: 1132 - DNA, 74 bp, Artificial Sequence
  source: /note=“Description of Artificial Sequence Synthetic primer”
caactgctgagagaccgagaagtaggaatccctccagaacaatccttggaaactgctaaggcagtaaaggagcg

SEQ ID NO: 1133 - DNA, 78 bp, Artificial Sequence
  source: /note=“Description of Artificial Sequence Synthetic primer”
ctgcatgtgattgaataagaaacaagaaagtgaccacaccaaagcctccctggctggtgtacagggatcaggtccaca

SEQ ID NO: 1134 - DNA, 71 bp, Artificial Sequence
  source: /note=“Description of Artificial Sequence Synthetic primer”
cgcttctatggcgctgagattgtgtcagccctggactacctgcactcggagaagaacgtggtgtaccggga

SEQ ID NO: 1135 - DNA, 71 bp, Artificial Sequence
  source: /note=“Description of Artificial Sequence Synthetic primer”
tcctgccacccttcaaacctcaggtcacgtccgaggtcgacacaaggtacttcgatgatgaatttaccgcc

SEQ ID NO: 1136 - DNA, 75 bp, Artificial Sequence
  source: /note=“Description of Artificial Sequence Synthetic primer”
ttgtctctgccttggactatctacattccggaaagattgtgtaccgtgatctcaagttggagaatctaatgctgg

SEQ ID NO: 1137 - DNA, 67 bp, Artificial Sequence
  source: /note=“Description of Artificial Sequence Synthetic primer”
tgggagggatgaaggaaattatctggacgatgctctcgtgagacaggatgcccaggacctgtatgag

SEQ ID NO: 1138 - DNA, 69 bp, Artificial Sequence
  source: /note=“Description of Artificial Sequence Synthetic primer”
ggacagcaggaatgtgtttctccatacaggtcacggggagccaatggttcagaaacaaatcgagtgggt

SEQ ID NO: 1139 - DNA, 68 bp, Artificial Sequence
  source: /note=“Description of Artificial Sequence Synthetic primer”
gatgaagcctttcgcaagttcctgaagggcctggcttcccgaaagccccttgtgctgtgtggagacct

SEQ ID NO: 1140 - DNA, 74 bp, Artificial Sequence
  source: /note=“Description of Artificial Sequence Synthetic primer”
ggaaacacactggaggacaaggctcgggaactcatcagccgcatcaaacagagtgaactttctgccaagatgcg

SEQ ID NO: 1141 - DNA, 67 bp, Artificial Sequence
  source: /note=“Description of Artificial Sequence Synthetic primer”
gtttatgccatcggcaccgtactggatcctggccaccgactatgagaactatgccctcgtgtattcc

SEQ ID NO: 1142 - DNA, 75 bp, Artificial Sequence
  source: /note=“Description of Artificial Sequence Synthetic primer”
gcctcaagagctggttcgagcccctggtggaagacatgcagcgccagtgggccgggctggtggagaaggtgcagg

SEQ ID NO: 1143 - DNA, 66 bp, Artificial Sequence
  source: /note=“Description of Artificial Sequence Synthetic primer”
gaggtcctggagtgcgtgagcctggtggagctgacctcgcttaagggcagggagaagctggcacct

SEQ ID NO: 1144 - DNA, 73 bp, Artificial Sequence
  source: /note=“Description of Artificial Sequence Synthetic primer”
ggtcctccgtcggttctctcattagtccacggtctggtcttcagctacccgccttcgtctccgagtttgcgac

SEQ ID NO: 1145 - DNA, 67 bp, Artificial Sequence
  source: /note=“Description of Artificial Sequence Synthetic primer”
agctgcagaagagctgcacatttgacgagcagcgaacagccacgatcatggaggagttggcagatgc

SEQ ID NO: 1146 - DNA, 66 bp, Artificial Sequence
  source: /note=“Description of Artificial Sequence Synthetic primer”
cagcagatgtggatcagcaagcaggagtatgacgagtccggcccctccatcgtccaccgcaaatgc

SEQ ID NO: 1147 - DNA, 73 bp, Artificial Sequence
  source: /note=“Description of Artificial Sequence Synthetic primer”
gggtcaggtgcctcgagatcgggcttgggcccagagcatgttccagatcccagagtttgagccgagtgagcag

SEQ ID NO: 1148 - DNA, 81 bp, Artificial Sequence
  source: /note=“Description of Artificial Sequence Synthetic primer”
cgttgtcagcacttggaatacaagatggttgccgggtcatgttaattgggaaaaagaacagtccacaggaagaggttgaac

SEQ ID NO: 1149 - DNA, 66 bp, Artificial Sequence
  source: /note=“Description of Artificial Sequence Synthetic primer”
ccattcccaccattctacctgaggccaggacgtctggggtgtggggattggtgggtctatgttccc

SEQ ID NO: 1150 - DNA, 70 bp, Artificial Sequence
  source: /note=“Description of Artificial Sequence Synthetic primer”
ccgccgtggacacagactccccccgagaggtctttttccgagtggcagctgacatgttttctgacggcaa

SEQ ID NO: 1151 - DNA, 83 bp, Artificial Sequence
  source: /note=“Description of Artificial Sequence Synthetic primer”
cctggagggtcctgtacaatctcatcatgggactcctgcccttacccaggggccacagagcccccgagatggagcccaattag

SEQ ID NO: 1152 - DNA, 80 bp, Artificial Sequence
  source: /note=“Description of Artificial Sequence Synthetic primer”
ggctcttgtgcgtactgtccttcgggctggtgacagggaagacatcactgagcctgccatctgtgctcttcgtcatctga

SEQ ID NO: 1153 - DNA, 73 bp, Artificial Sequence
  source: /note=“Description of Artificial Sequence Synthetic primer”
cagatggacctagtacccactgagatttccacgccgaaggacagcgatgggaaaaatgcccttaaatcatagg

SEQ ID NO: 1154 - DNA, 69 bp, Artificial Sequence
  source: /note=“Description of Artificial Sequence Synthetic primer”
aattaccaagcagccgaagaccacccacgaatggttatcttacgactgttacgttacattgtccgcctg

SEQ ID NO: 1155 - DNA, 70 bp, Artificial Sequence
  source: /note=“Description of Artificial Sequence Synthetic primer”
cagcgacaactctggacaagtcagtcccccagagtctccaactgtgaccacttcctggcagtctgagagc

SEQ ID NO: 1156 - DNA, 70 bp, Artificial Sequence
  source: /note=“Description of Artificial Sequence Synthetic primer”
cttttgtggaactctatgggaacaatgcagcagccgagagccgaaagggccaggaacgcttcaaccgctg

SEQ ID NO: 1157 - DNA, 74 bp, Artificial Sequence
  source: /note=“Description of Artificial Sequence Synthetic primer”
tgtactggcgaagaatatttggtaaagcagggcatcgatctctcaccctggggcttgtggaagaatcacgtggc

SEQ ID NO: 1158 - DNA, 71 bp, Artificial Sequence
  source: /note=“Description of Artificial Sequence Synthetic primer”
ggactgtgaggtcaacaacggttccagcctcagggatgagtgcatcacaaacctactggtgtttggcttcc

SEQ ID NO: 1159 - DNA, 76 bp, Artificial Sequence
  source: /note=“Description of Artificial Sequence Synthetic primer”
cctgcaaaagggaacaagagcccttcgcctccagatggctcccctgccgccacccccgagatcagagtcaaccacg

SEQ ID NO: 1160 - DNA, 65 bp, Artificial Sequence
  source: /note=“Description of Artificial Sequence Synthetic primer”
tcagggggctagaaatctgttgctatgggcccttcaccaacatgcccacagatcaactggaatgg

SEQ ID NO: 1161 - DNA, 70 bp, Artificial Sequence
  source: /note=“Description of Artificial Sequence Synthetic primer”
agttcgtgctttgcaagatggtgcagagctttatgaagcagtgaagaatgcagcagacccagcttacctt

SEQ ID NO: 1162 - DNA, 68 bp, Artificial Sequence
  source: /note=“Description of Artificial Sequence Synthetic primer”
ccgaggttaatccagcacgtatggggccaagtgtaggctcccagcaggaactgagagcgccatgtctt

SEQ ID NO: 1163 - DNA, 82 bp, Artificial Sequence
  source: /note=“Description of Artificial Sequence Synthetic primer”
tcaacagaaggctgaaccactagaaagactacagtcccagcaccgacaattccaagctcgagtgtctcggcaaactctgttg

SEQ ID NO: 1164 - DNA, 73 bp, Artificial Sequence
  source: /note=“Description of Artificial Sequence Synthetic primer”
ctgaagcagatggttcatcatttcctgggctgttaaacaaagcgaggttaaggttagactcttgggaatcagc

SEQ ID NO: 1165 - DNA, 69 bp, Artificial Sequence
  source: /note=“Description of Artificial Sequence Synthetic primer”
gtcagcgtggtagcggtattctccgcggcagtgacagtaattgtttttgcctctttagccaagacttcc

SEQ ID NO: 1166 - DNA, 65 bp, Artificial Sequence
  source: /note=“Description of Artificial Sequence Synthetic primer”
tcagctgtgagctgcggataccgcccggcaatgggacctgctcttaacctcaaacctaggaccgt

SEQ ID NO: 1167 - DNA, 72 bp, Artificial Sequence
  source: /note=“Description of Artificial Sequence Synthetic primer”
atcctagccctggtttttggcctcctttttgctgtcaccagcgtcgcgttccttgtgcagatgagaaggcag

SEQ ID NO: 1168 - DNA, 78 bp, Artificial Sequence
  source: /note=“Description of Artificial Sequence Synthetic primer”
cactaaggtttgagacagttccagaaagaacccaagctcaagacgcaggacgagctcagttgtagagggctaattcgc

SEQ ID NO: 1169 - DNA, 66 bp, Artificial Sequence
  source: /note=“Description of Artificial Sequence Synthetic primer”
tcgttggagatcagagtggaagttcaccatcacaccacctacagcccaggtggttggcgtgcttaa

SEQ ID NO: 1170 - DNA, 74 bp, Artificial Sequence
  source: /note=“Description of Artificial Sequence Synthetic primer”
gtggctcaacattgtgttcccatttcagctgatcagtgggcctccaaggaggggctgtaaaatggaggccattg

SEQ ID NO: 1171 - DNA, 84 bp, Artificial Sequence
  source: /note=“Description of Artificial Sequence Synthetic primer”
ttcaggttgttgcaggagaccatgtacatgactgtctccattattgatcggttcatgcagaataattgtgtgcccaagaagatg

SEQ ID NO: 1172 - DNA, 69 bp, Artificial Sequence
  source: /note=“Description of Artificial Sequence Synthetic primer”
gcatgttcgtggcctctaagatgaaggagaccatccccctgacggccgagaagctgtgcatctacaccg

SEQ ID NO: 1173 - DNA, 82 bp, Artificial Sequence
  source: /note=“Description of Artificial Sequence Synthetic primer”
atgctgtggctccttcctaactggggctttcttgacatgtaggttgcttggtaataacctttttgtatatcacaatttgggt

SEQ ID NO: 1174 - DNA, 69 bp, Artificial Sequence
  source: /note=“Description of Artificial Sequence Synthetic primer”
atccaaggccatgactggtgtggaacaatggccatacagggctgttgcccaggccctagaggtcattcc

SEQ ID NO: 1175 - DNA, 66 bp, Artificial Sequence
  source: /note=“Description of Artificial Sequence Synthetic primer”
gtgtgctagcgtactcccgcctcaaggaactgacgctcgaggacctaaagataaccggcaccatgc

SEQ ID NO: 1176 - DNA, 75 bp, Artificial Sequence
  source: /note=“Description of Artificial Sequence Synthetic primer”
tgtatttcaagacctctgtgcacttatttatgaacctgccctgctcccacagaacacagcaattcctcaggctaa

SEQ ID NO: 1177 - DNA, 65 bp, Artificial Sequence
  source: /note=“Description of Artificial Sequence Synthetic primer”
agatgaagtggaaggcgcttttcaccgcggccatcctgcaggcacagttgccgattacagaggca

SEQ ID NO: 1178 - DNA, 71 bp, Artificial Sequence
  source: /note=“Description of Artificial Sequence Synthetic primer”
agtgggactgattgccgtgggtgtcggggcacagcttgtcctgagtcagaccataatccagggggctaccc

SEQ ID NO: 1179 - DNA, 74 bp, Artificial Sequence
  source: /note=“Description of Artificial Sequence Synthetic primer”
tggttcccagccctgtgtccacctccaagcccagattcagattcgagtcatgtacacaacccagggtggaggag

SEQ ID NO: 1180 - DNA, 68 bp, Artificial Sequence
  source: /note=“Description of Artificial Sequence Synthetic primer”
gagagcgacgcggttgttgtagctgccgctgcggccgccgcggaataataagccgggatctaccatac

SEQ ID NO: 1181 - DNA, 68 bp, Artificial Sequence
  source: /note=“Description of Artificial Sequence Synthetic primer”
tggattggagttctgggaatgtactggccgtggcactggacaacagtgtgtacctgtggagtgcaagc

SEQ ID NO: 1182 - DNA, 85 bp, Artificial Sequence
  source: /note=“Description of Artificial Sequence Synthetic primer”
aaacgagcagtttgccatcagacgcttccagtctatgccggtgaggctgctgggccacagccccgtgcttcggaacatcaccaac

SEQ ID NO: 1183 - DNA, 67 bp, Artificial Sequence
  source: /note=“Description of Artificial Sequence Synthetic primer”
gaggcacagtattgcccagctggatccagaggccttgggaaacattaagaagctctccaaccgtctc

SEQ ID NO: 1184 - DNA, 81 bp, Artificial Sequence
  source: /note=“Description of Artificial Sequence Synthetic primer”
tgagtgtcccccggtatcttccccgccctgccaatcccgatgaaattggaaattttattgatgaaaatctgaaagcggctg

SEQ ID NO: 1185 - DNA, 67 bp, Artificial Sequence
  source: /note=“Description of Artificial Sequence Synthetic primer”
aagccctatccgatgtacccggccacaacatccctggtgaacgtcgtgcccaaactcaatgccacag

SEQ ID NO: 1186 - DNA, 69 bp, Artificial Sequence
  source: /note=“Description of Artificial Sequence Synthetic primer”
cggcgatcaagaagctgtccgggcctctgatctccgatttcttcgccaagcgcaagagatcagcgcctg

SEQ ID NO: 1187 - DNA, 77 bp, Artificial Sequence
  source: /note=“Description of Artificial Sequence Synthetic primer”
tgacaatcagcacacctgcattcaccgctcggaagagggcctgagctgcatgaataaggatcacggctgtagtcaca

SEQ ID NO: 1188 - DNA, 63 bp, Artificial Sequence
  source: /note=“Description of Artificial Sequence Synthetic primer”
taaattcactcgtggtgtggacttcaattggcaagcccaggccctattggccctacaagaggc

SEQ ID NO: 1189 - DNA, 83 bp, Artificial Sequence
  source: /note=“Description of Artificial Sequence Synthetic primer”
ggatgctggtgacctcttcttccctcacgttgcaacaggaattaaaggctaaaagaaaacgaagagttacttggtgccttggc

SEQ ID NO: 1190 - DNA, 68 bp, Artificial Sequence
  source: /note=“Description of Artificial Sequence Synthetic primer”
ctcccgtcaacagcgttctttccaaacactggaccaggagtgcatccagatgaaggccagactcaccc

SEQ ID NO: 1191 - DNA, 76 bp, Artificial Sequence
  source: /note=“Description of Artificial Sequence Synthetic primer”
ctgaaggagctccaagacctcgctctccaaggcgccaaggagagggcacatcagcagaagaaacacagcggttttg

SEQ ID NO: 1192 - DNA, 76 bp, Artificial Sequence
  source: /note=“Description of Artificial Sequence Synthetic primer”
aaggaagtggtccctctgtggcaagtgatgaagtctccagctttgcctcagctctcccagacagaaagactgcgtc

SEQ ID NO: 1193 - DNA, 82 bp, Artificial Sequence
  source: /note=“Description of Artificial Sequence Synthetic primer”
gataaattggtacaagggatcagcttttcccagcccacatgtcctgatcatatgcttttgaatagtcagttacttggcaccc

SEQ ID NO: 1194 - DNA, 78 bp, Artificial Sequence
  source: /note=“Description of Artificial Sequence Synthetic primer”
atgtggaacccccacctacttggcgcctgaagttcttgtttctgttgggactgctgggtataaccgtgctgtggactg

SEQ ID NO: 1195 - DNA, 86 bp, Artificial Sequence
  source: /note=“Description of Artificial Sequence Synthetic primer”
ggatatttccgtggctcttattcaaactctccatcaaatcctgtaaactccagagcaaatcaagatttttctgccttgatgagaag

SEQ ID NO: 1196 - DNA, 66 bp, Artificial Sequence
  source: /note=“Description of Artificial Sequence Synthetic primer”
tcattgaccacagtggcgcccgccttggtgagtatgaggacgtgtcccgggtggagaagtacacga

SEQ ID NO: 1197 - DNA, 76 bp, Artificial Sequence
  source: /note=“Description of Artificial Sequence Synthetic primer”
ccccaggatacctaccactacctgcccttcagcctgccccaccggaggcctcacttcttctttcccaagtcccgca

SEQ ID NO: 1198 - DNA, 86 bp, Artificial Sequence
  source: /note=“Description of Artificial Sequence Synthetic primer”
gacatttccagtcctgcagtcaatgcctctctgccccaccctttgttcagtgtggctggtgccacgacaaatgtgtgcgatcggag

SEQ ID NO: 1199 - DNA, 84 bp, Artificial Sequence
  source: /note=“Description of Artificial Sequence Synthetic primer”
tccctccactcggaaggactatcctgctgccaagagggtcaagttggacagtgtcagagtcctgagacagatcagcaacaaccg

SEQ ID NO: 1200 - DNA, 64 bp, Artificial Sequence
  source: /note=“Description of Artificial Sequence Synthetic primer”
tccaccctcctggctttggccagcatggcgaagacgaaaggaaacaaggtgaacgtgggagtga

SEQ ID NO: 1201 - DNA, 68 bp, Artificial Sequence
  source: /note=“Description of Artificial Sequence Synthetic primer”
gtggccatccagctgaccttcctgcgcctgatgtccaccgaggcctcccagaacatcacctaccactg

SEQ ID NO: 1202 - DNA, 80 bp, Artificial Sequence
  source: /note=“Description of Artificial Sequence Synthetic primer”
cagccaagaactggtataggagctccaaggacaagaaacacgtctggctaggagaaactatcaatgctggcagccagttt

SEQ ID NO: 1203 - DNA, 66 bp, Artificial Sequence
  source: /note=“Description of Artificial Sequence Synthetic primer”
gagagcaagcgagacattctgttcctctttgacggctcagccaatcttgtgggccagttccctgtt

SEQ ID NO: 1204 - DNA, 81 bp, Artificial Sequence
  source: /note=“Description of Artificial Sequence Synthetic primer”
cgacagttgcgatgaaagttctaatctcttccctcctcctgttgctgccactaatgctgatgtccatggtctctagcagcc

SEQ ID NO: 1205 - DNA, 79 bp, Artificial Sequence
  source: /note=“Description of Artificial Sequence Synthetic primer”
tctgcagagttggaagcactctatggtgacatcgatgctgtggagctgtatcctgcccttctggtagaaaagcctcggc

SEQ ID NO: 1206 - DNA, 78 bp, Artificial Sequence
  source: /note=“Description of Artificial Sequence Synthetic primer”
acctctgtggtccgtaggagccactatgaggagggccctgggaagaatttgccattttcagtggaaaacaagtggtcg

SEQ ID NO: 1207 - DNA, 67 bp, Artificial Sequence
  source: /note=“Description of Artificial Sequence Synthetic primer”
aacttcaaggtcggagaaggctttgaggaggagaccgtggacggacgcaagtgcaggagtttagcca

SEQ ID NO: 1208 - DNA, 76 bp, Artificial Sequence
  source: /note=“Description of Artificial Sequence Synthetic primer”
gtgctacgccaccctgttcggacccaaaggcgtgaacatcgggggcgcgggctcctacatctacgagaagcccctg

SEQ ID NO: 1209 - DNA, 69 bp, Artificial Sequence
  source: /note=“Description of Artificial Sequence Synthetic primer”
gatgtgattgaggtgcatggaaaacatgaagagcgccaggatgaacatggtttcatctccagggagttc

SEQ ID NO: 1210 - DNA, 74 bp, Artificial Sequence
  source: /note=“Description of Artificial Sequence Synthetic primer”
tgcagcggctgattgacagtcagatggagacctcgtgccaaattacatttgagtttgtagaccaggaacagttg

SEQ ID NO: 1211 - DNA, 72 bp, Artificial Sequence
  source: /note=“Description of Artificial Sequence Synthetic primer”
agcttttccggaatctgttccatcgccagggcttctcctatgactacgtgttcgactggaacatgctcaaat

SEQ ID NO: 1212 - DNA, 78 bp, Artificial Sequence
  source: /note=“Description of Artificial Sequence Synthetic primer”
tggcagaactacctgcaagaaaaaccagcacctgcgtctggatgactgtgacttccaaaccaaccacaccttgaagca

SEQ ID NO: 1213 - DNA, 80 bp, Artificial Sequence
  source: /note=“Description of Artificial Sequence Synthetic primer”
gtacatgatcccctgtgagaaggtgtccaccctgcccgcgatcacactgaagctgggaggcaaaggctacaagctgtccc

SEQ ID NO: 1214 - DNA, 74 bp, Artificial Sequence
  source: /note=“Description of Artificial Sequence Synthetic primer”
gggaggcttatctcactgagtgagcagaatctggtagactgctctgggcctcaaggcaatgaaggctgcaatgg

SEQ ID NO: 1215 - DNA, 67 bp, Artificial Sequence
  source: /note=“Description of Artificial Sequence Synthetic primer”
tgtctcactgagcgagcagaatctggtggactgttcgcgtcctcaaggcaatcagggctgcaatggt

SEQ ID NO: 1216 - DNA, 72 bp, Artificial Sequence
  source: /note=“Description of Artificial Sequence Synthetic primer”
tgaccgcttctaccccaatgacttgtgggtggttgtgttccagtttcagcacatcatggttggccttatcct

SEQ ID NO: 1217 - DNA, 77 bp, Artificial Sequence
  source: /note=“Description of Artificial Sequence Synthetic primer”
ggtgcctactccattgtggcgggcgtgtttgtgtgcctgctggagtacccccgggggaagaggaagaagggctccac

SEQ ID NO: 1218 - DNA, 71 bp, Artificial Sequence
  source: /note=“Description of Artificial Sequence Synthetic primer”
ccagctttgtgcctgtcactattcctcatgccaccactgccaacacctctgtcttgggctaccacattccc

SEQ ID NO: 1219 - DNA, 73 bp, Artificial Sequence
  source: /note=“Description of Artificial Sequence Synthetic primer”
ccgtgttcaagaggaagctcactgccttgtggaggagttgagaaaaaccaaggcttcaccctgtgatcccact

SEQ ID NO: 1220 - DNA, 79 bp, Artificial Sequence
  source: /note=“Description of Artificial Sequence Synthetic primer”
agaacaaggacaacatagatccttacatatacacaccctttggaagtggacccagaaactgcattggcatgaggtttgc

SEQ ID NO: 1221 - DNA, 70 bp, Artificial Sequence
  source: /note=“Description of Artificial Sequence Synthetic primer”
ccgtgtggctcgctttctgcagtgccgcttcctctttgcggggccctggttactcttcagcgaaatctcc

SEQ ID NO: 1222 - DNA, 73 bp, Artificial Sequence
  source: /note=“Description of Artificial Sequence Synthetic primer”
cacaatggcggctctgaagagttggctgtcgcgcagcgtaacttcattcttcaggtacagacagtgtttgtgt

SEQ ID NO: 1223 - DNA, 62 bp, Artificial Sequence
  source: /note=“Description of Artificial Sequence Synthetic primer”
caagcagtcaaggagaaccagaagcggcgggagacagaagaaaagatgaggcgagcaaaact

SEQ ID NO: 1224 - DNA, 68 bp, Artificial Sequence
  source: /note=“Description of Artificial Sequence Synthetic primer”
tccaattccagcatcactgtggagaaaagctgtttgtctccccagcatactttatcgccttcactgcc

SEQ ID NO: 1225 - DNA, 75 bp, Artificial Sequence
  source: /note=“Description of Artificial Sequence Synthetic primer”
cagtgcttccatggacaagtccttgtcaaaactggcccatgctgatggagatcaaacatcaatcatccctgtcca

SEQ ID NO: 1226 - DNA, 83 bp, Artificial Sequence
  source: /note=“Description of Artificial Sequence Synthetic primer”
tgcacagagggtgtgggttacaccaatgcttccaacaatttgtttgcttgcctcccatgtacagcttgtaaatcagatgaaga

SEQ ID NO: 1227 - DNA, 84 bp, Artificial Sequence
  source: /note=“Description of Artificial Sequence Synthetic primer”
ctctgagacagtgcttcgatgactttgcagacttggtgccctttgactcctgggagccgctcatgaggaagttgggcctcatgg

SEQ ID NO: 1228 - DNA, 76 bp, Artificial Sequence
  source: /note=“Description of Artificial Sequence Synthetic primer”
agacatcagctcctggttcaacgaggccattgacttcatagactccatcaagaatgctggaggaagggtgtttgtc

SEQ ID NO: 1229 - DNA, 74 bp, Artificial Sequence
  source: /note=“Description of Artificial Sequence Synthetic primer”
cagaggatgacgaggatgatgacattgacctgtttggcagtgacaatgaggaggaggacaaggaggcggcacag

SEQ ID NO: 1230 - DNA, 62 bp, Artificial Sequence
  source: /note=“Description of Artificial Sequence Synthetic primer”
tgtcgatggacttccagaaccacctgggcagctgccaaaagtgtgatccaagctgtcccaat

SEQ ID NO: 1231 - DNA, 82 bp, Artificial Sequence
  source: /note=“Description of Artificial Sequence Synthetic primer”
gatctaagatggcgactgtcgaaccggaaaccacccctactcctaatcccccgactacagaagaggagaaaacggaatctaa

SEQ ID NO: 1232 - DNA, 67 bp, Artificial Sequence
  source: /note=“Description of Artificial Sequence Synthetic primer”
aagccgcggttgaatgtgccatgaccctctccctctctggatggcaccatcattgaagctggcgtca

SEQ ID NO: 1233 - DNA, 67 bp, Artificial Sequence
  source: /note=“Description of Artificial Sequence Synthetic primer”
ctcggatcctagccctcgtgcctccatggactcgagtgtaccgagtacagagggatattccaatgcc

SEQ ID NO: 1234 - DNA, 76 bp, Artificial Sequence
  source: /note=“Description of Artificial Sequence Synthetic primer”
tggtccatcgccagttatcacatctgtatgcggaacctcaaaagagtccctggtgtgaagcaagatcgctagaaca

SEQ ID NO: 1235 - DNA, 81 bp, Artificial Sequence
  source: /note=“Description of Artificial Sequence Synthetic primer”
cggttatgtcatgccagatacacacctcaaaggtactccctcctcccgggaaggcaccctttcttcagtgggtctcagttc

SEQ ID NO: 1236 - DNA, 86 bp, Artificial Sequence
  source: /note=“Description of Artificial Sequence Synthetic primer”
tggctcttaatcagtttcgttacctgcctctggagaatttacgcattattcgtgggacaaaactttatgaggatcgatatgccttg

SEQ ID NO: 1237 - DNA, 67 bp, Artificial Sequence
  source: /note=“Description of Artificial Sequence Synthetic primer”
gtccaggtggatgtgaaagatccccagcaggccctcaaggagctggctaagatgtgtatcctggccg

SEQ ID NO: 1238 - DNA, 67 bp, Artificial Sequence
  source: /note=“Description of Artificial Sequence Synthetic primer”
acggatcacagtggaggaagcgctggctcacccctacctggagcagtactatgacccgacggatgag

SEQ ID NO: 1239 - DNA, 70 bp, Artificial Sequence
  source: /note=“Description of Artificial Sequence Synthetic primer”
acccccagaccggatcaggcaagctggccctcatgtccccttcacggtgtttgaggaagtctgccctaca

SEQ ID NO: 1240 - DNA, 68 bp, Artificial Sequence
  source: /note=“Description of Artificial Sequence Synthetic primer”
cgtggtgcccctctatgacctgctgctggagatgctggacgcccaccgcctacatgcgcccactagcc

SEQ ID NO: 1241 - DNA, 91 bp, Artificial Sequence
  source: /note=“Description of Artificial Sequence Synthetic primer”
ggattgctcaacaaccatgctgggcatctggaccctcctacctctggttcttacgtctgttgctagattatcgtccaaaagtgttaatgcc

SEQ ID NO: 1242 - DNA, 80 bp, Artificial Sequence
  source: /note=“Description of Artificial Sequence Synthetic primer”
gcactttgggattctttccattatgattctttgttacaggcaccgagaatgttgtattcagtgagggtcttcttacatgc

SEQ ID NO: 1243 - DNA, 66 bp, Artificial Sequence
  source: /note=“Description of Artificial Sequence Synthetic primer”
gcctcttcctgttcgacggctcgcccacctacgtactggcctacacccagagctaccgggcaaagc

SEQ ID NO: 1244 - DNA, 90 bp, Artificial Sequence
  source: /note=“Description of Artificial Sequence Synthetic primer”
ggctattcctcattttctctacaaagtggcctcagtgaacatgaagaaggtagcctcctggaggagaatttcggtgacagtctacaatcc

SEQ ID NO: 1245 - DNA, 67 bp, Artificial Sequence
  source: /note=“Description of Artificial Sequence Synthetic primer”
aaggaaagggtgcctcatcccagcaacctgtccttgtgggtgatgatcactgtgctgcttgtggctc

SEQ ID NO: 1246 - DNA, 74 bp, Artificial Sequence
  source: /note=“Description of Artificial Sequence Synthetic primer”
cacgggacattcaccacatcgactactataaaaagacaaccaacggccgactgcctgtgaagtggatggcaccc

SEQ ID NO: 1247 - DNA, 67 bp, Artificial Sequence
  source: /note=“Description of Artificial Sequence Synthetic primer”
ccagtggagcgcttccatgacctgcgtcctgatgaagtggccgatttgtttcagacgacccagagag

SEQ ID NO: 1248 - DNA, 72 bp, Artificial Sequence
  source: /note=“Description of Artificial Sequence Synthetic primer”
ggttccagctttctgtagctgtaagcattggtggccacaccacctccttacaaagcaactagaacctgcggc

SEQ ID NO: 1249 - DNA, 73 bp, Artificial Sequence
  source: /note=“Description of Artificial Sequence Synthetic primer”
gcgtatgatttcccgaatgagtcaaaatgttgatatgcccaaacttcatgatgcaatgggtacgaggtcactg

SEQ ID NO: 1250 - DNA, 67 bp, Artificial Sequence
  source: /note=“Description of Artificial Sequence Synthetic primer”
cgagccctttgatgacttcctgttcccagcatcatccaggcccagtggctctgagacagcccgctcc

SEQ ID NO: 1251 - DNA, 82 bp, Artificial Sequence
  source: /note=“Description of Artificial Sequence Synthetic primer”
ccaccccgagcaaatctgtcctccccagaacccctgaatcctggaggctcacgcccccagccaaagtagggggactggattt

SEQ ID NO: 1252 - DNA, 80 bp, Artificial Sequence
  source: /note=“Description of Artificial Sequence Synthetic primer”
ggataattcagacaacaacaccatctttgtgcaaggcctgggtgagaatgttacaattgagtctgtggctgattacttca

SEQ ID NO: 1253 - DNA, 69 bp, Artificial Sequence
  source: /note=“Description of Artificial Sequence Synthetic primer”
gaagcgcagatcatgaagaagctgaagcacgacaagctggtccagctctatgcagtggtgtctgaggag

SEQ ID NO: 1254 - DNA, 67 bp, Artificial Sequence
  source: /note=“Description of Artificial Sequence Synthetic primer”
cctccaactcctagcctcaagtgatcctcctgtctcaacctcccaagtaggattacaagcatgcgcc

SEQ ID NO: 1255 - DNA, 73 bp, Artificial Sequence
  source: /note=“Description of Artificial Sequence Synthetic primer”
gtgctggtgacgaatccacattcatctcaatggaaggatcctgccttaagtcaacttatttgtttttgccggg

SEQ ID NO: 1256 - DNA, 70 bp, Artificial Sequence
  source: /note=“Description of Artificial Sequence Synthetic primer”
accctcgacaagaccacactttgggacttgggagctggggctgaagttgctctgtacccatgaactccca

SEQ ID NO: 1257 - DNA, 84 bp, Artificial Sequence
  source: /note=“Description of Artificial Sequence Synthetic primer”
aagggcaatcacagtgttaaaagaagacatgctgaaatgttgcaggctgctcctatgttggaaaattcttcattgaagttctcc

SEQ ID NO: 1258 - DNA, 74 bp, Artificial Sequence
  source: /note=“Description of Artificial Sequence Synthetic primer”
attccacccatggcaaattccatggcaccgtcaaggctgagaacgggaagcttgtcatcaatggaaatcccatc

SEQ ID NO: 1259 - DNA, 75 bp, Artificial Sequence
  source: /note=“Description of Artificial Sequence Synthetic primer”
caaaggagctcactgtggtgtctgtgttccaaccactgaatctggaccccatctgtgaataagccattctgactc

SEQ ID NO: 1260 - DNA, 73 bp, Artificial Sequence
  source: /note=“Description of Artificial Sequence Synthetic primer”
ttgggaaatatttgggcattggtctggccaagtctacaatgtcccaatatcaaggacaaccaccctagcttct

SEQ ID NO: 1261 - DNA, 83 bp, Artificial Sequence
  source: /note=“Description of Artificial Sequence Synthetic primer”
gcatgggaaccatcaaccagcaggccatggaccaacttcactatgtgacagagctgacagatcgaatcaaggcaaactcctca

SEQ ID NO: 1262 - DNA, 71 bp, Artificial Sequence
  source: /note=“Description of Artificial Sequence Synthetic primer”
ctgttgcaggaaggcattgatcatctcctggcccagcatgttgctcatctctttattagagacccactgac

SEQ ID NO: 1263 - DNA, 72 bp, Artificial Sequence
  source: /note=“Description of Artificial Sequence Synthetic primer”
cgctccagacctatgatgacttgttagccaaagactgccactgcatatgagcagtcctggtccttccactgt

SEQ ID NO: 1264 - DNA, 70 bp, Artificial Sequence
  source: /note=“Description of Artificial Sequence Synthetic primer”
ctccgacgtggctttccagtggcccacagcatctatggaatcccatctgtcatcaattctgccaattacg

SEQ ID NO: 1265 - DNA, 67 bp, Artificial Sequence
  source: /note=“Description of Artificial Sequence Synthetic primer”
ggagctctgcagtaaccacagaacaggccccatgctgacgtccctcctcaagagctggatggcattg

SEQ ID NO: 1266 - DNA, 67 bp, Artificial Sequence
  source: /note=“Description of Artificial Sequence Synthetic primer”
ggtgaaggttgtctcttccgagggccttctgaagacagggctcttgaacagacaagtggaagggctg

SEQ ID NO: 1267 - DNA, 66 bp, Artificial Sequence
  source: /note=“Description of Artificial Sequence Synthetic primer”
tacccttccatgtgctggatccgggactccctggtcagctacatcaccaacctgggcctcttcagc

SEQ ID NO: 1268 - DNA, 67 bp, Artificial Sequence
  source: /note=“Description of Artificial Sequence Synthetic primer”
gcttatgaccgaccccaagctcatcacctggtctccggtgtgtcgcaacgatgttgcctggaacttt

SEQ ID NO: 1269 - DNA, 67 bp, Artificial Sequence
  source: /note=“Description of Artificial Sequence Synthetic primer”
ccatctgcatccatcttgtttgggctccccacccttgagaagtgcctcagataataccctggtggcc

SEQ ID NO: 1270 - DNA, 66 bp, Artificial Sequence
  source: /note=“Description of Artificial Sequence Synthetic primer”
gacaaggacggcagcaaggtgacaacagtggtggcaactcctgggcagggtccagacaggccacaa

SEQ ID NO: 1271 - DNA, 68 bp, Artificial Sequence
  source: /note=“Description of Artificial Sequence Synthetic primer”
gtgatcccaagcccacaatagaggtcagtgggaaaaagtacaccgccccacacatcctgatcgccaca

SEQ ID NO: 1272 - DNA, 86 bp, Artificial Sequence
  source: /note=“Description of Artificial Sequence Synthetic primer”
aagctatgaggaaaagaagtacacgatgggggacgctcctgattatgacagaagccagtggctgaatgaaaaattcaagctgggcc

SEQ ID NO: 1273 - DNA, 76 bp, Artificial Sequence
  source: /note=“Description of Artificial Sequence Synthetic primer”
gagaccctgctgtcccagaaccagggaggcaagaccttcattgtgggagaccagatctccttcgctgactacaacc

SEQ ID NO: 1274 - DNA, 73 bp, Artificial Sequence
  source: /note=“Description of Artificial Sequence Synthetic primer”
cccactcagtagccaagtcacaatgtttggaaaacagcccgtttacttgagcaagactgataccacctgcgtg

SEQ ID NO: 1275 - DNA, 66 bp, Artificial Sequence
  source: /note=“Description of Artificial Sequence Synthetic primer”
tcctgtgctctggaagcccttgagcccttctgggaggttcttgtgagatcaactgagaccgtggag

SEQ ID NO: 1276 - DNA, 70 bp, Artificial Sequence
  source: /note=“Description of Artificial Sequence Synthetic primer”
cggtgtgagaagtgcagcaagccctgtgcccgagtgtgctatggtctgggcatggagcacttgcgagagg

SEQ ID NO: 1277 - DNA, 82 bp, Artificial Sequence
  source: /note=“Description of Artificial Sequence Synthetic primer”
tgaacataaagtctgcaacatggaaggtattgcactgcacaggccacattcacgtatatgataccaacagtaaccaacctca

SEQ ID NO: 1278 - DNA, 73 bp, Artificial Sequence
  source: /note=“Description of Artificial Sequence Synthetic primer”
tccaggatgttaggaactgtgaagatggaagggcatgaaaccagcgactggaacagctactacgcagacacgc

SEQ ID NO: 1279 - DNA, 67 bp, Artificial Sequence
  source: /note=“Description of Artificial Sequence Synthetic primer”
ggacgaatacgaccccactatagaggattcctaccggaagcaggtggtcattgatggggagacgtgc

SEQ ID NO: 1280 - DNA, 70 bp, Artificial Sequence
  source: /note=“Description of Artificial Sequence Synthetic primer”
ctgctgcgacagtccactacctttttcgagagtgactcccgttgtcccaaggcttcccagagcgaacctg

SEQ ID NO: 1281 - DNA, 63 bp, Artificial Sequence
  source: /note=“Description of Artificial Sequence Synthetic primer”
ggtccgcttcgtctttcgagagtgactcccgcggtcccaaggctttccagagcgaacctgtgc

SEQ ID NO: 1282 - DNA, 72 bp, Artificial Sequence
  source: /note=“Description of Artificial Sequence Synthetic primer”
gcaggtgtgattgctggacttaatgtgctaagaatcatcaatgagcccacggctgctgccattgcctatggt

SEQ ID NO: 1283 - DNA, 84 bp, Artificial Sequence
  source: /note=“Description of Artificial Sequence Synthetic primer”
ggctagtagaactggatcccaacaccaaactcttaattagacctaggcctcagctgcactgcccgaaaagcatttgggcagacc

SEQ ID NO: 1284 - DNA, 70 bp, Artificial Sequence
  source: /note=“Description of Artificial Sequence Synthetic primer”
ggccactaaagatgctggccagatatctggactgaatgtgcttcgggtgattaatgagcccacagctgct

SEQ ID NO: 1285 - DNA, 84 bp, Artificial Sequence
  source: /note=“Description of Artificial Sequence Synthetic primer”
ccgactggaggagcataaaagcgcagccgagcccagcgccccgcacttttctgagcagacgtccagagcagagtcagccagcat

SEQ ID NO: 1286 - DNA, 71 bp, Artificial Sequence
  source: /note=“Description of Artificial Sequence Synthetic primer”
caaaaggcagaggctgataagaacgacaagtctgtgaaggatctggtcatcttgctttatgaaactgcgct

SEQ ID NO: 1287 - DNA, 70 bp, Artificial Sequence
  source: /note=“Description of Artificial Sequence Synthetic primer”
agaaccgcaaggtgagcaaggtggagattctccagcacgtcatcgactacatcagggaccttcagttgga

SEQ ID NO: 1288 - DNA, 79 bp, Artificial Sequence
  source: /note=“Description of Artificial Sequence Synthetic primer”
cacgcagaaaaccacacttctcaaaccttcactcaacacttccttccccaaagccagaagatgcacaaggaggaacatg

SEQ ID NO: 1289 - DNA, 83 bp, Artificial Sequence
  source: /note=“Description of Artificial Sequence Synthetic primer”
gcatggtagccgaagatttcacagtcaaaatcggagattttggtatgacgcgagatatctatgagacagactattaccggaaa

SEQ ID NO: 1290 - DNA, 73 bp, Artificial Sequence
  source: /note=“Description of Artificial Sequence Synthetic primer”
gtggacagcaccatgaacatgttgggcgggggaggcagtgctggccggaagcccctcaagtcgggtatgaagg

SEQ ID NO: 1291 - DNA, 68 bp, Artificial Sequence
  source: /note=“Description of Artificial Sequence Synthetic primer”
acgcaccgggtgtctgatcccaagttccaccccctccattcaaagataatcatcatcaagaaagggca

SEQ ID NO: 1292 - DNA, 69 bp, Artificial Sequence
  source: /note=“Description of Artificial Sequence Synthetic primer”
tggacaagtacgggatgaagctgccaggcatggagtacgttgacggggactttcagtgccacaccttcg

SEQ ID NO: 1293 - DNA, 74 bp, Artificial Sequence
  source: /note=“Description of Artificial Sequence Synthetic primer”
tctgcgtggttcctttctcagccgcttctgactgctgattctcccgttcacgttgcctaataaacatccttcaa

SEQ ID NO: 1294 - DNA, 72 bp, Artificial Sequence
  source: /note=“Description of Artificial Sequence Synthetic primer”
cctgaaccttccaaagatggctgaaaaagatggatgcttccaatctggattcaatgaggagacttgcctggt

SEQ ID NO: 1295 - DNA, 71 bp, Artificial Sequence
  source: /note=“Description of Artificial Sequence Synthetic primer”
gcggtgattcggaaattcgcgaattcctctggtcctcatccaggtgcgcgggaagcaggtgcccaggagag

SEQ ID NO: 1296 - DNA, 70 bp, Artificial Sequence
  source: /note=“Description of Artificial Sequence Synthetic primer”
aaggaaccatctcactgtgtgtaaacatgacttccaagctggccgtggctctcttggcagccttcctgat

SEQ ID NO: 1297 - DNA, 68 bp, Artificial Sequence
  source: /note=“Description of Artificial Sequence Synthetic primer”
ccgctccgtcactgatgtctacctgctgaacctagccttggccgacctactctttgccctgaccttgc

SEQ ID NO: 1298 - DNA, 66 bp, Artificial Sequence
  source: /note=“Description of Artificial Sequence Synthetic primer”
ctcaggattttctcgcatccaaatgtgctcccagtgctaggtgcctgccagtctccacctgctcct

SEQ ID NO: 1299 - DNA, 63 bp, Artificial Sequence
  source: /note=“Description of Artificial Sequence Synthetic primer”
agccatcactctcagtgcagccaggtcctatcgtggcccctgaggagaccctgactctgcagt

SEQ ID NO: 1300 - DNA, 67 bp, Artificial Sequence
  source: /note=“Description of Artificial Sequence Synthetic primer”
gccaggatactggagtccatcacagtgagctccctgatggctacaccccaggaccccaagggtcaag

SEQ ID NO: 1301 - DNA, 67 bp, Artificial Sequence
  source: /note=“Description of Artificial Sequence Synthetic primer”
ggatggagttcgcctcctacgtgatcacagacctgacccagctgcggaagatcaagtccatggagcg

SEQ ID NO: 1302 - DNA, 74 bp, Artificial Sequence
  source: /note=“Description of Artificial Sequence Synthetic primer”
ccacagctcaccttctgtcaggtgtccatcccagctccagccagctcccagagaggaagagactggcactgagg

SEQ ID NO: 1303 - DNA, 74 bp, Artificial Sequence
  source: /note=“Description of Artificial Sequence Synthetic primer”
tcagaattggatttggctcatttgtggaaaagactgtgatgccttacattagcacaacaccagctaagctcagg

SEQ ID NO: 1304 - DNA, 74 bp, Artificial Sequence
  source: /note=“Description of Artificial Sequence Synthetic primer”
tgaggaagaaggagaggaatactaattatccattccttttggccctgcagcatgtcatgctcccagaatttcag

SEQ ID NO: 1305 - DNA, 68 bp, Artificial Sequence
  source: /note=“Description of Artificial Sequence Synthetic primer”
gaggacgaaggcctctacacctgccaggcatgcagtgttcttggctgtgcaaaagtggaggcattttt

SEQ ID NO: 1306 - DNA, 80 bp, Artificial Sequence
  source: /note=“Description of Artificial Sequence Synthetic primer”
cggactttgggtgcgacttgacgagcggtggttcgacaagtggccttgcgggccggatcgtcccagtggaagagttgtaa

SEQ ID NO: 1307 - DNA, 71 bp, Artificial Sequence
  source: /note=“Description of Artificial Sequence Synthetic primer”
tggaggttgtaagccaatgttgtgaggcttcaagttcagacatcactgagaaatcagatggacgtaaggca

SEQ ID NO: 1308 - DNA, 67 bp, Artificial Sequence
  source: /note=“Description of Artificial Sequence Synthetic primer”
ctaaggcacttgctggaagggcagaatgccagtgtgcttgcctatggacccacaggagctgggaaga

SEQ ID NO: 1309 - DNA, 73 bp, Artificial Sequence
  source: /note=“Description of Artificial Sequence Synthetic primer”
aattcctgctccaaaagaaagtcttcgaagccgctccactcgcatgtccactgtctcagagcttcgcatcacg

SEQ ID NO: 1310 - DNA, 66 bp, Artificial Sequence
  source: /note=“Description of Artificial Sequence Synthetic primer”
ccacagggttgaagaaccagaagccagttcctgctgttcctgtccagaagtctggcacatcaggtg

SEQ ID NO: 1311 - DNA, 78 bp, Artificial Sequence
  source: /note=“Description of Artificial Sequence Synthetic primer”
gcccagaggctccatcgtccatcctcttcctccccagtcggctgaactctccccttgtctgcactgttcaaacctctg

SEQ ID NO: 1312 - DNA, 70 bp, Artificial Sequence
  source: /note=“Description of Artificial Sequence Synthetic primer”
caaacagagggtggcagaagtgctcaatgaccctgagaacatggagaagcgcaggagccgtgagagcctc

SEQ ID NO: 1313 - DNA, 72 bp, Artificial Sequence
  source: /note=“Description of Artificial Sequence Synthetic primer”
agccgaggctttgttgaagaagcttcatatccagtaccggcgatcgggcacagaagctgtgctcatagccca

SEQ ID NO: 1314 - DNA, 71 bp, Artificial Sequence
  source: /note=“Description of Artificial Sequence Synthetic primer”
atgtgccagtgagcttgagtccttggagaaacacaagcacctgctagaaagtactgttaaccaggggctca

SEQ ID NO: 1315 - DNA, 83 bp, Artificial Sequence
  source: /note=“Description of Artificial Sequence Synthetic primer”
ggcctgctgagatcaaagactacagtccctacttcaagaccattgaggacctgaggaacaagattctcacagccacagtggac

SEQ ID NO: 1316 - DNA, 73 bp, Artificial Sequence
  source: /note=“Description of Artificial Sequence Synthetic primer”
cgaggattggttcttcagcaagacagaggaactgaaccgcgaggtggccaccaacagtgagctggtgcagagt

SEQ ID NO: 1317 - DNA, 77 bp, Artificial Sequence
  source: /note=“Description of Artificial Sequence Synthetic primer”
tgagcggcagaatcaggagtaccagcggctcatggacatcaagtcgcggctggagcaggagattgccacctaccgca

SEQ ID NO: 1318 - DNA, 69 bp, Artificial Sequence
  source: /note=“Description of Artificial Sequence Synthetic primer”
tcagtggagaaggagttggaccagtcaacatctctgttgtcacaagcagtgtttcctctggatatggca

SEQ ID NO: 1319 - DNA, 66 bp, Artificial Sequence
  source: /note=“Description of Artificial Sequence Synthetic primer”
cttgctggccaatgcctacatctacgttgtccagctgccagccaagatcctgactgcggacaatca

SEQ ID NO: 1320 - DNA, 80 bp, Artificial Sequence
  source: /note=“Description of Artificial Sequence Synthetic primer”
actcaagcggaaattgaagcagataggtcttatcagcacagtctccgcctcctggattcagtgtctcggcttcagggagt

SEQ ID NO: 1321 - DNA, 67 bp, Artificial Sequence
  source: /note=“Description of Artificial Sequence Synthetic primer”
agcgatgaagatggtcgcgccctggacgcggttctactccaacagctgctgcttgtgctgccatgtc

SEQ ID NO: 1322 - DNA, 67 bp, Artificial Sequence
  source: /note=“Description of Artificial Sequence Synthetic primer”
gcttcaggtgttgtgactgcagtgcctccctgtcgcaccagtactatgagaaggatgggcagctctt

SEQ ID NO: 1323 - DNA, 66 bp, Artificial Sequence
  source: /note=“Description of Artificial Sequence Synthetic primer”
ctttgggccaggaggaatctgttactcgaatccacccaggaactccctggcagtggattgtgggag

SEQ ID NO: 1324 - DNA, 67 bp, Artificial Sequence
  source: /note=“Description of Artificial Sequence Synthetic primer”
agaagctgtccctgcaagagcaggatgcagcgattgtgaagaacatgaagtctgagctggtacggct

SEQ ID NO: 1325 - DNA, 75 bp, Artificial Sequence
  source: /note=“Description of Artificial Sequence Synthetic primer”
ccgggagcagggaatcaccctgcgcgggagcgccgaaatcgtggccgagttcttctcattcggcatcaacagcat

SEQ ID NO: 1326 - DNA, 76 bp, Artificial Sequence
  source: /note=“Description of Artificial Sequence Synthetic primer”
ctgtcatgtggcagaccttccatccgaaccacggcttgggaagactacatttggttccaggcaccagtgacattta

SEQ ID NO: 1327 - DNA, 66 bp, Artificial Sequence
  source: /note=“Description of Artificial Sequence Synthetic primer”
gcccagtggagaaattcgtctttgagatcacccagcctccactgctgtccatcagctcagactcgc

SEQ ID NO: 1328 - DNA, 72 bp, Artificial Sequence
  source: /note=“Description of Artificial Sequence Synthetic primer”
cctcagaaattgccaggacttctttcccgtgatcttcagcaaagcctccgagtacttgcagctggtctttgg

SEQ ID NO: 1329 - DNA, 74 bp, Artificial Sequence
  source: /note=“Description of Artificial Sequence Synthetic primer”
aggactccagcaaccaagaagaggaggggccaagcaccttccctgacctggagtctgagttccaagcagcactc

SEQ ID NO: 1330 - DNA, 67 bp, Artificial Sequence
  source: /note=“Description of Artificial Sequence Synthetic primer”
cggaccaccaggtcagagccaattcgcagagcagggaagagtggtacctcaacacccactacccctg

SEQ ID NO: 1331 - DNA, 67 bp, Artificial Sequence
  source: /note=“Description of Artificial Sequence Synthetic primer”
gccctccaatgtccttatcaacaaggagggccatgtgaagatgtgtgactttggcatcagtggctac

SEQ ID NO: 1332 - DNA, 72 bp, Artificial Sequence
  source: /note=“Description of Artificial Sequence Synthetic primer”
gccggtcaggcacacaaggggcccttggagcgtggactggttggttttgccattttgttgtgtgtatgctgc

SEQ ID NO: 1333 - DNA, 77 bp, Artificial Sequence
  source: /note=“Description of Artificial Sequence Synthetic primer”
ccctcaaccggcaaatccgcgaggaggtggcgagtgcagtgagcagctcctacaggaatgaattcagggcatggacg

SEQ ID NO: 1334 - DNA, 76 bp, Artificial Sequence
  source: /note=“Description of Artificial Sequence Synthetic primer”
tgagtggaaaagcctgacctatgatgaagtcatcagctttgtgccaccaccccttgaccaagaagagatggagtcc

SEQ ID NO: 1335 - DNA, 83 bp, Artificial Sequence
  source: /note=“Description of Artificial Sequence Synthetic primer”
caacacccgtacatcaatgtctggtatgatccttctgaagcagaagctccaccaccaaagatccctgacaagcagttagatga

SEQ ID NO: 1336 - DNA, 69 bp, Artificial Sequence
  source: /note=“Description of Artificial Sequence Synthetic primer”
gaccttggaacctttggaacctgctgtcaacaggtcttacagggctgcttgaaccctcataggcctagg

SEQ ID NO: 1337 - DNA, 67 bp, Artificial Sequence
  source: /note=“Description of Artificial Sequence Synthetic primer”
cacaagctgaccttccgcgagaacgccaaagccaagacagaccacggggcggagatcgtgtacaagt

SEQ ID NO: 1338 - DNA, 77 bp, Artificial Sequence
  source: /note=“Description of Artificial Sequence Synthetic primer”
cagatggccactttgagaacattttagctgacaacagtgtgaacgaccagaccaaaatccttgtggttaatgctgcc

SEQ ID NO: 1339 - DNA, 75 bp, Artificial Sequence
  source: /note=“Description of Artificial Sequence Synthetic primer”
cttcggaaactggacatcaaaaacgaagacgatgtgaaatcgttgtctcgagtgatgatccatgttttcagcgac

SEQ ID NO: 1340 - DNA, 75 bp, Artificial Sequence
  source: /note=“Description of Artificial Sequence Synthetic primer”
gacttttgcccgctacctttcattccggcgtgacaacaatgagctgttgctcttcatactgaagcagttagtggc

SEQ ID NO: 1341 - DNA, 82 bp, Artificial Sequence
  source: /note=“Description of Artificial Sequence Synthetic primer”
tgatggtcctatgtgtcacattcatcacaggtttcataccaacacaggcttcagcacttcctttggtgtgtttcctgtccca

SEQ ID NO: 1342 - DNA, 71 bp, Artificial Sequence
  source: /note=“Description of Artificial Sequence Synthetic primer”
cgctcagccagatgcaatcaatgccccagtcacctgctgttataacttcaccaataggaagatctcagtgc

SEQ ID NO: 1343 - DNA, 69 bp, Artificial Sequence
  source: /note=“Description of Artificial Sequence Synthetic primer”
gtgaaatgaaacgcaccacactggacagccctttggggaagctggagctgtctggttgtgagcagggtc

SEQ ID NO: 1344 - DNA, 78 bp, Artificial Sequence
  source: /note=“Description of Artificial Sequence Synthetic primer”
ccaacgcttgccaaatcctgacaattcagaaccagctctctgtgaccccaatttgagttttgatgctgtcactaccgt

SEQ ID NO: 1345 - DNA, 86 bp, Artificial Sequence
  source: /note=“Description of Artificial Sequence Synthetic primer”
ccatgatggagaggcagacatcatgatcaactttggccgctgggagcatggcgatggatacccctttgacggtaaggacggactcc

SEQ ID NO: 1346 - DNA, 67 bp, Artificial Sequence
  source: /note=“Description of Artificial Sequence Synthetic primer”
gagaaccaatctcaccgacaggcagctggcagaggaatacctgtaccgctatggttacactcgggtg

SEQ ID NO: 1347 - DNA, 78 bp, Artificial Sequence
  source: /note=“Description of Artificial Sequence Synthetic primer”
gccatgctggctcagtctgagctgtgggccacatcagctagtggctcttctcatgcatcagttaggtgggtctgggtg

SEQ ID NO: 1348 - DNA, 79 bp, Artificial Sequence
  source: /note=“Description of Artificial Sequence Synthetic primer”
tcatggtgcccgtcaatgctgtgatggcgatgaagaccaagacgtatcaggtggcccacatgaagagcaaagacaatcg

SEQ ID NO: 1349 - DNA, 65 bp, Artificial Sequence
  source: /note=“Description of Artificial Sequence Synthetic primer”
aggggatgacttggacacatctgccattcgacatgactgcaattttgacaaagccatgcagtttt

SEQ ID NO: 1350 - DNA, 91 bp, Artificial Sequence
  source: /note=“Description of Artificial Sequence Synthetic primer”
tcatcctggcgatctacttcctctggcagaacctaggtccctctgtcctggctggagtcgctttcatggtcttgctgattccactcaacgg

SEQ ID NO: 1351 - DNA, 82 bp, Artificial Sequence
  source: /note=“Description of Artificial Sequence Synthetic primer”
tgattaccatcatggctcagattggctcctatgttcctgcagaagaagcgacaattgggattgtggatggcattttcacaag

SEQ ID NO: 1352 - DNA, 71 bp, Artificial Sequence
  source: /note=“Description of Artificial Sequence Synthetic primer”
ggccaggatctgtggtggtacaattgactctggccttccgagaaggtaccatcaatgtccacgacgtggag

SEQ ID NO: 1353 - DNA, 78 bp, Artificial Sequence
  source: /note=“Description of Artificial Sequence Synthetic primer”
gaaggaatgggaatcagtcatgagctaatcaccctggagatcagctcccgagatgtcccggatctgactctaatagac

SEQ ID NO: 1354 - DNA, 74 bp, Artificial Sequence
  source: /note=“Description of Artificial Sequence Synthetic primer”
gccgagatcgccaagatgttgccagggaggacagacaatgctgtgaagaatcactggaactctaccatcaaaag

SEQ ID NO: 1355 - DNA, 85 bp, Artificial Sequence
  source: /note=“Description of Artificial Sequence Synthetic primer”
cggtacttctcagggctaatatatacgtactctggcctcttctgcgtggtggtcaacccctataaacacctgcccatctactcgg

SEQ ID NO: 1356 - DNA, 79 bp, Artificial Sequence
  source: /note=“Description of Artificial Sequence Synthetic primer”
gtgaggcagcgcgactctggcgactggccggccatgccttcccgggctgaggactatgaagtgttgtacaccattggca

SEQ ID NO: 1357 - DNA, 73 bp, Artificial Sequence
  source: /note=“Description of Artificial Sequence Synthetic primer”
cagaccaaggagatggacctcagcgtggtgcggctcatgtttacagcttttcttccggatagcactggcagct

SEQ ID NO: 1358 - DNA, 68 bp, Artificial Sequence
  source: /note=“Description of Artificial Sequence Synthetic primer”
ctgccgggatggcttctatgaggctgagctctgcccggaccgctgcatccacagtttccagaacctgg

SEQ ID NO: 1359 - DNA, 67 bp, Artificial Sequence
  source: /note=“Description of Artificial Sequence Synthetic primer”
cactgacacccgcaacaccacccatggttcggactctgtggtttcagccagcagagagattgcagcc

SEQ ID NO: 1360 - DNA, 66 bp, Artificial Sequence
  source: /note=“Description of Artificial Sequence Synthetic primer”
ctgcttctttcccgagcttggaacttcgttatccgcgatgcgtttcctggcagctacattcctgct

SEQ ID NO: 1361 - DNA, 73 bp, Artificial Sequence
  source: /note=“Description of Artificial Sequence Synthetic primer”
ggctgtggctgaggctgtagcatctctgctggaggtgagacactctgggaactgatttgacctcgaatgctcc

SEQ ID NO: 1362 - DNA, 69 bp, Artificial Sequence
  source: /note=“Description of Artificial Sequence Synthetic primer”
cggacctgaatgtaatgcaagaatgaacagaacaagcaaaatgaccagctgcttagtcacctggcaaag

SEQ ID NO: 1363 - DNA, 67 bp, Artificial Sequence
  source: /note=“Description of Artificial Sequence Synthetic primer”
caaaggaagagcaacggaagaaacgcgagcaagaacgaaaggagaagaaagcaaaggttttgggaat

SEQ ID NO: 1364 - DNA, 65 bp, Artificial Sequence
  source: /note=“Description of Artificial Sequence Synthetic primer”
tggagactctcagggtcgaaaacggcggcagaccagcatgacagatttctaccactccaaacgcc

SEQ ID NO: 1365 - DNA, 66 bp, Artificial Sequence
  source: /note=“Description of Artificial Sequence Synthetic primer”
cggtggaccacgaagagttaacccgggacttggagaagcactgcagagacatggaagaggcgagcc

SEQ ID NO: 1366 - DNA, 67 bp, Artificial Sequence
  source: /note=“Description of Artificial Sequence Synthetic primer”
tcactaccagctgacatccggctgcctgagggctacctggagaagctgaccctcaatagccccatct

SEQ ID NO: 1367 - DNA, 66 bp, Artificial Sequence
  source: /note=“Description of Artificial Sequence Synthetic primer”
ccagctctccttccagctacagatcaatgtccctgtccgagtgctggagctaagtgagagccaccc

SEQ ID NO: 1368 - DNA, 66 bp, Artificial Sequence
  source: /note=“Description of Artificial Sequence Synthetic primer”
gagaagcacgccatgaaacaggccgtcatggaaatgatgagtcagaagattcagcagctcacagcc

SEQ ID NO: 1369 - DNA, 74 bp, Artificial Sequence
  source: /note=“Description of Artificial Sequence Synthetic primer”
agagccagttgctgtagaactcaaatctctgctgggcaaggatgttctgttcttgaaggactgtgtaggcccag

SEQ ID NO: 1370 - DNA, 71 bp, Artificial Sequence
  source: /note=“Description of Artificial Sequence Synthetic primer”
gacattgtggtaggggaagggactcattttctcatcccgtgggtacagaaaccaattatctttgactgccg

SEQ ID NO: 1371 - DNA, 83 bp, Artificial Sequence
  source: /note=“Description of Artificial Sequence Synthetic primer”
ataccaatcaccgcacaaacccaggctatttgttaagtccagtcacagcgcaaagaaacatatgcggagaaaatgctagtgtg

SEQ ID NO: 1372 - DNA, 67 bp, Artificial Sequence
  source: /note=“Description of Artificial Sequence Synthetic primer”
ctgctcaaggacaccgtctacacggacttcgatgggacccgagtgtatagccctccagagtggatcc

SEQ ID NO: 1373 - DNA, 67 bp, Artificial Sequence
  source: /note=“Description of Artificial Sequence Synthetic primer”
tggggacattccctttgagagggaccaggagattctggaagctgagctccacttcccagcccatgtc

SEQ ID NO: 1374 - DNA, 76 bp, Artificial Sequence
  source: /note=“Description of Artificial Sequence Synthetic primer”
cccatggatgctcctctgaagagactttcctcattgactgccgaggccccatgaatcaatgtctggtagccaccgg

SEQ ID NO: 1375 - DNA, 66 bp, Artificial Sequence
  source: /note=“Description of Artificial Sequence Synthetic primer”
ccaagttctgggtggtggaccagacccacttctacctgggcagtgccaacatggactggcgttcac

SEQ ID NO: 1376 - DNA, 69 bp, Artificial Sequence
  source: /note=“Description of Artificial Sequence Synthetic primer”
aatgaatacagtattcccaagcacatcaaccccgtggccgcctccctcatccagaagatgcttcagaca

SEQ ID NO: 1377 - DNA, 77 bp, Artificial Sequence
  source: /note=“Description of Artificial Sequence Synthetic primer”
cttacggttttcgtggagaagccttggggtcaatttgttgtatagctgaggttttaattacaacaagaacggctgct

SEQ ID NO: 1378 - DNA, 75 bp, Artificial Sequence
  source: /note=“Description of Artificial Sequence Synthetic primer”
gatgtggactgccattcaaaccaggaagataccggatgtaaatttcgagttttgcctcagccaactaatctcgca

SEQ ID NO: 1379 - DNA, 66 bp, Artificial Sequence
  source: /note=“Description of Artificial Sequence Synthetic primer”
ccacataccgtccagcctatctactggagaacgaagaagaggagcggaactcccgcacatgacctc

SEQ ID NO: 1380 - DNA, 78 bp, Artificial Sequence
  source: /note=“Description of Artificial Sequence Synthetic primer”
gcaatcatggaacttgacgatactctaaaatactctttcttgcagtttgacccagcacctcgtagaggcgagccacat

SEQ ID NO: 1381 - DNA, 85 bp, Artificial Sequence
  source: /note=“Description of Artificial Sequence Synthetic primer”
gcatcaggctgtcattatggtgtccttacctgtgggagctgtaaggtcttctttaagagggcaatggaagggcagcacaactact

SEQ ID NO: 1382 - DNA, 67 bp, Artificial Sequence
  source: /note=“Description of Artificial Sequence Synthetic primer”
aggactgggacccatgaacattcctttggtatcagacccgaagcgcaccattgctcaggattatggg

SEQ ID NO: 1383 - DNA, 69 bp, Artificial Sequence
  source: /note=“Description of Artificial Sequence Synthetic primer”
ggtgtccttcgccagatcactgttaatgatttgcctgtgggacgctccgtggatgaggctctgcggctg

SEQ ID NO: 1384 - DNA, 70 bp, Artificial Sequence
  source: /note=“Description of Artificial Sequence Synthetic primer”
caagcaatgcgtcatcaatgtccccagcctctgcggaatggatcacactgagaagagggggcggatttac

SEQ ID NO: 1385 - DNA, 68 bp, Artificial Sequence
  source: /note=“Description of Artificial Sequence Synthetic primer”
ctgacacttgccgcagagaatccctttctcacccacctcatctgcaccttccagaccaaggaccacct

SEQ ID NO: 1386 - DNA, 66 bp, Artificial Sequence
  source: /note=“Description of Artificial Sequence Synthetic primer”
gggttctagacgcccctcccaagcgttcctggccttctgaactccatacagcctctacagccgtcc

SEQ ID NO: 1387 - DNA, 68 bp, Artificial Sequence
  source: /note=“Description of Artificial Sequence Synthetic primer”
ctccacctatgagcgtctgtctctgtgggcttgggatgttaacaggagccaaaaggagggaaagtgtg

SEQ ID NO: 1388 - DNA, 86 bp, Artificial Sequence
  source: /note=“Description of Artificial Sequence Synthetic primer”
gccctcccagtgtgcaaataagggctgctgtttcgacgacaccgttcgtggggtcccctggtgcttctatcctaataccatcgacg

SEQ ID NO: 1389 - DNA, 81 bp, Artificial Sequence
  source: /note=“Description of Artificial Sequence Synthetic primer”
tggctaagtgaagatgacaatcatgttgcagcaattcactgtaaagctggaaagggacgaactggtgtaatgatatgtgca

SEQ ID NO: 1390 - DNA, 81 bp, Artificial Sequence
  source: /note=“Description of Artificial Sequence Synthetic primer”
cgcttgcctaactcatactttcccgttgacacttgatccacgcagcgtggcactgggacgtaagtggcgcagtctgaatgg

SEQ ID NO: 1391 - DNA, 74 bp, Artificial Sequence
  source: /note=“Description of Artificial Sequence Synthetic primer”
ggctactctgatctatgttgataaggaaaatggagaaccaggcacccgtgtggttgctaaggatgggctgaagc

SEQ ID NO: 1392 - DNA, 65 bp, Artificial Sequence
  source: /note=“Description of Artificial Sequence Synthetic primer”
gggacactgcgggacaagagcggttccggagtctcaccactgcatttttcagagacgccatgggc

SEQ ID NO: 1393 - DNA, 67 bp, Artificial Sequence
  source: /note=“Description of Artificial Sequence Synthetic primer”
ctgaaggaccctacgctcggtggcctggcacctcactttgagaagagtgagcacactggctttgcat

SEQ ID NO: 1394 - DNA, 78 bp, Artificial Sequence
  source: /note=“Description of Artificial Sequence Synthetic primer”
gcgacagctcctctagttccaccatgtccgcgggcggagacttcgggaatccgctgaggaaattcaagctggtgttcc

SEQ ID NO: 1395 - DNA, 71 bp, Artificial Sequence
  source: /note=“Description of Artificial Sequence Synthetic primer”
gaggagtggtgacagtctgcaaaatcaatacacaggaacctgaggagaccctggactttgatttctgcagc

SEQ ID NO: 1396 - DNA, 67 bp, Artificial Sequence
  source: /note=“Description of Artificial Sequence Synthetic primer”
agctagcctcagtgacacacatgacaggttgcactgccgacgttgtgtcaacagccgtcagatccgg

SEQ ID NO: 1397 - DNA, 73 bp, Artificial Sequence
  source: /note=“Description of Artificial Sequence Synthetic primer”
cgtcgtatgcgagagtctgtttccaggatgcctgttagttctcagcacagatattctacacctcacgccttca

SEQ ID NO: 1398 - DNA, 84 bp, Artificial Sequence
  source: /note=“Description of Artificial Sequence Synthetic primer”
ggtgtcagatataaatgtgcaaatgccttcttgctgtcctgtcggtctcagtacgttcactttatagctgctggcaatatcgaa

SEQ ID NO: 1399 - DNA, 67 bp, Artificial Sequence
  source: /note=“Description of Artificial Sequence Synthetic primer”
tgtggatgctggattgatttcaccactggtgcagctgctaaatagcaaagaccaggaagtgctgctt

SEQ ID NO: 1400 - DNA, 69 bp, Artificial Sequence
  source: /note=“Description of Artificial Sequence Synthetic primer”
agtgggagacacctgacctttctcaagctgagattgagcagaagatcaaggagtacaatgcccagatca

SEQ ID NO: 1401 - DNA, 77 bp, Artificial Sequence
  source: /note=“Description of Artificial Sequence Synthetic primer”
cgaagcccttacaagtttcctagttcacccttacggattcctggagggaacatctatatttcacccctgaagagtcc

SEQ ID NO: 1402 - DNA, 66 bp, Artificial Sequence
  source: /note=“Description of Artificial Sequence Synthetic primer”
cccagtgtacgaggaacaagacagaccgagatctccaaccggacctagcaactccttcctcgctaa

SEQ ID NO: 1403 - DNA, 66 bp, Artificial Sequence
  source: /note=“Description of Artificial Sequence Synthetic primer”
gggctgggtgagaatgtgatggagaggaagaagccggccctggtatccattccggaggatgttgtg

SEQ ID NO: 1404 - DNA, 68 bp, Artificial Sequence
  source: /note=“Description of Artificial Sequence Synthetic primer”
cctacaagtcctggggcattggagccccaagcagtgttaatcctggctactgtgtgagcctgacctca

SEQ ID NO: 1405 - DNA, 67 bp, Artificial Sequence
  source: /note=“Description of Artificial Sequence Synthetic primer”
gcgaggagctctacttgctctgcgacaaggtgcagaaagaggacatatcagtggtgttcagcagggc

SEQ ID NO: 1406 - DNA, 67 bp, Artificial Sequence
  source: /note=“Description of Artificial Sequence Synthetic primer”
aagcatgaacaggacttgaccatctttccaacccctggggaagacatttgcaactgacttggggagg

SEQ ID NO: 1407 - DNA, 68 bp, Artificial Sequence
  source: /note=“Description of Artificial Sequence Synthetic primer”
cccgttcggtctgaggaaggccgggacatggcgaaccggatcagtgcctttggctaccttgagtgctc

SEQ ID NO: 1408 - DNA, 74 bp, Artificial Sequence
  source: /note=“Description of Artificial Sequence Synthetic primer”
ccagacgagcgattagaagcggcagcttgtgaggtgaatgatttgggggaagaggaggaggaggaagaggagga

SEQ ID NO: 1409 - DNA, 73 bp, Artificial Sequence
  source: /note=“Description of Artificial Sequence Synthetic primer”
tgtgcacataggaatgagcttcagatgcagttggccagcaaagagagtgatattgagcaattgcgtgctaaac

SEQ ID NO: 1410 - DNA, 74 bp, Artificial Sequence
  source: /note=“Description of Artificial Sequence Synthetic primer”
gatctggcactgtggttcctgcatgaagacagtggctggcggtgcctggacgtacaataccacttccgctgtca

SEQ ID NO: 1411 - DNA, 75 bp, Artificial Sequence
  source: /note=“Description of Artificial Sequence Synthetic primer”
ccattctatcatcaacgggtacaaacgagtcctggccttgtctgtggagacggattacaccttcccacttgctga

SEQ ID NO: 1412 - DNA, 67 bp, Artificial Sequence
  source: /note=“Description of Artificial Sequence Synthetic primer”
ctgtcttcctgttggccctgacaatcatagccagcacctgggctctgacgcccactcactacctcac

SEQ ID NO: 1413 - DNA, 81 bp, Artificial Sequence
  source: /note=“Description of Artificial Sequence Synthetic primer”
gctcattatgaaaaacatcccaaactttaaaatgcgaaattattggttggtgtgaagaaagccagacaacttctgtttctt

SEQ ID NO: 1414 - DNA, 66 bp, Artificial Sequence
  source: /note=“Description of Artificial Sequence Synthetic primer”
gctctgttgtgtcctgttgccggctctggccttcctgtgactgactgtgaagtggcttctccgtac

SEQ ID NO: 1415 - DNA, 71 bp, Artificial Sequence
  source: /note=“Description of Artificial Sequence Synthetic primer”
cgaggagatgcctgtggacaggatcctggaggcagagcttgctgtggaacagaagagtgaccagggcgttg

SEQ ID NO: 1416 - DNA, 77 bp, Artificial Sequence
  source: /note=“Description of Artificial Sequence Synthetic primer”
acaccaaaatgccatctcaaatggaacacgccatggaaaccatgatgtttacatttcacaaattcgctggggataaa

SEQ ID NO: 1417 - DNA, 67 bp, Artificial Sequence
  source: /note=“Description of Artificial Sequence Synthetic primer”
cttctgagcccgtctcccggacaggttgaggaagctgctccagaagcgcctcggaaggggagctctc

SEQ ID NO: 1418 - DNA, 86 bp, Artificial Sequence
  source: /note=“Description of Artificial Sequence Synthetic primer”
cgcgagcccctcattatacactgggcagcctccccacagcgcatcgaggaatgcgtgctctcaggcaaggatgtcaacggcgagtg

SEQ ID NO: 1419 - DNA, 70 bp, Artificial Sequence
  source: /note=“Description of Artificial Sequence Synthetic primer”
gagagagccagtctgatccagaaggccaagctggcagagcaggccgaacgctatgaggacatggcagcct

SEQ ID NO: 1420 - DNA, 77 bp, Artificial Sequence
  source: /note=“Description of Artificial Sequence Synthetic primer”
cagtggagaccagttgggtagtggtgactgggtacgctacaagctctgcatgtgtgctgatgggacgctcttcaagg

SEQ ID NO: 1421 - DNA, 73 bp, Artificial Sequence
  source: /note=“Description of Artificial Sequence Synthetic primer”
tccgcaagacacctcctggagggcctcctgcagaaggacaggacaaagcggctcggggccaaggatgacttca

SEQ ID NO: 1422 - DNA, 71 bp, Artificial Sequence
  source: /note=“Description of Artificial Sequence Synthetic primer”
tgcattcgttggtttctcttatgcacctccttcagaagacttatttttgtgagcagtttgccattcagaaa

SEQ ID NO: 1423 - DNA, 71 bp, Artificial Sequence
  source: /note=“Description of Artificial Sequence Synthetic primer”
ccaacaccttcttggcttctgggacctgtgttcttgctgagcaccctctccggtttgggttgggataacag

SEQ ID NO: 1424 - DNA, 72 bp, Artificial Sequence
  source: /note=“Description of Artificial Sequence Synthetic primer”
agctggggtgtctgtttcatgtggaatacctgacttcaggtcaagggatggtatttatgctcgccttgctgt

SEQ ID NO: 1425 - DNA, 67 bp, Artificial Sequence
  source: /note=“Description of Artificial Sequence Synthetic primer”
gtggggagcccatcatctcgccaagccatcacaggctctgcatacacatgcactcagtgtggactgg

SEQ ID NO: 1426 - DNA, 64 bp, Artificial Sequence
  source: /note=“Description of Artificial Sequence Synthetic primer”
tctgccagtgctgaattctttgctgacattgccctggctcctatggaagctgctaaggttcgaa

SEQ ID NO: 1427 - DNA, 67 bp, Artificial Sequence
  source: /note=“Description of Artificial Sequence Synthetic primer”
cccaactcaggtccttggtaaatcctgcaagccaatcccagtcatgctccttggggtgaccctcttg

SEQ ID NO: 1428 - DNA, 70 bp, Artificial Sequence
  source: /note=“Description of Artificial Sequence Synthetic primer”
agatgcatacttggaagcacagtcatatcacactgggaggcaatgcaatgtggttacctggtcctaggtt

SEQ ID NO: 1429 - DNA, 70 bp, Artificial Sequence
  source: /note=“Description of Artificial Sequence Synthetic primer”
gcgcagaggccagttaaagtagatcacctcctcgaacccactccggttccccgcaacccacagctcagct

SEQ ID NO: 1430 - DNA, 76 bp, Artificial Sequence
  source: /note=“Description of Artificial Sequence Synthetic primer”
ggctggccaaacataagcagctgcactgcgatgcccagtctagaaaatctttcagctgtaaatactgtgacaagga

SEQ ID NO: 1431 - DNA, 66 bp, Artificial Sequence
  source: /note=“Description of Artificial Sequence Synthetic primer”
agtgacaaatgttggaggagcagtggtgacgggtgtgacagcagtagcccagaagacagtggaggg

SEQ ID NO: 1432 - DNA, 80 bp, Artificial Sequence
  source: /note=“Description of Artificial Sequence Synthetic primer”
acccaccatggatgtcttcaagaagggcttctccatcgccaaggagggcgtggtgggtgcggtggaaaagaccaagcagg

SEQ ID NO: 1433 - DNA, 70 bp, Artificial Sequence
  source: /note=“Description of Artificial Sequence Synthetic primer”
tgaagagaggcatgttggagacttgggcaatgtgactgctgacaaagatggtgtggccgatgtgtctatt

SEQ ID NO: 1434 - DNA, 74 bp, Artificial Sequence
  source: /note=“Description of Artificial Sequence Synthetic primer”
tgaggagtggtattttggcaagatcaccagacgggagtcagagcggttactgctcaatgcagagaacccgagag

SEQ ID NO: 1435 - DNA, 74 bp, Artificial Sequence
  source: /note=“Description of Artificial Sequence Synthetic primer”
atacagcaccaatggaaagatcaccttcgacgactacatcgcctgctgcgtcaaactgagggctcttacagaca

SEQ ID NO: 1436 - DNA, 81 bp, Artificial Sequence
  source: /note=“Description of Artificial Sequence Synthetic primer”
gggctcagctttcagaagtgctgagttggcagttttcttctgtcaccaaaagaggtctcaatgtggaccagctgaacatgt

SEQ ID NO: 1437 - DNA, 70 bp, Artificial Sequence
  source: /note=“Description of Artificial Sequence Synthetic primer”
tcacatgccactttggtgtttcataatctcctgggagagattgaccagcagtatagccgcttcctgcaag

SEQ ID NO: 1438 - DNA, 76 bp, Artificial Sequence
  source: /note=“Description of Artificial Sequence Synthetic primer”
caagagggactcggactgcagcagcctctgcacctctgagagcatggactatggtaccaatctctccactgacctg

SEQ ID NO: 1439 - DNA, 66 bp, Artificial Sequence
  source: /note=“Description of Artificial Sequence Synthetic primer”
ggactcggagacgctgtgcaggagggccgtcaagatcctcaagaagaagaagttgcgaaggatccc

SEQ ID NO: 1440 - DNA, 69 bp, Artificial Sequence
  source: /note=“Description of Artificial Sequence Synthetic primer”
catcttccaggaggaccactctctgtggcaccctggactacctgccccctgaaatgattgaaggtcgga

SEQ ID NO: 1441 - DNA, 71 bp, Artificial Sequence
  source: /note=“Description of Artificial Sequence Synthetic primer”
aatacccaacgcacaaatgaccgcacgttctctgccccgtttcttgccccagtgtggtttgcattgtctcc

SEQ ID NO: 1442 - DNA, 90 bp, Artificial Sequence
  source: /note=“Description of Artificial Sequence Synthetic primer”
cctggaggctgcaacatacctcaatcctgtcccaggccggatcctcctgaagcccttttcgcagcactgctatcctccaaagccattgta

SEQ ID NO: 1443 - DNA, 80 bp, Artificial Sequence
  source: /note=“Description of Artificial Sequence Synthetic primer”
tgttttgattcccgggcttaccaggtgagaagtgagggaggaagaaggcagtgtcccttttgctagagctgacagctttg

SEQ ID NO: 1444 - DNA, 65 bp, Artificial Sequence
  source: /note=“Description of Artificial Sequence Synthetic primer”
cacccttggactggaaaactcacacccggtctggacacagaaagagaaccaacagctcatcaagg

SEQ ID NO: 1445 - DNA, 66 bp, Artificial Sequence
  source: /note=“Description of Artificial Sequence Synthetic primer”
gatcctcgcgtgagacagatcaagatcaagaccggcgtggtgaagcggttggtcaaagaaaaagtg

SEQ ID NO: 1446 - DNA, 68 bp, Artificial Sequence
  source: /note=“Description of Artificial Sequence Synthetic primer”
ctgttttcctggaggactgcagtgactgcgtgctggcagtggcctgccaacagctccgcatacacagt

SEQ ID NO: 1447 - DNA, 66 bp, Artificial Sequence
  source: /note=“Description of Artificial Sequence Synthetic primer”
cagccaggtgtacgagacattgctcacctacagtgacgtcgtgggcgcggatgtgctggacgaggt

SEQ ID NO: 1448 - DNA, 66 bp, Artificial Sequence
  source: /note=“Description of Artificial Sequence Synthetic primer”
tcccgagagaggaaagcatgatgggagccacgaagggactgtgtattttaaatgcaggcacccgac

SEQ ID NO: 1449 - DNA, 74 bp, Artificial Sequence
  source: /note=“Description of Artificial Sequence Synthetic primer”
cctggttgaagcctgttaatgctttcaacgtgtggaaaacccagcgggcctttagcaaatatgagaagtctgca

SEQ ID NO: 1450 - DNA, 68 bp, Artificial Sequence
  source: /note=“Description of Artificial Sequence Synthetic primer”
ccagtgtgtgtaacagggtcacaagaattcgacagccagatgctccaagagggtggcccaaggctata

SEQ ID NO: 1451 - DNA, 68 bp, Artificial Sequence
  source: /note=“Description of Artificial Sequence Synthetic primer”
gccaactgctttcatttgtgagggatctgaaccaatacagagcagacataaaggaaatgggcctgagt

SEQ ID NO: 1452 - DNA, 85 bp, Artificial Sequence
  source: /note=“Description of Artificial Sequence Synthetic primer”
catccgcaaagtgactgaagagaacaaagagttggccaatgagctgaggcggcctcccctatgctatcacaacggagttcagtac

SEQ ID NO: 1453 - DNA, 84 bp, Artificial Sequence
  source: /note=“Description of Artificial Sequence Synthetic primer”
gccgggaagaccgtaattgtggctgcactggatgggaccttccagaggaagccatttggggccatcctgaacctggtgccgctg

SEQ ID NO: 1454 - DNA, 72 bp, Artificial Sequence
  source: /note=“Description of Artificial Sequence Synthetic primer”
aatccaagggggagagtgatgacttccatatggactttgactcagctgtggctcctcgggcaaaatctgtac

SEQ ID NO: 1455 - DNA, 66 bp, Artificial Sequence
  source: /note=“Description of Artificial Sequence Synthetic primer”
gtgatgccttccctgtgggcgaggtgaagatgctggagaagcagacgaacccacccgactacctga

SEQ ID NO: 1456 - DNA, 82 bp, Artificial Sequence
  source: /note=“Description of Artificial Sequence Synthetic primer”
ctatatgcagccagagatgtgacagccaccgtggacagcctgccactcatcacagcctccattctcagtaagaaactcgtgg

SEQ ID NO: 1457 - DNA, 74 bp, Artificial Sequence
  source: /note=“Description of Artificial Sequence Synthetic primer”
tgctgttgctgagtctgttgccagtccccagaagaccatgtctgtgttgagctgtatctgtgaagccaggcaag

SEQ ID NO: 1458 - DNA, 81 bp, Artificial Sequence
  source: /note=“Description of Artificial Sequence Synthetic primer”
ggtgtcgatattgtcatgaaccatcacctgcaggaaacaagtttcacaaaagaagcctacaagaagtacatcaaagattac

SEQ ID NO: 1459 - DNA, 66 bp, Artificial Sequence
  source: /note=“Description of Artificial Sequence Synthetic primer”
gacgctggtctggtgaagatgtccaggaaaccacgagcctccagcccattgtccaacaaccaccca

SEQ ID NO: 1460 - DNA, 73 bp, Artificial Sequence
  source: /note=“Description of Artificial Sequence Synthetic primer”
cttcacagtgctcctgcagtctctctgtgtggctgtaacttacgtgtactttaccaacgagctgaagcagatg

SEQ ID NO: 1461 - DNA, 65 bp, Artificial Sequence
  source: /note=“Description of Artificial Sequence Synthetic primer”
gcctcggtgtgcctttcaacatcgccagctacgccctgctcacgtacatgattgcgcacatcacg

SEQ ID NO: 1462 - DNA, 66 bp, Artificial Sequence
  source: /note=“Description of Artificial Sequence Synthetic primer”
ctggtcagccttcagggaccctgagcaccgcctggtctctttcctgtggccagcccagaactgaag

SEQ ID NO: 1463 - DNA, 76 bp, Artificial Sequence
  source: /note=“Description of Artificial Sequence Synthetic primer”
tgcttgtcagttgtcaacaccttatggccaacctgcctgtttccagcagcaacagcatcaaatacttgccactcca

SEQ ID NO: 1464 - DNA, 68 bp, Artificial Sequence
  source: /note=“Description of Artificial Sequence Synthetic primer”
tgtcaccccgactcaacgtgagacgcaccgcccggactcaccatgcgtgaatgcatctcagtccacgt

SEQ ID NO: 1465 - DNA, 62 bp, Artificial Sequence
  source: /note=“Description of Artificial Sequence Synthetic primer”
agctcaacatgcgtgagtgtatctctatccacgtggggcaggcaggagtccagatcggcaat

SEQ ID NO: 1466 - DNA, 71 bp, Artificial Sequence
  source: /note=“Description of Artificial Sequence Synthetic primer”
ctcttacatcgaccgcctaagagtcgcgctgtaagaagcaacaacctctcctcttcgtctccgccatcagc

SEQ ID NO: 1467 - DNA, 71 bp, Artificial Sequence
  source: /note=“Description of Artificial Sequence Synthetic primer”
gaggagggtgagttctccaaggcccatgaggatatgactgccctggagaaggattacaaggaggtgggcat

SEQ ID NO: 1468 - DNA, 71 bp, Artificial Sequence
  source: /note=“Description of Artificial Sequence Synthetic primer”
gtcccttcgcctccttcaccgccgcagaccccttcaagttctagtcatgcgtgagtgcatctccatccacg

SEQ ID NO: 1469 - DNA, 68 bp, Artificial Sequence
  source: /note=“Description of Artificial Sequence Synthetic primer”
cgccctacctataccaacctcaaccgcctcatcagtcagattgtgtcctcaatcactgcttctctccg

SEQ ID NO: 1470 - DNA, 73 bp, Artificial Sequence
  source: /note=“Description of Artificial Sequence Synthetic primer”
cgaggacgaggcttaaaaacttctcagatcaatcgtgcatccttagtgaacttctgttgtcctcaagcatggt

SEQ ID NO: 1471 - DNA, 70 bp, Artificial Sequence
  source: /note=“Description of Artificial Sequence Synthetic primer”
cgccctcctgcagtatttatggcctcgtcctcccccacctaggccacgtgtgagctgctcctgtctctgt

SEQ ID NO: 1472 - DNA, 84 bp, Artificial Sequence
  source: /note=“Description of Artificial Sequence Synthetic primer”
acactgactggcatcctgctttccagtgcctgccagcctccagaagagccaggtgcctgactagtacatggggagctacagagc

SEQ ID NO: 1473 - DNA, 70 bp, Artificial Sequence
  source: /note=“Description of Artificial Sequence Synthetic primer”
gtggcctagagccttcagtcactggggaaagcagggaagcagtgtgaactctttattcactcccagcctg

SEQ ID NO: 1474 - DNA, 73 bp, Artificial Sequence
  source: /note=“Description of Artificial Sequence Synthetic primer”
acaggccccatgcatcctccctgcctcactcccctcagcccctgccgaccttagcttatctgggagagaaaca

SEQ ID NO: 1475 - DNA, 80 bp, Artificial Sequence
  source: /note=“Description of Artificial Sequence Synthetic primer”
ccctatggccctgaatggtgcactggtttaattgtgttggtgtcggcccctcacaaatgcagccaagtcatgtaattagt

SEQ ID NO: 1476 - DNA, 75 bp, Artificial Sequence
  source: /note=“Description of Artificial Sequence Synthetic primer”
agtggaatccttccctttccaactctacctccctcactcagctcctttcccctgatcagagaaagggatcaaggg

SEQ ID NO: 1477 - DNA, 75 bp, Artificial Sequence
  source: /note=“Description of Artificial Sequence Synthetic primer”
ggaaggaaagaagcatggtctactttaggtgtgcgctgggtctctggtgctcttcactgttgcctgtcacttttt

SEQ ID NO: 1478 - DNA, 67 bp, Artificial Sequence
  source: /note=“Description of Artificial Sequence Synthetic primer”
gatgccgagggaaatcatcaccctacagttgggccagtgcggcaatcagattgggttcgagttctgg

SEQ ID NO: 1479 - DNA, 64 bp, Artificial Sequence
  source: /note=“Description of Artificial Sequence Synthetic primer”
gcgctgcggaagatcatccccacgctgccctcggacaagctgagcaagattcagaccctcaagc

SEQ ID NO: 1480 - DNA, 67 bp, Artificial Sequence
  source: /note=“Description of Artificial Sequence Synthetic primer”
cagtgtggaggggatggaggagcctgacatccagtgggtgaaggatggggctgtggtccagaacttg

SEQ ID NO: 1481 - DNA, 69 bp, Artificial Sequence
  source: /note=“Description of Artificial Sequence Synthetic primer”
agttgtcgtgtgttctggattcattccggcaccaccatgtcgaaggtttcctttaagatcacgctgacg

SEQ ID NO: 1482 - DNA, 70 bp, Artificial Sequence
  source: /note=“Description of Artificial Sequence Synthetic primer”
gtggatgtgccctgaaggacaagccaggcgtctacacgagagtctcacacttcttaccctggatccgcag

SEQ ID NO: 1483 - DNA, 89 bp, Artificial Sequence
  source: /note=“Description of Artificial Sequence Synthetic primer”
tggcttcaggagctgaataccctcccaggcacacacaggtgggacacaaataagggttttggaaccactattttctcatcacgacagca

SEQ ID NO: 1484 - DNA, 71 bp, Artificial Sequence
  source: /note=“Description of Artificial Sequence Synthetic primer”
ctgctgtcttgggtgcattggagccttgccttgctgctctacctccaccatgccaagtggtcccaggctgc

SEQ ID NO: 1485 - DNA, 71 bp, Artificial Sequence
  source: /note=“Description of Artificial Sequence Synthetic primer”
tgacgatggcctggagtgtgtgcccactgggcagcaccaagtccggatgcagatcctcatgatccggtacc

SEQ ID NO: 1486 - DNA, 83 bp, Artificial Sequence
  source: /note=“Description of Artificial Sequence Synthetic primer”
cctcagcaagacgttatttgaaattacagtgcctctctctcaaggccccaaaccagtaacaatcagttttgccaatcacactt

SEQ ID NO: 1487 - DNA, 67 bp, Artificial Sequence
  source: /note=“Description of Artificial Sequence Synthetic primer”
cggttggtgacttgtctgcctcctgctttgggaagactgaggcatccgtgaggcagggacaagtctt

SEQ ID NO: 1488 - DNA, 72 bp, Artificial Sequence
  source: /note=“Description of Artificial Sequence Synthetic primer”
tgcccttaaaggaaccaatgagtccctggaacgccagatgcgtgaaatggaagagaactttgccgttgaagc

SEQ ID NO: 1489 - DNA, 84 bp, Artificial Sequence
  source: /note=“Description of Artificial Sequence Synthetic primer”
ggttgtgctctacgagcttatgactggctcactgccttacagccacattggctgccgtgaccagattatctttatggtgggccg

SEQ ID NO: 1490 - DNA, 68 bp, Artificial Sequence
  source: /note=“Description of Artificial Sequence Synthetic primer”
ctctccagtgtgggcaccagccggccagaacagatgcgagcagtccatgactctgggagctacaccgc

SEQ ID NO: 1491 - DNA, 75 bp, Artificial Sequence
  source: /note=“Description of Artificial Sequence Synthetic primer”
gtatcaggaccacatgcagtacatcggagaaggcgcgaagacaggcatcaaagaatgccagtatcaattccgaca

SEQ ID NO: 1492 - DNA, 77 bp, Artificial Sequence
  source: /note=“Description of Artificial Sequence Synthetic primer”
gcagttggaagacacaggaaagtatccccaaattgcagatttatcaacggcttttatcttgaaaatagtgccacgca

SEQ ID NO: 1493 - DNA, 75 bp, Artificial Sequence
  source: /note=“Description of Artificial Sequence Synthetic primer”
caggtcaggcagaggaagtcatgtgcattgcatgagctaaacctatctgaatgaattgatttggggcttgttagg

SEQ ID NO: 1494 - DNA, 66 bp, Artificial Sequence
  source: /note=“Description of Artificial Sequence Synthetic primer”
tggtcagatgctgctgaagtatatccttaggccgctggcatcttgcccatcccttcatgctgtgat

SEQ ID NO: 1495 - DNA, 66 bp, Artificial Sequence
  source: /note=“Description of Artificial Sequence Synthetic primer”
gagggagcagacagtgggtaccacgatctccgtaaccatttgcatgtgacttagcaagggctctga

SEQ ID NO: 1496 - DNA, 66 bp, Artificial Sequence
  source: /note=“Description of Artificial Sequence Synthetic primer”
tagaggccatcaaaattggcctcaccaaggccctgactcagatggaggaagcccagaggaaacgga